SEQUENCE LISTING (166 sequences)

SEQ ID NO: 1 - DNA, 22 bp, Artificial Sequence
  Primer for human BRCA1 sequence
gagtagaggttagagggtaggt

SEQ ID NO: 2 - DNA, 25 bp, Artificial Sequence
  Primer for human BRCA1 sequence
caaaacatattccaattcctatcac

SEQ ID NO: 3 - DNA, 23 bp, Artificial Sequence
  Primer for human BRCA1 sequence
tcaatacccccaacctaatcctc

SEQ ID NO: 4 - DNA, 23 bp, Artificial Sequence
  Primer for human BRCA2 sequence
gggggatttggagtaggtatagg

SEQ ID NO: 5 - DNA, 25 bp, Artificial Sequence
  Primer for human BRCA2 sequence
cacttccccaaaacaacaatattcc

SEQ ID NO: 6 - DNA, 24 bp, Artificial Sequence
  Primer for human BRCA2 sequence
aacccactaccaccaccactaacc

SEQ ID NO: 7 - DNA, 22 bp, Artificial Sequence
  Primer for human PSEN1 sequence
gatttattgagtggtgggagag

SEQ ID NO: 8 - DNA, 24 bp, Artificial Sequence
  Primer for human PSEN1 sequence
tgataggtgttaaatttaggatgg

SEQ ID NO: 9 - DNA, 21 bp, Artificial Sequence
  Primer for human PSEN1 sequence
cccctcatcttttaaaacacc

SEQ ID NO: 10 - DNA, 21 bp, Artificial Sequence
  Primer for human PSEN2 sequence
ggggtggagagaggagagtgt

SEQ ID NO: 11 - DNA, 25 bp, Artificial Sequence
  Primer for human PSEN2 sequence
aaatacaattacttcactcaacacc

SEQ ID NO: 12 - DNA, 26 bp, Artificial Sequence
  Primer for human PSEN2 sequence
aactctataacctcaatttcttcatc

SEQ ID NO: 13 - DNA, 26 bp, Artificial Sequence
  Primer for human HD sequence
ggttttttggttagttattggtagag

SEQ ID NO: 14 - DNA, 27 bp, Artificial Sequence
  Primer for human HD sequence
gtaggttagggttgttaattatgttgg

SEQ ID NO: 15 - DNA, 27 bp, Artificial Sequence
  Primer for human HD sequence
caatacaacaactcctcaaccacaacc

SEQ ID NO: 16 - DNA, 25 bp, Artificial Sequence
  Primer for human DM1 sequence
gtggatgggtaaattgtaggtttgg

SEQ ID NO: 17 - DNA, 26 bp, Artificial Sequence
  Primer for human DM1 sequence
aacattcccaactacaaaaacccttc

SEQ ID NO: 18 - DNA, 24 bp, Artificial Sequence
  Primer for human DM1 sequence
cttttcctcccccaaccctaattc

SEQ ID NO: 19 - DNA, 30 bp, Artificial Sequence
  Primer for human DM1 sequence
ataaaatttaagttaggatgggagatatag

SEQ ID NO: 20 - DNA, 29 bp, Artificial Sequence
  Primer for human DM1 sequence
ataaataaaccaaaacaatactttaccta

SEQ ID NO: 21 - DNA, 30 bp, Artificial Sequence
  Primer for human DM1 sequence
tttggtatttagtagttgtttaataaagtt

SEQ ID NO: 22 - DNA, 30 bp, Artificial Sequence
  Primer for human DM1 sequence
tacaaaatatcatactctaatcactaaacc

SEQ ID NO: 23 - DNA, 21 bp, Artificial Sequence
  Human DNA adaptors
cgtggagactgactaccagat

SEQ ID NO: 24 - DNA, 25 bp, Artificial Sequence
  Human DNA adaptors
agttacatctggtagtcagtctcca

SEQ ID NO: 25 - DNA, 21 bp, Homo sapiens
aagaggagggttaaattgtta

SEQ ID NO: 26 - DNA, 18 bp, Artificial Sequence
  Primer based on human sequence
tcaaactaaaaccaaaac

SEQ ID NO: 27 - DNA, 23 bp, Artificial Sequence
  Primer based on human sequence
attaacttaaaaaaaccctaacc

SEQ ID NO: 28 - DNA, 25 bp, Artificial Sequence
  Primer based on human sequence
tgtttttatgaaagaagtaaatatt

SEQ ID NO: 29 - DNA, 16 bp, Artificial Sequence
  Primer based on human sequence
aatcctcatccaacaa

SEQ ID NO: 30 - DNA, 24 bp, Artifical Sequence
  Primer based on human sequence
tccttattattttcttcattaaac

SEQ ID NO: 31 - DNA, 22 bp, Artificial Sequence
  Primer based on human sequence
gatgttttattgagtttaggtt

SEQ ID NO: 32 - DNA, 17 bp, Artificial Sequence
  Primer based on human sequence
ttgggagtagaaggttt

SEQ ID NO: 33 - DNA, 23 bp, Artificial Sequence
  Primer based on human sequence
aactaattaataactctatccaa

SEQ ID NO: 34 - DNA, 21 bp, Artificial Sequence
  Primer based on human sequence
gggttagatattatttagttt

SEQ ID NO: 35 - DNA, 18 bp, Artificial Sequence
  Primer based on human sequence
aaaaataaaaacaaaccc

SEQ ID NO: 36 - DNA, 22 bp, Artificial Sequence
  Primer based on human sequence
actaaaactaaaactaaaacac

SEQ ID NO: 37 - DNA, 20 bp, Artificial Sequence
  Primer based on human sequence
tagggttttttgggagagtt

SEQ ID NO: 38 - DNA, 19 bp, Artificial Sequence
  Primer based on human sequence
ttattttagttttggtttt

SEQ ID NO: 39 - DNA, 24 bp, Artificial Sequence
  Primer based on human sequence
attacccacaaaaacctatataaa

SEQ ID NO: 40 - DNA, 20 bp, Artificial Sequence
  Primer based on human sequence
gtgtttggggatttaagttt

SEQ ID NO: 41 - DNA, 18 bp, Artificial Sequence
  Primer based on human sequence
tcaaacatacacacctta

SEQ ID NO: 42 - DNA, 20 bp, Artificial Sequence
  Primer based on human sequence
accctttttccaaatctaac

SEQ ID NO: 43 - DNA, 25 bp, Artificial Sequence
  Primer based on human sequence
tttgagtaagattagattaagaggt

SEQ ID NO: 44 - DNA, 16 bp, Artificial Sequence
  Primer based on human sequence
gggatattttggttat

SEQ ID NO: 45 - DNA, 23 bp, Artificial Sequence
  Primer based on human sequence
acaaccctaactaccccaaaaac

SEQ ID NO: 46 - DNA, 21 bp, Artificial Sequence
  Primer based on human sequence
ggtagagtttgagtttaggtt

SEQ ID NO: 47 - DNA, 16 bp, Artificial Sequence
  Primer based on human sequence
tagatattaggtggat

SEQ ID NO: 48 - DNA, 20 bp, Artificial Sequence
  Primer based on human sequence
accaaaccaaaccctaaaac

SEQ ID NO: 49 - DNA, 23 bp, Artificial Sequence
  Primer based on human sequence
ttgggaagtgtggtttgtaggaa

SEQ ID NO: 50 - DNA, 23 bp, Artificial Sequence
  Primer based on human sequence
ttgggaagtgtggtttgtaggaa

SEQ ID NO: 51 - DNA, 19 bp, Artificial Sequence
  Primer based on human sequence
cacctttaaacctcctatt

SEQ ID NO: 52 - DNA, 19 bp, Artificial Sequence
  Primer based on human sequence
cacctttaaacctcctatt

SEQ ID NO: 53 - DNA, 24 bp, Artificial Sequence
  Primer based on human sequence
acctccaaatataaccaccatctc

SEQ ID NO: 54 - DNA, 24 bp, Artificial Sequence
  Primer based on human sequence
acctccaaatataaccaccatctc

SEQ ID NO: 55 - DNA, 23 bp, Artificial Sequence
  Primer based on human sequence
ttttttttgtttaggagtttttt

SEQ ID NO: 56 - DNA, 19 bp, Artificial Sequence
  Primer based on human sequence
ggtaaaggtagagaaagga

SEQ ID NO: 57 - DNA, 23 bp, Artificial Sequence
  Primer based on human sequence
ctaaaactaaaccaaccaccctc

SEQ ID NO: 58 - DNA, 19 bp, Artificial Sequence
  Primer based on human sequence
gaagggtttttagtattgt

SEQ ID NO: 59 - DNA, 17 bp, Artificial Sequence
  Primer based on human sequence
aaaaaaactaaaattac

SEQ ID NO: 60 - DNA, 19 bp, Artificial Sequence
  Primer based on human sequence
aaaaactactaaccctctc

SEQ ID NO: 61 - DNA, 22 bp, Artificial Sequence
  Primer based on human sequence
gttaggtatttgtagaggagtt

SEQ ID NO: 62 - DNA, 13 bp, Artificial Sequence
  Primer based on human sequence
ctaattccctctc

SEQ ID NO: 63 - DNA, 20 bp, Artificial Sequence
  Primer based on human sequence
tcacctccaactacttcctc

SEQ ID NO: 64 - DNA, 23 bp, Artificial Sequence
  Primer based on human sequence
tagttgagtgattttggttgaat

SEQ ID NO: 65 - DNA, 17 bp, Artificial Sequence
  Primer based on human sequence
tacaaaaaacacccaaa

SEQ ID NO: 66 - DNA, 21 bp, Artificial Sequence
  Primer based on human sequence
ctctactctaactacaaacta

SEQ ID NO: 67 - DNA, 20 bp, Artificial Sequence
  Primer based on human sequence
gaaggtatgaagaggtaagt

SEQ ID NO: 68 - DNA, 16 bp, Artificial Sequence
  Primer based on human sequence
aaattcccaccaaaaa

SEQ ID NO: 69 - DNA, 25 bp, Artificial Sequence
  Primer based on human sequence
aaaattctccctttacaatatttaa

SEQ ID NO: 70 - DNA, 24 bp, Artificial Sequence
  Primer based on human sequence
aagatagtagggtttggtgagagg

SEQ ID NO: 71 - DNA, 25 bp, Artificial Sequence
  Primer based on human sequence
ataattagtaattaggtttttatat

SEQ ID NO: 72 - DNA, 23 bp, Artificial Sequence
  Primer based on human sequence
tctcttctccctctctcctctct

SEQ ID NO: 73 - DNA, 20 bp, Artificial Sequence
  Primer based on human sequence
atacaacaaaactaatctcc

SEQ ID NO: 74 - DNA, 17 bp, Artificial Sequence
  Primer based on human sequence
gagttgtgtttttttag

SEQ ID NO: 75 - DNA, 20 bp, Artificial Sequence
  Primer based on human sequence
ggtttgtgttgaatgggttt

SEQ ID NO: 76 - DNA, 15 bp, Artificial Sequence
  Primer based on human sequence
tgaatgggttttgat

SEQ ID NO: 77 - DNA, 15 bp, Artificial Sequence
  Primer based on human sequence
gggttttatttgtaa

SEQ ID NO: 78 - DNA, 22 bp, Artificial Sequence
  Primer based on human sequence
tcatcccttcacctaacaaaaa

SEQ ID NO: 79 - DNA, 22 bp, Artificial Sequence
  Primer based on human sequence
ggattttagggagaggataggg

SEQ ID NO: 80 - DNA, 25 bp, Artificial Sequence
  Primer based on human sequence
ctaaactattcctataaataacatt

SEQ ID NO: 81 - DNA, 18 bp, Artificial Sequence
  Primer based on human sequence
ccccaccccctacacttt

SEQ ID NO: 82 - DNA, 24 bp, Artificial Sequence
  Primer based on human sequence
agtgtgtatggaatgaaatgtggt

SEQ ID NO: 83 - DNA, 16 bp, Artificial Sequence
  Primer based on human sequence
cccactcccattttta

SEQ ID NO: 84 - DNA, 21 bp, Artificial Sequence
  Primer based on human sequence
ccctcccaaattactctacca

SEQ ID NO: 85 - DNA, 26 bp, Artificial Sequence
  Primer based on human sequence
ttttagttttagaaataaaatagaaa

SEQ ID NO: 86 - DNA, 25 bp, Artificial Sequence
  Primer based on human sequence
ataatctcttactcaacaaaaactc

SEQ ID NO: 87 - DNA, 21 bp, Artificial Sequence
  Primer based on human sequence
ggagagttgaatttagagagt

SEQ ID NO: 88 - DNA, 27 bp, Artificial Sequence
  Primer based on human sequence
ttataaatttaggaggtgtagggattt

SEQ ID NO: 89 - DNA, 26 bp, Artificial Sequence
  Primer based on human sequence
cccaaaactcaacaaaaaaaataaat

SEQ ID NO: 90 - DNA, 19 bp, Artificial Sequence
  Primer based on human sequence
tggggttttgaaggattga

SEQ ID NO: 91 - DNA, 17 bp, Artificial Sequence
  Primer based on human sequence
attttgtattaggtatt

SEQ ID NO: 92 - DNA, 24 bp, Artificial Sequence
  Primer based on human sequence
taaggttttagtgtggggtggttt

SEQ ID NO: 93 - DNA, 24 bp, Artificial Sequence
  Primer based on human sequence
aaacaaatataatccccaccttca

SEQ ID NO: 94 - DNA, 17 bp, Artificial Sequence
  Primer based on human sequence
agttattttgtgaatgt

SEQ ID NO: 95 - DNA, 23 bp, Artificial Sequence
  Primer based on human sequence
gttaagtattgggatattaagtt

SEQ ID NO: 96 - DNA, 17 bp, Artificial Sequence
  Primer based on human sequence
gatttttagaaaggttt

SEQ ID NO: 97 - DNA, 24 bp, Artificial Sequence
  Primer based on human sequence
ataactcaataacctaataactaa

SEQ ID NO: 98 - DNA, 24 bp, Artificial Sequence
  Primer based on human sequence
tagttattgggagttatattaatt

SEQ ID NO: 99 - DNA, 14 bp, Artificial Sequence
  Primer based on human sequence
gggaggttgtttgt

SEQ ID NO: 100 - DNA, 20 bp, Artificial Sequence
  Primer based on human sequence
ctccaacaacctaacaccta

SEQ ID NO: 101 - DNA, 24 bp, Artificial Sequence
  Primer based on human sequence
tgtggggagggtgtaataaaagta

SEQ ID NO: 102 - DNA, 17 bp, Artificial Sequence
  Primer based on human sequence
ggggagggtgtaataaa

SEQ ID NO: 103 - DNA, 22 bp, Artificial Sequence
  Primer based on human sequence
cactcccattccctttcaaata

SEQ ID NO: 104 - DNA, 24 bp, Artificial Sequence
  Primer based on human sequence
gagtgggatttgggttataggagt

SEQ ID NO: 105 - DNA, 18 bp, Artificial Sequence
  Primer based on human sequence
cttaccctataccccaaa

SEQ ID NO: 106 - DNA, 25 bp, Artificial Sequence
  Primer based on human sequence
acacaaaactaaatcaaaataaacc

SEQ ID NO: 107 - DNA, 20 bp, Artificial Sequence
  Primer based on human sequence
ggagattttgtttggggtat

SEQ ID NO: 108 - DNA, 16 bp, Artificial Sequence
  Primer based on human sequence
gagtagtttttttagg

SEQ ID NO: 109 - DNA, 17 bp, Artificial Sequence
  Primer based on human sequence
caaccccttttcctccc

SEQ ID NO: 110 - DNA, 22 bp, Artificial Sequence
  Primer based on human sequence
actcccaaaattactttaaacc

SEQ ID NO: 111 - DNA, 18 bp, Artificial Sequence
  Primer based on human sequence
ttttaagaaggtgtggag

SEQ ID NO: 112 - DNA, 19 bp, Artificial Sequence
  Primer based on human sequence
ggggttttaagaaggtgtg

SEQ ID NO: 113 - DNA, 21 bp, Artificial Sequence
  Primer based on human sequence
gtttgaaggggaaggttagtt

SEQ ID NO: 114 - DNA, 13 bp, Artificial Sequence
  Primer based on human sequence
agggaaggagagg

SEQ ID NO: 115 - DNA, 25 bp, Artificial Sequence
  Primer based on human sequence
aaaatcctctacaaataaaactcta

SEQ ID NO: 116 - DNA, 25 bp, Artificial Sequence
  Primer based on human sequence
ggttgttatggtttttgtttttaag

SEQ ID NO: 117 - DNA, 17 bp, Artificial Sequence
  Primer based on human sequence
aagggatgagaaaggtg

SEQ ID NO: 118 - DNA, 25 bp, Artificial Sequence
  Primer based on human sequence
aaatactcatttccctacatattac

SEQ ID NO: 119 - DNA, 24 bp, Artificial Sequence
  Primer based on human sequence
ggtagtgtgttaggggtattttgt

SEQ ID NO: 120 - DNA, 24 bp, Artificial Sequence
  Primer based on human sequence
cctctaaaaaaataacacttactc

SEQ ID NO: 121 - DNA, 20 bp, Artificial Sequence
  Primer based on human sequence
actatcccttcccacacctc

SEQ ID NO: 122 - DNA, 24 bp, Artificial Sequence
  Primer based on human sequence
aggagggtgattttttttttatta

SEQ ID NO: 123 - DNA, 16 bp, Artificial Sequence
  Primer based on human sequence
gggtgggaggagtaga

SEQ ID NO: 124 - DNA, 22 bp, Artificial Sequence
  Primer based on human sequence
aaacccaaaaacacaaccaatc

SEQ ID NO: 125 - DNA, 24 bp, Artificial Sequence
  Primer based on human sequence
gggttattaattaattatttgtgg

SEQ ID NO: 126 - DNA, 21 bp, Artificial Sequence
  Primer based on human sequence
aaatttatcctacactaccct

SEQ ID NO: 127 - DNA, 23 bp, Artificial Sequence
  Primer based on human sequence
actcctattttcctccctaatcc

SEQ ID NO: 128 - DNA, 21 bp, Artificial Sequence
  Primer based on human sequence
cgtggagactgactaccagat

SEQ ID NO: 129 - DNA, 25 bp, Artificial Sequence
  Primer based on human sequence
agttacatctggtagtcagtctcca

SEQ ID NO: 130 - DNA, 24 bp, Artificial Sequence
  Primer based on human sequence
ttgggaggattatttgagtttagg

SEQ ID NO: 131 - DNA, 19 bp, Artificial Sequence
  Primer based on human sequence
aaatgtttaggaggaaaag

SEQ ID NO: 132 - DNA, 20 bp, Artificial Sequence
  Primer based on human sequence
acttcttccaaaacccaaaa

SEQ ID NO: 133 - DNA, 750 bp, Homo sapiens
  chr1219130982-219131732
aaacgaggctaaaatttgcagactgggaaatctctggtcgaggacagtggggattgagggtggctagggaattccgcagaacaggcacttttgctccgcagagagaggcggcggggtgcagacgcactaaggccaactcgcctagcgttcttacccgccctgcggtgctcttccgtgttccaagagaggctcgggcagatcttagcgctccgcacccccgcataagtgagggactggagaaagcacagaaagctttggacttctgtcctggttcttgacgttgaccactagggaagtgaccttgagtgagtcattcaacctccctaccagttggtttccacctacaacgatgcccaaatggtacgcagaccagagtttcggccgcctgcatcgagtcctgcttgtgaacgcgttctcttgcctctctgtgatgaccttagcatcggggatcctaatgggtcctcgaccagatgattcgcagctccaggtccactcccggggttactacctggtacttctgggtcgtgttgcacagcctcctactgtgtttataatgctacctaccctgcgatgctataaataaccacaagggccacaataattatacttgtcgttcattcagtaaacacttttgggggggcatttcctgtgccaagcactgagctaggtgctagagcttctccaaccatgcagcagagtccagccctgctctcaagaagccgagggtgcctttttcctcgaaggaatcgt

SEQ ID NO: 134 - DNA, 1854 bp, Homo sapiens
  chr161527998-1528616
atacatcacgcagaaggtcatgttcgagagaggagcagctcagattccctgcccagggctctgcgctctgccctggaggggaggaaagaccagaatgaagggggaggagggatgcagagctgccctgggaggagcatagcccagaatggcatgaccgaggtgggcttgagcaccaggcacagctgcacgtgtagttgtggtgagggtgagtgaccaaggttcctgggagctgggaataagcagacgcacccctgacctgtcctctcgccctcagccctggcctctcagccttccttcctcgcagctccaggcacacagccctgtgccacacaccatggcaggggctctgtgcagcagccgtgccccacaagccactctgcagcctccaagtccccagggtgcatggctcaccacaagggcacccacagacccaccgccctgccaggaaggaggcactcctgcccgagccttcctgtctgagccacgctcccgcctgccccaagaaagctgtttggagctggcgttcactgcttgggtgttatacgggttcctggggtgctgcctgtgtgcgtggctctgcgtggctctgcgtggctgtgcgtggtagcagctgactttaaagtcagctgctaccacgcacagccacgcagagccacgcagagccacgcacacaggcagcaccccaggaacccgtataacacccaagcagtgaacgccagctccaaacagctttcttggggcaggcgggagcgtggctcagacaggaaggctcgggcaggagtgcctccttcctggcagggcggtgggtctgtgggtgcccttgtggtgagccatgcaccctggggacttggaggctgcagagtggcttgtggggcacggctgctgcacagagcccctgccatggtgtgtggcacagggctgtgtgcctggagctgcgaggaaggaaggctgagaggccagggctgagggcgagaggacaggtcaggggtgcgtctgcttattcccagctcccaggaaccttggtcactcaccctcaccacaactacacgtgcagctgtgcctggtgctcaagcccacctcggtcatgccattctgggctatgctcctcccagggcagctctgcatccctcctcccccttcattctggtctttcctcccctccagggcagagcgcagagccctgggcagggaatctgagctgctcctctctcgaacatgaccttctgcgtgatgtttaaacatcacgcagaaggtcatgttcgagagaggagcagctcagattccctgcccagggctctgcgctctgccctggaggggaggaaagaccagaatgaagggggaggagggatgcagagctgccctgggaggagcatagcccagaatggcatgaccgaggtgggcttgagcaccaggcacagctgcacgtgtagttgtggtgagggtgagtgaccaaggttcctgggagctgggaataagcagacgcacccctgacctgtcctctcgccctcagccctggcctctcagccttccttcctcgcagctccaggcacacagccctgtgccacacaccatggcaggggctctgtgcagcagccgtgccccacaagccactctgcagcctccaagtccccagggtgcatggctcaccacaagggcacccacagacccaccgccctgccaggaaggaggcactcctgcccgagccttcctgtctgagccacgctcccgcctgccccaagaaagctgtttggagctggcgttcactgcttgggtgttatacgggttcctggggtgctgcctgtgtgcgtggctctgcgtggctctgcgtggctgtgcgtggtagcagctgacttt

SEQ ID NO: 135 - DNA, 118 bp, Homo sapiens
  No BLAT hit
aaaaatgattttgtgctttttcaagcacactagccatatagaaaggacagctggaccttggtggatggttgcttatgtgtttcctgctaggtaagaggtaggcactggaaatgccatc

SEQ ID NO: 136 - DNA, 225 bp, Homo sapiens
  chr1184674077-184674150
cttaaagcacagatgacaatattagtctcacatagaaacacatctttgagaacaataaaatggaaaatagctgagtactcagctattttccattttattgttctcaaagatgtgtttctatgtgagactaatattgtcatctgtgctttcttaaagcacagatgacaatattagtctcacatagaaacacatctttgagaacaataaaatggaaaatagctgagt

SEQ ID NO: 137 - DNA, 520 bp, Homo sapiens
  misc_feature (348)..(348): n is a, c, g, or t
taacccagacgagcccaggacgccctatccctgaaccccatcgtggcgtgcaggtctgagtaggctctgggccatgccggggtacctggcttggaggctctcttcagtctcgggagagatatgcttatcccaggcggagcggagaaggctgggggatgactgatagggggagtcgggaggggatgttgagccctgaggtgttgcactgagcacggatccagttcctcttggctttgccacaaacacagtgaccttatgtagagtccttagcctcggtttccccgtttggaaaaatcattacggcaggcgggcagagctgagtgtgagctcacgcttagagcgggcggngaacactgncatggngctccccctcccactgcttccccgaggaaaaaatcccacctgctaatataattatggaaaaacacaagcacagaaaatttggtttcagcaaacaacgtgcctcagtgcccagggcctgcctggccctcagcacagcgagcaggctggccactggcat

SEQ ID NO: 138 - DNA, 2243 bp, Homo sapiens
  misc_feature (649)..(649): n is a, c, g, or t
agccgcgcacctagagccgtctgactgtgccaacgctgtcgattctgggggccctttctttgctattttctcgctgagcctcagcgagggtgtgtgcaggggtgagggcggggcggggaggctcgggcggcagtccccagtctctccttgctggcccatcctgctccaactctcccagccacggggcttcctggtgggggggcggagagtagaggagacgactaggggtagggctgccggagcttgtgtttcagtctggctggtccagcagatgcttttggggaagcaagatcccgagaccttcagagtcacctcgcagtgtaacctgtagcctgtagagatgacttctacccggcctccccctcagtgagcagggccttccctttctcctggctggctccaggaaggcagaggtgggggtttccagaagcccctcacccccaccttacattgtttcccttcatctacaggagctcttggagtccaggctgagggtcttgaagaaggaactggaggactgtgaggtgttccggtccacggaaaagaaggagagcaaggagctgctggtgagccaggcacccgcaggccccccgtgggacattacagaggcctgagaactcagcaccagggctcggtgtgtgtggtgttnggagtgtgtgctatggaaccgcagaatcgatttcagaaagataatagagtccatattatatagggtgtccacataattgttgtacaaaccagagcttttaaaagctctggtttgtacaacaattatgtggacaccctatataatatggactctattatctttctgaaatcgattctgcggttccatagcacacactccaacaccacacacaccgagccctggtgctgagttctcaggcctctgtaatgtcccacggggggcctgcgggtgcctggctcaccagcagctccttgctctccttcttttccgtggaccggaacacctcacagtcctccagttccttcttcaagaccctcagcctggactccaagagctcctgtagatgaagggaaacaatgtaaggtgggggtgaggggcttctggaaacccccacctctgccttcctggagccagccaggagaaagggaaggccctgctcactgagggggaggccgggtagaagtcatctctacaggctacaggttacactgcgaggtgactctgaaggtctcgggatcttgcttccccaaaagcatctgctggaccagccagactgaaacacaagctccggcagccctacccctagtcgtctcctctactctccgcccccccaccaggaagccccgtggctgggagagttggagcaggatgggccagcaaggagagactggggactgccgcccgagcctccccgccccgccctcacccctgcacacaccctcgctgaggctcagcgagaaaatagcaaagaaagggcccccagaatcgacagcgttggcacagtcagacggctctaggtgcgcggctagccgcgcacctagagccgtctgactgtgccaacgctgtcgattctgggggccctttctttgctattttctcgctgagcctcagcgagggtgtgtgcaggggtgagggcggggcggggaggctcgggcggcagtccccagtctctccttgctggcccatcctgctccaactctcccagccacggggcttcctggtgggggggcggagagtagaggagacgactaggggtagggctgccggagcttgtgtttcagtctggctggtccagcagatgcttttggggaagcaagatcccgagaccttcagagtcacctcgcagtgtaacctgtagcctgtagagatgacttctacccggcctccccctcagtgagcagggccttccctttctcctggctggctccaggaaggcagaggtgggggtttccagaagcccctcacccccaccttacattgtttcccttcatctacaggagctcttggagtccaggctgagggtcttgaagaaggaactggaggactgtgaggtgttccggtccacggaaaagaaggagagcaaggagctgctggtgagccaggcacccgcaggccccccgtgggacattacagaggcctgagaactcagcaccagggctcggtgtgtgtggtgttnggagtgtgtgctatggaaccgcagaatcgatttcagaaagataatagagtccatattatatagggtgtccacataattgttgtacaaaccagagctttt

SEQ ID NO: 139 - DNA, 3105 bp, Homo sapiens
  chr1587238245-87239779
aatagctgcagagcactgaaaatactgcttgatgcttgataaggagcaagcactgggaaaatatgacctgttactactctggtgagcagttcacacagtgaagtcttacaccatctcagtggctctttctcctctctcactgcctacgagacaaagtggtcaccactgcctcctcttcacagttgaggaaactgaggccaagagagtggaggtcagtgaactacccaaggcgttatctttgtcaaatatctcgaacttagagaaagttcaggtttcctgatgaccagccctttgcttttcccaggcgccccagcctagggacccctccacccaagaacaataagcctgggcttggagggagggtaggtgccacctccctgccaggtgcatgtccgcactgggccttccgtgagtacaagtcccccaggtaccagcaggaggcagcatcgcccgcgaggtctgcacgagcgctgctcccagcgagaaatttgacatccctgtccctgtttgcacaaggcttgatctccgcctttgaaacctgcagcccccaggagaatgaaaaccggaggtgtttcccaatccgctggcccgggcggcgtcgagggagagaagccagggcccctctcaatcgctgccccctactccagggagcacaggcactcccgcgggtcagcgctggacaccaaggtcccaacaggtttacaggctgcctgtggcctgtggctgccaacgagctggtggcaggactgggtccaagtgccgtgctgtttccatactggctgcgcacaagcctacgtttctccctgagggtcgccatctcccacccagggctctgccctccaccctccctacccttctggcctcgctgtccctggggcaagaacccatttccccgaaatcagactgctgccttggaccgggacagctcgcggcccccgagagctctagccgtcgaggagctgcctggggacggtacgtggcttaggggtcgctgggcttctggggctgtgtgcgggccgggttcctcccttaccgctttgggtgccctgcgggtccccacacctcccgggcagggtccggggaaggggcaggaggcgtgggccgggcactgcggaggcccgggctgagatgggctgtgatcgcgggcgaggggcgcccggggcacaagtggggagggagcggcgcggggatgcgggcgccggaccctcgtgcccaccgtgagggagcagagcccgagcggaccccgtggcctccgagaccaccgtccgcctccagcccccttcctgccagggtccgcttggctctctcccctgtcgtctgtccagggtcggctgcctccagtgccgtccgcggcgccccaggccctgcccctgggtatctgttggatgctccgggggccccgggagtcctgggaggggctgagggcaacctgcgaggcggctggcggacctgcgcctctggaccgcagctggccggctctgggcgcctggggcaaaggcaactgatgcctatgggttccagacctcgctcaccggggttcttgccccagggacagcgaggccagaagggtaaatagctgcagagcactgaaaatactgcttgatgcttgataaggagcaagcactgggaaaatatgacctgttactactctggtgagcagttcacacagtgaagtcttacaccatctcagtggctctttctcctctctcactgcctacgagacaaagtggtcaccactgcctcctcttcacagttgaggaaactgaggccaagagagtggaggtcagtgaactacccaaggcgttatctttgtcaaatatctcgaacttagagaaagttcaggtttcctgatgaccagccctttgcttttcccaggcgccccagcctagggacccctccacccaagaacaataagcctgggcttggagggagggtaggtgccacctccctgccaggtgcatgtccgcactgggccttccgtgagtacaagtcccccaggtaccagcaggaggcagcatcgcccgcgaggtctgcacgagcgctgctcccagcgagaaatttgacatccctgtccctgtttgcacaaggcttgatctccgcctttgaaacctgcagcccccaggagaatgaaaaccggaggtgtttcccaatccgctggcccgggcggcgtcgagggagagaagccagggcccctctcaatcgctgccccctactccagggagcacaggcactcccgcgggtcagcgctggacaccaaggtcccaacaggtttacaggctgcctgtggcctgtggctgccaacgagctggtggcaggactgggtccaagtgccgtgctgtttccatactggctgcgcacaagcctacgtttctccctgagggtcgccatctcccacccagggctctgccctccaccctccctacccttctggcctcgctgtccctggggcaagaaccccggtgagcgaggtctggaacccataggcatcagttgcctttgccccaggcgcccagagccggccagctgcggtccagaggcgcaggtccgccagccgcctcgcaggttgccctcagcccctcccaggactcccggggcccccggagcatccaacagatacccaggggcagggcctggggcgccgcggacggcactggaggcagccgaccctggacagacgacaggggagagagccaagcggaccctggcaggaagggggctggaggcggacggtggtctcggaggccacggggtccgctcgggctctgctccctcacggtgggcacgagggtccggcgcccgcatccccgcgccgctccctccccacttgtgccccgggcgcccctcgcccgcgatcacagcccatctcagcccgggcctccgcagtgcccggcccacgcctcctgccccttccccggaccctgcccgggaggtgtggggacccgcagggcacccaaagcggtaagggaggaacccggcccgcacacagccccagaagcccagcgacccctaagccacgtaccgtccccaggcagctcctcgacggctagagctctcgggggccgcgagctgtcccggtccaaggcagcagtctgatttcggggaaat

SEQ ID NO: 140 - DNA, 49 bp, Homo sapiens
  No BLAT hit
ctttactcggcgtttcatctgtggtgcaccacgcgctgggtcggttact

SEQ ID NO: 141 - DNA, 1386 bp, Homo sapiens
  chr107001858-7002320
aaataacaaatattctaacgtgtggcaggacagagtcgcttttttgctttggaaaggaaagtgcacagcttgtgtgaacattcacccactaccctagctctgcgccctgccccggctcctaaggcgaacgcccacgttacctccgctgttgcttcagagggctgctgccatctatgggcgtctgtcagcatctgcgcaggtgcgatgtctccgagtcgagacacatagggagaatacggggcacagaaggggagcgtatggaagtcaggggacaaacagctcaagagccatcctctaagcctgcgacccagctctttccaacgatccttcgggtgaaataagatgatccacttcaacagatccatggtgggaaaaggagaaacccccccaaaaaatcacacccctgcatatcagttagactcctggggctctaaccatcctgacttttcaaatgagaacattaatgttctcatttgaaaagtcaggatggttagagccccaggagtctaactgatatgcaggggtgtgattttttgggggggtttctccttttcccaccatggatctgttgaagtggatcatcttatttcacccgaaggatcgttggaaagagctgggtcgcaggcttagaggatggctcttgagctgtttgtcccctgacttccatacgctccccttctgtgccccgtattctccctatgtgtctcgactcggagacatcgcacctgcgcagatgctgacagacgcccatagatggcagcagccctctgaagcaacagcggaggtaacgtgggcgttcgccttaggagccggggcagggcgcagagctagggtagtgggtgaatgttcacacaagctgtgcactttcctttccaaagcaaaaaagcgactctgtcctgccacacgttagaatatttgttatttaaataacaaatattctaacgtgtggcaggacagagtcgcttttttgctttggaaaggaaagtgcacagcttgtgtgaacattcacccactaccctagctctgcgccctgccccggctcctaaggcgaacgcccacgttacctccgctgttgcttcagagggctgctgccatctatgggcgtctgtcagcatctgcgcaggtgcgatgtctccgagtcgagacacatagggagaatacggggcacagaaggggagcgtatggaagtcaggggacaaacagctcaagagccatcctctaagcctgcgacccagctctttccaacgatccttcgggtgaaataagatgatccacttcaacagatccatggtgggaaaaggagaaacccccccaaaaaatcacacccctgcatatcagttagactcctggggctctaaccatcctgacttttcaaatgagaacatt

SEQ ID NO: 142 - DNA, 2010 bp, Homo sapiens
  chr626379151-26379819
cttatgttcttggatcgcagactaaccgagaagagaggattcaaaaatgaagtgattcatccaatcagattttccagtaattcaccaatcacatcgttggactcttcagccaatggttttgttgcgcgggacttttgaaaatcaacaaggccaattagaatgtttctgagtatatataagggcagtttccactctgccaagtggctgagtttttctttcaggtagtctgagatggcccgcaccaagcagactgcacgcaagtccaccggtggcaaagcgccgcgcaagcagctggccactaaggcggctcggaaaagcgcgccggccaccggcggcgtgaagaaacctcatcgctaccgtcccggcaccgtggctctgcgcgagattcgccgctatcagaagtcgactgagctgctgatccgcaagttgcctttccaacgcctggtgcgagaaatcgctcaggacttcaagacagatctgcgctttcagagttccgcggtgatggccctgcaggaggcctgcgaggcctacttggtggggctctttgaggataccaacctgtgtgccatccatgctaagcgagtgactatcatgcccaaggacattcagctcgctcgccgcattcgtggggagagagcgtagaggtttccgggcagtaattctatccaactagttggatagaattactgcccggaaacctctacgctctctccccacgaatgcggcgagcgagctgaatgtccttgggcatgatagtcactcgcttagcatggatggcacacaggttggtatcctcaaagagccccaccaagtaggcctcgcaggcctcctgcagggccatcaccgcggaactctgaaagcgcagatctgtcttgaagtcctgagcgatttctcgcaccaggcgttggaaaggcaacttgcggatcagcagctcagtcgacttctgatagcggcgaatctcgcgcagagccacggtgccgggacggtagcgatgaggtttcttcacgccgccggtggccggcgcgcttttccgagccgccttagtggccagctgcttgcgcggcgctttgccaccggtggacttgcgtgcagtctgcttggtgcgggccatctcagactacctgaaagaaaaactcagccacttggcagagtggaaactgcccttatatatactcagaaacattctaattggccttgttgattttcaaaagtcccgcgcaacaaaaccattggctgaagagtccaacgatgtgattggtgaattactggaaaatctgattggatgaatcacttcatttttgaatcctctcttctcggttagtctgcgatccaagaacgtcttaygttcttggatcgcagactaaccgagaagagaggattcaaaaatgaagtgattcatccaatcagattttccagtaattcaccaatcacatcgttggactcttcagccaatggttttgttgcgcgggacttttgaaaatcaacaaggccaattagaatgtttctgagtatatataagggcagtttccactctgccaagtggctgagtttttctttcaggtagtctgagatggcccgcaccaagcagactgcacgcaagtccaccggtggcaaagcgccgcgcaagcagctggccactaaggcggctcggaaaagcgcgccggccaccggcggcgtgaagaaacctcatcgctaccgtcccggcaccgtggctctgcgcgagattcgccgctatcagaagtcgactgagctgctgatccgcaagttgcctttccaacgcctggtgcgagaaatcgctcaggacttcaagacagatctgcgctttcagagttccgcggtgatggccctgcaggaggcctgcgaggcctacttggtggggctctttgaggataccaacctgtgtgccatccatgctaagcgagtgactatcatgcccaaggacattcagctcgctcgccgcattcgtggggagagagcgtagaggtttccgggcagtaattctatccaact

SEQ ID NO: 143 - DNA, 2241 bp, Homo sapiens
  chr232117579-32118327
agaacgaacggcttgggcgcgggtaatcagctccctttcccccactttctcacttattctacgtacttgggactgtcgtagagtttccagaccccatgtaggcgcccagtcgtggactgtcccactctgctgctctactgctcgtggtgctcccgcgcccaggtgggggtgaggggcggggtcgggggctgggctgcactccatcctggagtcttcgtgttgaaatctcccaggtaactcgtggctcttcttgttttccaagactggtatccggggactgtgacttgcagggtccgccatggagccagagcagatgctggagggacaaacgcaggtatcaggaggccgtggattcagggtggcggtgtgcagggaggcagcgagaaaggggggacttatatctctgtgaccggtgttttgttttcaaggttgcagaaaatcctcactctgagtacggtctcacagacaacgttgaggtaactcgccaagttcctatgcctcatctgtgttgtctcttgctagctggagaaaacttagatctgcttttactaggtggctatgcacaagggaaatctttgttatcgtgaactgaatagtatcaagttatgggtagcttgttttattagcagtactaatgcttcttggtgtacagacgtacaaattacgtttgagtcaaaggattgaacttttattgcgaaacacatattcttgagggatactgtctgaatcatttttcttggtggctttaaagctaccaagaaaaatgattcagacagtatccctcaagaatatgtgtttcgcaataaaagttcaatcctttgactcaaacgtaatttgtacgtctgtacaccaagaagcattagtactgctaataaaacaagctacccataacttgatactattcagttcacgataacaaagatttcccttgtgcatagccacctagtaaaagcagatctaagttttctccagctagcaagagacaacacagatgaggcataggaacttggcgagttacctcaacgttgtctgtgagaccgtactcagagtgaggattttctgcaaccttgaaaacaaaacaccggtcacagagatataagtcccccctttctcgctgcctccctgcacaccgccaccctgaatccacggcctcctgatacctgcgtttgtccctccagcatctgctctggctccatggcggaccctgcaagtcacagtccccggataccagtcttggaaaacaagaagagccacgagttacctgggagatttcaacacgaagactccaggatggagtgcagcccagcccccgaccccgcccctcacccccacctgggcgcgggagcaccacgagcagtagagcagcagagtgggacagtccacgactgggcgcctacatggggtctggaaactctacgacagtcccaagtacgtagaataagtgagaaagtgggggaaagggagctgattacccgcgcccaagccgttcgttctagaacgaacggcttgggcgcgggtaatcagctccctttcccccactttctcacttattctacgtacttgggactgtcgtagagtttccagaccccatgtaggcgcccagtcgtggactgtcccactctgctgctctactgctcgtggtgctcccgcgcccaggtgggggtgaggggcggggtcgggggctgggctgcactccatcctggagtcttcgtgttgaaatctcccaggtaactcgtggctcttcttgttttccaagactggtatccggggactgtgacttgcagggtccgccatggagccagagcagatgctggagggacaaacgcaggtatcaggaggccgtggattcagggtggcggtgtgcagggaggcagcgagaaaggggggacttatatctctgtgaccggtgttttgttttcaaggttgcagaaaatcctcactctgagtacggtctcacagacaacgttgaggtaactcgccaagttcctatgcctcatctgtgttgtctcttgctagctggagaaaacttagatctgcttttactaggtggctatgcacaagggaaatctttgttatcgtgaactgaatagtatcaagttatgggtagcttgttttattagcagtactaatgcttcttggtgtacagacgtacaaattacgtttgagtcaaaggattgaacttttattgcgaaacacatattcttgagggatactgtctgaatcatttttcttggtrgcttt

SEQ ID NO: 144 - DNA, 2021 bp, Homo sapiens
  chr9116389648-116390322
aaagaggccagggagcggacagaggagtagagggcagtactgcgcagtagactgcacaggcgcagtcgaggagctccgctcctttctcctcagccccgccccgccccatttctgccgaatcacagaagttcaagagcggtcagctgatgctacgctgtgcggtcacgtgacaacgggggcgaagcgcaggcgcaaggagcaagcgcagattgtgggcggctgtgtcagctgacccaaggggccttcgaggtgccttaggccgcttgccttgctctcagaatcgctgccgccatggctagtcagtctcaggggattcagcagctgctgcaggccgagaagcgggcagccgagaaggtgtccgaggcccgcaaaagtgagtttcagggtggggctgcccggcgtggcgcgagtcgaagaaagaccgctgggcctcaggtggtgggtagattaggcccaaaaaactgcggggtgcggacgtgcgtatagtcgcggaaggttgtgtgtttcgaagctttgaggagctgaggctctggaaggcttgcggatcgcctggaagccacgatgtgagatggtaaattgggcgtggaaacaataatgggttaccgtccttggggcctggaggcatgtgaaatgggctctccaaacggagctctttgttcttcttctctcccactagtgtgagagaagaagaacaaagagctccgtttggagagcccatttcacatgcctccaggccccaaggacggtaacccattattgtttccacgcccaatttaccatctcacatcgtggcttccaggcgatccgcaagccttccagagcctcagctcctcaaagcttcgaaacacacaaccttccgcgactatacgcacgtccgcaccccgcagttttttgggcctaatctacccaccacctgaggcccagcggtctttcttcgactcgcgccacgccgggcagccccaccctgaaactcacttttgcgggcctcggacaccttctcggctgcccgcttctcggcctgcagcagctgctgaatcccctgagactgactagccatggcggcagcgattctgagagcaaggcaagcggcctaaggcacctcgaaggccccttgggtcagctgacacagccgcccacaatctgcgcttgctccttgcgcctgcgcttcgcccccgttgtcacgtgaccgcacagcgtagcatcagctgaccgctcttgaacttctgtgattcggcagaaatggggcggggcggggctgaggagaaaggagcggagctcctcgactgcgcctgtgcatctactgcgcagtactgccctctactcctctgtccgctccctggcctctttaaagaggccagggagcggacagaggagtagagggcagtactgcgcagtagactgcacaggcgcagtcgaggagctccgctcctttctcctcagccccgccccgccccatttctgccgaatcacagaagttcaagagcggtcagctgatgctacgctgtgcggtcacgtgacaacgggggcgaagcgcaggcgcaaggagcaagcgcagattgtgggcggctgtgtcagctgacccaaggggccttcgaggtgccttaggccgcttgccttgctctcagaatcgctgccgccatggctagtcagtctcaggggattcagcagctgctgcaggccgagaagcgggcagccgagaaggtgtccgaggcccgcaaaagtgagtttcagggtggggctgcccggcgtggcgcgagtcgaagaaagaccgctgggcctcaggtggtgggtagattaggcccaaaaaactgcggggtgcggacgtgcgtatagtcgcggaaggttgtgtgtttcgaagctttgaggagctgaggctctggaaggcttgcggatcgcctggaagccacgatgtgagatggtaaattgggcgtggaaacaataatgggttaccgtccttggggcctggaggcatgtgaaatgggctctccaaacggagctctttgttcttcttctctcmcact

SEQ ID NO: 145 - DNA, 1359 bp, Homo sapiens
  chr2213109241-213109694
aaaccagctgaactttccctgccaggtgcggcagggtgggggtgggggaggatgaaaaacgaagtcccccggggcatttacagtttccaatggatcaccttagtatgcgtgttgcaattataccagggatgcacccgagagcttttctttgtgcggccctttcatctggatcactttgtgttttgaaaaagtgaggctcgcctttgtgcccccagaatttcaagtacagagtcacacacggaacaacaatggcacgctaatcctcgcgctctaaatgggctcacttgtgggtctgacaattcgcccagggaaacgagaagagacgaagcgagggcgcgagctagtgtcgcctgagacgaggaagaggacagctgcagaactaatttggattacattccttgcagaaattctgaaaagggcttttcggcatagctcaactcggagccgcacagtactgtgcggctccgagttgagctatgccgaaaagcccttttcagaatttctgcaaggaatgtaatccaaattagttctgcagctgtcctcttcctcgtctcaggcgacactagctcgcgccctcgcttcgtctcttctcgtttccctgggcgaattgtcagacccacaagtgagcccatttagagcgcgaggattagcgtgccattgttgttccgtgtgtgactctgtacttgaaattctgggggcacaaaggcgagcctcactttttcaaaacacaaagtgatccagatgaaagggccgcacaaagaaaagctctcgggtgcatccctggtataattgcaacacgcatactaaggtgatccattggaaactgtaaatgccccgggggacttcgtttttcatcctcccccacccccaccctgccgcacctggcagggaaagttcagctggtttaaaccagctgaactttccctgccaggtgcggcagggtgggggtgggggaggatgaaaaacgaagtcccccggggcatttacagtttccaatggatcaccttagtatgcgtgttgcaattataccagggatgcacccgagagcttttctttgtgcggccctttcatctggatcactttgtgttttgaaaaagtgaggctcgcctttgtgcccccagaatttcaagtacagagtcacacacggaacaacaatggcacgctaatcctcgcgctctaaatgggctcacttgtgggtctgacaattcgcccagggaaacgagaagagacgaagcgagggcgcgagctagtgtcgcctgagacgaggaagaggacagctgcagaactaatttggattacattccttgcagaaattctgaaaagggcttttcggcatagctcaactcggagccgcacagt

SEQ ID NO: 146 - DNA, 551 bp, Homo sapiens
  chr3106570470-106570674
atctgataacaactgtgtgcgtgtggacgtttgcatgttaccaaccgacctcacccttgtacctcccttcttggctcattccctggctccgaagtgtcctttcctccatcagacagcggaatctccaaaaagtgaaatccacactgtgtcactcccaaagccttctcgcctttccgcctacctccccagcgttgttacactaggacacctgcttaagcaggtgtcctagtgtaacaacgctggggaggtaggtggaaatgggagacccccgtgggactggtacagcggggatttcctttttcgagattccactgtccgatggaggaaaggacactttatctgataacaactgtgtgcgtgtggacgtttgcatgttaccaaccgacctcacccttgtacctcccttcttggctcattccctggctccraagtgtcctttcctccatcrgacagyggaatctcsaaaaagtgaaatccmcrctgtrycastcccamrgssktctcscmtttccrcctacctccccagcgttgttacactaggacacctgctt

SEQ ID NO: 147 - DNA, 2138 bp, Homo sapiens
  chr1657221393-57222125
tagccaggaagagaacttgcgtcccctacgtctctggaaggcgtctctggaaggtgtcccttcttcaatagacagaactaaaacagaaagttcccttgaccaaagcgaagcaagcgaggacaaggtggccacctgggtgactacaacccccagcatgcactgctccttctagggcgcagcgacgaacgaagacgcattacttctgcgatgggaacggggtagaggagcgcgactacacatcccaggatgcaaagcgccccaactctagtcacaacctggcttgctttctggctggcgttccccgccaaagggacactaggggaggggattgtttcggttgggaatccccgaagtctactggaccgtgacatttcgaaagaggaaggaaagtggaggaaagctgtttcactgtaattcaaagtagttcccgcgcagggtggcgaggagttatgggccccttccataatttattgttcctcaaaggcaatgccaagccccactggtcgcagccctccagggcaggtgggaagtacgtgaagccccaaatcgaagttatcagcatctccctattggccctcgcctcgagcgcaggcgcgagggctctgactccggtcccctgggcctggtgagcggcaattggccaaagtccgcgaagtaggaggccggattggtagttgctagcaggagcctgatttcttcctcgacttctgcaagtgatagagtgtgattggttaaccaatcacactctatcacttgcagaagtcgaggaagaaatcaggctcctgctagcaactaccaatccggcctcctacttcgcggactttggccaattgccgctcaccaggcccaggggaccggagtcagagccctcgcgcctgcgctcgaggcgagggccaatagggagatgctgataacttcgatttggggcttcacgtacttcccacctgccctggagggctgcgaccagtggggcttggcattgcctttgaggaacaataaattatggaaggggcccataactcctcgccaccctgcgcgggaactactttgaattacagtgaaacagctttcctccactttccttcctctttcgaaatgtcacggtccagtagacttcggggattcccaaccgaaacaatcccctcccctagtgtccctttggcggggaacgccagccagaaagcaagccaggttgtgactagagttggggcgctttgcatcctgggatgtgtagtcgcgctcctctaccccgttcccatcgcagaagtaatgcgtcttcgttcgtcgctgcgccctagaaggagcagtgcatgctgggggttgtagtcacccaggtggccaccttgtcctcgcttgcttcgctttggtcaagggaactttctgttttagttctgtctattgaagatagccaggaagagaacttgcgtcccctacgtctctggaaggcgtctctggaaggtgtcccttcttcaatagacagaactaaaacagaaagttcccttgaccaaagcgaagcaagcgaggacaaggtggccacctgggtgactacaacccccagcatgcactgctccttctagggcgcagcgacgaacgaagacgcattacttctgcgatgggaacggggtagaggagcgcgactacacatcccaggatgcaaagcgccccaactctagtcacaacctggcttgctttctggctggcgttccccgccaaagggacactaggggaggggattgtttcggttgggaatccccgaagtctactggaccgtgacatttcgaaagaggaaggaaagtggaggaaagctgtttcactgtaattcaaagtagttcccgcgcagggtggcgaggagttatgggccccttccataatttattgttcctcaaaggcaatgccaagccccactggtcgcagccctccagggcaggtgggaagtacgtgaagccccaaatcgaagttatcagcatctccctattggccctcgcctcgagcgcaggcgcgagggctctgactccggtcccctgggcctggtgagcggcaattggccaaagtccgcgaagtaggaggccggattggtagttgctagcaggagcctgatttcttcctcgacttctgcaagtgatagagtgtgattggtt

SEQ ID NO: 148 - DNA, 1140 bp, homo sapiens
  chr1230739052-30739432
agtgcgcggacttggggagcaggtgggaattttacttacagttcaaatctgagttcagcttttttccaaacccagccctttttaggtgttgctgtggagcacgctgcccaccgcctcctcctgtcgggttggcaggactgttttgacaccgattggccgagttttgcggagcgctgggcagtgctgttgggagagtgctctgggaagaataggcagttttcatggtgatactgctttatctaagacacatcaaagtaagatgtgttggagaaatttcttatgaatgctaaaatgtgtaagacttctttcttttctccgtctcctgttcttcctaattagccttgacgtctttccagcgttacccccacacttcaaaatttaaattttgaagtgtgggggtaacgctggaaagacgtcaaggctaattaggaagaacaggagacggagaaaagaaagaagtcttacacattttagcattcataagaaatttctccaacacatcttactttgatgtgtcttagataaagcagtatcaccatgaaaactgcctattcttcccagagcactctcccaacagcactgcccagcgctccgcaaaactcggccaatcggtgtcaaaacagtcctgccaacccgacaggaggaggcggtgggcagcgtgctccacagcaacacctaaaaagggctgggtttggaaaaaagctgaactcagatttgaactgtaagtaaaattcccacctgctccccaagtccgcgcactagtgcgcggacttggggagcaggtgggaattttacttacagttcaaatctgagttcagcttttttccaaacccagccctttttaggtgttgctgtggagcacgctgcccaccgcctcctcctgtcgggttggcaggactgttttgacaccgattggccgagttttgcggagcgctgggcagtgctgttgggagagtgctctgggaagaataggcagttttcatggtgatactgctttatctaagacacatcaaagtaagatgtgttggagaaatttcttatgaatgctaaaatgtgtaagacttctttcttttctccgtctcctgttcttcctaattagccttgacgtctttccagcgttacccccacacttcaaaattt

SEQ ID NO: 149 - DNA, 567 bp, homo sapiens
  chr12107478405-107478972
agagtgggagttctggaatgggatcaaagtacaaggcctgacttgcaggaagtgttatcttgagcaagagactttcctccacgagcctcagtttcctccagtaaaatggggatagataacagggcttccttcacagggtcgtggcgaggattcaatgagataacgtttgcaaggcaatgggcaggcagtaagcactcaaatccgacaggagctactgtcacgaaaaacgggtttgcagccttggcggctttatcgccgccgaggactagggaggcgggccaggacatggggacccgaggacctgaaagactggaagatgcccgctgcccacccccgggcccacctgctcctccagtctctcaatctccagctggtttttctcctcctcttcgtcatattcccactcgtactccccgggggagctctccgcggaggaagccatggcgtactcatccccatcgctctcgctcacgccttcctcctgctgatcccacgctggccccatggtcttgtatgtcgcagcggccacagcccgcgataacacctttctccttgtcctagccgcct

SEQ ID NO: 150 - DNA, 2303 bp, Homo sapiens
  chr177970249-7971017
tagtttccttctggaggccacggttactacttagcgctaagaacagagcattggcagtcacagatacaatccgaggacgagacagagacccagaccctggcgcaaataaaacaggttgaaagaaacgggagggacagctccagggtgagttccagagacagagacccagatagactaaggccgagacccagattcactcagcgaacctgagccccgagcccacaggccccggcagcgaccgcggacagagccaggaggaggcgagcggtgggaggcggagcgcgccggaagggagcggaccgcggggagccccgccgccccgcccagccggcccgccccgccccgctcacaagtggtcgccgcaggtggctgtgcgcccacccgcccgccctcgggccgacgcgtgcgggatcaaagcggccgggaaggggcggggaggggaggtggcaggaacctggaagtgtcagcgagcttcccaggggatggcaggagcgtgggagcgcagagtcccgggaggcgacagagactcggagagacgacgagataccagagacccgagagagccagaggcggagatgcagacgccaagacgcggagagacgcggaatcagagatgcggagggaggagaccaccaacaccagagagacccagggcagaagtcgctgaagatctggaaaaaacgtgagagatacagagatggaggagctaagagacactcagagacccggaaaggaggcgagacaaaaaaggcatagacccatagactagtctatgggtctatgccttttttgtctcgcctcctttccgggtctctgagtgtctcttagctcctccatctctgtatctctcacgttttttccagatcttcagcgacttctgccctgggtctctctggtgttggtggtctcctccctccgcatctctgattccgcgtctctccgcgtcttggcgtctgcatctccgcctctggctctctcgggtctctggtatctcgtcgtctctccgagtctctgtcgcctcccgggactctgcgctcccacgctcctgccatcccctgggaagctcgctgacacttccaggttcctgccacctcccctccccgccccttcccggccgctttgatcccgcacgcgtcggcccgagggcgggcgggtgggcgcacagccacctgcggcgaccacttgtgagcggggcggggcgggccggctgggcggggcggcggggctccccgcggtccgctcccttccggcgcgctccgcctcccaccgctcgcctcctcctggctctgtccgcggtcgctgccggggcctgtgggctcggggctcaggttcgctgagtgaatctgggtctcggccttagtctatctgggtctctgtctctggaactcaccctggagctgtccctcccgtttctttcaacctgttttatttgcgccagggtctgggtctctgtctcgtcctcggattgtatctgtgactgccaatgctctgttcttagcgctaagtagtaaccgtggcctccagaaggaaactgtacagtttccttctggaggccacggttactacttagcgctaagaacagagcattggcagtcacagatacaatccgaggacgagacagagacccagaccctggcgcaaataaaacaggttgaaagaaacgggagggacagctccagggtgagttccagagacagagacccagatagactaaggccgagacccagattcactcagcgaacctgagccccgagcccacaggccccggcagcgaccgcggacagagccaggaggaggcgagcggtgggaggcggagcgcgccggaagggagcggaccgcggggagccccgccgccccgcccagccggcccgccccgccccgctcacaagtggtcgccgcaggtggctgtgcgcccacccgcccgccctcgggccgacgcgtgcgggatcaaagcggccgggaaggggcggggaggggaggtggcaggaacctggaagtgtcagcgagcttcccaggggatggcaggagcgtgggagcgcagagtcccgggaggcgacagagactcggagagacgacgagataccagagacccgagagagccagaggcggagatgcagacgccaagacgcggagagacgcggaatcagagatgcggagggaggagaccaccaacaccagagagacccagggcagaagtcgctgaagatctggaaaaaacgtgagagatacagagatggaggagctaagagacactcagagacccggaaaggaggcgagacaaaaaaggcatagacccatagact

SEQ ID NO: 151 - DNA, 1209 bp, Homo sapiens
  chr2213109241-213109694
aaaccagctgaactttccctgccaggtgcggaagggtgagagagggggaggatgaaaaacaaagacccacggaggatttacagtttccaagggatcaccttactaggcgagttgcaattataccagggatgcaccccagagcttttctatgtgcgggcctttcatcagaatcactttgcgtttggaaaaagcgaagctcggctttgagcccccagaattgacaagtacagagtcacgcacgcaacaacaatgacacgctaatcctcgcgctctagatgggctcacttgtgggtctgacaattactgtgcggatccgagttgagctatgccgaaaagcccttttcagaatttctgcaaggaatgtaatccaaattatttctgcagatgtcctcttcctcgtctcacgcgacactagctcgcgccctcgcttcgtctcttctcgtttccctggctgaattgtctgacccactagtgagcccatttagagtgcgaggattatcgtgccattgttgttccgtgcgtgactctgtacttgaaattctgggggcagaaaggcgagcctcactttttcaaaacacaaagtgatccagatgaaagggccgcacaaagaaaagctctcgggtgcatccctggtataattgcaacacgcatactaaggtgatccattggaaactgtaaatgccccgggggacttcgtttttcatcctcccccacccccaccctgccgcacctggcagggaaagttcagctggtttaaaccagctgaactttccctgccaggtgcggmagggtgrgrgwgggggaggatgaaaaacraagwcccmcggrgsatttacagtttccaakggatcaccttastakgcgwgttgcaattataccagggatgcacccsagagcttttctwtgtgcggscctttcatcwgratcactttgygtttkgaaaaagygargctcgsctttswgcccccagaattgwcaagtacagagtcacgcacgsaacaacaatgrcacgmtaatcctcgcrctctaratgggctcactwgtgggtcwgacaattcagccagggaaacgagaagagacgaagcgagggcgcgagctagtgtcgcgtgagacgaggaagaggacatctgcagaaataatttggattacattccttgcagaaattctgaaaagggcttttcggcatagctcaactcggatccgcacagt

SEQ ID NO: 152 - DNA, 133 bp, Homo sapiens
  No BLAT hit
cttgggtaaactatagtcctctctcctccccctgcaccacctctgattctgaatctctcgccggaccccgccgagcccatctcacccctctggctcccctcttacccatcccccctgccagagctcgtcgccg

SEQ ID NO: 153 - DNA, 325 bp, Homo sapiens
  misc_feature (186)..(186): n is a, c, g, or t
aaactcatcacttgtctaccgtctgtcttcccgagtacagggtacttctctgacttgttcctgcttccagaaaagtgcctggcacagagtactcaaacacccagcgaatccaggatgacaccaagcccgcacgtgcaccacccagtcccccctgctcctcccgggacacgtcccgccccacggtgnccggcgcatgcgccccctgnccgncgaggnaattccacccccaccccggtcttttcctgctcgtccccgngccctcgcccaggaaacccctggcccctgccctctcctgagtgcccccgggccaaaggggtgccctcgn

SEQ ID NO: 154 - DNA, 2016 bp, Homo sapiens
  chr1192866208-92866881
aggcttgacaaagcagtaaggtgaaggcagagaaacagtggaggaggctatcacaatttcctcatcaataaagatgagggctgtggggaaggagagaggggtatgtctgagagatgtttaggataagggagaacaatctgatgtagagaatgacagagaggggtggtatctaggagatccccagacttctggcttggcagcattggcgagagggacagtcttgtatcatccagcaagtaagtaaagcaggccaagatgaggtggcttggaagtgggcaagagtttggcttgttctcccactatgtgcagacagaacagccgtgcatgatgcgatgtcagaggtcagggttgggctcagcacagaggtgttgaaagccaggtcctatctgtaccctgttttgttgacagtggggaacacatgaacacattctagttagaggactaagatgagacatgggtttctatgaaatcattccctggaaattgaggaatcaggaggctggtggaagggaggcatctgggtggagatgctgaggactcggtgatggcagggagaacatacaggcatgggtatgcgtgcagtgtctgcagcctctattttatttgggggtggggagtgatacagattctagagtcatcacagaggagaatgagctgcccttgataagctatatagcttaccaagggctgctcattctcctctgtgttgactctagaatctgtatcactccccacccccaaataaaatagaggctgcagacactgcacgcatacccatgcctgtatgttctccctgccatcaccgagtcctcagcatctccacccagatgcctcccttccaccagcctcctgattcctcaatttccagggaatgatttcatagaaacccatgtctcatcttagtcctctaactagaatgtgttcatgtgttccccactgtcaacaaaacagggtacagataggacctggctttcaacacctctgtgctgagcccaaccctgacctctgacatcgcatcatgcacggctgttctgtctgcacatagtgggagaacaagccaaactcttgcccacttccaagccacctcatcttggcctgctttacttacttgctggatgatacaagactgtccctctcgccaatgctgccaagccagaagtctggggatctcctagataccacccctctctgtcattctctacatcagattgttctcccttatcctaaacatctctcagacatacccctctctccttccccacagccctcatctttattgatgaggaaattgtgatagcctcctccactgtttctctgccttcaccttactgctttgtcaagcctaggcttgacaaagcagtaaggtgaaggcagagaaacagtggaggaggctatcacaatttcctcatcaataaagatgagggctgtggggaaggagagaggggtatgtctgagagatgtttaggataagggagaacaatctgatgtagagaatgacagagaggggtggtatctaggagatccccagacttctggcttggcagcattggcgagagggacagtcttgtatcatccagcaagtaagtaaagcaggccaagatgaggtggcttggaagtgggcaagagtttggcttgttctcccactatgtgcagacagaacagccgtgcatgatgcgatgtcagaggtcagggttgggctcagcacagaggtgttgaaagccaggtcctatctgtaccctgttttgttgacagtggggaacacatgaacacattctagttagaggactaagatgagacatgggtttctatgaaatcattccctggaaattgaggaatcaggaggctggtggaagggaggcatctgggtggagatgctgaggactcggtgatggcagggagaacatacaggcatgggtatgcgtgcagtgtctgcagcctctattttatttgggggtggggagtgatacagattctagagtcawcacagaggagaatgagcwgcccttgrtaagctat

SEQ ID NO: 155 - DNA, 2324 bp, Homo sapiens
  misc_feature (597)..(597): n is a, c, g, or t
atgaccaccacgacacctttgccgtcggctgccggctccacgcccacagtcttgcggtgaatcagtccgttgtagcggaaggaattgcgggccttcaagttattgggctcctgggaaggaggcggcacaaggcatcgtgaataagggacccggagacatgcgaaggtcccgccctctgacccagagtcaagagactcaggaccctcctcccgcaggagccacgcatccgggccccacttacagtgctgtaggtctgcttattcctcttgatcaggaaactggagcagttccgcacgaccatccattgcagatgcgcagacatggctgcggctgcggggaaacgggcacagggaaagcgtcagacacagagaactaaagcagcgttaggccaccggccacgctcggacagcagaggaagagagcgactgggaacttgggtgaatgggagacccctgagggcagacagcgacctagaactcttgtcaggcgggactgagtgacctaaggcggcgggaccgaattcaggcagccgagactagctgcggcgaatgagagtgaggggaaaataacttgaaaattcacaacgacttcacgggngaggggttggggtccgccaatgacccttgaggcgcaaggggagaaggcaggcagaggactgagagggagtgcgcggcgggccggagatggcgaccacgcgaggaagacgaacgctcacctccctccgcagcggcgacctgagacggaaagaggaaggcgaggggcgaggcgacgccttaaggcgtcgcctcgcccctcgccttcctctttccgtctcaggtcgccgctgcggagggaggtgagcgttcgtcttcctcgcgtggtcgccatctccggcccgccgcgcactccctctcagtcctctgcctgccttctccccttgcgcctcaagggtcattggcggaccccaacccctccccgtgaagtcgttgtgaattttcaagttattttcccctcactctcattcgccgcagctagtctcggctgcctgaattcggtcccgccgccttaggtcactcagtcccgcctgacaagagttctaggtcgctgtctgccctcaggggtctcccattcacccaagttcccagtcgctctcttcctctgctgtccgagcgtggccggtggcctaacgctgctttagttctctgtgtctgacgctttccctgtgcccgtttccccgcagccgcagccatgtctgcgcatctgcaatggatggtcgtgcggaactgctccagtttcctgatcaagaggaataagcagacctacagcactgtaagtggggcccggatgcgtggctcctgcgggaggagggtcctgagtctcttgactctgggtcagagggcgggaccttcgcatgtctccgggtcccttattcacgatgccttgtgccgcctccttcccaggagcccaataacttgaaggcccgcaattccttccgctacaacggactgattcaccgcaagactgtgggcgtggagccggcagccgacggcaaaggtgtcgtggtggtcatatgaccaccacgacacctttgccgtcggctgccggctccacgcccacagtcttgcggtgaatcagtccgttgtagcggaaggaattgcgggccttcaagttattgggctcctgggaaggaggcggcacaaggcatcgtgaataagggacccggagacatgcgaaggtcccgccctctgacccagagtcaagagactcaggaccctcctcccgcaggagccacgcatccgggccccacttacagtgctgtaggtctgcttattcctcttgatcaggaaactggagcagttccgcacgaccatccattgcagatgcgcagacatggctgcggctgcggggaaacgggcacagggaaagcgtcagacacagagaactaaagcagcgttaggccaccggccacgctcggacagcagaggaagagagcgactgggaacttgggtgaatgggagacccctgagggcagacagcgacctagaactcttgtcaggcgggactgagtgacctaaggcggcgggaccgaattcaggcagccgagactagctgcggcgaatgagagtgaggggaaaataacttgaaaattcacaacgacttcacgggngaggggttggggtccgccaatgacccttgaggcgcaaggggagaaggcaggcagaggactgagagggagtgcgcggcgggccggagatggcgaccacgcgaggaagacgaacgctcacctccctccgcagcggcgacctgagacggaaagaggaaggcgaggggcgaggcgacgcctt

SEQ ID NO: 156 - DNA, 2085 bp, Homo sapiens
  chr1219131734-219132429
aaaattgtgtgtcacacagcaagtccttagtttgactctttctttatagcttttcacccccatttttttcagctacaaaactgtgctcttggcctggaagagtagtcagcacaggctgcagggtccctgatggaggcaagaaaaaggagacctggcttagatcgctgaagctgacccaaagtggcctcaaagagtcatctcctgggcgctcccgaccggcaaccgactgacgtagcgatcagcttgggggaggtgggggccaaggggcccgcgctgaccgtgaactgaatgaatattcggcgggcaggttggggatccgagaccacggaagacccaggaccttcctatgctacacccaaagccattgagaacggggcgagaccctgtgacccccgaaggtaggaggccttggagcgagatctgagacccagaccacgagagcctggctgcgtagagtctcagggacacctctttggaggcgtgcgcggcccgcatttcagcaggtagtgaaattcaacagtggccggattgtgatgacggatgcaagtcgggactgccagataggggatcctattcgctcattagttcattcttttgatctttcgttcacttgctcatttcttgagcttctaccacgtgctgggtgcactcgtcacgcggattctgagaaacaaaaataaacagccccgacaaatatttgtcggggctgtttatttttgtttctcagaatccgcgtgacgagtgcacccagcacgtggtagaagctcaagaaatgagcaagtgaacgaaagatcaaaagaatgaactaatgagcgaataggatcccctatctggcagtcccgacttgcatccgtcatcacaatccggccactgttgaatttcactacctgctgaaatgcgggccgcgcacgcctccaaagaggtgtccctgagactctacgcagccaggctctcgtggtctgggtctcagatctcgctccaaggcctcctaccttcgggggtcacagggtctcgccccgttctcaatggctttgggtgtagcataggaaggtcctgggtcttccgtggtctcggatccccaacctgcccgccgaatattcattcagttcacggtcagcgcgggccccttggcccccacctcccccaagctgatcgctacgtcagtcggttgccggtcgggagcgcccaggagatgactctttgaggccactttgggtcagcttcagcgatctaagccaggtctcctttttcttgcctccatcagggaccctgcagcctgtgctgactactcttccaggccaagagcacagttttgtagctgaaaaaaatgggggtgaaaagctataaagaaagagtcaaactaaggacttgctgtgtgacacacaattttaaaattgtgtgtcacacagcaagtccttagtttgactctttctttatagcttttcacccccatttttttcagctacaaaactgtgctcttggcctggaagagtagtcagcacaggctgcagggtccctgatggaggcaagaaaaaggagacctggcttagatcgctgaagctgacccaaagtggcctcaaagagtcatctcctgggcgctcccgaccggcaaccgactgacgtagcgatcagcttgggggaggtgggggccaaggggcccgcgctgaccgtgaactgaatgaatattcggcgggcaggttggggatccgagaccacggaagacccaggaccttcctatgctacacccaaagccattgagaacggggcgagaccctgtgacccccgaaggtaggaggccttggagcgagatctgagacccagaccacgagagcctggctgcgtagagtctcagggacacctctttggaggcgtgcgcggcccgcatttcagcaggtagtgaaattcaacagtggccggattgtgatgacggatgcaagtcgggactgccagataggggatcctattcgctcattagttcattcttttgatctttcgttcacttgctcatttcttgagcttctaccacgtgctgggtgcactcgtcacgcggattctgagaaacaaaaataaacagccccgacaaat

SEQ ID NO: 157 - DNA, 501 bp, Homo sapiens
  chr13109899692-109899859
gctcattctccagcaggggaacaggattagtgaaaaccgggtgaggaagtaggtgagtccaaagctcgaataaagaaggaactaatgtggttgccaattctgctggtggcctgggagcgaggaggcctcacttccctgcaccccactctgcccctcagccccagcttaagctggggctgaggggcagagtggggtgcagggaagtgaggcctcctcgctcccaggccaccagcagaattggcaaccacattagttccttctttattcgagctttggactcacctacttcctcacccggttttcactaatcctgttcccctgctggagaatgagcgctcattctccagcaggggaacaggattagtgaaaaccgggtgaggaagtaggtgagtccaaagctcgaataaagaaggaactaatgtggttgccaattctgctggtggcctgggagcgaggaggcctcacttccctgcaccccactctgcccctcagccccagctt

SEQ ID NO: 158 - DNA, 896 bp, Homo sapiens
  chr1756828619-56828930
agacctcggcgagcaacgaatgtgcccaggctaagctcaggggacgtgtgtgggcccgcgctatgggcccgctgtgaggttgccgccaggccgacgatcactgactggacgtgaggagtgtaggtggagaaggacactccctggacagagtcgcttcccatcagggcttccccggcggaccgcacctcccggagccctggcgccagccagtccggattagcggccggaccggactcccccgcggacccaggtctggagtgcactgggacgggccgggccgctgcgggctggggtcaccgaggccgcgggccccaccccaaccagacctgggaccgcgggggagcccggtccggccgctaaaccgggctggctggcgccagggctccgggaggtgcggtccggcggggaagccgggatgggaagcgactctgtccagggagtgtccttcaccaccacactcctcgcgtccaggcagtgatcgacggcctggcggcaccctcgcagcgggcccatagcacggggccacacacgtcccctgagcttagcctgggcacattcgtctgccaccgagggctagmcctcggygagcagacgaatgtgcccaggctaagctcaggggacgtgtgtggscccgygctatgggcccgctgygaggktgccgccaggccgwcgatcactgmctggacgygaggagtgtaggtggwgaaggacactccctggacagagtcgcttcccatcmsggcttccccgscggaccgcacctcccggagccctggcgccagccagyccggwttagcggccggaccggrctcccccgcggwcccaggtctggttggggtggggcccgcggcctcggtgaccccagcccgcagcggcccggcccgtcccagtgcact

SEQ ID NO: 159 - DNA, 325 bp, Homo sapiens
  chr1446575272-46575381
cataatttctgatttacagagaatatttatgagaagttagattgattttactgtaacttactttattgtgctgaaatatatttatgttagaatttagtgaaaaccattatgatggttttcactaaattctaacataaatatatttcagcacaataaagtaagttacagtaaaatcaatctaacttctcataaatattctctgtaaatcagaaatcataatggttttcactaaattctaacataaatatatttcagcacaataaagtaagttacagtaaaatcaatctaacttctcataaatattctctgtaaatcagaaattatg

SEQ ID NO: 160 - DNA, 890 bp, Homo sapiens
  chr7129920209-129920497
tagtatttttcagatttcagtaacaagcgcgagtgagaacggcgatgtgaccaaactgtcatgttgcgcagggattgttcaccttggtttcgcgggttttcaaagtggttcgtctcgcggcgacgccatcaggtgggcggcaggttgggtggtattattacgggagtgtgtttttctgtttacaagatgaagaaatcaaggctcagaaatttttggaaagtttgttactggagtttgcttattgtctccgctcatggctcatcaaatagttgggattttggccaaaaaatgtttgctagcactttttttttggccaaaatcccaactatttgatgagccatgagcggagacaataagcaaactccagtaacaaactttccaaaaatttctgagccttgatttcttcatcttgtaaacagaaaaacacactcccgtaataataccacccaacctgccgcccacctgatggcgtcgccgcgagacgaaccactttgaaaacccgcgaaaccaaggtgaacaatccctgcgcaacatgacagtttggtcacatcgccgttctcactcgcgcttgttactgaaatctgaaaaatacttagtatttttcagatttcagtaacaagcgcgagtgagaacggcgatgtgaccaaactgtcatgttgcgcagggattgttcaccttggtttcgcgggttttcaaagtggttcgtctcgcggcgacgccatcaggtgggcggcaggttgggtggtattattacgggagtgtgtttttctgtttacaagatgaagaaatcaaggctcagaaatttttggaaagtttgttactggagtttgcttattgtctccgctcatggctcatcaaatagttgggattttggccaaaaaawrwktgct

SEQ ID NO: 161 - DNA, 2777 bp, Homo sapiens
  chr10102972869-102973961
ataaaataaggagaaaacgcaattcaattcagcaaatgtttattgcgcgtcttttctgcgccaggccgcgcaggggggctgggaaaatggaaacgaatcagattcggttcctgccctcaaggagctcctagtctagtgggcaagactcgcagcacccccagcgctagtagaagcggaccgagggacagggccgtgccagttgcaaaacactgagaggcagctaccagggtgttggcggcttcacagaggaggtggcttgggacccgggccttgaaaggcgaaaagtagttcgttgtttccataggccagagaagagagccagtggaaggaaggaccagagctgcggcggggggaaggggtaacgaagtgtacgtgagagcctcggctgaacccgagctaaggaggccacgcgagagcgccctgcgcgctcggtcccgcacaatgtgggaagtggattcgagcctctccgcagtgtcctctgctctgaggaattcgctgcctccatcaggttccgggtgggcttccggagcccagccgacagctctgttgaggccgcggactagcactggactctgcctggactagcacccgactctgcctggactagcacccgggcttggggcttatctctaggtcccattgcgcagggaggcggcgaggaagtcagcagctctgggcaaaagctgaggggcttggctgtaggcggaggcgaggatctgggatccagggaggcctgggctctaagcccactctctctttagctcccccagttctgaagaagtctgctacctgcgcgcggcccggggacctcggatggtcttggctgtgcctcaggcgctgccgctgggaccggcgatgtgagagagccacgaactacgccagaagaatgggcgtagggcccacagcccctcacacaacccaggccaagctccctcgtacactcagaccaggagaagaaacagacacgcaatacacaagccccgctcccttggagcctacacttgggcgaacacaccactgctgtttgcagtatcccgagacaggcccggcaggcgtgagctaagaggcctcgggcccgaacttgggatttgaggctctctcacatcgccggtcccagcggcagcgcctgaggcacagccaagaccatccgaggtccccgggccgcgcgcaggtagcagacttcttcagaactgggggagctaaagagagagtgggcttagagcccaggcctccctggatcccagatcctcgcctccgcctacagccaagcccctcagcttttgcccagagctgctgacttcctcgccgcctccctgcgcaatgggacctagagataagccccaagcccgggtgctagtccaggcagagtcgggtgctagtccaggcagagtccagtgctagtccgcggcctcaacagagctgtcggctgggctccggaagcccacccggaacctgatggaggcagcgaattcctcagagcagaggacactgcggagaggctcgaatccacttcccacattgtgcgggaccgagcgcgcagggcgctctcgcgtggcctccttagctcgggttcagccgaggctctcacgtacacttcgttaccccttccccccgccgcagctctggtccttccttccactggctctcttctctggcctatggaaacaacgaactacttttcgcctataaaataaggagaaaacgcaattcaattcagcaaatgtttattgcgcgtcttttctgcgccaggccgcgcaggggggctgggaaaatggaaacgaatcagattcggttcctgccctcaaggagctcctagtctagtgggcaagactcgcagcacccccagcgctagtagaagcggaccgagggacagggccgtgccagttgcaaaacactgagaggcagctaccagggtgttggcggcttcacagaggaggtggcttgggacccgggccttgaaaggcgaaaagtagttcgttgtttccataggccagagaagagagccagtggaaggaaggaccagagctgcggcggggggaaggggtaacgaagtgtacgtgagagcctcggctgaacccgagctaaggaggccacgcgagagcgccctgcgcgctcggtcccgcacaatgtgggaagtggattcgagcctctccgcagtgtcctctgctctgaggaattcgctgcctccatcaggttccgggtgggcttccggagcccagccgacagctctgttgaggccgcggactagcactggactctgcctggactagcacccgactctgcctggactagcacccgggcttggggcttatctctaggtcccattgcgcagggaggcggcgaggaagtcagcagctctgggcaaaagctgaggggcttggctgtaggcggaggcgaggatctgggatccagggaggcctgggctctaagcccactctctctttagctcccccagttctgaagaagtctgctacctgcgcgcggcccggggacctcggatggtcttggctgtgcctcaggcgctgccgctgggaccggcgatgtgagagagcctcaaatcccaagttcgggcccgaggcctcttagctcacgcctgccgggcctgtctcgggatactgcaaacagcagtggtgtgttcgcccaagtgtaggctccaagggagcggggcttgtgtattgcgtgtctgtttcttctcctggtctgagtgtacgagggagcttggcctgggttgtgtgaggggctgtgggccctacgcccattcttctggcgtagttcgt

SEQ ID NO: 162 - DNA, 1494 bp, Homo sapiens
  chr2031113089-31113596
actcaggtctccaaagccgcagtcaacaccacccccaagagaagggagaggagaaatccagagcccctagtgtccgcccgaccctgtcatctgggttgaaccgtccacctggcagccgcacccacatgtgtctccctctgctcaggctgctgcccacaccactctttggggtcgtggaacagatgctcttcaaggtgcttccgggactggtgagtgtgcgggccgtgtgccagcatgccctctcccaggactgggcctctttccccctctggttttgggtgaagtttggcgccaggcaggattaggctgtacttcccttccacacccacctggtaatcccatccgggcctgctgaccggccttcctggcgcgcctgctagtctgggggtggatgacacgatcctttcggcctgggtcccctgctcccaaatcgtaggctccttcttacccctgctctgtttctgccatttgtattagtaccgactttatgtttttcttaagaaaaacataaagtcggtactaatacaaatggcagaaacagagcaggggtaagaaggagcctacgatttgggagcaggggacccaggccgaaaggatcgtgtcatccacccccagactagcaggcgcgccaggaaggccggtcagcaggcccggatgggattaccaggtgggtgtggaagggaagtacagcctaatcctgcctggcgccaaacttcacccaaaaccagagggggaaagaggcccagtcctgggagagggcatgctggcacacggcccgcacactcaccagtcccggaagcaccttgaagagcatctgttccacgaccccaaagagtggtgtgggcagcagcctgagcagagggagacacatgtgggtgcggctgccaggtggacggttcaacccagatgacagggtcgggcggacactaggggctctggatttctcctctcccttctcttgggggtggtgttgactgcggctttggagacctgagtactcaggtctccaaagccgcagtcaacaccacccccaagagaagggagaggagaaatccagagcccctagtgtccgcccgaccctgtcatctgggttgaaccgtccacctggcagccgcacccacatgtgtctccctctgctcaggctgctgcccacaccactctttggggtcgtggaacagatgctcttcaaggtgcttccgggactggtgagtgtgcgggccgtgtgccagcatgccctctcccaggactgggcctctttccccctctggttttgggtgaagtttggcgccaggcaggattaggctgtacttcccttccacacccacctggtaatcccatccgggcctgctgaccggccttcctggcgcgcctgctagtctgggggtggatgacacgatcctttcggcctgggtcccctgctcccaaatcgtaggctccttcttacccctgctctgtttctgccatttgtattagtaccgactttatgtttttctt

SEQ ID NO: 163 - DNA, 95 bp, Homo sapiens
  chr2154892928-154892961
tagcagcaggatgatcaacatgatacaatattgtatcatgttgatcatcctgctgctagggtaccctagcagcaggatgatcaacatgatacaat

SEQ ID NO: 164 - DNA, 1080 bp, Homo sapiens
  chr2138886792-38887152
aggaaaaaatccctggtaaatgtgttttcttgaacagaaggctcagtgtaaagcaggagagaagtcatatttatagttgcttttactttcttgatgtcatccgcctttcaaatgctgtctgttttccttcagatctctcgtttttacagggggcccccaggcagaccctggttcccacactgagggacatggcgctgttttgctgctgtgtgttttgttctggtggaagctggcagccacctcaactctgagatcagctcaaatgctttacatgcaaaacggcaaggctgagaaagaactgaaagtttccttgggtccaagaacgtcttgggctttacaaactggtgcccacaggcacatatgtgcctgtgggcaccagtttgtaaagcccaagacgttcttggacccaaggaaactttcagttctttctcagccttgccgttttgcatgtaaagcatttgagctgatctcagagttgaggtggctgccagcttccaccagaacaaaacacacagcagcaaaacagcgccatgtccctcagtgtgggaaccagggtctgcctgggggccccctgtaaaaacgagagatctgaaggaaaacagacagcatttgaaaggcggatgacatcaagaaagtaaaagcaactataaatatgacttctctcctgctttacactgagccttctgttcaagaaaacacatttaccagggattttttcctaggaaaaaatccctggtaaatgtgttttcttgaacagaaggctcagtgtaaagcaggagagaagtcatatttatagttgcttttactttcttgatgtcatccgcctttcaaatgctgtctgttttccttcagatctctcgtttttacagggggcccccaggcagaccctggttcccacactgagggacatggcgctgttttgctgctgtgtgttttgttctggtggaagctggcagccacctcaactctgagatcagctcaaatgctttacatgcaaaacggcaaggctgagaaagaactgaaagtttccttgggtccaagaacgtcttgggctttacaaactggtgcccacaggcacat

SEQ ID NO: 165 - DNA, 60 bp, Homo sapiens
  Partial sequence of WDR18
ggtaccccggctctagagctactaactgatcgccggagaccctgggccttggaagaagtc

SEQ ID NO: 166 - DNA, 60 bp, Homo sapiens
  Partial sequence of RPL39
cccctccgaaaaattaaggagccgattgggcgggactaaccctaagggagggcaggggaa

(19) United States
(12) Patent Application Publication
(10) Pub. No.: US 2010/0234242 A1
(43) Pub. Date: Sep. 16, 2010

(54) DNA Methylation Changes Associated with Major Psychosis

(76) Inventors: Arturas Petronis, Toronto (CA); Jonathan Mill, Toronto (CA); James Flanagan, Toronto (CA); Sun-Chong Wang, Toronto (CA)

Correspondence Address: BRINKS, HOFER, GILSON & LIONE, P.O. BOX 1340, MORRISVILLE, NC, 27560, US

(21) Appl. No.: 12/524,114

(22) PCT Filed: Jan. 23, 2008
(86) PCT No.: PCT/CA08/00129

§ 371 Date: May 28, 2010

Related U.S. Application Data
(60) Provisional application No. 60/886,188, filed on Jan. 23, 2007.
(60) Provisional application No. 60/997,968, filed on Oct. 5, 2007.

(51) Int. Cl. C40B 30/06; C12Q 1/68; C40B 40/06; C07D 239/47; C07H 21/00

(52) U.S. Cl. 506/10; 435/6; 506/16; 544/317; 536/23.1

(57) ABSTRACT

The present invention provides a method of identifying one or more epigenetic markers associated with psychosis-associated diseases such as bipolar disease or schizophrenia, the method comprising a) obtaining a first group of samples comprising genomic DNA from a plurality of bipolar or schizophrenic subjects and a second group of samples comprising genomic DNA from a plurality of control subjects; b) performing DNA methylation analysis to determine methylation differences in one or more DNA regions between the first group and second group of samples, wherein a methylation difference in a DNA region is indicative of an epigenetic marker associated with bipolar disease or schizophrenia. The invention also provides one or more epigenetic markers associated with psychosis-associated diseases such as bipolar disease or schizophrenia.

### FIELD OF INVENTION

The present invention relates to identification of epigenetic abnormalities. More particularly, the present invention relates to diagnosis of diseases based on DNA methylation differences, and identification and isolation of nucleotide sequences variable modification of which are associated with such diseases.

### BACKGROUND OF THE INVENTION

Epigenetics refers to the regulation of various genomic functions that are controlled by partially stable modifications of DNA and chromatin proteins, which are critical to the proper functioning of the genome. Recent evidence suggests that epigenetic signals can pass from one generation to the next, contrary to the previous belief that epigenetic signals were lost during fertilisation and early development.

Phenotypic differences between individuals have traditionally been attributed to genetic (DNA sequence) variation and environmental differences. Over the last several decades, documentation of DNA sequence variants has been one of the top priorities in biomedical research. Numerous major international projects including the Human Genome sequencing project (1,2) the creation of single nucleotide polymorphisms (SNP) databases (dbSNP, now called Entrez SNP) and the Haplotype Map3 have contributed significantly to the understanding of the position, degree, and structure of DNA polymorphisms. However, SNPs and other DNA sequence differences are relatively rare, and DNA sequences of two unrelated individuals can exhibit 99.5% identity. Furthermore, only a small fraction of these polymorphisms are functional (i.e. polymorphisms that change amino acid sequence in the protein or have an impact on gene expression). Sequencing of chimpanzee's (pan troglodytes) genome revealed 98.67% DNA sequence identity to the human genome, and again, only a fraction of such polymorphisms appear to result in structural or functional gene differences (4). Such findings raise the question as to whether the low DNA sequence variation across unrelated individuals and our closest related species sufficient to account for all major differences in physiological and psychological phenotypic outcomes.

One potential, although poorly investigated, source of phenotypic differences is epigenetic variation. Epigenetics refers to the regulation of various genomic functions that are controlled by partially stable modifications of DNA and chromatin proteins (see, for example (5)). Epigenetic signals are required for the proper functioning of the genome, as seen in Dnmtl knockout mice that die in early embryogenesis (6), several rare pediatric syndromes, and cancer (7). One important feature of epigenetic regulation is the partial epigenetic stability, or metastability. Epigenetic profiles in different cells of the same organism can be quite different and developmental programs, environmental factors or stochastic events in the nucleus of a cell, can induce this variation. The first systematic effort to document DNA methylation differences and similarities across different genome regions has been recently launched and called the Human Epigenome Project. The pilot study of the MHC locus on chomosome 6 investigated seven cell types (adipose, brain, breast, lung, liver, prostate, and muscle) across 32 individuals (8). In this study only 5% (14/253 amplicons) of the tested loci showed significant inter-individual variability. The Human Epigenome Project as well as other smaller scale studies have primarily investigated epigenetic variation in somatic cells. However, there has been very little effort to document epigenetic variation in the germline, apart from imprinted genes (9,10) and isolated cases of germ cell epimutations (11,12).

There are several reasons to believe that the germline may contain substantial epigenetic variation. Epigenetic reprogramming during gametogenesis, fertilization, and embryogenesis involves dramatic chromatin remodelling (13). Methylation reprogramming during gametogenesis involves the erasure and reestablishment of methylation of imprinted genes and other non-imprinted genes and then a second wave of reprogramming during fertilization (paternal) and embryogenesis (maternal) (13). This process is thought to ensure that both gametes acquire the appropriate sex-specific epigenetic state and establish the epigenetic state required for early embryonic development and toti- or pluripotency, and in addition allow the erasure of epimutations that adult germ cells may have inherited or developed during their lifetime (14,15). In parallel to DNA methylation, chromatin changes during spermatogenesis involve the compaction of the haploid genome by replacement of the core histones through transition proteins to the much smaller basic protamines 1 and 2 (16). However, a number of testis-specific histones and histone variants, such as TSH2B, histones H2A, H3 and H4, variants of H2B and CENP-A, are present to some extent in the mature spermatozoa (17-19). How these remaining histones are arranged and to what extent inter-individual variability in histone placement and modification can affect development and phenotype is yet to be investigated. Despite dramatic changes, not all epigenetic signals are erased in the germline, and recent studies in mice have suggested that this phenomenon could underlie epigenetic inheritance (20,21). Therefore, there is ample opportunity during these phases of reprogramming to either maintain or generate substantial epigenetic variability in the germ cells.

Substantial progress has been made in recent years with respect to the diagnosis and treatment of diseases in which a single defective gene is responsible. Traditional linkage studies have effectively isolated the causal gene and allowed for the further development of diagnostic tests and furthered research into treatments such as gene therapy for conditions such as cystic fibrosis, Duchennes muscular dystrophy, Huntington's disease and fragile X syndrome. However, similar progress has not been made in complex diseases caused by mutations in multiple genes. Traditional linkage studies in complex diseases has only succeeded in isolating chromosome regions that contain several hundred genes. The ability to screen such a large number of genes is clearly a time consuming and daunting task.

There is a need in the art to identify nucleotide sequences associated with psychosis-associated diseases, for example, but not limited to bipolar disorder and schizophrenia. There is also a need in the art to identify epigenetic nucleotide sequences that are differentially methylated in diseases states such as bipolar disorder and schizophrenia.

### SUMMARY OF THE INVENTION

The present invention relates to detection of epigenetic abnormalities and diagnosis of diseases associated with epigenetic abnormalities, and identification and isolation of nucleotide sequences that are associated with such diseases.

According to the present invention there is provided a method of identifying one or more epigenetic markers associated with psychosis-associated diseases, for example, but not limited to schizophrenia or bipolar disease, the method comprising,

a) obtaining a first group of samples comprising genomic DNA from a plurality of subjects having a psychosis-associated disease and a second group of samples comprising genomic DNA from a plurality of control subjects;

b) performing DNA methylation analysis to determine methylation differences in one or more DNA regions between the first group and second group of samples, wherein a methylation difference in a DNA region is indicative of an epigenetic marker associated with psychois associated disease, for example, but not limited to schizophrenia or bipolar disease.

The present invention also provides a method as defined above, wherein the DNA methylation analysis is DNA microarray analysis. However, other types of DNA methylation analysis alone or in combination with microarray analysis may be used in the method of the present invention.

Also provided by the present invention is a method as described above, wherein the samples are blood, brain, sperm or any other tissue or sample that provides genomic DNA.

The present invention also provides a method of as defined above, wherein DNA microarray analysis comprises hybridization of differentially epigenetically modified DNA from each subject of said first and second groups to a genomic microarray.

The present invention further contemplates a method as defined above, wherein the differences comprise hypermethylation differences, hypomethylation differences or both.

Also provided by the present invention is a method as defined above wherein said step of performing identifies a set of epigenetic markers, the set providing an increased correlation of association with psychosis-associated disease, for example, bipolar disorder or schizophrenia as compared to a single epigenetic marker.

The present invention also provides a method as defined above that further comprises identifying one or more genes associated with the epigenetic markers.

Also provided by the present invention is a method of determining the risk of a subject having or developing a psychosis-associated disease, for example, but not limited to bipolar disorder or schizophrenia comprising,

a) obtaining a genomic DNA sample from the subject,

b) determining the methylation status of one or more epigenetic markers in the genomic DNA sample from the subject, and;

c) comparing the methylation status of said one or more epigenetic markers to the methylation status of a control group of epigenetic markers associated with one or more psychosis-associated diseases, for example, but not limited to bipolar disorder or schizophrenia, wherein similar or identical methylation profiles are indicative of an increased risk of having or developing psychosis-associated diseases, for example bipolar disorder or schizophrenia.

The present invention also provides one or more epigenetic markers associated with bipolar disease or schizophrenia. In a preferred embodiment, but not wishing to be limiting, the epigenetic markers are identified by a method as defined above.

The present invention also provides one or more markers associated with bipolar disease or schizophrenia, wherein each of said one or more markers comprises a methylated cytosine.

The present invention also provides a nucleotide sequence array comprising one or more epigenetic markers associated with bipolar disease or schizophrenia. In a further embodiment, which is not meant to be limiting in any manner, the present invention provides one or more nucleotide sequence arrays, wherein each array consists of a plurality of markers associated with bipolar disease or schizophrenia.

In a further embodiment, the present invention provides a set of epigenetic markers associated with bipolar disease or schizophrenia, the markers comprising a plurality of nucleotide sequences that are differentially epigenetically modified and that are positively associated with bipolar disorder or schizoprenia.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1. A. Intra-individual variability of DNA methylation. A. DNA methylation profiles of the promoter CpG islands of BRCA1 and PSEN2 determined based on sequencing 60 clones of bisulphite modified sperm DNA. BRCA1 locus covered 32 CpGs PSEN2 region included 45 CpGs. Nine monomorphic (unmethylated) CpGs (BRCA1 or PSEN2) were excluded from the figure. Each individual is represented with individual CpG dinucleotides, left to right (black-methylated cytosines, white-unmethylated cytosines), and individual clones, top to bottom. Like the presented BRCA1 and PSEN2 cases, a substantial proportion of clones in other loci (HD, DM1, BRCA2, and PSEN1) revealed unique DNA methylation profiles. B. Estimates of the proportion of unique methylation profiles in the promoter regions of the six analyzed genes. Y axis: proportion of clones carrying unique methylation profiles over the total number of sequenced clones; X axis: proportion of unique profiles that contain at least 1, 2, and 3 differences (left, middle, and right bar, respectively) compared to the other profiles at the same locus of the same individual.

FIG. 2. Inter-individual variability of DNA methylation in 6 human disease genes. Bisulphite modification-based mapping of methylated cytosines in BRCA1, BRCA2, HD, DM1, PSEN1, and PSEN2. 30 individual clones were sequenced from 3 to 7 individuals. Analysis for each gene is represented in two panels. The left panels are a graphical profile of the percentage of methylation (Y-axis, ranging from 0%- 40%) for every CpG dinucleotide (X-axis, ranging from 32 to 108 CpG dinucleotides) out of the total number of clones for each individual. The right panels represent Euclidean distances (Y-axis) of pairwise comparisons between individual methylation profiles (X-axis). The blue line (middle straight horizontal line) is the mean distance and red lines (upper and lower straight horizontal lines) are ±2SD from the mean, both obtained for each gene from the permutation study (see Examples). Pair-wise comparisons are annotated as e.g. “16” for the comparison of the Euclidian distance of individual 1 with individual 6. Primed individual numbers (e.g. 4′) represent a second set of 30 clones from those individuals. The error bars on some data points represent standard deviation from 100,000 permutations of 30 clone groups from the individuals where 60 clones were sequenced.

FIG. 3. Chromosomal view of methylation variability by CpG Island microarray analysis. A. The unmethylated fraction of genomic DNA extracted from sperm samples (N=21) was hybridized individually (Cy5) versus the pooled reference control (Cy3). The coefficient of variance (CV) of Cy5/Cy3 ratio was calculated for each spot across the 21 individuals and mapped to the corresponding genomic location. Each chromosome ideogram is overlaid with red bars representing the position of each clone on the CpG island microarray. The bars highlighted in green are the loci that showed 90th percentile of variance (the top 10% of loci exhibiting the largest degree of DNA methylation variation) B. Screenshot of the custom annotation track on the USCS genome browser (available at http://www.epigenomics.ca). Shown is Chr 6, which includes the major histocompatibility complex locus that was screened for epigenetic variability by the Human Epigenome Project pilot study.

FIG. 4. Age related DNA methylation changes in the sperm. Individuals were ordered with increasing age (top left panel) and gene specific DNA methylation dynamics was investigated using the individual ages (sperm DNA-HpaII: age range 24-56 yr) as a covariate. Pearson correlation was calculated for each locus, and the one-tailed p-value of the coefficient was obtained. 105 loci were identified in the sperm DNA-HpaII data set as significantly (p<0.05) correlated (r>0.5) or inversely correlated (r<−0.5) with age. Since unmethylated fraction of DNA was interrogated, positive correlation indicates decreasing DNA methylation with age, while inverse correlation reflects increasing methylation with age. The genes CTNNA2, EED, CALM1, CDH13 and STMN2 are shown as examples. Other genes for the sperm DNA-HpaII dataset and the sperm DNA-HHA and brain DNA-HpaII data sets are presented herein.

FIG. 5. Repetitive element analysis in sperm DNA-HpaII and brain DNA -HpaII data sets. A. The microarray loci that contain a single repetitive element were separated into each repeat class and the mean CV (+/±SD) was calculated. The repeat classes include DNA transposons (N=209), LINEs (N=771), low complexity repeats (N=461), long terminal repeats −LTRs (N=360), satellites (N=208), simple repeats (N=346), SINEs (N=1058), snRNA (N=30) and tRNA (N=40) and the non-repetitive loci (N=6976) are presented for comparison. The satellite repeats were the only class to show significantly increased variability in the sperm DNA-HpaII (p=6.12E−17) and less significantly in the brain DNA-HpaII (p=0.0027) data sets. B. The breakdown of the satellite repeats into specific satellite repeat classes revealed a number of repeat classes with increased variability, predominantly the centromeric satellite repeats, including (GAATTC)n (p=8.44E−17, N=55), ALR/Alpha (p=4.08E−25, N=119), CER (p=0.0026, N=6) and HSATII repeats (p=3.91E−5, N=19), but not in BSR/Beta repeats (p>0.05, N=7).

FIG. 6. Methylation sensitive single nucleotide primer extension (MS-SNuPE) analysis of densities of methylated cytosines in CpG dinucleotides of selected genes. Genomic DNA from each of 11 individuals was treated with sodium bisulphite and then PCR amplified for each gene. The genes NELL2, SCAM1, NEIL2, MKL2, CDH13 and OLR1 are represented. The methylation status of CpG dinucleotides within each of the restriction enzyme sites was interrogated using the primer extension reactions. Methylation of each of the CpG dinucleotides is represented as a percentage of methylated PCR products: completely unmethylated (white circles), partially methylated (half circles) or completely methylated (black circles).

FIG. 7. Methylation profiles of CDH13. Methylation status of 16 CpG sites surrounding CDH13 C/G SNP across 30 clones sequenced in each of five tested individuals. 77% (67/87) of the G alleles are methylated (4 or more methylated CpGs) while 78% (49/63) of the C (bisulphite converted to T) alleles are unmethylated. The first seven CpG dinucleotides interrogated by MS-SnuPE in FIG. 6 are represented in this figure as CpGs #5, 6, 9, 10, 13, 15, and 16. CpG 9 is the third MS-SnuPE primer that was predominantly unmethylated in all individuals. Each individual is represented with single CpG dinucleotides, left to right (black-methylated, white-unmethylated), and individual clones, top to bottom.

FIG. 8. Chromosomal view of brain-HpaII (A) and sperm-HHA (B) data sets. The unmethylated fraction of genomic DNA enriched from brain DNA (N=22) or sperm samples (N=25) and each was hybridized individually (Cy5) versus the pooled samples (Cy3). The coefficient of variance (CV) of ratio Cy5/Cy3 was calculated for each spot across the 22 or 25 individuals and mapped to the corresponding genomic location. Each chromosome ideogram is overlaid with red bars representing the position of each clone on the array. The bars highlighted in green are the loci that showed statistically significant variance (90th percentile).

FIG. 9 shows a Volcano plot of BP Case vs Control t-test significance (FDR corrected p-value) plotted on the y-axis against the BP Case fold-change on the X-Axis.

FIG. 10 shows results of microarray loci suggesting that ERBB4 has significantly higher methylation than controls.

FIG. 11 shows the nucleotide sequences of loci identified by the method of the present invention.

FIG. 12 shows volcano plots illustrating differential methylation profiles of psychosis subjects versus unaffected individuals.

FIG. 13 shows a volcano plot illustrating the differential methylation profile of bipolar disorder subjects versus unaffected individuals.

FIG. 14 shows results confirming methylation differences in two genes nominated from microarray analysis.

FIG. 15 shows categories for various diagnostic groups.

FIG. 16 shows graphical results of partial correlation network analysis illustrating connection between nodes.

FIG. 17 shows DNA methylation differences associated with psychosis.

FIG. 18 shows correlation between DNA methylation in the promoter region of a protein kinase and lifetime antipsychotic use in schizophrenia samples.

FIG. 19 shows verification of microarray-based DNA methylation profiling results.

FIG. 20 shows network analysis of DNA methylation microarray data.

FIG. 21 shows the association of BDNF genotype with DNA methylation at nearby exonic CpG sites.

### DETAILED DESCRIPTION

The following description is of a preferred embodiment.

According to an embodiment of the present invention, there is provided a method of identifying one or more epigenetic markers associated with a psychosis-associated disease, the method comprising,

a) obtaining a first group of samples comprising genomic DNA from a plurality of subjects having psychosis-associated disease and a second group of samples comprising genomic DNA from a plurality of control subjects;

b) performing DNA methylation analysis to determine methylation differences in one or more DNA regions between the first group and second group of samples, wherein a methylation difference in a DNA region is indicative of an epigenetic marker associated with psychosis-associated disease.

In a preferred embodiment, which is not meant to be limiting in any manner, the psychosis-associated disease is bipolar disorder or schizophrenia.

In the context of the present invention, by the term “epigenetic marker” it is meant a nucleotide sequence that is differentially epigenetically modified in psychosis-associated disease, for example, but not limited to bipolar disorder or schizophrenia, as compared to the nucleotide sequence in a normal or control state. The epigenetic marker may be hypermethylated or hypomethylated in the disorder or disease state relative to the normal or control state. In general, the epigenetic marker comprises between about 5 and about 10000 nucleotides, for example, but not limited to 5, 7, 9, 11, 15, 17, 21, 25, 50, 75, 100, 200, 300, 400, 500, 600, 700, 800, 900 or 1000, 2000, 3000, 4000, 5000, 6000, 7000, 8000, 9000, 10000 nucleotides, or any amount therein between. Further, the epigenetic marker may comprise a range of sizes as defined by any two of the values listed or any two amounts therein between.

As used herein, the term DNA methylation refers to the addition of a methyl group to the cyclic carbon 5 of a cytosine nucleotide. A family of conserved DNA methyltransferases catalyzes this reaction.

By the term “DNA methylation analysis” it is meant any technique, method or combination thereof that may be employed to determine methylation differences between samples comprising genomic DNA. Such techniques also may be employed to determine methylation profiles of nucleotide sequences, for example information such as, but not limited to methylation status of a plurality of nucleotides over a specified DNA region. The methods and techniques may comprise, but are not limited to:
methylation-sensitive restriction enzymes, for example, but not limited to as described in Issa J. P., et al. (1994) Nature Genetics 7:536-40. The terms “restriction endonucleases” and “restriction enzymes” refer to bacterial enzymes, each of which cut double stranded DNA at or near a specific nucleotide sequence. The process of cutting or cleaving the DNA is referred to as restriction digestion. The products of a restriction digestion are referred to as restriction products. A restriction enzyme used in the present invention may yield restriction products having blunt-ends or overhanging “sticky” ends. Specifically, a restriction enzyme can symmetrically cut both strands of a double stranded DNA fragment to produce a blunt-ended fragment, or a restriction enzyme may assymetrically cleave the two strands of a DNA fragment to produce a DNA fragment that has a single stranded overhang. In general, a methylation-sensitive restriction enzyme used in the present invention will recognize and cleave a non-methylated sequence, while it will not cleave a corresponding methylated sequence. Methylation of plant and mammalian DNA occurs at CG or CNG sequences. This methylation may interfere with the cleavage by some restriction endonucleases. Endonucleases that are sensitive and not sensitive to m5CG or mSCNG methylation, as well as isoschizomers of methylation-sensitive restriction endonucleases that recognize identical sequences but differ in their sensitivity to methylation, can be extremely useful for studying the level and distribution of methylation in eukaryotic DNA. Examples of methylation-sensitive restriction enzymes, and corresponding restriction site sequences, that can be used according to the present invention include, but are not limited to: AatII (GACGTC); Bsh1236I (CGCG); Bsh1285I (CGRYCG); BshTI (ACCGGT); Bsp68I (TCGCGA); Bsp119I (TTCGAA); Bsp143II (RGCGCY); Bsu15I (ATCGAT); Cfr10I (RCCGGY); Cfr42I (CCGCGG); CpoI (CGGWCCG); Eco47III (AGCGCT); Eco52I (CGGCCG); Eco72I (CACGTG); Eco105I (TACGTA); EheI (GGCGCC); Esp3I (CGTCTC); FspAI (RTGCGCAY); Hin1I (GRCGYC); Hin6I (GCGC); HpaII (CCGG); Kpn2I (TCCGGA); MluI (ACGCGT); NotI (GCGGCCGC); NsbI (TGCGCA); PauI (GCGCGC); PdiI (GCCGGC); Pfl23II (CGTACG); Psp1406I (AACGTT); PvuI (CGATCG); SalI (GTCGAC); SmaI (CCCGGG); SmuI (CCCGC); TaiI (ACGT); or TauI (GCSGC).
methylation-sensitive arbitrarily primed PCR (Liang G, et al. (2002) Identification of DNA methylation differences during tumorigenesis by methylation-sensitive arbitrarily primed polymerase chain reaction. Methods 27(2):150-5);
sequencing of sodium bisulfite-induced modifications of genomic DNA (Frommer M, et al. (1992) A genomic sequencing protocol that yields a positive display of 5-methylcytosine residues in individual DNA strands);
methylation-specific PCR based on differential hybridization of PCR primer to DNA initially modified by bisulfite treatment (Herman J G, et al. (1996) Methylation-specific PCR: A novel PCR assay for methylation status of CpG islands. Proc Natl Acad Sci USA 93:9821-26; Fan X, et al. (Improvement of the methylation specific PCR technical conditions for the detection of p16 promoter hypermethylation in small amounts of tumor DNA. Oncology Rep 9:181-3); or
methylation-sensitive single nucleotide primer extension based on bisulfite-modification of DNA followed by differential incorporation of labelled nucleotides to a primer that is designed to hybridise immediately upstream of a methylation site (Gonzalgo and Jones (1997) Rapid quantitation of methylation differences at specific sites using methylation-sensitive single nucleotide primer extension (Ms-SNuPe) Nucleic Acids Research 25:2529-31).

In a preferred embodiment, the DNA methylation analysis comprises microarray analysis.

Methylation of genomic sequences can be determined by using both methylation-sensitive restriction enzyme analysis, and genomic sequencing. Various restriction enzymes are available that digest demethylated sequences, while leaving methylated sequences intact. An advantage of methylation-sensitive restriction enzyme analysis is that it produces DNA fragments that have 5′ and 3′ ends that were demethylated at the time of digestion. As a result it is a quick method of localizing demethylated sequences within a particular restriction sequence within a larger DNA sequence, such as a locus, chromosome, or even a whole genome. Methylation-sensitive restriction enzyme analysis, as well as examples of various methylation-sensitive restriction enzymes, are described in greater detail below.

Methylation-sensitive DNA sequencing, while not as quick a method as restriction enzyme analysis, can provide specific sequence information with regards to any methylation site, regardless of its inclusion within a restriction enzyme site. Maxam and Gilbert chemical cleavage sequencing protocols have been modified and developed to determine methylation status of sequences within a gene, with the absence of a band in all tracks of a sequencing gel indicating the presence of a 5-methylcytosine residue (Church and Gilbert (1984) Proc Natl Acad Sci USA 81:1991-95; Saluz and Jost (1989) Proc Natl Acad Sci USA 86:2602-6; Pfeifer GP, et al. (1989) Science 246:810-13).

Another method of methylation-sensitive DNA sequencing involves exposing genomic DNA to sodium bisulfite (Frommer M, et al. (1992) A genomic sequencing protocol that yields a positive display of 5-methylcytosine residues in individual DNA strands) under conditions where cytosine residues are converted to uracil residues, while 5-methylcytosine residues remain nonreactive. One or both strands of the bisulfite-modified genomic DNA can then be PCR amplified using pairs of strand specific primers. As the bisulfite reaction protocol produces single DNA strands that can no longer achieve 100% complementary basepairing (for example reacting double stranded DNA consisting of 5′-TCTC-3′ base paired to 5′-GAGA-3′ with sodium bisulfite yields single strands of 5′-TUTU-3′ and 5′-GAGA-3′ such that 100% complementary base pairing can no longer be achieved), pairs of PCR primers can be designed such that they anneal in a strand-specific fashion and produce PCR products for each of the single bisulfite-modified DNA strands. The PCR products can then be subject to any combination of assays available to skilled persons including, without limitation, sequencing, cloning, methylation-specific PCR, Ms-SNuPe, or microarrays. Bisulfate-modified DNA templates can be conveniently produced using the EZ DNA methylation Kit™ developed by Zymo Research.

The combination of methylation-specific technology and array technology may be particularly useful for high throughput applications. For example, but not wishing to be limiting, fragments of bisulfite-modified DNA could be analysed using microarrays having probes that were specific for identified hypomethylated sequences. As another example, an array of primers could be developed for analysing each potential demethylation site by Ms-SNuPe assay within a DNA sequence, such as a locus, chromosome, or even a whole genome.

The techniques as described above may be employed in the methods of the present invention as described herein and throughout. Further, the techniques and methods as described can also be used in diagnosis of psychosis-associated diseases such as, without limitation, bipolar disorder or schizophrenia. For example, but not to be considered limiting in any manner, once one or more than one hypo- or hyper-methylated sequence has been correlated with a disease state, DNA obtained from a subject having the disease can be treated with sodium bisulfite, followed by Ms-SNuPe or methylation-specific PCR using primers that are specific for the correlated hypo- or hyper-methylated sequence(s). As another example, diagnosis of disease can be achieved by digesting DNA, from a diseased sample, with a methylation-sensitive restriction enzyme that yields a different size fragment when digesting DNA from a diseased sample compared to DNA obtained from a normal sample; determination of the disease-specific restriction fragment size can be achieved through any standard method including, Southern analysis.

It will be understood that diagnostic methods of the present invention may be used to identify or confirm bipolar disorder or schizophrenia in a subject, or may be used to identify a predisposition of a subject to develop bipolar disorder or schizophrenia. As such, the diagnostic methods of the present invention encompass pre-diagnosis of bipolar disorder and/or schizophrenia.

Accordingly, the present invention is also directed to a method of determining the risk of a subject having or developing a psychosis-associated disease, for example, but not limited to bipolar disorder or schizophrenia comprising a) obtaining a genomic DNA sample from the subject; b) determining the methylation status of one or more epigenetic markers in the genomic DNA sample from the subject, and; C) comparing the methylation status of the one or more epigenetic markers in the subject to the methylation status of a control group of epigenetic markers associated with a psychosis-associated disease, for example, bipolar disorder or schizphrenia wherein the presence of one or more epigenetic markers having substantially similar or identical methylation profiles are indicative of an increased risk of the subject having or developing a psychosis-associated disease, for example, bipolar disorder or schizophrenia.

The strength of correlation between the presence of a particular epigenetic marker with a particular methylation profile and bipolar disorder or schizophrenia may vary. The strength of correlation can be expressed in terms of percentage of true positives (the number of people who develop bipolar disease or schizophrenia divided by the number of people who test positive for the marker). The diagnostic methods of the present invention can be successfully used in cases where strength of correlation between disease and epigenetic marker is lower than 100%, and could be as low as 50%, 40%, 30% or 20%, or even lower. The strength of correlation that is required for successful use of the diagnostic methods of the invention may depend on several factors that can be ascertained by persons skilled in the art, one of these factors being the strength of correlation provided by diagnostic methods that are available in the marketplace. For example, in a disease where no diagnostic method is currently available the diagnostic methods of the present invention may be useful even if providing a strength of correlation that is lower than 20%. Persons skilled in the art will recognize, that strength of correlation may include other factors in addition to the percentage of true positives, for example, a percentage of false positives (the number of people who do not develop a disease divided by the number of people who test positive). Again, as was the case for the desired percentage of true positives, the percentage of false positives that can be tolerated may depend on the number of false positives being generated by commercially available diagnostic methods.

Any biological sample that provides genomic DNA may be employed in the methods of the present invention. For example, but not to be considered limiting in any manner, samples that comprise genomic DNA may be derived from epithelial tissue, exocrine gland, endocrine gland, connective tissue, adipose tissue, cartilage, bone, blood, muscle tissue comprising smooth, skeletal or cardiac muscle tissue, nervous tissue comprising, but not limited to brain tissue, sperm and the like. Samples comprising germ cell genomic DNA are particularly preferred if additional information concerning the ability of the epigenetic sequence to transmit from one generation to the next is desired.

DNA can be extracted from the samples using standard techniques, known in the art, for isolating DNA from various samples such as cells, tissues, or organs, or other suitable specimens. Standard techniques for isolating DNA have are disclosed in reference textbooks or manuals such as, but not limited to Sambrook, Fritsch, and Maniatis, Molecular Cloning: A Laboratory Manual (1989), Cold Spring Harbor.

The method as defined above may further comprise the step of identifying one or more genes associated with the epigenetic marker(s), for example, but not limited to one or more genes upstream and/or downstream of the epigenetic marker(s). Techniques for analysing expression profiles of surrounding genes including, but not limited to, Northern, ELISA, reporter construct assays, microarray assay of RNA levels, dot blots, quantitative PCR, are well known to persons skilled in the art. Any number of standard and available techniques may be used to determine the genes proximal to an epigenetic marker.

The present invention also provides one or more epigenetic markers associated with psychosis-associated diseases, for example, but not limited to bipolar disorder or schizophrenia. In a preferred embodiment, but without wishing to be limiting, the markers are identified by the method as described herein.

The present invention also provides one or more epigenetic markers associated with a psychosis-associated disease, for example, but not limited to bipolar disease, or schizophrenia wherein each of said one or more markers comprises at least one methylated cytosine. In an alternate embodiment, but not to be limiting, it is also contemplated that the one or more epigenetic markers may not comprise a methylated cytosine.

The present invention also provides a nucleotide sequence array comprising one or more epigenetic markers associated with a psychosis-associated disease, for example, bipolar disease or schizophrenia. In a further embodiment, which is not meant to be limiting in any manner, the present invention provides one or more nucleotide sequence arrays, wherein each array consists of a plurality of epigenetic markers associated with bipolar disease or schizophrenia.

In a further embodiment, the present invention provides a set of epigenetic markers associated with a psychosis-associated disease, for example, but not limited to bipolar disease or schizophrenia, the markers comprising a plurality of nucleotide sequences that are differentially epigenetically modified and that are positively associated with the the psychosis-associated disease. In a preferred embodiment, the set of epigenetic markers provides an improved correlation of association with bipolar disorder and/or schizophrenia as compared to a single epigenetic marker.

The present invention will be further illustrated in the following examples.

We have shown that there is large epigenetic variation in germ cells and propose without wishing to be bound by theory or limiting in any manner, that this variation may be required if epigenetic signals are involved in heritable diseases. Inherited epigenetic misregulation of genes, or epimutations, may contribute in whole or in part to the unexplained heritability of many non-medelian diseases. An epimutation could occur in any gene as a germline event that predisposes an individual to disease and can be transmitted to offspring. Such an epimutation, for example, but not limited to, methylation of a specific disease-related gene promoter may be present in the mature sperm of an affected individual or “carrier”.

### Example 1

### Intra and Inter-Individual Epigenetic Variation in Human Germ Cells

The intra- and inter-individual epigenetic variation detectable in mature sperm of healthy individuals was estimated. For this, two different laboratory strategies were employed. The first approach focussed on promoter regions of several disease related genes, such as PSEN1, PSEN2, BRCA1, BRCA2, DM1 and HD, in healthy individuals, using bisulphite modification based mapping of methylated cytosines, and measured epigenetic “distances” between individuals. The second strategy was to perform a microarray-based epigenetic profiling of sperm DNA using a CpG island microarray, which provides genome-wide information on methylation variability across different unique and repetitive DNA sequences. Several loci of interest identified in the microarray experiments were further investigated using methylation sensitive single nucleotide polymorphism extension reaction (MS-SNuPE).

Materials and Methods

Samples

Two sperm sample sets were used in this study. The first sample set was received from the Fairfax Cryobank, Genetics & IVF Institute (Fairfax, Virginia) and consisted of 25 sperm samples from healthy Caucasian sperm donors with an average age of 27 yr (22-35 yr.). The second set of sperm samples was collected at the Centre for Addiction and Mental Health (Toronto, Canada) from 21 healthy Caucasian individuals with an average age of 39 yr (24-56 yr.). This study was approved by an institutional ethics board, and informed consent was obtained for all participants. Some aspects of sperm DNA data analysis required a non-sperm tissue of reference, and for this purpose post-mortem brain tissues were used. These brain samples were from 22 Caucasian males with an average age at death of 46 yr (31-66 yr). Extraction of DNA was performed using standard salt and phenol/chloroform extraction as known in the art.

Bisulphite Modification-Based Mapping of Methylated Cytosines

Bisulphite modification-based mapping of methylated cytosines was performed as described (22). Briefly, genomic DNA (700 ng) was digested with BglII (Fermentas) for 1 hour at 37° C., denatured at 100° C. for 5 min, chilled on ice, and then incubated at 50° C. for 15 minutes in 0.3M NaOH. The DNA was then mixed with 2% LMP agarose (SeaPlaque Agarose, FMC) and dropped into ice-cold mineral oil to form 7 beads of approximately 10 μL and finally the beads were placed into a freshly prepared solution containing 2.5 M sodium bisulphite (pH 5.0) plus 1 mM hydroquinone (both from Sigma). The beads were then incubated on ice for 30 minutes followed by incubation at 50° C. for 3.5 hrs. The beads were washed in four changes of TE (pH 8.0) for one hour and then desulphonated in 0.2 M NaOH for 30 min. Following desulphonation the beads were washed a second time in three changes of TE for 30 min. Prior to amplification the beads were washed in H₂O for 30 mins. PCR amplification of the target sequences consisted of 5 μL of agarose beads containing the bisulphite treated DNA, 2 mM MgCl₂, 0.2 mM dNTPs, 0.4 μM each of forward and reverse primer, 250 ng/mL BSA, 2.5 U Taq polymerase (New England Biolabs) in 1× PCR buffer to a total volume of 50 μL. PCR was performed using either a semi-nested or fully nested approach with the first PCR consisting of one cycle of 97° C. for 4 min, 53° C. for 2 min and 72° C. for 2 minutes, followed by 24 cycles of 94° C. for 45 s, 53° C. for 1 min and 72° C. for 1 min The second PCR used 5 μL of the first PCR as template and consisted of one cycle of 97° C. for 2 min, 53° C. for 2 min and 72° C. for 1 min, followed by 24 cycles of 94° C. for 45 sec, 55° C. for 45 sec and 72° C. for 1 min. CpG islands in the 5′ promoter sequences were analyzed in 6 genes: PSEN1 (Entrez gene GeneID:5663; chr14:72,672,525-72,673,163), PSEN2 (GeneID:5664; chr1:223,365,273-223,365,990), BRCA1 (GeneID:672; chr17:38,530,561-38,531,181), BRCA2 (GeneID: 675; chr13:31,787,367-31,788,153), HD (GeneID: 3064; chr4:3,113,281-3,113,816), DM1 (GeneID: 1760; chr19:50,964,670-50,965,254). An intronic CpG island within the CDH13 gene was also analysed by bisulphite genomic sequencing (GeneID: 1012; chr16:81,218,597-81,218,988). Nucleotide positions are according to May 2004 Genome (hg17) version.

The primers used for amplification of bisulphite modified DNA fragments were:

### For BRCA1:

| | |
|---|---|
| prBRCA1_for | (5′-gagtagaggttagagggtaggt-3′), |
| prBRCA1_rev1 | (5′-caaaacatattccaattcctatcac-3′), |
| prBRCA1_rev2 | (5′-tcaatacccccaacctaatcctc-3′). |

### For BRCA2:

| | |
|---|---|
| prBRCA2_for | (5′-gggggatttggagtaggtatagg-3′), |
| prBRCA2_rev1 | (5′-cacttccccaaaacaacaatattcc-3′), |
| prBRCA2_rev2 | (5′-aacccactaccaccaccactaacc-3′). |

### For PSEN1:

| | |
|---|---|
| prPSEN1for_1 | (5′-gatttattgagtggtgggagag-3′), |
| prPSEN1for_2 | (5′-tgataggtgttaaatttaggatgg-3′), |
| prPSEN1rev | (5′-cccctcatatttaaaacacc-3′). |

### For PSEN2:

| | |
|---|---|
| prPSEN2_for | (5′-ggggtggagagaggagagtgt-3′), |
| prPSEN2_rev1 | (5′-aaatacaattacttcactcaacacc-3′), |
| prPSEN2_rev2 | (5′-aactctataacctcaatttatcatc-3′). |

### For HD:

| | |
|---|---|
| prHDfor_1 | (5′-ggttattggttagttattggtagag-3′), |
| prHDfor_2 | (5′-gtaggttagggttgttaattatgttgg-3′), |
| prHDrev_1 | (5′-caatacaacaactcctcaaccacaacc-3′). |

### For DM1:

| | |
|---|---|
| prDM1for_1 | (5′-gtggatgggtaaattgtaggtttgg-3′), |
| prDM1rev_1 | (5′-aacatteccaactacaaaaaccatc-3′), |
| prDM1rev_2 | (cttttcctcccccaaccctaattc-3′). |

For CDH13 (clone 44g5 on the CpG island microarray):

CDH13 44g5F1
(5′-ataaaatttaagttaggatgggagatatag-3′);
CDH13 44g5R1
(5′-ataaataaaccaaaacaatactttaccta-3′);
CDH13 44g5F2
(5′-tttggtatttagtagttgtttaataaagtt-3′);
CDH13 44g5R2
(5′-tacaaaatatcatactctaatcactaaacc-3′).

PCR products were electrophoresed on an agarose gel, DNA fragments were excised, cleaned using Qiagen Gel Extraction Kit (Qiagen), and cloned into the pGEM-T vector (Promega). 30 clones from each PCR product (locus/individual) were sequenced. In order to evaluate the degree of intraindividual variation, an additional 30 clones were sequenced from separate bisulphite reactions in 5 cases: two BRCA1, one BRCA2, and two PSEN2. A total of 1,020 clones were analyzed which required over 1,500 sequencing reactions as some longer fragments had to be sequenced from both ends.

Analyses of DNA Methylation Variation in Bisulphite Modification-Based Experiments

The degree of epigenetic diversity within and across the individuals was evaluated using the concept of epigenetic “distance” (23). Each of the 30 sequenced clones was binary coded, with 0 for an unmethylated cytosine and 1 for a methylated cytosine. Each clone was therefore represented by a row vector of n “0” and “1” where n is the number of cytosines in the tested region.

Estimation of intra-individual variation. Unique methylation profiles were identified for each set of 30 clones. For example, a set of clones 0101, 0101, 0111, and 1100 exhibits three types of methylation profiles (½, 3, and 4), and therefore the proportion of unique methylation profiles is ¾. This was performed for every set of 30 clones, and then the mean and SD of the proportion of unique clones across individuals were calculated for each locus. In the second round of analysis, because of possible imperfect C to T conversion of bisulphite treatment, two clones different by a single position were called identical. Using the above example, profiles 0101 and 0111 are now treated as identical, and the degree of uniqueness is 2/4. In the final analysis, the tolerance was increased to 2 differences, i.e. the clones that exhibited two or fewer differences were treated as identical.

Comparison of DNA methylation “distances” across individuals. The average methylation intensity vector for each locus/individual was calculated from the sum of the methylated cytosines divided by 30 for each different cytosine position. The degree of epigenetic dissimilarity was measured by Euclidean distance using the following equation:

$${D}_{12} = {( {\sum }_{i = 1}^{n} {( {m}_{1 i} - {m}_{2 i} )}^{2} )}^{1 / 2} ,$$

where m1 is the average methylation vector of individual 1, m2 is the average methylation vector of individual 2, and d₁₂ is the Euclidean DNA methylation distance between individuals 1 and 2. The larger the distance, the more dissimilar the two individuals' methylation profiles are to each other. With this metric, we calculated the distances between all possible pairs of individuals for each promoter locus of BRCA1, BRCA2, HD, DM1, PSEN1 and PSEN2. To test statistical significance of methylation differences the following analysis was performed: for each locus, all clones from all individuals were pooled together, and two sets of 30 randomly selected clones from the pool formed the methylation profiles of two pseudoindividuals. The epigenetic distance between the two pseudo-individuals was then calculated with the same procedure as above, and this procedure was repeated 100,000 times generating 100,000 distances, the density distribution of which was plotted and the mean and +/−2SD were calculated. The (one tailed) p-value of a distance was then obtained by finding the area under the distribution curve from the left up to the calculated distance. An epigenetic distance in two real individuals with p<0.05 (i.e. >2SD) indicates that difference in DNA methylation of two individuals is statistically significant.

Microarray-Based DNA Methylation Analysis

Microarrays. Genome-wide epigenetic profiling was performed using the 12,192 CpG island microarrays (24) purchased from University Health Network Microarray Facility, Toronto (http://data.microarrays.ca/cpg/index.htm).

Enrichment of Unmethylated DNA.

We used our developed technology for enrichment of the unmethylated DNA fraction and epigenetic profiling described in detail in (25). The general principle of the DNA methylation profiling consists of interrogation of the unmethylated fraction of genomic DNA on the microarray. Intensity of hybridization inversely correlates with the DNA methylation status at the genomic locus homologous to a specific DNA fragment on the array. Briefly, methylation-sensitive restriction enzymes were used to digest 1 μg of genomic DNA, and two enzyme scenarios were used in this project. First, sperm DNA samples from 25 individuals were analysed using methylation sensitive enzymes HpaII, Hin6I and AciI (designated sperm DNA-HHA array set). This enzyme “cocktail” strategy, however, is not ideal for GC-rich regions such as CpG islands as these three enzymes would generate DNA fragments too small for efficient amplification and hybridisation. Therefore, a single digestion approach with HpaII alone was used on a second set of sperm DNA samples from 21 individuals (designated sperm DNAHpaII array set). DNA adaptors (annealing product of two primers: U-CG1a: 5′-cgtggagactgactaccagat-3′ and U-CG1b: 5′-agttacatctggtagtc agtctcca-3′) were ligated to the restricted DNA fragments, followed by treatment with McrBC (New England Biolabs) that will cleave the fragments containing two or more methylated cytosines thereby further enriching the unmethylated fraction. Adaptor-PCR amplification of the ligated products using primers complementary to the adaptor sequence consisted of 250 ng of ligated DNA, 2.5 mM MgCl₂, 0.2 mM aminoallyl-dNTPs [15 mM aminoallyl-dUTP, 10 mM dTTP and 25 mM each dCTP, dGTP and dATP], 200 pmol primer U-CG1b, 5 U Taq polymerase (New England Biolabs) in 1× PCR reaction-buffer (Sigma) to a final volume of 100 μl. PCR conditions are adjusted in such a way that only fragments less than 1.5 kb (i.e. short, digested and therefore unmethylated) will amplify preferentially. Cycling consisted of 72° C. for 5 min, 95° C. 1 min, then 25 cycles of 95° C. for 40 sec and 68° C. for 2 min 30 sec, followed by a final extension of 72° C. for 5 min. Equal amounts of amplicons from each sample were mixed to form the pooled control, which was labeled with Cy3 and co-hybridized against each individual amplicon labeled with Cy5. Hybridization was performed at 42° C. using standard procedure (25).

For comparison to the sperm DNA methylation profiles, DNA samples from post-mortem brains of 22 individuals who did not have any known brain disease were subjected to the same microarray-based DNA methylation profiling using a single digestion approach with HpaII (designated brain DNA-HpaII array set).

Microarray Data Processing and Analysis.

Methylation differences between the individuals and the pooled control were analyzed by the ratio of hybridization intensities of Cy5 (individual samples) over Cy3 (pooled control). As we have learned from our previous analyses of arrays used for DNA methylation analysis, such ratios show normal distribution, therefore the data can be treated similarly to classical microarray experiments. The array data were normalised in two steps, firstly, a global intensity normalization to adjust the Cy5:Cy3 ratio to 1:1 across the entire array, followed by block-by-block LOWESS normalization. The data was trimmed to remove spots with ambiguous genome locations, including spots with no sequence or annotation (647 spots), spots with >30% repetitive elements (2706 spots), and translocation hotspots (633 spots). The spots for which the microarray clones represented identical sequences were averaged resulting in approximately 4970 unique loci. Coefficient of variation (CV) was calculated for each remaining spot by the standard deviation in Cy5/Cy3 divided by the mean of the Cy5/Cy3 across all individuals. The sperm DNA-HHA experiments were performed in duplicate and the data were averaged ratios. The sperm DNA-HpaII and the brain DNA-HpaII data sets consisted of one array per individual where we opted for increased biological replicates rather than increased technical replicates for the number of microarrays available.

The age covariate analysis for the CpG island microarray experiment was performed by using a correlation coefficient between two series of quantities to measure the linear relationship between the series. Pearson correlation coefficient was calculated between the mean fold-change (log Cy5/Cy3) across individuals and the ages across individuals for each spot on the microarray. A large absolute value (|r|>0.5) of the coefficient indicates that the methylation intensity at the locus covariates with age in a positive or negative way. To test their statistical significance, the age across individuals were permuted, and again, the coefficient was computed using the permuted age series. For each spot, the permutation is repeated 5,000 times to get 5,000 coefficients. The one-tailed p-value of the coefficient is then obtained by finding the fraction of times the coefficients were larger (or smaller) than the original coefficient. Benjamani-Hochberg correction was used to correct for multiple testing. The autocorrelation clustering analysis for the CpG island microarray experiment was performed using the autocorrelation function ACF(x), which measures how strongly two methylation intensities “X” loci apart influence each other.

Measurement of Densities of metC in the Selected Loci

Further analysis of a selected set of DNA fragments identified as the most variable was performed using the methylation sensitive single nucleotide primer extension (MS SNuPE) reaction on the ABI SnapShot platform accommodated for measuring the C/T ratios in the bisulphite treated genomic DNA (26). Briefly, genomic DNA was digested with NdeI (Fermentas) followed by treatment with sodium bisulphite as described above. The loci of interest were amplified using nested PCR.

Typical PCR amplification consisted of 95° C. for 1 min, then 40 cycles of 95° C. for 30 s, 50° C. for 30 s and 72° C. for 40 s, followed by a final extension of 72° C. for 5 min. Quantitative interrogation of bisulphite induced transition C toT at CpG dinucleotides in such amplicons was performed with primers targeted to the CpG dinucleotides within the restriction sites for HpaII, Hin6I or AciI.

Results

Intra- and Inter-Individual DNA Methylation Differences in the Promoters of BRCA1, BRCA2, HD, DM1, PSEN1, and PSEN2

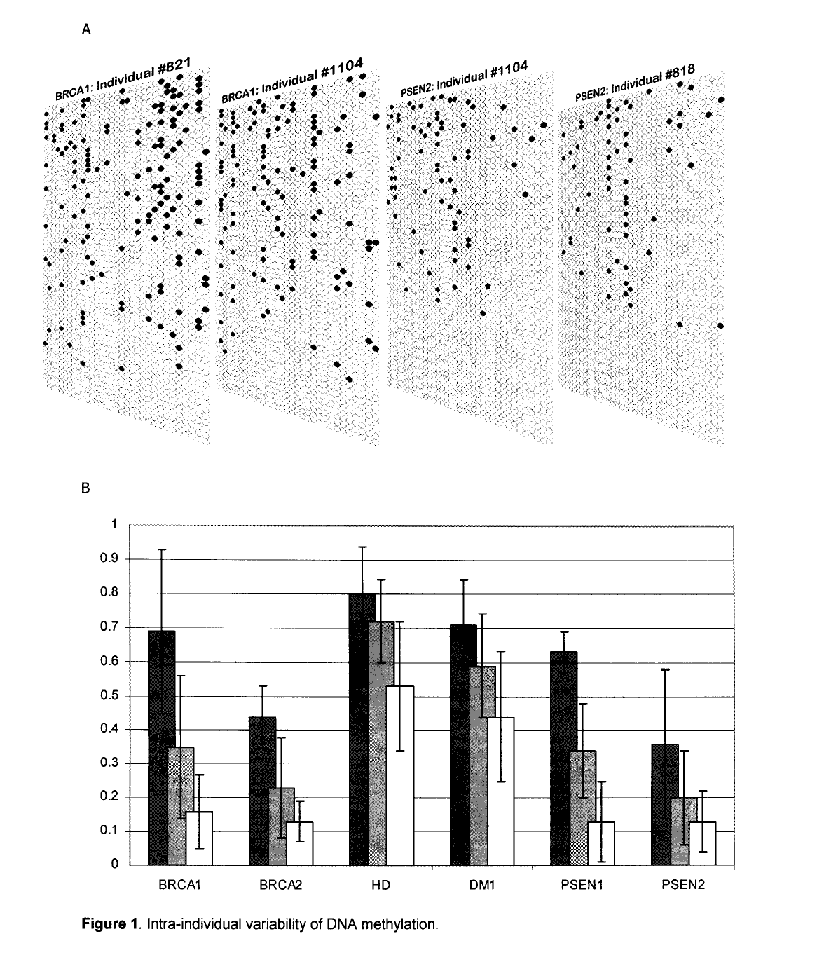

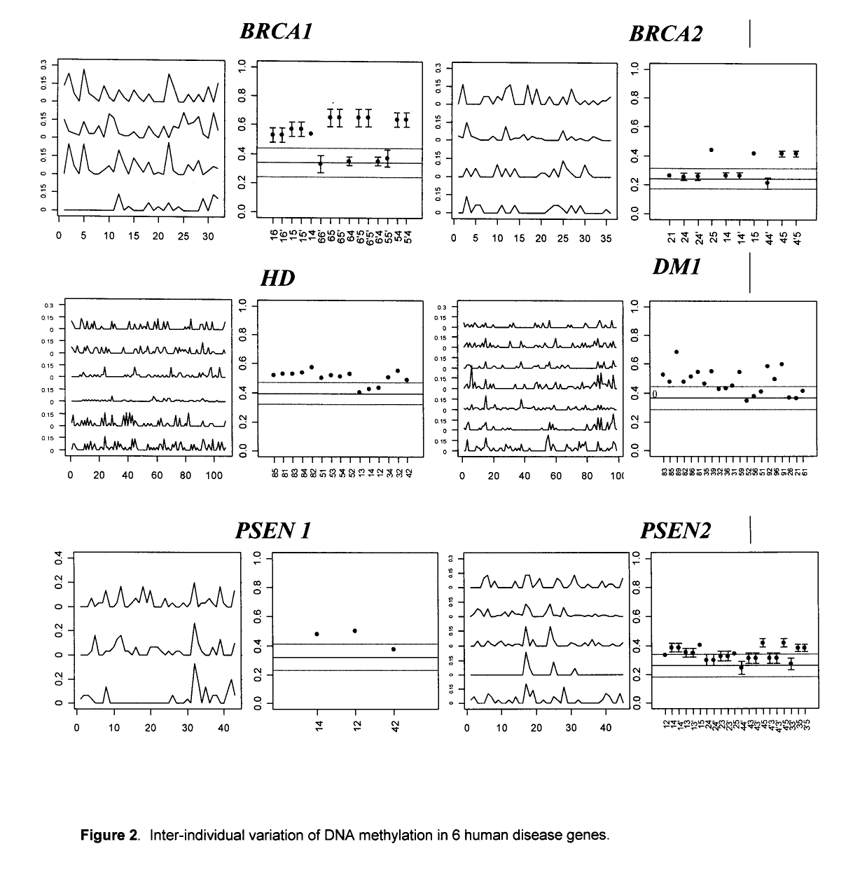

The bisulphite modification-based mapping of methylated cytosines for all of these genes demonstrated that numerous individual clones (representing individual sperm cells) demonstrated quite different DNA methylation profiles within individuals (FIG. 1a). This was confirmed by the analysis of the degree of uniqueness of DNA methylation profiles (FIG. 1b). In the case of HD, about 80% of all clones exhibited unique patterns of metC distribution. This estimate did not change dramatically when potential bisulphite modification induced artefacts were taken into account: on average 72% of clones were different from each other when one metC difference was tolerated, and up to 53%—when two differences were allowed. The latter situation is a very conservative estimate of the degree of uniqueness as such a high artefactual C to T non-conversion rate is unrealistic; based on the non-conversion at non CpG sites, which in our experiments was always less than 1%. The lowest degree of intra-individual DNA methylation uniqueness was detected for PSEN2: 36%, 20%, and 13%, for 0, 1, and 2 levels of tolerance, respectively. This analysis of uniqueness is, however, related to the clone length and correlates specifically with the density of CpGs analysed (Pearson R=0.64, 0.93 and 0.98, respectively, for 0, 1 and 2 levels of tolerance), as more methylatable CpG sites allow more opportunity for variation. While the intra-individual analysis can show variability within an individual, significantly variable methylation patterns between individuals were also revealed (FIG. 2). The gene specific results were as follows: BRCA1, N=4, 32 CpGs analyzed, 5/6 pairwise comparisons exhibited statistically significant differences (Average p=2.53E−05); BRCA2, N=4, 36 CpGs, 3/6 pairwise comparisons were significant (Avg p=8.56E−07); PSEN1, N=3, 43 CpGs, 2/3 pairwise comparisons significant (Avg p=1.89E−04); PSEN2, N=5, 45 CpGs, 6/10 pairwise comparisons significant (Avg p=5.11E−03); DM1, N=7, 99 CpGs, 13/21 pairwise comparisons significant (Avg p=5.60E−04), and HD, N=6, 108 CpGs, 12/15 pairwise comparisons were significant (Avg p=1.66E−03). Overall, 67% (41/61) of the pair-wise comparisons were significantly different which suggests a high overall level of inter-individual variability in the methylation patterns of the tested genes. The five cases where 60 sequenced clones were available for BRCA1, BRCA2, and PSEN2, the comparisons were performed using randomly selected two groups of 30 clones (FIG. 2). As a validation of this statistical method the additional sets of 30 clones representing BRCA1, BRCA2, and PSEN2 were compared to the primary sets of 30 clones of the same individuals. In all cases the result (cf. pairs 55′ and 66′ in FIG. 2) showed that their profiles were not different, as expected since they are from the same individual.

DNA Methylation Differences Detected by the CpG Island Microarrays

This CpG island microarray contains 12,192 DNA fragments, however, unique sequences are represented by 4,970 distinct loci of which only about half met the commonly used the criteria for CpG island: GC content of 50% or greater, length greater than 200 by and observed/expected CG dinucleotide ratio greater than 0.627. While the rest of the unique loci failed to meet one or more of these criteria, as described in the Methods section, a two enzyme set—HHA and HpaII—strategy to increase the informativeness of our analysis was adopted.

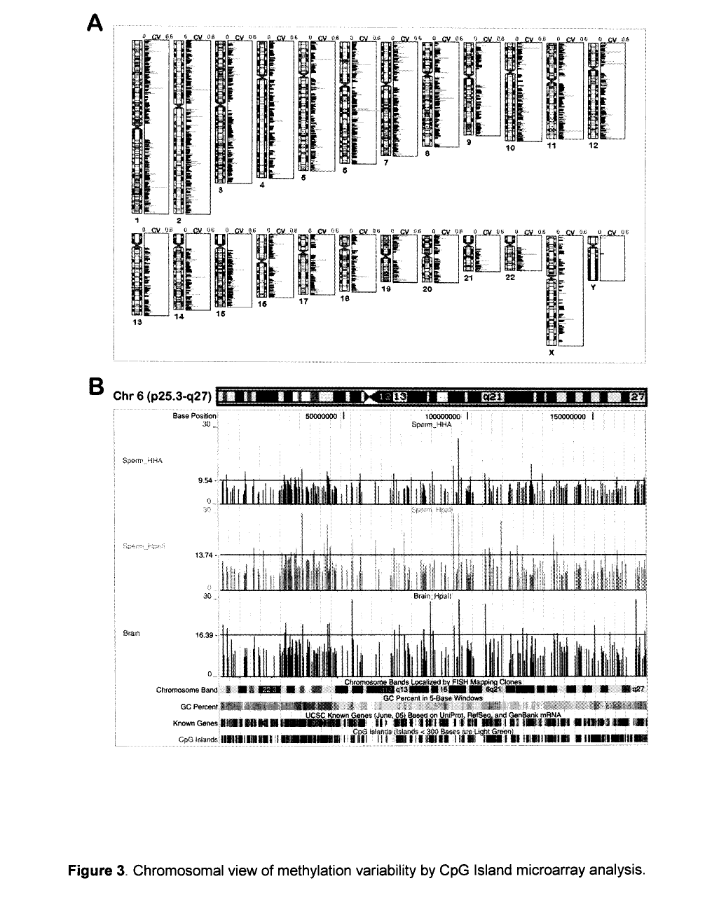

As a measure of methylation variation we have calculated the coefficient of variation (CV) across individuals for each array set. The CV is calculated by the standard deviation in Cy5/Cy3 ratio divided by the mean of the Cy5/Cy3 ratio, expressed as a percentage. The variation between individuals across the genome ranged from CV 2.1-30.5% (mean=6.7); 0.8-66.2% (mean=9.2); 2.1-97.4% (mean=10.9) for the sperm DNA-HHA; sperm DNA-HpaII and brain DNA-HpaII data sets, respectively (Table 1). The data for each locus was plotted on the genome (FIG. 3; FIG. 8) and is also available on the web as a custom annotation track using the USCS genome browser (FIG. 3b) (http://www.epigenomics.ca/, the site will be launched in January 2006). FIG. 3a depicts the sperm DNA-HpaII data set and highlights the highly variable regions on the genome. To assess if this distribution of highly variable spots is non-random, an autocorrelation analysis was performed, however, this did not identify any evidence for autocorrelation, most likely due to the large genomic distance between microarray clones (Avg 0.6 Mb). Other analyses included: testing if the detected variability is confounded by DNA sequence variation, comparison of DNA methylation variation in CpG islands vs non-CpG islands as well as across different classes of repetitive elements, and assessing if DNA methylation variation correlates with the GC content, clone length, or to particular chromosomal cytobands or with any genes of particular biological processes, pathways or molecular functions.

Exclusion of genetic confounding effects: single nucleotide polymorphisms (SNPs) and copynumber polymorphisms (CNPs). Any method that relies on restriction enzyme digestion to differentiate between methylated and unmethylated DNA can be influenced by single nucleotide polymorphisms within the enzyme restriction sites. Therefore, from each of the sperm DNA-HHA and sperm DNA-HpaII datasets we selected 150 highly variable loci and 150 conserved loci and performed in silico screening to identify all known SNPs within a 2 kb region of the selected clone that disrupt or create HpaII, Hin6I or AciI enzyme sites for the sperm DNA-HHA data set or just HpaII sites for the sperm DNA-HpaII data set (SNP annotation of the USCS genome browser: http://genome.ucsc.edu/). The c2 analysis revealed no association between the number of potentially disruptive enzyme restriction sites and the degree of variability in either data set (sperm DNA-HHA c2=0.12, p=0.729; and sperm DNA-HpaII c2=1.83, p=0.176). This suggests that the degree of variability in the sperm DNA microarray analysis is more dependent on DNA methylation differences than on DNA sequence differences. Recent reports have identified over 200 copy number polymorphisms (CNPs) that represent large duplications and deletions that contribute significantly to genomic variation between individuals (28-30). Like SNPs, CNPs could simulate DNA methylation variability in the microarray analysis. We have cross-referenced the CNPs identified in these studies with the CpG island microarray loci and identified 25 microarray loci that occur within known CNP regions. These include large CNPs in chromosome 3 (covering the genes OSTalpha, AB018337, UNQ3030, BC015560 and DLG1), chromosome 16 (BC008967, XYLT1, ARL61P, MIR16, MGC16943 and CDR2) and chromosome 17 (AY302137, BHD, RA11, FLJ20308, TOP3A and SMCR8) and smaller CNPs on chromsomes 1 (NEGRI), 2 (AK024244), 6 (RDBP), 8 (TSTA3), 9 (LHX2), 11 (TNNT3) and 14 (AK090461). Microarray results for these genes listed could therefore be influenced by deletions or duplications as much as by methylation variability, however, none of these loci appear in the list of highly variable (>90th percentile) loci.

CpG Island Analysis

Not all DNA fragments on the CpG island microarray met the criteria for CpG islands. The list of loci were divided into CpG island or not CpG islands (Table 2). A significantly increased DNA methylation variability was found in loci defined as CpG islands in the sperm DNA-HpaII data set (t-test, p=4.92E−06) and this was exemplified by a bias towards CpG islands in the 90th percentile (highly variable regions)(c2=24.34, p=5.81 E−07). In addition, when the CpG islands were split into promoter CpG islands and CpG islands not associated with known gene promoters, significantly higher variability in promoter CpG islands (c2=11.44, p=4.87 E−04) was detected. Analyses of methylation variability with other measures including GC percent alone and clone length, however, did not reveal any association. No evidence for higher DNA methylation variation was detected in the promoter CpG islands in the brain-HpaII data set and there also was no association with SNPs. Therefore, this sperm DNA-HpaII experiment appears to have revealed genuine increased methylation differences in the promoter CpG islands.

Cytoband Analysis

It has been well described that different cytobands could have evolved in different ways and the genes within each band could have evolutionary similarities (31,32). As these bands are based on GC content and Alu content, among other things, we sought to identify if methylation variability was one of the aspects that showed similarities within bands. The CpG island microarray annotation includes the division of loci into different cytobands including G bands (gneg) and the four classes of R bands (gpos25, gpos50, gpos75 and gpos 100). Mean CV for all of the loci within each of these cytobands were calculated and a Student's t-test was performed to identify statistically significant differences. In each of the data sets marginally significant association with certain cytobands were identified. In the sperm-HHA data set significant decrease in variability between gpos75 band loci (CV=6.51) and the other three R bands gpos25, gpos50 and gpos100 (Avg CV=6.83, Avg p=0.023) were detected. In the sperm-HpaII data set gpos25 exhibited lower degree of methylation compared to gpos50 (CV=8.97 and CV=9.50, respectively; p=0.041). While the significance of these statistical tests diminished when corrected for multiple testing, the result is suggestive of an increase in variability in the Alu rich cytobands, such as the gpos50 and gpos100 cytobands compared to the Alu poorer bands gpos25 and gpos75.

Age-Dependent DNA Methylation Changes in the Sperm

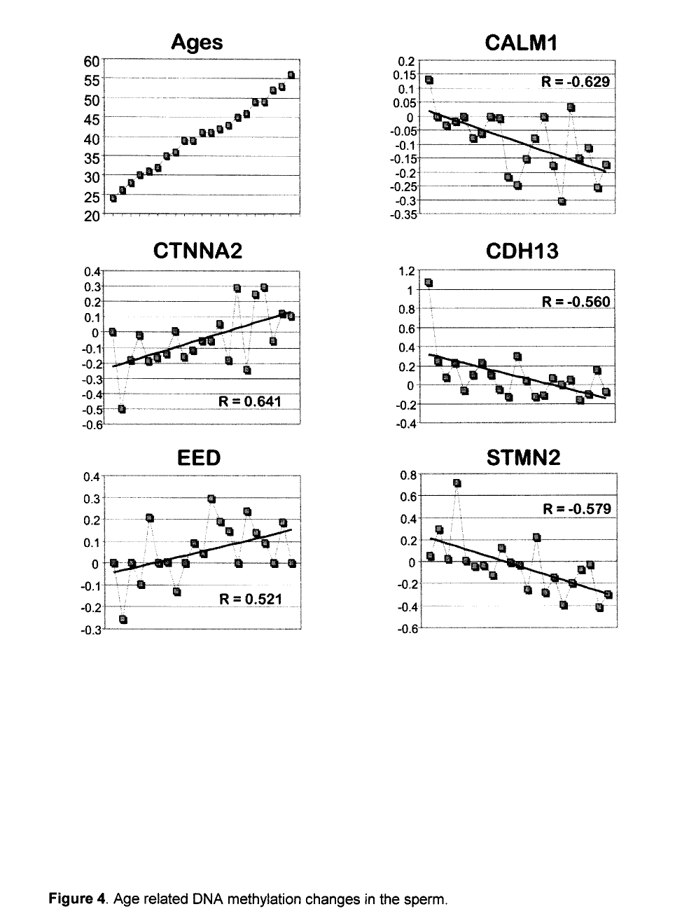

Methylation dynamics using age (sperm DNA-HHA age range: 22-35 yr; sperm DNA-HpaII age range: 24-56 yr) as a covariate was investigated. In the sperm DNA-HpaII and sperm DNA-HHA datasets 105 and 8 loci were found, respectively, whose absolute correlation coefficients were larger than 0.5 and p-value <0.05. Numerous genes were identified in the germ cell data that corresponded to genes involved in spermatogenesis and development (e.g. INSM1, TZFP, EED), neurogenesis (e.g CALM1, STMN2, ARHGEF9, ARX) or disease related genes (e.g. MAF, DCC, CDH13). A number of examples are shown in FIG. 4. The lists of genes for each data set is provided (see Tables 2A,B).

DNA Methylation in the Repetitive Elements

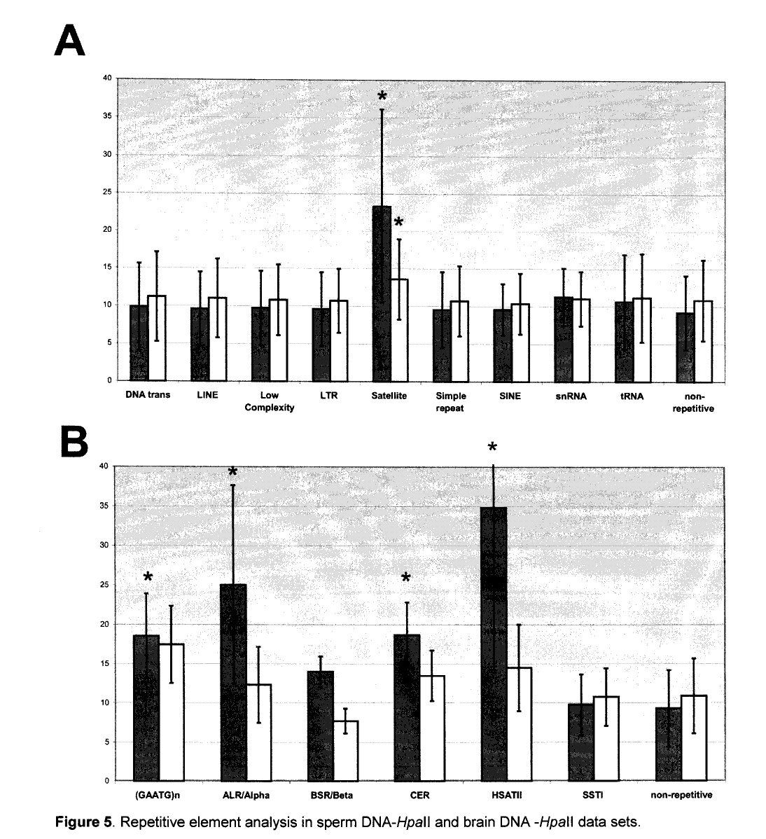

All the above analyses were performed on unique DNA sequences, however, the CpG island microarray also contains a large number of clones containing repetitive elements, which as a rule are heavily methylated (33). While it is difficult to directly distinguish between methylation- and copy number differences, one possible approach is to compare methylation of repetitive elements in the sperm to that in other tissues. For this reason, the sperm DNA-HpaII data set was analyzed in comparison to the brain DNA-HpaII data set. This analysis revealed the average overall repetitive element CV of 10.5 in the sperm compared to the overall average CV in non-repetitive elements of 9.6. The breakdown of CV for each type of repetitive elements represented on the microarray is shown in FIG. 5. This analysis identified that satellite DNA repeats were statistically more variable than other repetitive elements in the sperm DNA-HpaII data set (p=6.12E−17). In comparison, this effect was far less pronounced in the brain DNAHpaII data set (p=0.0027) (FIG. 5a). When the satellite repeats were further separated into specific repeat classes, a number of repeat classes, predominantly centromeric or pericentromeric satellite repeats, were-identified as responsible for this increase in inter-individual variability including (GAATTC)n (CV=18.5) ALR/Alpha (human alpha repetitive DNA, CV=25.0), CER (human D22Z3 centromeric repetitive DNA ; CV=18.7) and HSATII repeats (human satellite II DNA, CV=34.8) (FIG. 5b).

Validation of the Microarray Data Using Bisulphite Modification-Based metC/C Analysis

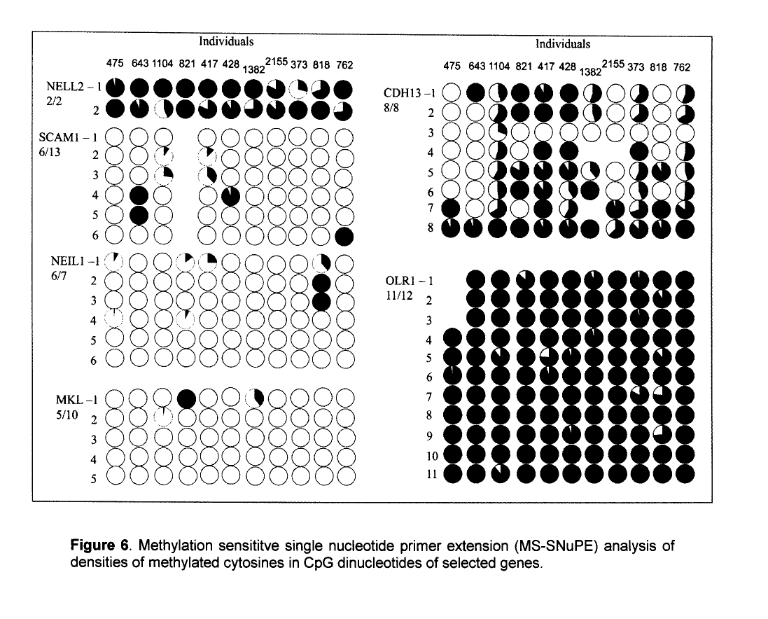

For validation of the microarray data 12 loci that were detected as variable in the CpG island microarray analysis (Table 3) were analysed using the methylation sensitive single nucleotide primer extension (MSSNuPE) reaction on the ABI SNapShot platform 26 at the CpG dinucleotides in the HpaII as well as Hin6I or AciI restriction sites. Initially such loci were selected based on increased variability (>90th percentile) in the sperm DNA-HHA data set; in addition, a number of these loci were also highly variable in the sperm DNA-HpaII data set (CDH13, SCAM1, MKL2, and DIRAS3). Each of the 12 loci selected were initially resequenced to confirm the identity of the sequence. DNA samples from 11 individuals were treated with sodium bisulphite, PCR amplified, and primer extension reactions were performed to interrogate 65 CpG dinucleotides within the 12 sequences. Examples of 6 loci are presented in FIG. 6. This analysis revealed variable levels of methylation differences in at least one enzyme restriction site in 11/12 loci tested. It should be noted here that DNA methylation differences in a single restriction site may be sufficient to generate significant differences in the microarray analysis. Only one locus (DIRAS3) showed no methylation differences between the 11 individuals, however, we were only able to test 5 out of 20 CpG sites at this locus, thus methylation variation in the untested CpG sites cannot be ruled out. To assess the replicability of the assay the MS-SnuPE/SNaPshot experiment was repeated on 5 loci in 5 individuals. Consistently with published data (26), the results in this second round of experiments were within 5% of the first experiment on average (range 1.7-9.9%).

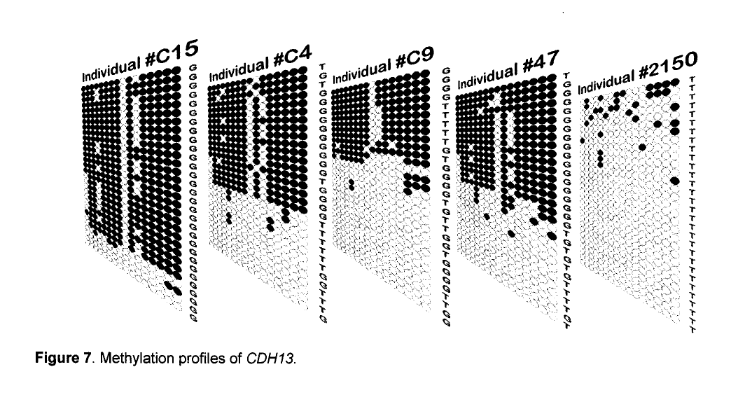

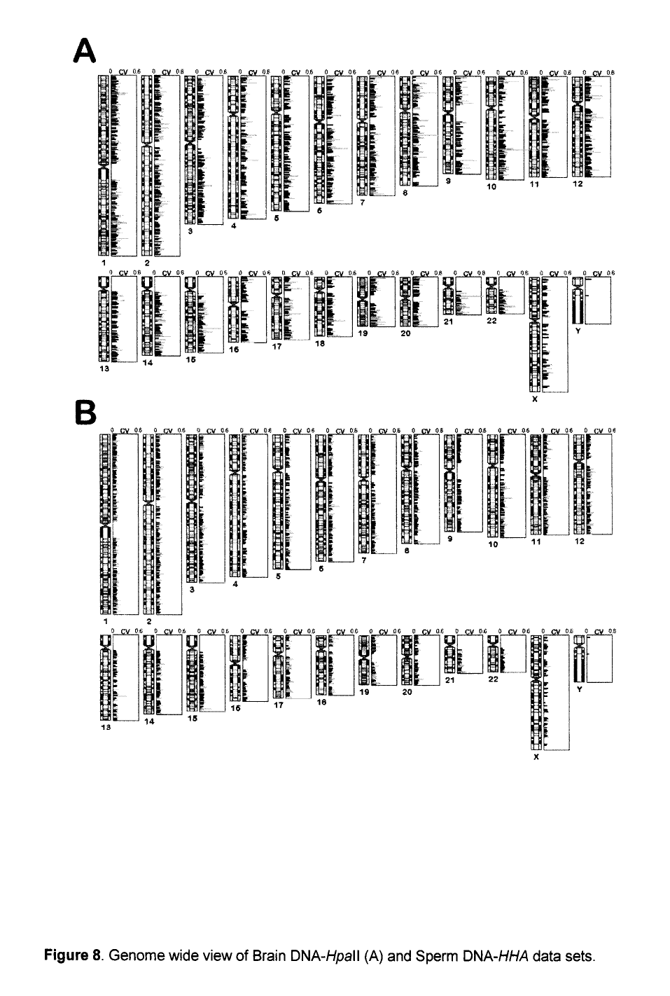

Finally, as further validation of the MS-SNuPE method and microarray results we have performed bisulphite genomic sequencing of 30 clones from 5 individuals on a locus within the gene encoding cadherin 13, CDH13, (UHNhscpg0004063). This analysis revealed a clear-cut bimodal distribution of epialleles, with the majority of clone sequences being either mostly methylated across all 16 CpG dinucleotides tested or predominantly unmethylated. In addition, this sequencing analysis identified a single nucleotide polymorphism, C/G; out of the 5 individuals, one was homozygous C, one homozygous G and the other three were C/G heterozygous (FIG. 7). Of particular interest is the substantially higher density of methylated cytosines on the G allele, while C alleles predominantly exhibit low degree of methylation. Counting all clones across all 5 individuals together it was found that 67/87 (77%) of the sequences with the G allele were methylated in comparison to only 14/63 (22%) sequences containing the C allele that were methylated. (c2=40.4, p=2.08E−10). Given that the microarray analysis suggested that promoter CpG islands were significantly more variable we also performed bisulphite genomic sequencing of the CDH13 promoter CpG island which was not represented on the CpG microarray. This analysis, however, found that the promoter CpG island of CDH13 is predominantly unmethylated in all individuals with only solitary methylation sites present in 1-3 clones for each of the individuals.

Discussion

The present example provides an in-depth analysis of epigenetic variability in the germline. The results suggest that i) male germline exhibits locus-, cell-, and age-dependant DNA methylation differences, and ii) DNA methylation variation is significant across unrelated individuals that by far exceed DNA sequence variation. These findings are interesting from both basic molecular biology and biomedical points of view. First, our study contributes to the understanding of epigenetic peculiarities of gene regulatory regions in the germline. It has been generally accepted that CpG islands are predominantly unmethylated (34), which implies that DNA methylation differences would not be expected there. From our studies, even relatively low densities of methylated cytosines in the CpG islands are sufficient to generate unique epigenetic profiles in the DNA regions that do not exhibit any DNA sequence variation, both in different cells of the same individual and also across individuals. Fine mapping of methylated cytosines of relatively short DNA fragments of BRCA1, BRCA2, PSEN1, PSEN2, DM1, and HD suggest that each sperm cell is unique not only in terms of DNA sequence but also in epigenomic profile, and variation of the latter by far exceeds the former.

At the genome wide level, unexpectedly, promoter CpG islands exhibited larger inter-individual variation compared to other single copy DNA sequences, including the non-promoter CpG islands. This epigenetic phenomenon seems to be discordant with a general rule that functionally important loci exhibit a low degree of DNA variation, as is seen in the case of SNPs being less common in promoters and exonic sequences than in introns and intergenic regions. In addition, promoter CpG-rich regions are often highly conserved between species, for instance the mouse genome contains 15,500 CpG islands of which approximately 10,000 are highly conserved (35). Therefore, if the epigenetic variability were just “noise” of little functional relevance, one would expect more variability in these less biologically important regions such as introns and intergenic sequences. Evidence for the opposite—increased epigenetic variability in the regions that directly control gene activity—may indicate some peculiarities of DNA methylation machinery during gametogenesis that may or may not be of functional importance in the somatic cells.

Our study has also identified a larger degree of inter-individual variability of centromeric satellite repeats. Although we cannot strictly rule out the possibility of DNA copy differences, which are common in centromeric satellite repeats (36), the fact that the germ cell data set showed substantially larger CV in comparison to the brain DNA data set suggest that germline satellite methylation differences in the germ cells could be a genuine biological phenomenon. Inter-individual methylation variability in satellite repeats is consistent with current knowledge (37) and may contribute to phenotypic variability in ICF syndrome, a disease that is associated with methylation defects in pericentromeric satellites (38). In addition, microRNAs (or siRNAs) regulate gene expression, heterochromatin formation and genome stability and often arise from demethylation of tandem repeats that are common in pericentromeric sequences (39). Therefore, inter-individual methylation variability in tandem repeats that give rise to microRNAs could also be involved in the variability in gene expression that results in inherited phenotypic variation. A recent study has described increased interindividual variability in the methylation of Alu repeats (40) in whole blood DNA. However, Sandovici et al noted that the parental origin differences in methylation were identified only for Alu elements in pericentromeric chromosomal bands, which is consistent with our results. Second, epigenetic variation within- and across-germline samples could be of significant interest in human morbid genetics that thus far has nearly exclusively concentrated on DNA sequence differences. Inherited epigenetic variation may provide the basis for new hypotheses and experimental designs in the studies of various human diseases where the traditional DNA sequence based studies are reaching the limit of explanatory power. For example, although Huntington's disease is caused by trinucleotide repeat expansion in the HD gene, the correlation between the number of trinucleotide repeats and age of onset for later HD cases (>50 years) is low (41). Epigenetic status of HD promoter region may contribute to the steady state HD mRNA levels and therefore the production of toxic polyglutamine- containing proteins.

HD genes containing identical trinucleotide repeat expansion but differential DNA methylation and chromatin compaction in the promoter region may exhibit significant differences in terms of their pathogenic potential reflected in the age at disease onset and severity.

The role of differential germline epigenetic modification in complex non-Mendelian disease may be even more important. Despite significant effort over the last several decades, DNA sequence- based risk factors have been uncovered in only a small fraction of complex disease, such as familial breast cancer and early onset Alzheimer's disease. For a number of complex diseases, genetic epidemiological studies showed that DNA sequence differences account for only a small portion of phenotypic variance among relatives, while the substantial remaining fraction of phenotypic differences (in some cancers 58%-82% (42) are typically attributed to environment. Identification of causal environmental factors is very difficult because methodologically impeccable designs in epidemiological studies, as a rule, cannot be applied to humans (43). At the same time, there is an increasing body of evidence that environmental factors play a minimal role in a number of complex traits and disease conditions (44). In this context, epigenetic variation in the germline arises as a new molecular mechanism that may help understanding complex phenotypes that are not the outcome of DNA sequence variation or differential environment. The recent finding of germline epimutations of MLH1 in two individuals affected with multiple cancers (11) provides a good starting point for a systematic search for disease specific epimutations in the germline.

In our bisulphite modification-based analyses, the overwhelming majority of loci exhibited rather subtle DNA methylation differences (“shades of grey” type), while methylation of the CpG island within the gene encoding cadherin 13, CDH13, is clearly bimodal (“black or white” type). The cadherin gene is a putative mediator of cell-cell interaction in the heart and may act as a negative regulator of neural cell growth. The promoter of this gene is hypermethylated in numerous cancers (45-50). Of particular interest is the finding that DNA methylation profiles are associated with DNA alleles, where the C allele of CDH13 is predominantly unmethylated, while the G allele is predominantly methylated. To our knowledge, thus far, the only other example of a link between DNA sequence and epigenetic codes was demonstrated in Beckwith-Wiedemann syndrome, where loss of maternal allele-specific methylation was more common on the G allele at T382G SNP (CAGA haplotype) of the differentially methylated region KvDMR151. A number of genes in the sperm exhibited DNA methylation changes that correlate with age (FIG. 4). This finding is particularly interesting in the light of the evidence that older paternal age is associated with the risk for schizophrenia in the offspring (52,53). Although it has been hypothesized that such effects could be due to epigenetic changes in the paternal genome, no locus specific- and age dependent- epigenetic changes in human male germline have been identified thus far. In this study a number of genes that show age-related changes in their DNA methylation have been detected, and such include a number of important developmental genes. The embryonic ectoderm development gene, EED, is a polycomb group gene involved in maintaining the epigenetically regulated repressive state of developmental genes over successive cell generations (54). CTNNA2, or catenin is a neuronal cadherin associated protein and may play a major role in the folding and lamination of the cerebral cortex (55). CALM1, or calmodulin is a key calcium-modulated protein that functions in growth and the cell cycle as well as in signal transduction and the synthesis and release of neurotransmitters. STMN2, or stathmin-like 2 is neuronal growth-associated protein that shares significant amino acid sequence similarity with the phosphoprotein stathmin and CDH13 as described above is the heart cadherin and is hypermethylated in a number of cancers. All the above phenotype related aspects were discussed under the assumption that the epigenetic peculiarities of the germline are at least to some extent reflected in the somatic cells after birth. What proportion and to what extent these inherited epigenetic signals can “survive” the reprogramming that immediately follows fertilization as well as during the later stages of embryogenesis (13,56,57) remains unknown. The methylation clearing is not complete, and on a global DNA level is reduced to about 10% (58,59). That could represent 90% of all methylation for each gene being erased or it could mean 90% of methylated genes are completely cleared and 10% of genes retain their methylation or there could be numerous combinations of the two. It is also unknown what happens to the histone modifications through these phases of loss of DNA methylation signals. Since modifications of DNA and histones are codependent, even if the DNA methylation signals are erased, the histones may be able to carry on specific epigenetic messages to the next stage until the DNA gets remethylated. This concept of cellular memory through histone modifications has been demonstrated for polycomb group proteins through H3K27 trimethylation (60).

The second aspect that will determine biological importance of the epigenetic variation in the germline is trans-generational epigenetic inheritance and whether complex DNA methylation patterns can be inherited from parents and transmitted to offspring. There is already experimental evidence demonstrating epigenetic meiotic inheritance across different species, such as yeast (61), arabidopsis (62), drosophila (63,64), and mice (20,21). While there is no doubt that transgenerational epigenetic inheritance does exist, it is not clear if this is limited to a few loci or it is a common genome-wide phenomenon.

### Example 2

### Epigenetic Basis for Bipolar Disorder

In this study DNA methylation profiling using microarray analysis of 20 bipolar disease cases and controls was performed in order to identify potential disease specific epigenetic signals in sperm cells.

Materials and Methods

Samples: Sperm samples were collected at the Centre for Addiction and Mental Health (Toronto, Canada) from 20 bipolar disorder patients and 20 healthy controls. This study was approved by an institutional ethics board, and informed consent was obtained from all participants. Extraction of DNA was performed using standard salt and phenol/chloroform extraction techniques known in the art.

Microarray analysis: Microarray analysis was performed as previously described in Example 1. Briefly, the unmethylated fraction of DNA was enriched using the method developed in our laboratory (25; 65) and each individual case or control was hybridised to a 12,192 feature CpG island microarray in comparison to a reference sample (pooled controls). Each analysis was performed in triplicate with one dye-swap array. Data from each microarray was normalised (global intensity and Lowess) and log ratio Case/reference or control/reference was calculated. A Students t-test assuming unequal variance was performed to identify significant methylation differences between cases and controls. False Discovery Rate (FDR) was used to correct for multiple testing, using a cutoff of p=0.3 which assumes that 7/10 loci will be true positives.

Results

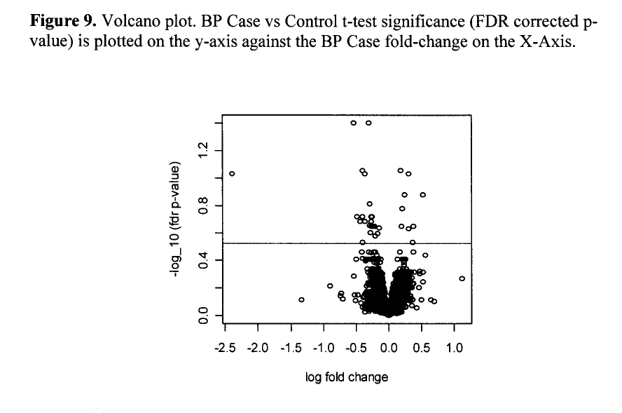

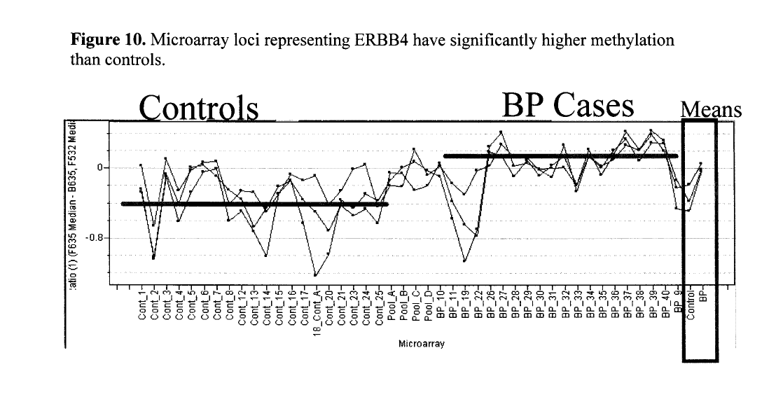

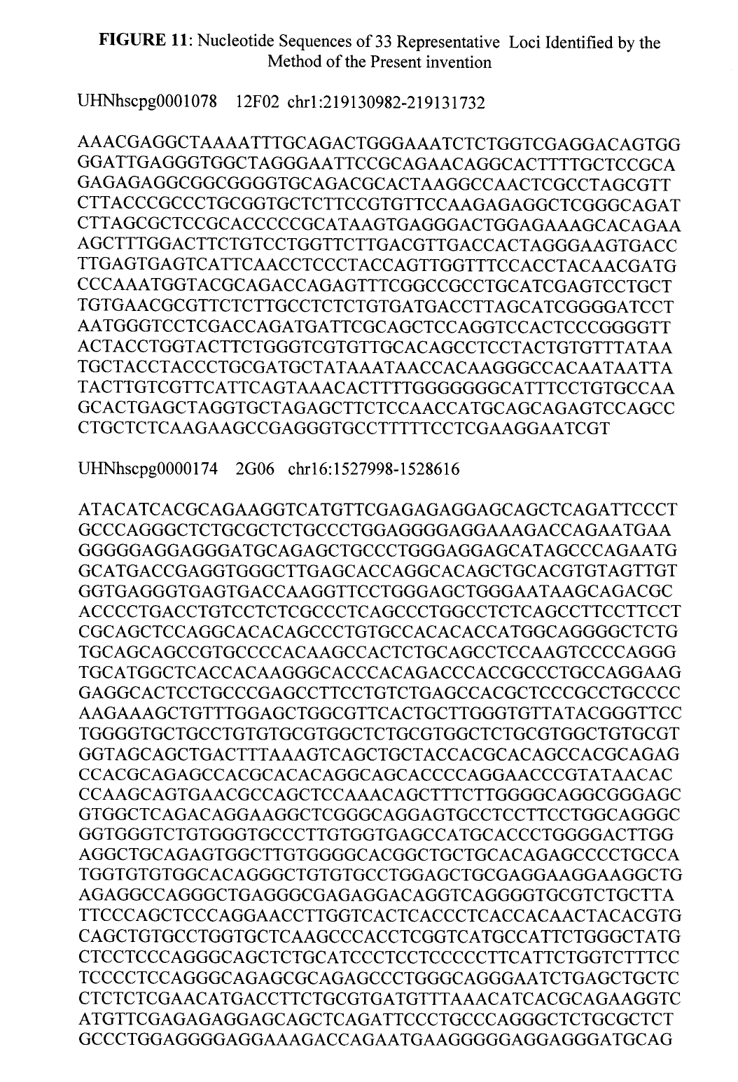

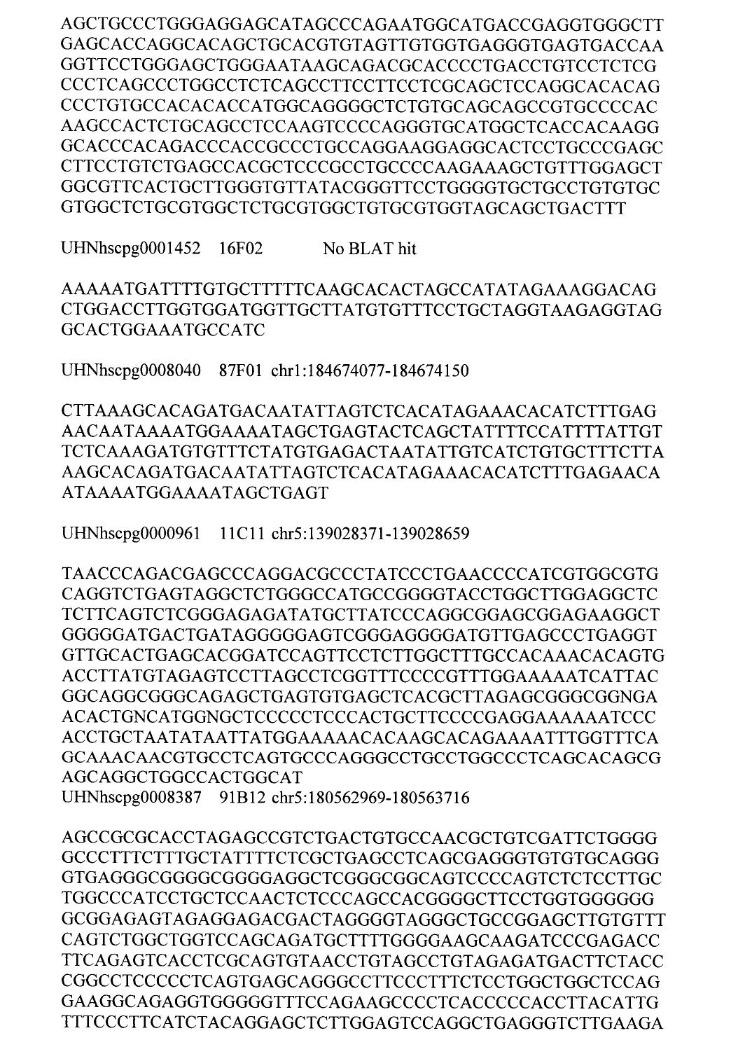

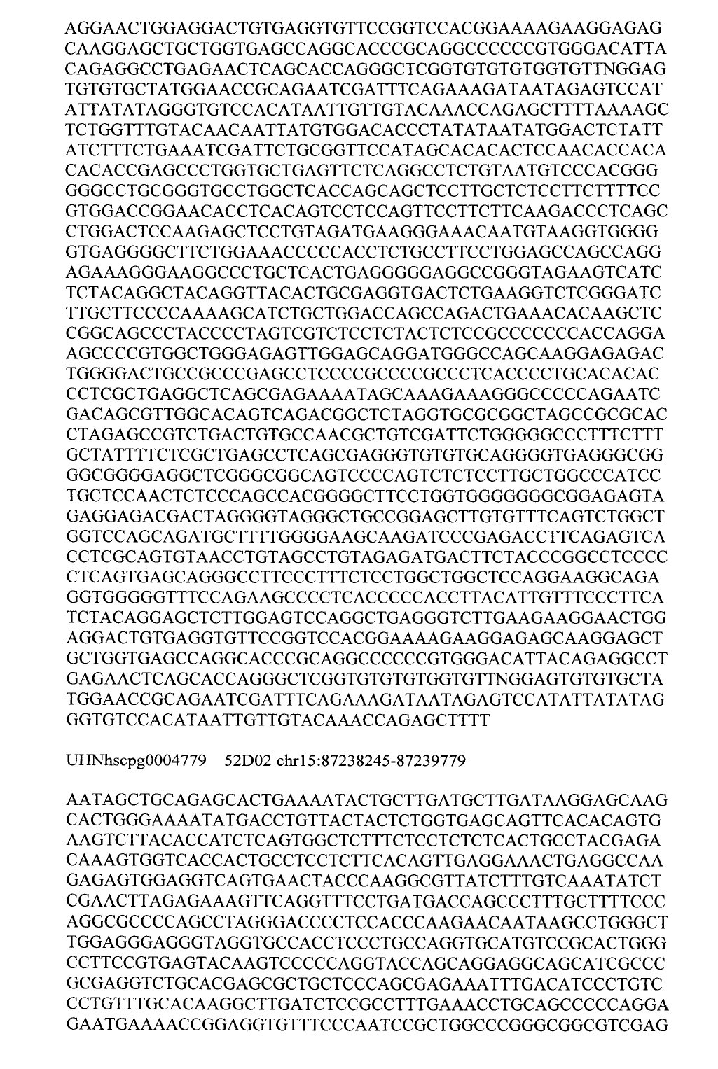

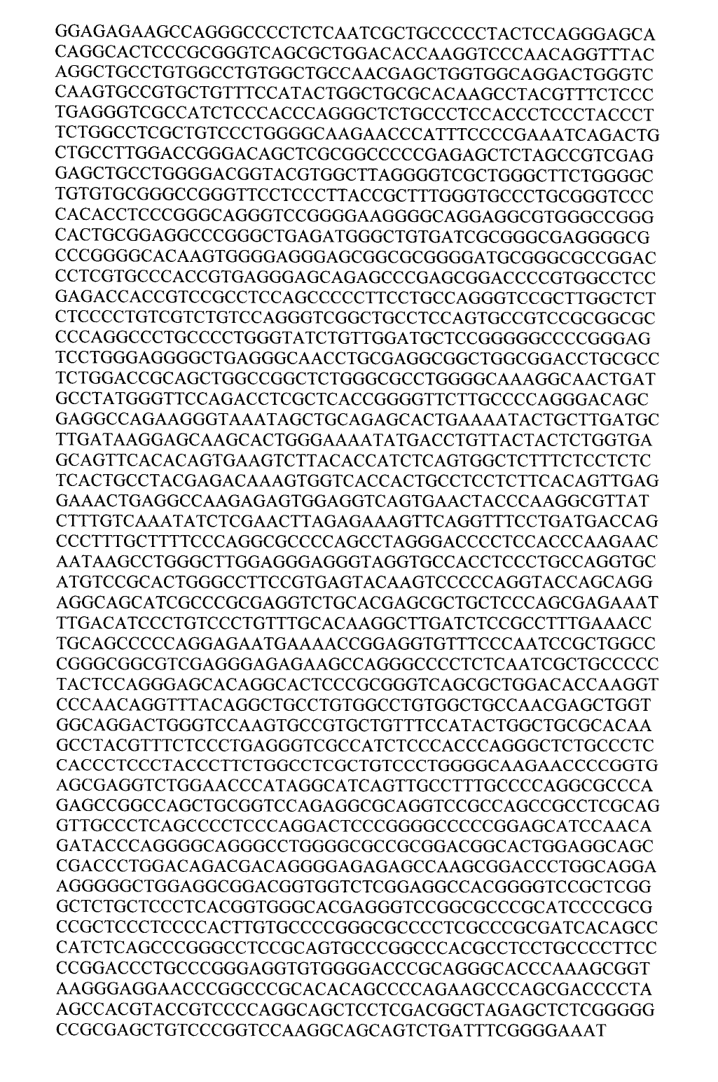

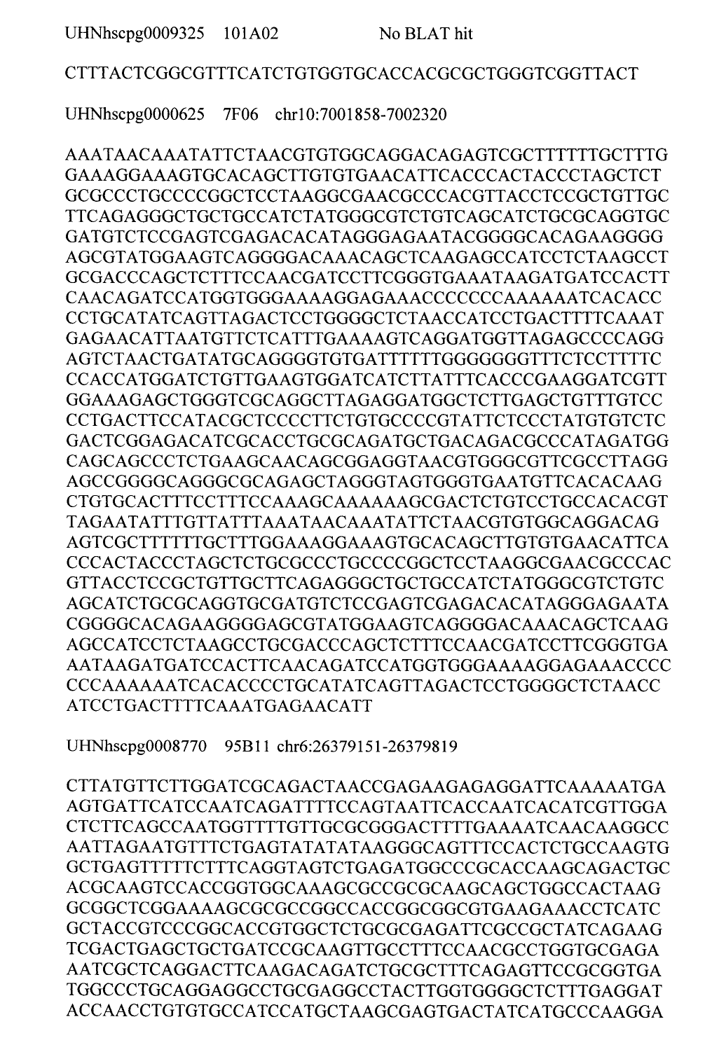

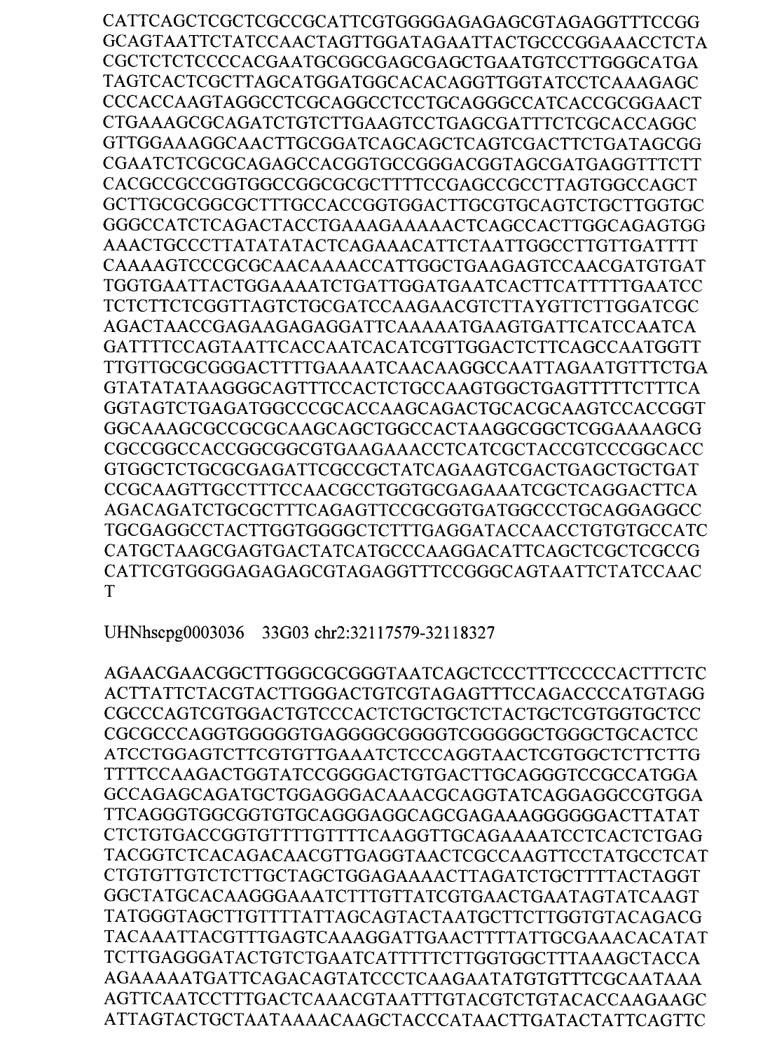

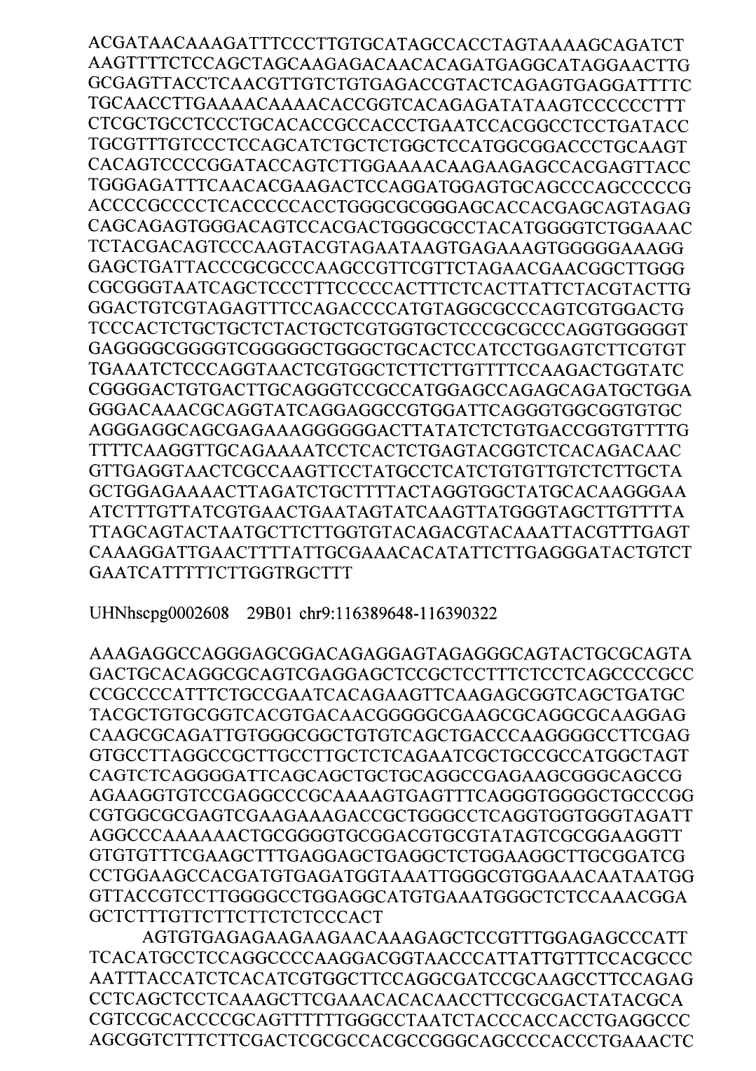

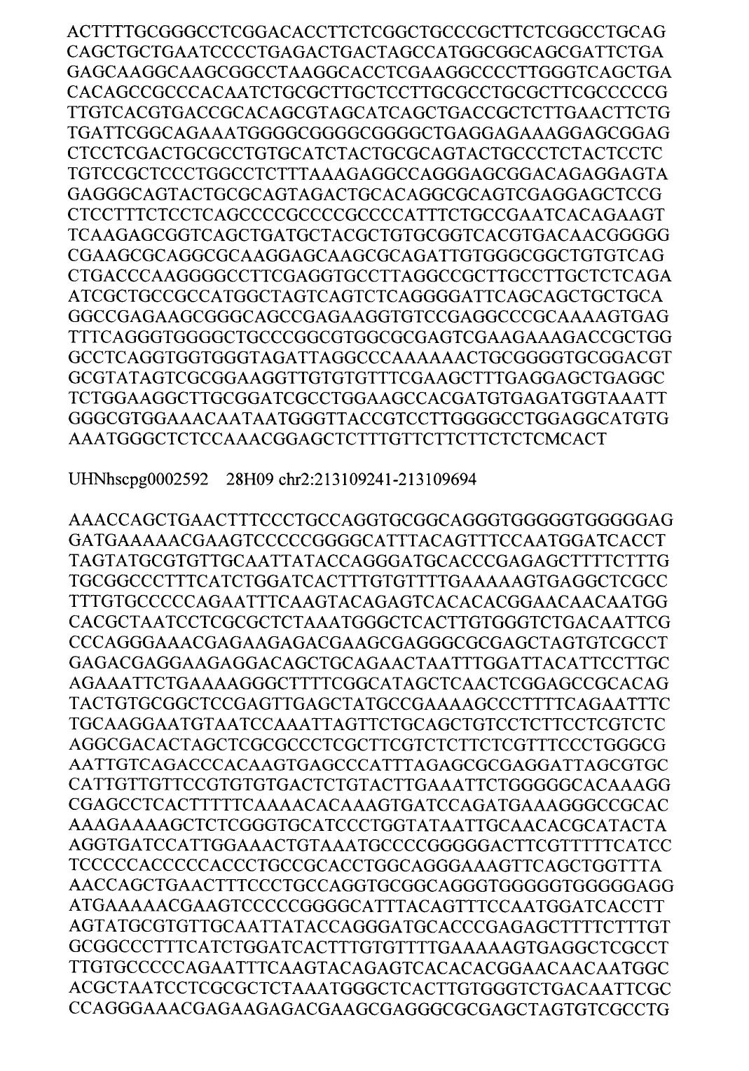

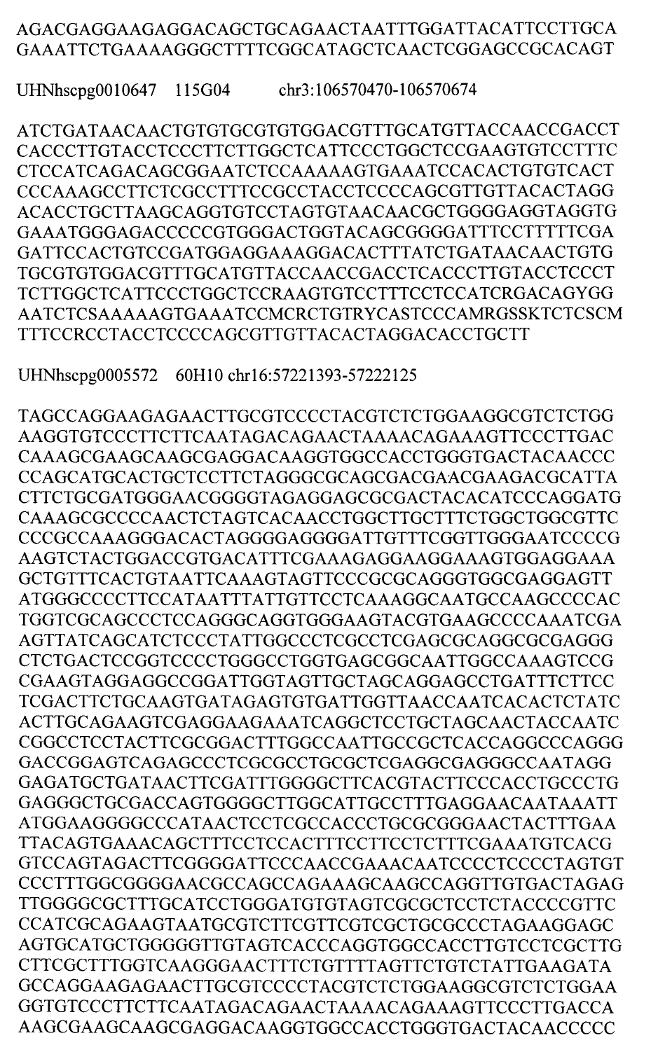

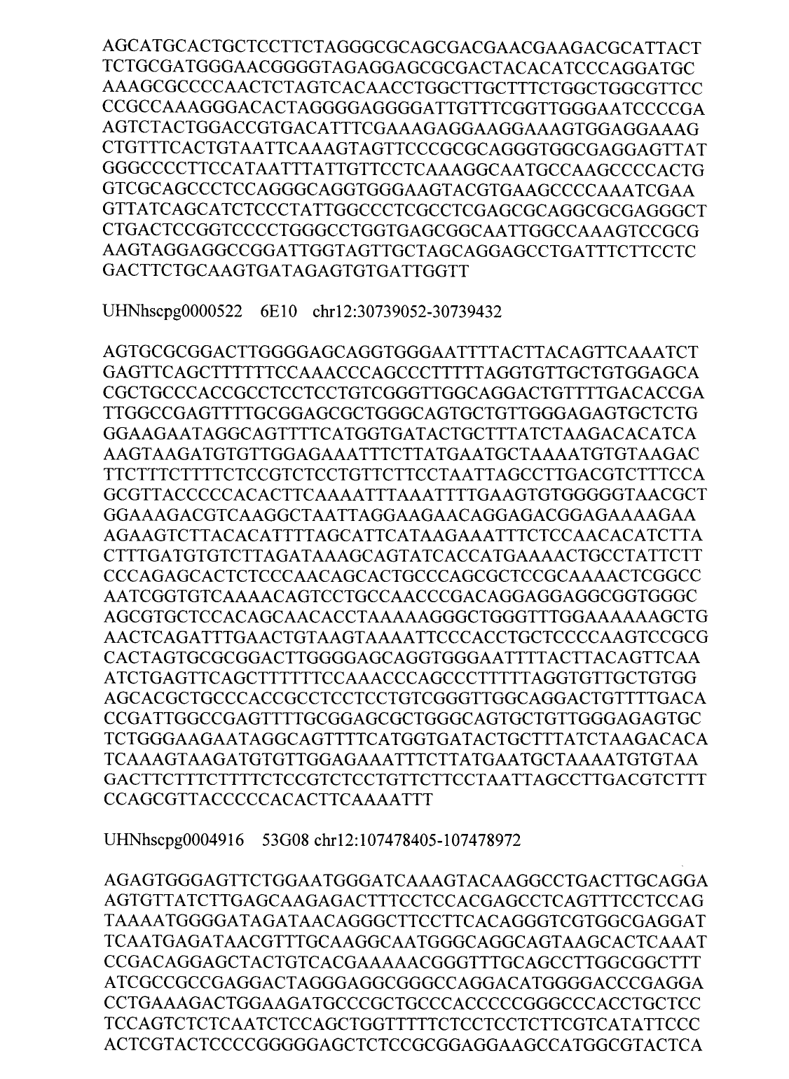

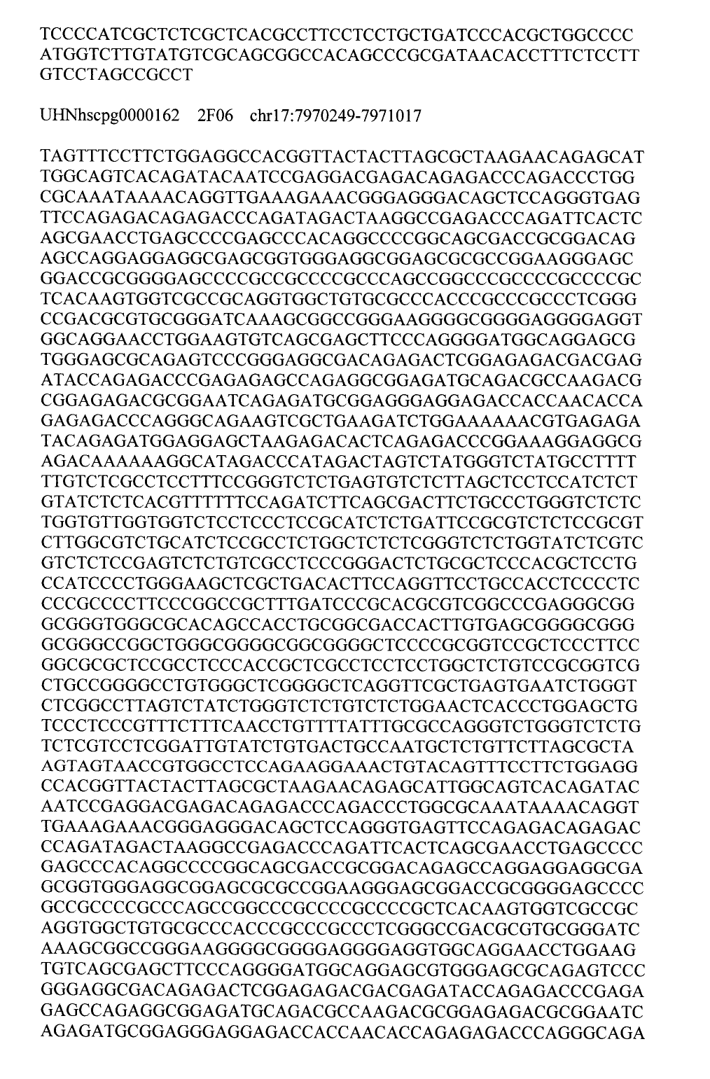

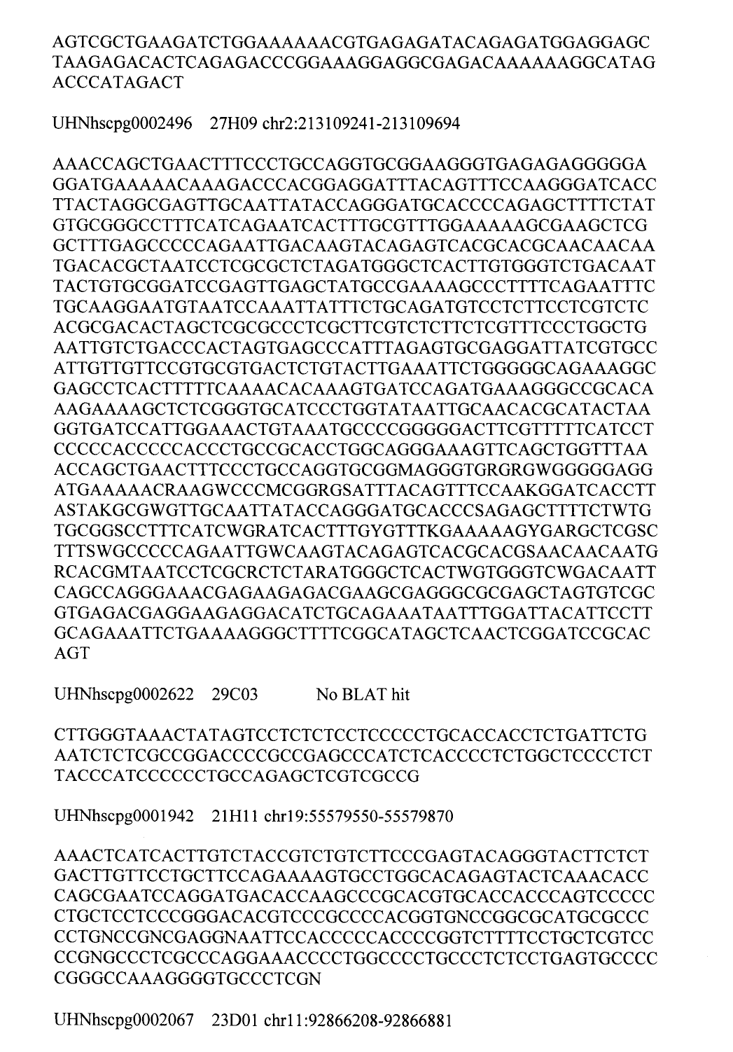

We have performed DNA methylation microarray analysis comparing sperm cell DNA from 20 bipolar cases to sperm cell DNA from 20 healthy controls. The case vs control t-test identified 582 significant loci (unadjusted P_{<}0.05). When we have applied the FDR multiple correction the number of significant loci (P<0.3) is reduced to 33 loci (Table 4 and FIG. 9). The nucleotides sequences can be obtained from the UHN website http://data.microarrays.ca/cpg/ using the UHNID. The sequences of the 33 loci described above are provided in FIG. 11. This includes CpG islands within genes such as, but not limited to the Neuregulin 1 receptor ERBB4 (FIG. 10) and the histone 1 family member HIST1H3G, among others. Additional information for other loci is provided in Table 5.

As will be appreciated by a person of skill in the art, the p value may be selected to increase or decrease the results contained in the data set obtained by the method of the present invention. However, the implications of using a higher p value (ie p=0.4) are that more of the selected loci are expected to be false positives. Similarly, a lower P value is expected to result in a lower number of false positives. The present invention contemplates using any P-value or a range of P-values.

The methylation differences in bipolar disorder cases compared to controls may be confirmed using one or more alternate methods known in the art, for example, but not limited to bisulphite-modification based analysis. The present study is the first to provide evidence for epimutations in bipolar disorder patients. Accordingly, the subject matter provided herein and throughout is useful for providing methods for identifying one or more epimutations in subjects that may be associated with a disease state, for example, but not limited to bipolar disorder. In addition, the regions of nucleotide sequences that comprise one or more epimutations may serve as useful diagnostic epigenetic biomarkers and can be employed in diagnostic tests and the like.

### Example 3

### DNA Methylation Changes Associated with Major Psychosis

Epigenetic misregulation is consistent with various non-Mendelian features of major psychosis-associated diseases. In this study 12,192-feature CpG-island microarrays were used to identify DNA methylation changes in the frontal cortex (N=95) and germline (N=40) associated with major psychosis-asscoiated diseases including schizophrenia and bipolar disease. Psychosis-associated brain DNA methylation differences were identified in over 100 loci, including several genes involved in glutamatergic and GABAergic neurotransmission, brain development, and other processes functionally-linked to disease etiology. DNA methylation changes in a significant proportion of these loci correspond to reported changes of steady-state mRNA level associated with psychosis. Gene ontology analysis highlighted epigenetic disruption to loci involved in mitochondrial function, brain development, and stress response. Methylome network analysis uncovered decreased epigenetic modularity in both the brain and the germline of affected individuals, suggesting that systemic epigenetic dysfunction may be associated with major psychosis.

Introduction

Schizophrenia (SZ) and bipolar disorder (BD) are etiologically related psychiatric conditions, together termed ‘major psychosis’ (PSY). Studies of PSY have focused primarily on the interplay between genetic and environmental risk factors. Twin and adoption studies highlight a clear inherited component to both disorders (1), but while replicated findings exist for a number of genes, association studies are characterized by non-replication, small effect-sizes, and significant heterogeneity (2). Several epidemiological, clinical, and molecular peculiarities associated with PSY are hard to explain using traditional gene- and environment-based approaches, including the non-complete concordance between monozygotic twins for both SZ (41-65%) and BD (˜60%)(1,3), which cannot be accounted for by only environmental factors (2,4). Other complexities of PSY include a fluctuating disease course with periods of remission and relapse, sexual dimorphism, peaks of susceptibility to disease coinciding with major hormonal rearrangements, and parent-of-origin effects (2). These observations have led to speculation about the importance of epigenetic factors in mediating susceptibility to both SZ and BD2.

Epigenetics refers to the heritable, but reversible, regulation of gene expression mediated principally through modifications of DNA and histones (5). Epigenetic processes are essential for normal cellular development and differentiation, and allow the regulation of gene function through non-mutagenic mechanisms. The impact of DNA methylation on gene activity has been explained by two proven mechanisms. The ‘critical site’ model puts an emphasis on the methylation of specific cytosines in transcription-factor binding sites, reducing binding affinity and thus the transcription of mRNA(6). The ‘methylation density’ model suggests that the proportion of methylated cytosines across a region, rather than at any specific position, controls chromatin conformation and thus the transcriptional potential of the gene(6).

The epigenetic model of PSY is based upon three general principles(2). First, that like the DNA sequence, the epigenetic profile of somatic cells is mitotically inherited, but unlike the DNA sequence epigenetic signals are dynamic. The epigenetic status of the genome is tissue-specific, developmentally-regulated, and influenced by both stochastic and environmental factors. Second, because epigenetic processes regulate gene expression, epigenetic metastability can have profound phenotypic effects. Genes, even those containing no mutations or disease predisposing polymorphisms, may be harmful if not expressed in the appropriate amount, at the right time of the cell cycle or in the right compartment of the nucleus. Third, some epigenetic signals, rather than being reset and erased during gametogenesis, may be transmitted meiotically across generations (7). This has obvious ramifications for the identification of the molecular substrate of inherited predisposition, in which heritable phenotypic variation is assumed to result exclusively from DNA sequence variants.

To date few studies have investigated the role of epigenetic factors in PSY. DNA methylation differences have been reported in the vicinity of both catechol-O-methyltransferase (COMT)(8) and reelin (RELN)(9), although these findings were not confirmed using fully quantitative methylation profiling methods (10,11). In this article we report findings from a comprehensive epigenomic study of PSY. Using DNA from the frontal cortex, a region previously implicated in the etiology of PSY12, derived from individuals with SZ, BD, and matched controls (CTRL), we examined DNA methylation utilizing two complementary approaches. First, we performed a microarray-based epigenomic scan of PSY using CpG-island microarrays following enrichment of the unmethylated fraction of brain DNA. Second, we performed a hypothesis-driven analysis of DNA methylation across candidate genes for which a priori evidence for a role in the etiology of PSY exists. In addition, to investigate whether epigenetic differences could be observed in the germline, we also used CpG-island microarrays to profile germline DNA methylation in BD patients and controls.

Materials and Methods

Samples: Post-mortem brain tissue of individuals with DSM-IV diagnosed SZ (n=35), BD (n=35) and matched controls (n=35) were provided by the Stanley Medical Research Institute (Array Collection). The samples consisted of frozen tissue sections, which were stored at −80° C. prior to DNA extraction. Additional information on the brain samples utilized in this study can be found at http://www.stanleyresearch.org/programs/brain_collection.asp. In addition, germline samples were available from male BD patients (n=20) and unaffected controls (n=20) from an ongoing study at the Centre for Addiction and Mental Health (Toronto, Canada). Extraction of all DNA was performed using a standard phenol/chloroform extraction method. The quality and quantity of DNA was assessed by spectrophotometry and agarose gel analysis, and subsequently stored at −20° C. until further use. Demographic data for the samples is summarized in Table 7.

Enrichment of unmethylated DNA and microarray hybridization: We used our developed technology for enrichment of the unmethylated DNA fraction and for epigenetic profiling using microarrays, described in detail elsewhere (15). In brief, the methylation-sensitive restriction enzyme HpaII (New England Biolabs) was used to digest 1 μg of genomic DNA. DNA adaptors (annealing products of two primers, U-CG1A and U-CG1B (see Table 8)) were ligated to the cleaved DNA fragments, followed by treatment with McrBC (New England Biolabs), which cleaves fragments containing two or more methylated cytosines, thereby further enriching the unmethylated fraction. Adaptor-PCR amplification of the ligated products, with the use of primers complementary to the adaptor sequence, consisted of 250 ng of ligated DNA, 2.5 mM MgCl₂, 0.2 mM aminoallyl-dNTPs (15mM aminoallyl-2′-deoxyuridine 5′-triphosphate, 10 mM 2′-deoxythymidine 5′-triphosphate, and 25 mM each of 2′-deoxycytidine 5′-triphosphate, 2′-deoxyguanosine 5′-triphosphate, and 2′-deoxyadenosine 5′-triphosphate), 200 pmol primer U-CG1B, and 5U Taq polymerase (New England Biolabs) in 1× PCR reaction buffer (Sigma), to a final volume of 100 μl. PCR conditions are adjusted in such a way that fragments <1.5 kb (i.e. those that are digested, short and thus unmethylated) will amplify preferentially. Cycling consisted of an initial cycle at 72° C. for 5 min and 95° C. for 1 min, 25 cycles at 95° C. for 40 s and 68° C. for 2 min 30 s, and a final extension at 72° C. for 5 min. Given that the role of epigenetic effects in disease etiology may be sex-specific, and considerable differences are observed in the course and prognosis of PSY between males and females, we split our sample according to gender. For the brain samples, equal amounts of amplicons from CTRL male samples were mixed to form a pooled male CTRL, and from CTRL female samples to form a pooled female CTRL. Individual samples were then co-hybridized with the relevant common reference pool sample. For the germline samples, all samples were co-hybridized with a common reference pool made by combining amplicons from all CTRL samples. Samples were hybridized on 12,192 CpG island microarrays obtained from the University Health Network Microarray Facility in Toronto. For the brain samples, good quality DNA extraction, enrichment and microarray hybridization was successful for 28 CTRL samples, 35 SZ samples, and 32 BD samples. For the germline samples, good quality DNA extraction, enrichment and microarray hybridization was successful for 19 CTRL samples and 20 BD samples.

Microarray data pre-processing: Initial array image processing and quality control was performed using GenePix Pro 6.0 (Molecular Devices). The array signals were background-corrected using NormExp and normalized using weighted block-by-block LOWESS normalization. Spots with ambiguous genome locations, including spots with no sequence or annotation, repetitive spots, and translocation hotspots were removed, leaving a total of 7,834 spots.

Normality Testing: Several analyses assumed data to be drawn from a normal distribution, hence the need for normality testing. Log intensity ratios for each spot were subjected to the Lilliefors test for normality. The resultant p-values for all spots were adjusted for multiple testing using Benjamini and Hochberg's FDR method (50).

Microarray data analysis: Limma was used to analyze each array spot for differential methylation between affected and unaffected samples. Each spot was assigned a raw p-value based on a moderated t-statistic. To correct for multiple testing, the set of raw p-values were converted to false discovery rates (FDR) according to Benjamini and Hochberg (50).

Gene ontology (GO) analysis: A novel gene ontological investigation approach was designed to determine if any common functional trends are associated with the genes exhibiting differences between groups. For each group interrogated, only those loci exhibiting a significance value of less than p=0.01 from a spot-wise t-test were selected in order to include only those loci likely to have a true DNA methylation difference between groups. Gene IDs within 1 kb of these array loci were obtained from the microarray annotation data (available at www.microarrays.ca) and cross referenced with the April 2007 build of the Gene Ontology Database (www.geneontology.org) to obtain gene ontology (GO) categories associated with each microarray locus. All loci and corresponding mean fold change values were sorted into categories based on their GO classification, and the distribution of each GO category was compared with a paired t-test and the more conservative Wilcoxon Signed Rank test. In both cases, p-values were adjusted with FDR to correct for multiple testing. Data was then sorted by FDR p-value, revealing the most significantly different GO categories.

Network analysis of microarray data: In order to investigate if DNA methylation is coordinated across different loci, we utilized a novel network-based approach. For brain samples, this analysis was performed on twenty male SZ samples and an equal number of male CTRL samples—the other diagnostic groups were not included in this analysis because of their small sample sizes. We identified the top 700 methylation variable spots across the samples in each group. The union of these two sets, consisting of 1041 spots, was chosen for network reconstruction. To find connections between methylation at specific genomic regions (nodes), their methylation log intensities were modeled by a linear combination of the methylation log intensities at the remaining spots. After regression, the correlation between the minimized residuals was calculated, measuring the direct association between the two spots. Estimation of correlation and p-value was accomplished by a regularized covariance estimator that addresses the issue of small size and large variable (20 <<1042) (51). As a control for the network analysis, in each of the 20 CTRL microarrays, we randomized the IDs of the 1041 spots and proceeded with the same estimator. A raw p-value of 10-7 was then chosen to cut-off the insignificant pair-wise correlations. A connection was drawn between a pair of spots whose correlation p-value survived the cut. The structure of each network was explored by calculating the transitivity (quantifying the connectivity between a spot's neighbors) and assortativity (quantifying the tendency of attachment between high connection spots). The modular structure of a network was detected by a partitioning algorithm (41) which maximizes the within-module connection densities at the expense of between-module connection densities. The analysis was repeated on the germline BD and CTRL samples.

Correlation with anti-psychotics used: Linear regression was performed on psychosis patients, with log intensity ratios for each spot as dependent variable and lifetime dosage of anti-psychotics applied as independent variable. Base-2 logarithm of the dosages were taken for the regression due to their wide spread. After the regression, p-values based on F-statistics were gathered for all spots and converted to FDR to control for multiple testing.

Bisulfite treatment of genomic DNA: Bisulfite treatment was performed using a standard protocol. Briefly, ˜500 ng genomic DNA was denatured in 0.3 M NaOH for 15 mM at 37° C. After adding freshly prepared 3.5M sodium metabisulfite (Sigma) and 1 mM Hydroquinone (Sigma) solution, samples were subjected to a 5-hour incubation at 55° C. under exclusion of light. The samples were then purified using Qiagen DNA purification columns (Qiagen). Recovered samples were desulfonated in 0.3M NaOH for 15 minutes at 37° C. and neutralized. DNA was precipitated overnight in ethanol at −20° C. and resuspended in 50 μl buffer EB (Qiagen). Bisulfite treated DNA was stored at −80° C. until needed.

Bisulfite primer design and PCR amplification: Primers were designed using either MethPrimer, available online at http://www.urogene.org/methprimer/index1.html, or Pyrosequencing Assay Design Software v1.0.6 (Biotage, Sweden). For loci nominated from microarray analyses, primers were designed, where possible, to span a region containing potentially informative HpaII sites in the vicinity of the significant clone on the CpG island microarray. Where necessary, larger regions were covered using several overlapping amplicons. For selected candidate genes, the primary focus of analysis was promoter CpG islands. In some cases (e.g. COMT and BDNF), additional exonic regions in the vicinity of known genetic polymorphisms were also investigated. Where candidate genes had been previously investigated by other groups (RELN and COMT), we ensured that the same regions were adequately covered by our analyses. A full list of primer sequences and annealing temperatures for each PCR reaction can be found in Table 8. PCR amplifications were performed using a standard hot-start PCR protocol in 250 volume reactions containing 3 μl of sodium-bisulfite treated DNA, 1 μM primers, and a master mix containing hot-start Taq polymerase (Sigma). All PCR reactions were checked on a 1.0% agarose gel to ensure successful amplification and specificity before proceeding with Pyrosequencing or MS-SNuPe.

Site-specific DNA methylation analysis using Pyrosequencing and MS-SNuPe: For Pyrosequencing analysis, bisulfite-PCR products were processed according to the manufacturer's standard protocol (Biotage, Uppsala, Sweden). Briefly, 4 μl of streptavidin-sepharose beads (Amersham Biosciences, Piscataway, N.J., USA) and 40 μl of binding buffer (10 mM Tris-HCl, 1 mM EDTA, 2 M NaCl) were mixed with 40 μl of PCR product for 10 min at room temperature. The reaction mixture was placed onto a MultiScreen-HV, Clear Plate (Millipore, Billerica, Mass., USA). After applying the vacuum, the beads were treated with a denaturation solution (0.2 N NaOH) for 1 min and washed twice with washing buffer (10 mM Tris-acetate at pH 7.6). The beads were then suspended with 24 μl of annealing buffer (20 mM Tris-acetate, 2 mM Mg-acetate at pH 7.6) containing 8 pmol of sequencing primer. The template-sequencing primer mixture was transferred onto a PSQ 96 Plate (Biotage), heated to 90° C. for 2 min followed by 60° C. for 10 min and finally cooled to room temperature. Sequencing reactions were performed with a PSQ 96 SNP Reagent Kit (Biotage) according to the manufacturer's instructions. The percentage methylation at each CpG site were calculated from the raw data using Pyro-Q-CpG Software (Biotage). MS-SNuPe analysis was performed using ABI SNaPshot reagents (Applied Biosystems) using a method developed in our laboratory. Extension products were separated on an ABI3100 Genetic Analyzer (Applied Biosystems). Methylation data from Pyrosequencing and MS-SNuPe analysis was analyzed with SPSS v14 (Lead Technologies, Inc) using standard t-tests and ANOVA.

Genotyping of COMT and BDNF SNPs: Non-synonymous SNPs in COMT (rs4680—val108/158met) and BDNF (rs6265—val66met) could be genotyped using the Pyrosequencing assays designed to cover these regions. In addition, genotypes were double-checked using the ABI TaqMan Allelic discrimination method utilizing Assay-on-Demand reagents provided by the manufacturer (Applied Biosystems) and the ABI 7900HT Sequence Detection System. DNA methylation at surrounding CpG sites was compared between samples grouped by genotype using standard t-tests.

Results

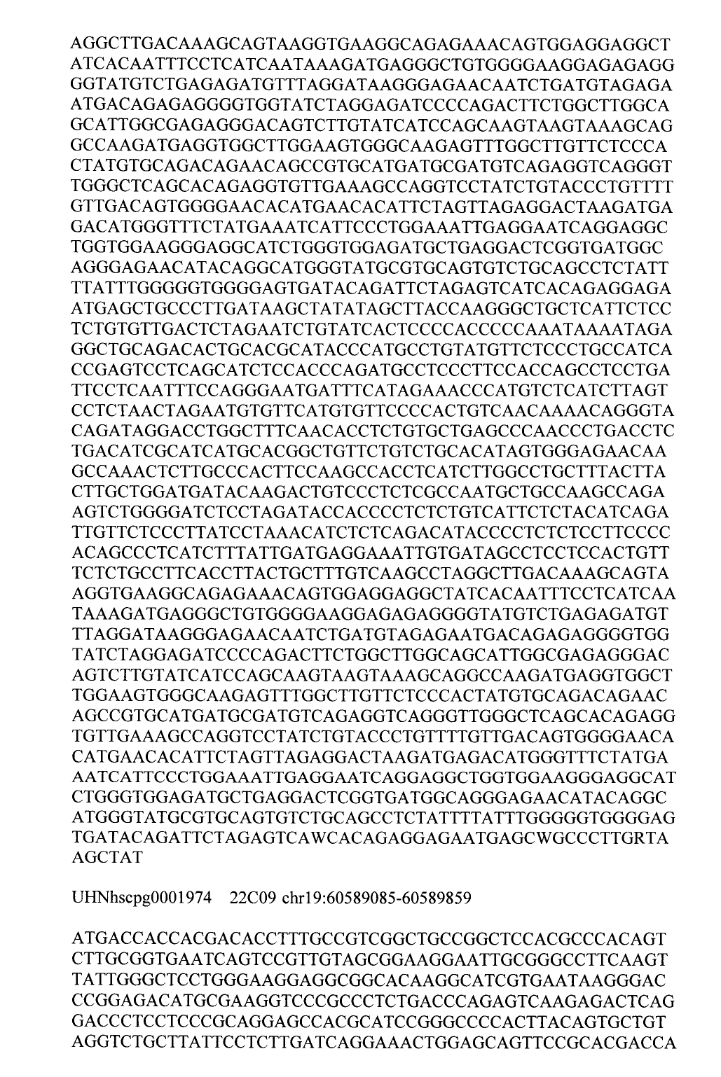

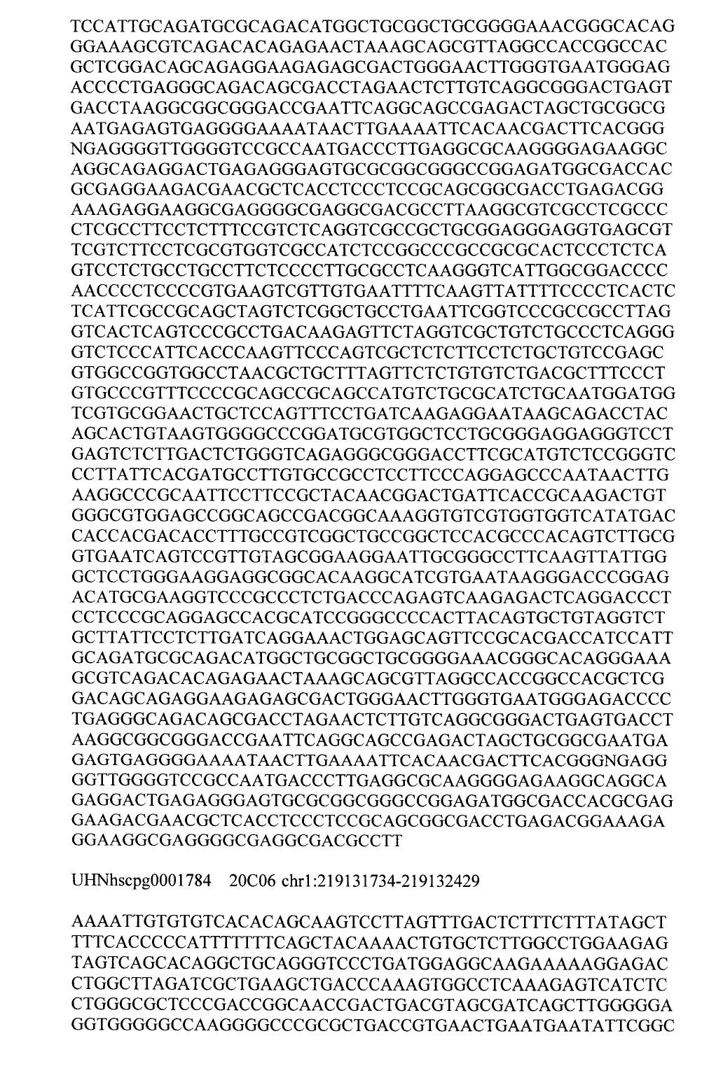

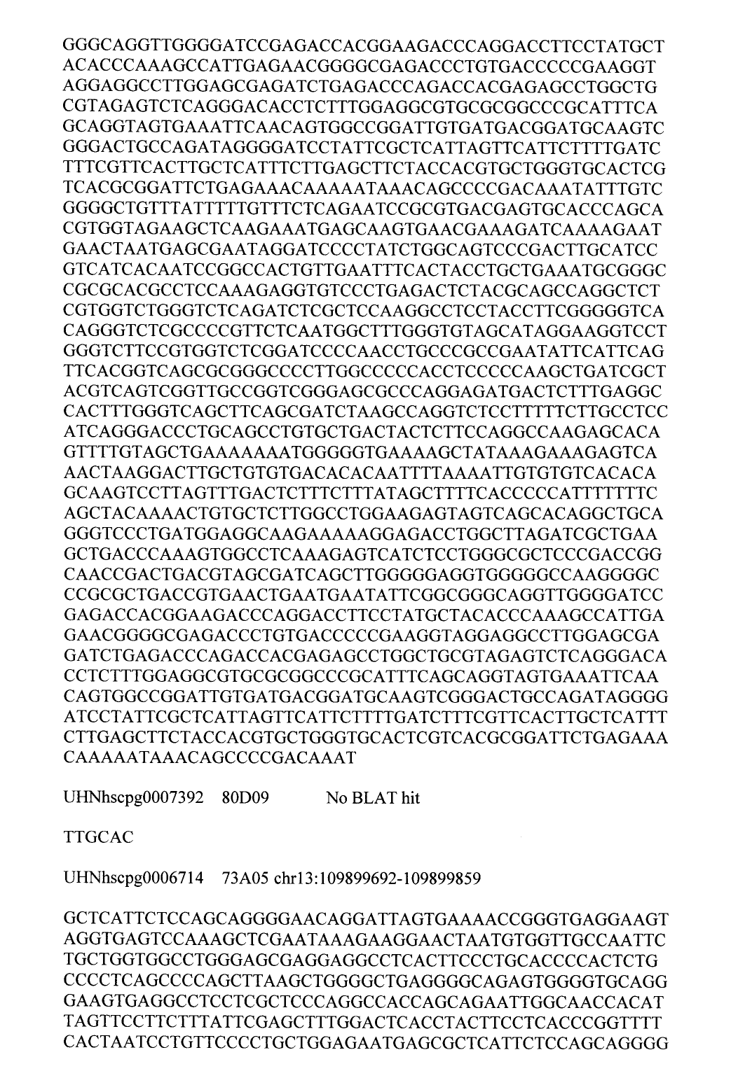

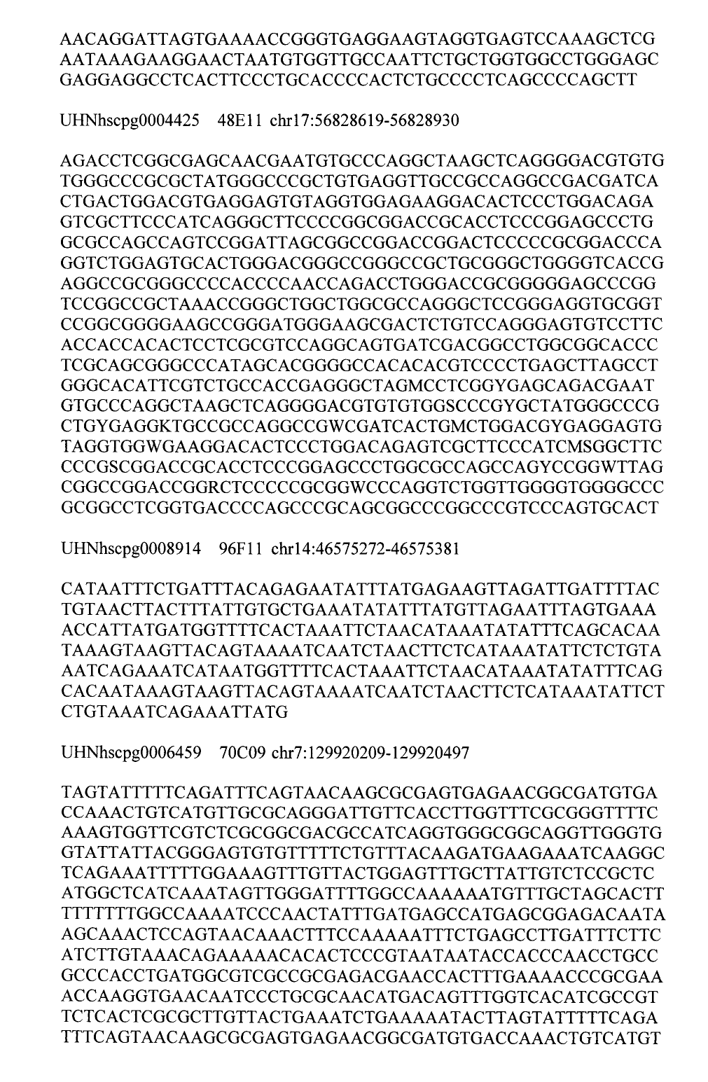

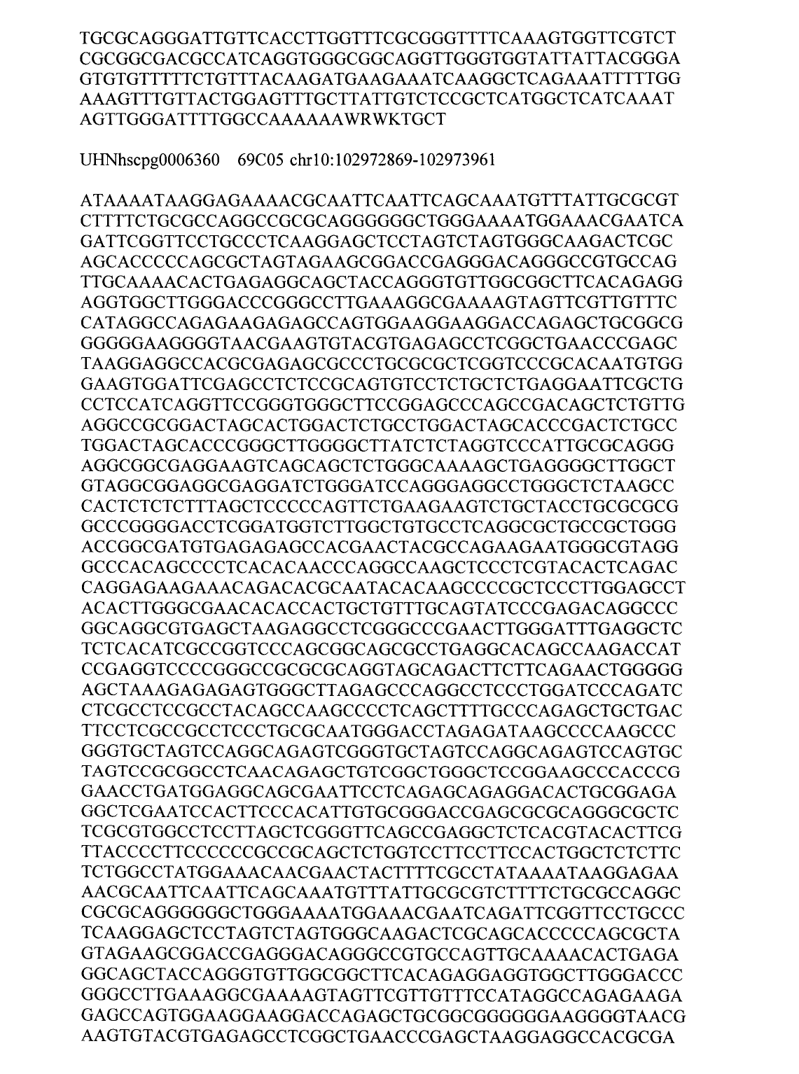

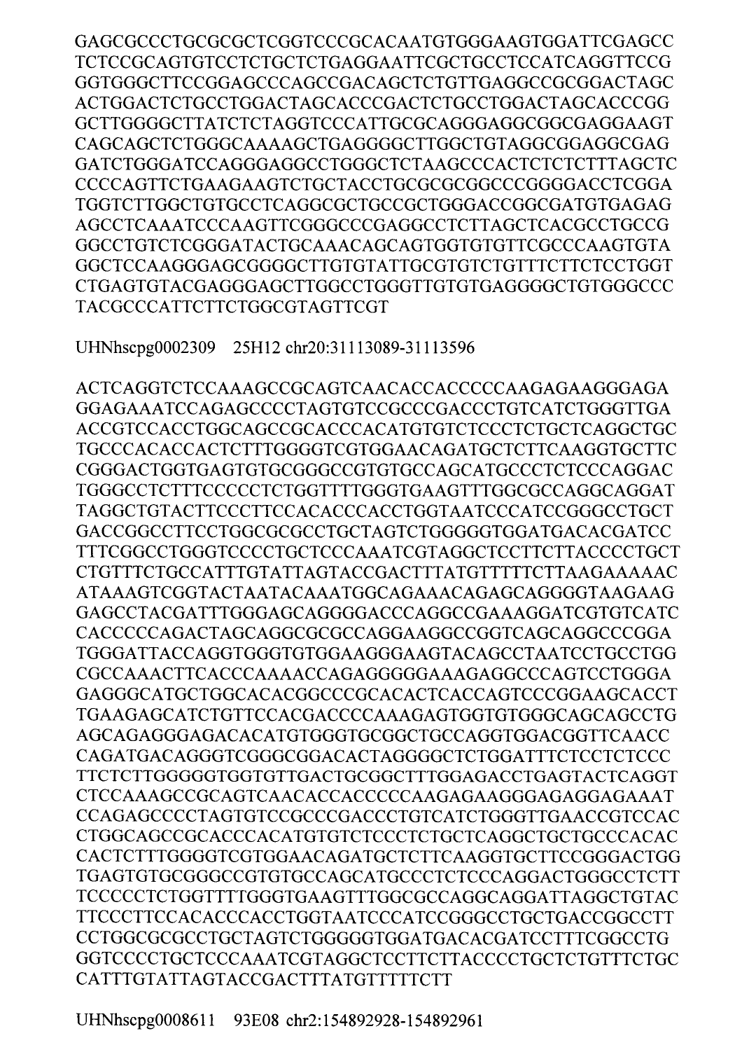

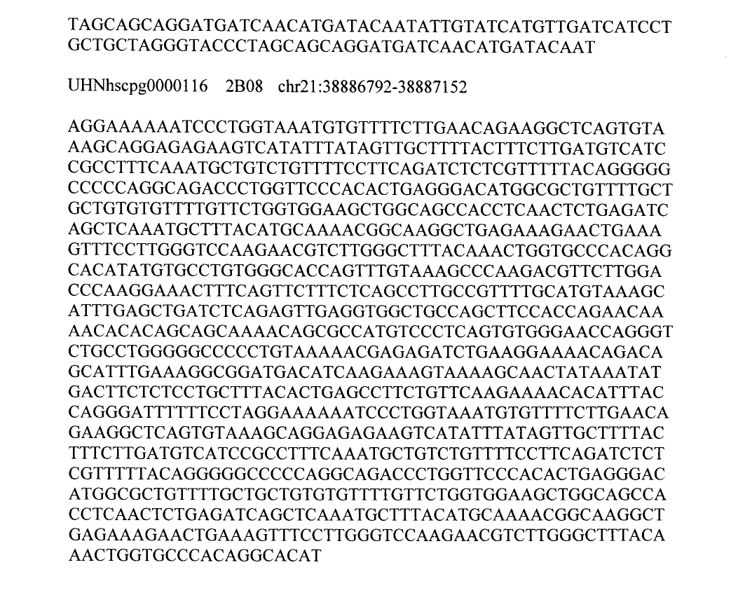

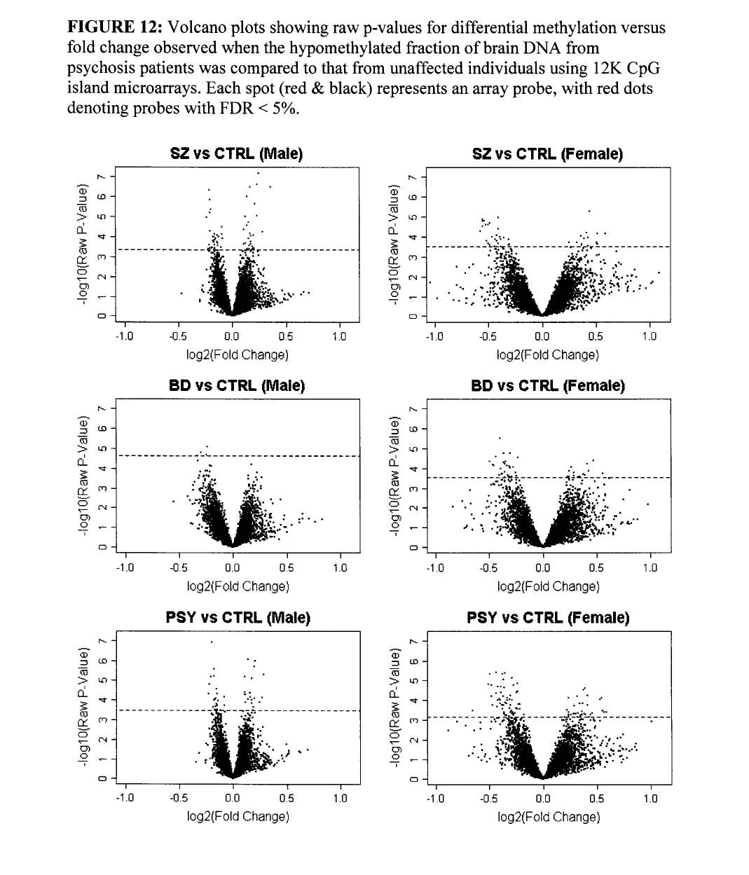

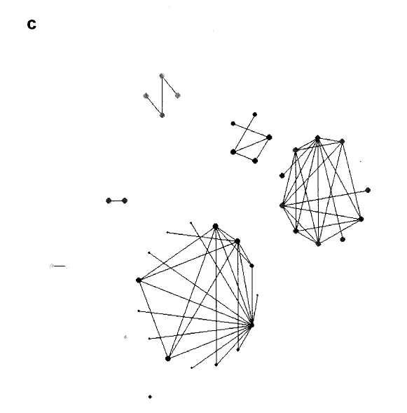

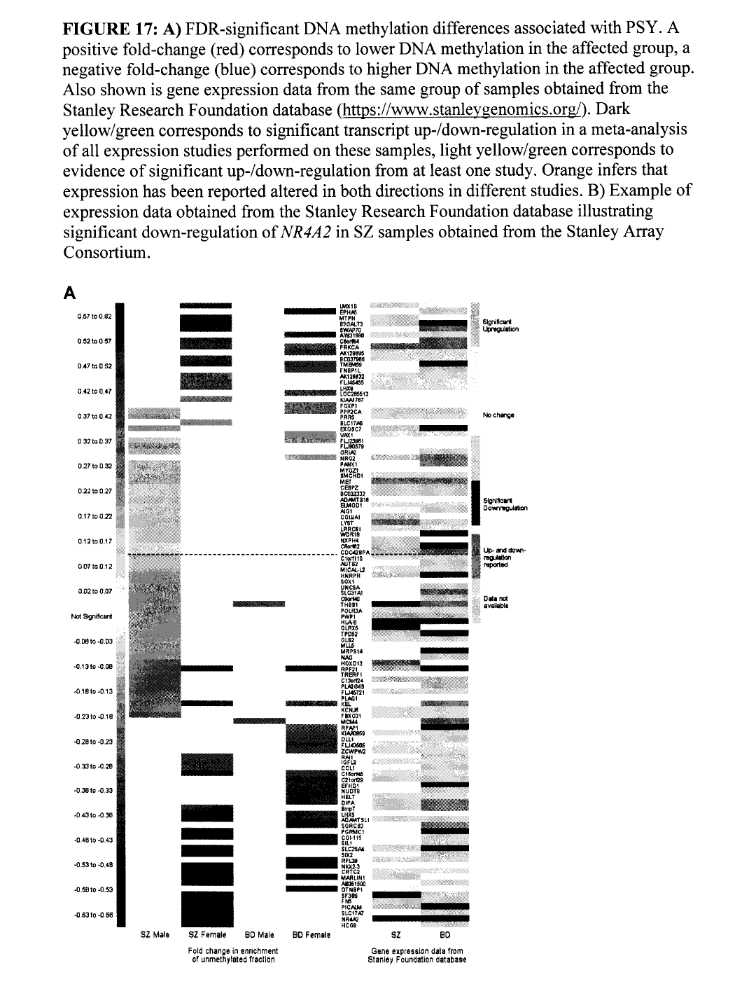

Methylomic profiling of brain DNA: FIG. 12 illustrates raw p-values for microarray signal intensity versus fold-change observed for comparisons between brain DNA from PSY patients and unaffected controls, matched for sex. Significant (FDR<0.05) mean differences were found for spots associated with a number of genes. Many of these loci are consistent with our knowledge about the neurobiological and genetic systems involved in PSY (Table 6 and Table 9), including several glutamatergic and GABAergic genes, loci involved in neuronal development, and loci highlighted in genetic linkage studies. The full list of FDR-significant loci are presented in Table 10 and FIG. 17A.

Gene expression data for the samples used in this study is available from the Stanley Medical Research Institute Online Genomics Database (https://www.stanleygenomics.org/) (13). FIG. 17b illustrates an example of the available expression data, highlighting downregulation of NR4A2, a gene found to be hypermethylated in female SZ samples. For NR4A2, 9 out of 24 studies show significantly reduced expression, with the overall analysis across all mRNA studies in the Stanley Array Collection being highly significant (p=<0.00001) (FIG. 17b). FIG. 17a summarizes the available expression data for all FDR-significant DNA methylation changes. 84% of the loci found to be hypermethylated in PSY samples, for which expression data is available (42 out of 50), are significantly down-regulated in at least one gene expression study with 28% (14 out of 50) showing significant down-regulation of expression across all mRNA studies performed on these samples.

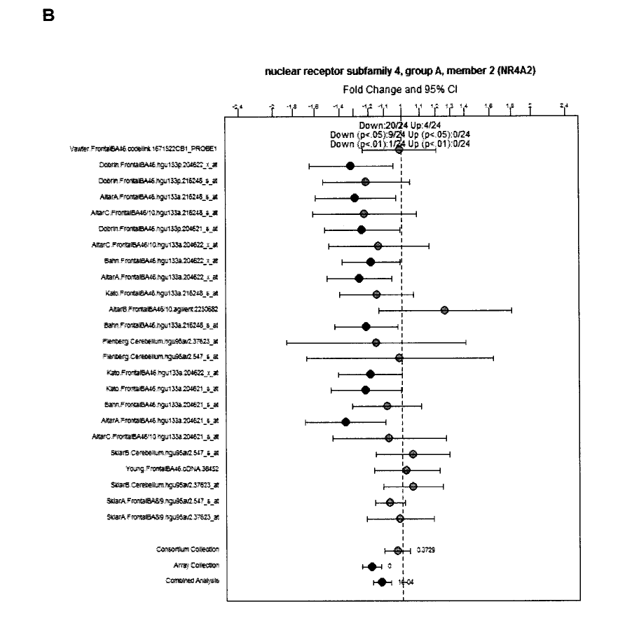

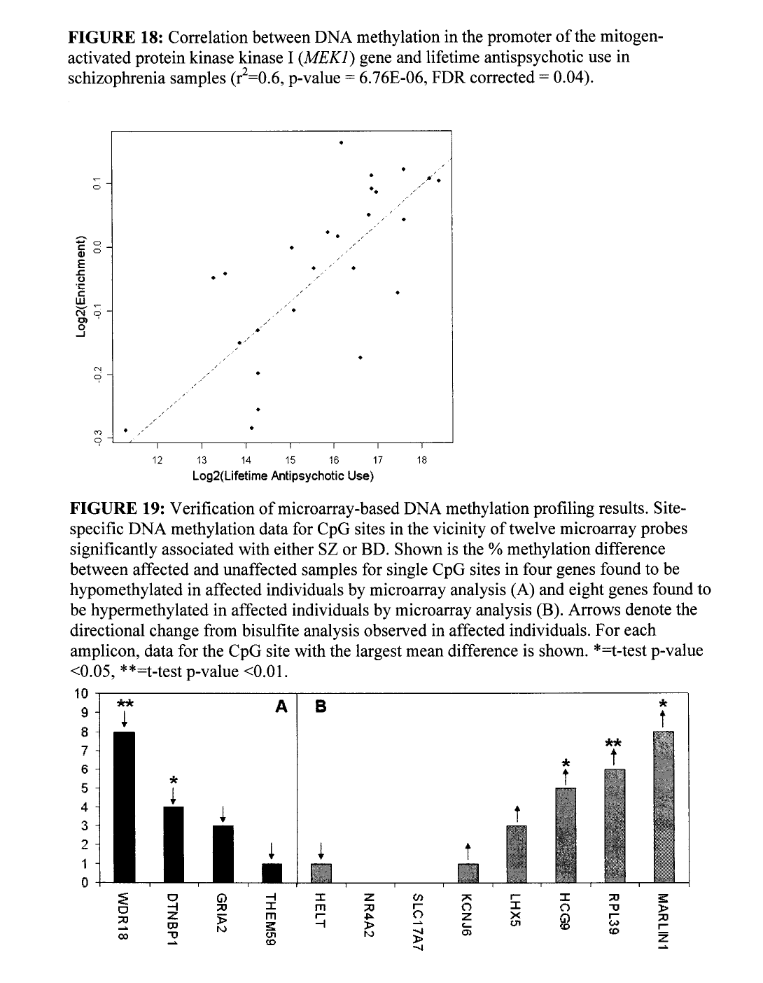

Analysis of demographic data, brain-tissue parameters, and lifetime antipsychotic use: No FDR-significant correlations were found between any of the available demographic variables (PMI, brain weight, brain pH, lifetime alcohol use, and lifetime illicit drug use) and DNA methylation. Methylation of a CpG island located ˜30 kb upstream of the gene encoding mitogen-activated protein kinase kinase I (MEK1) was found to be significantly correlated with lifetime antipsychotic use in male SZ samples (r2=0.6, p=6.76E−06, FDR=0.04), with higher lifetime antipsychotic use associated with lower DNA methylation (FIG. 18).

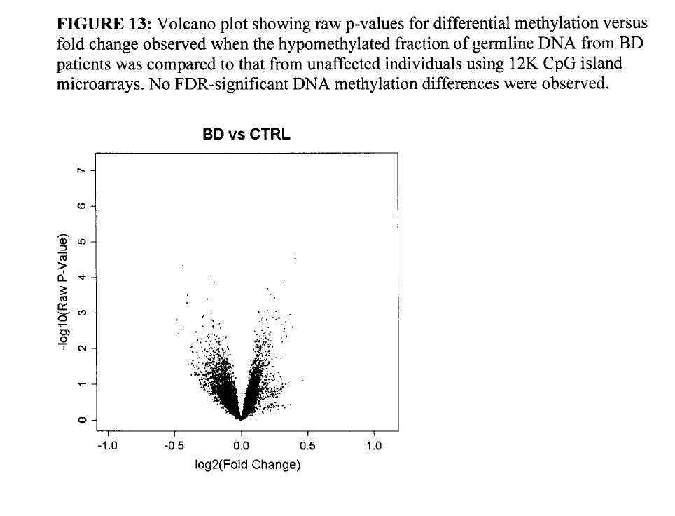

Methylomic profiling of germline DNA: FIG. 13 illustrates raw p-values for array signal intensity versus fold-change observed in our comparison of germline DNA from BD patients and unaffected CTRLs. No FDR-significant differences were observed between the two groups. A comparison of the largest psychosis-associated DNA methylation differences in the germline analysis with those in the brain DNA analysis, taking loci with a raw p-value <0.001, found no overlap between datasets.

Site-specific CpG methylation analysis in selected genes: validation of microarray methodology: Previous studies have shown that the methylation-sensitive restriction enzyme based enrichment protocol utilized in this study can be used to reliably measure real DNA methylation differences (14,15). Following microarray analysis, we tested a number of loci to further verify the microarray approach. From the genes listed in Table 6, we quantitatively measured site-specific CpG methylation upstream of DTNBP1 (n=30), GRIA2 (n=39), HCG9 (n=31), HELT (n=26), KCNJ6 (n=26), LHX5 (n=24), MARLIN-1 (n=28), NR4A2 (n=24), RPL39 (n=25), SLC17A7 (n=24), THEM59 (n=30), and WDR18 (n=29). Given that our enrichment strategy was based on differential cleavage of HpaII sites, we focused primarily on these and surrounding CpG positions located in or near genomic regions corresponding to specific microarray probes.

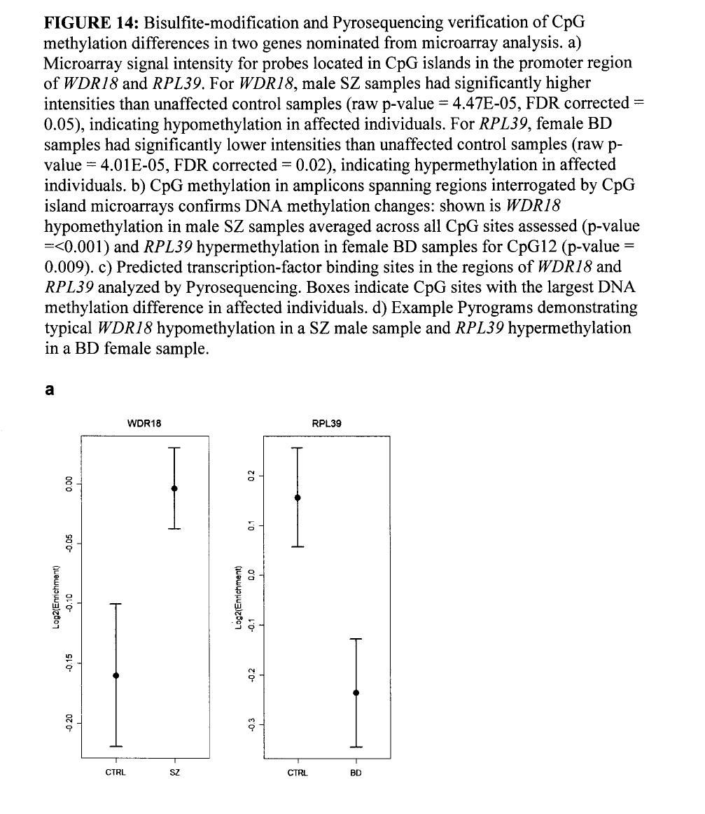

Our site-specific CpG analyses show good agreement with data obtained from microarray analysis. Two examples are shown in FIG. 14 for regions upstream of the genes WDR18 and RPL39. Microarray analysis (FIG. 14a) predicted these regions to be hypomethylated in SZ male samples and hypermethylated in BD female samples, respectively. FIG. 14B shows Pyrosequencing data confirming WDR18 hypomethylation in male SZ samples compared to controls (n=29, average methylation 17% vs 25%, p=<0.001). This region contains a putative binding site for the brain-expressed transcription-factor c-myb, known to be blocked by CpG methylation16(FIG. 14c). Pyrosequencing also verified RPL39 hypermethylation in BD female samples (n=25, 28% vs 22%, p=0.009), especially at a CpG located within putative binding sites for several brain-expressed transcription-factors that are known to be affected by DNA methylation, PAX-517 and NF-kB 18 (FIG. 14c).

FIG. 19 highlights DNA methylation changes observed in the genes selected from Table 6 for site-specific CpG methylation analysis. In addition to WDR18 and RPL39, we were able to confirm DNA methylation differences in MARLIN-1, postulated to be hypermethylated in affected female samples from microarray analysis. Average methylation of a HpaII site located in the genomic region spanning the microarray clone, and falling in a putative binding site for a Pbx1/Meis1 heterodimer, was 84% in unaffected controls, 93% in SZ females, and 91% in the combined PSY female group. Interestingly, while only 13% of unaffected control samples were fully methylated, 71% of SZ females, and 47% of the combined female PSY group were fully methylated.

No significant DNA methylation differences were detected in 7 of the 12 ‘positive’ microarray regions tested. However, even in these regions, changes were consistently in the direction predicted by our microarray analysis (FIG. 19). Five confirmed loci out of 12 tested (˜40%) is a relatively good rate of verification given that no DNA methylation differences were observed in any of the 12 ‘negative’ array regions tested (data not shown). This data suggest that that a substantial proportion of epigenetic differences detected in our microarray experiments are real, and the conclusions drawn from the full array dataset are likely to be based on genuine DNA methylation changes.

Gene ontology analysis of brain methylomic data: The top 60 GO categories for each diagnostic group can be seen in FIG. 15. Table 11 lists all significant GO categories with a p<0.01. GO categories detected by this analysis included genes involved in the epigenetic regulation of transcription and development. Of particular interest to the etiology of psychosis were the FDR-significant associations for “response to stress” in male BD samples, and “brain development” in both female BD samples and female SZ samples. In addition, consistent with the postulated link between mitochondrial function and psychosis 19, several mitochondrial GO categories, involving loci on the nuclear genome, show significantly different distributions in the affected individuals compared to controls.

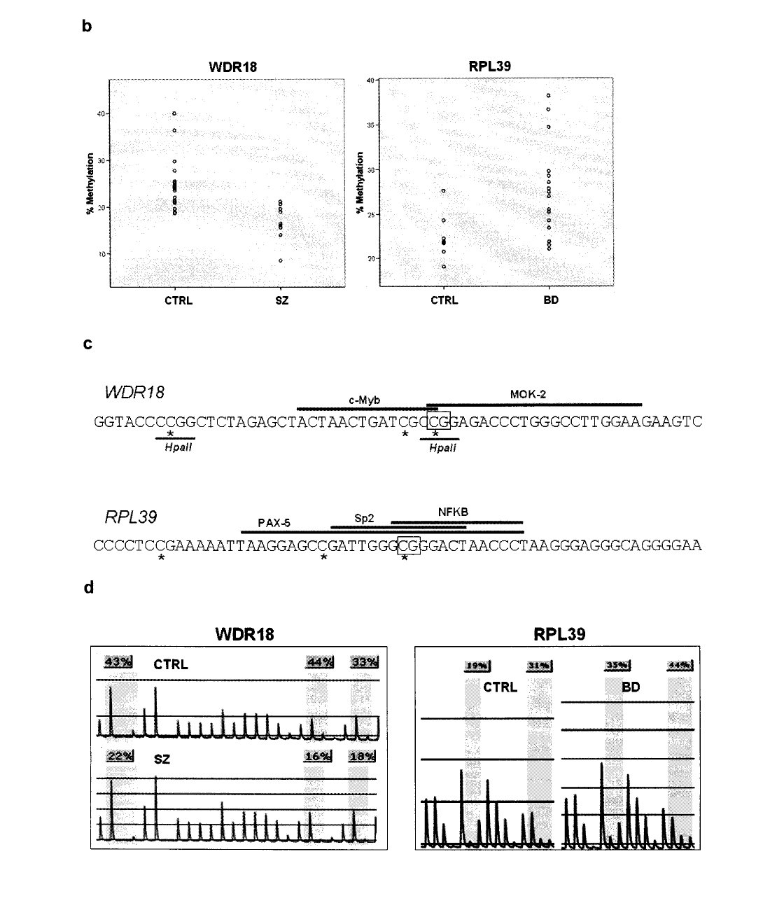

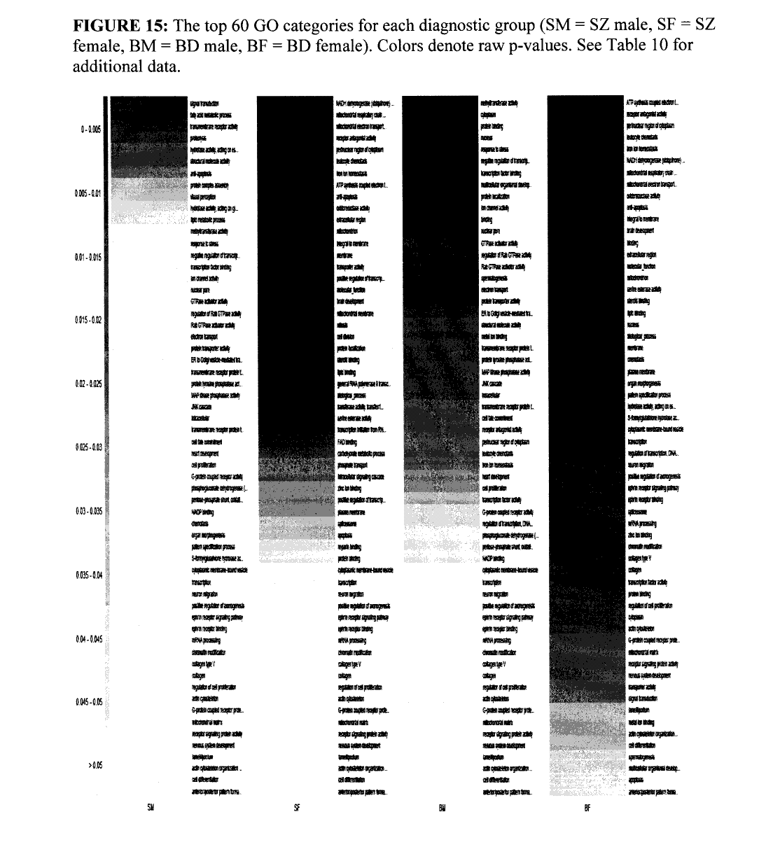

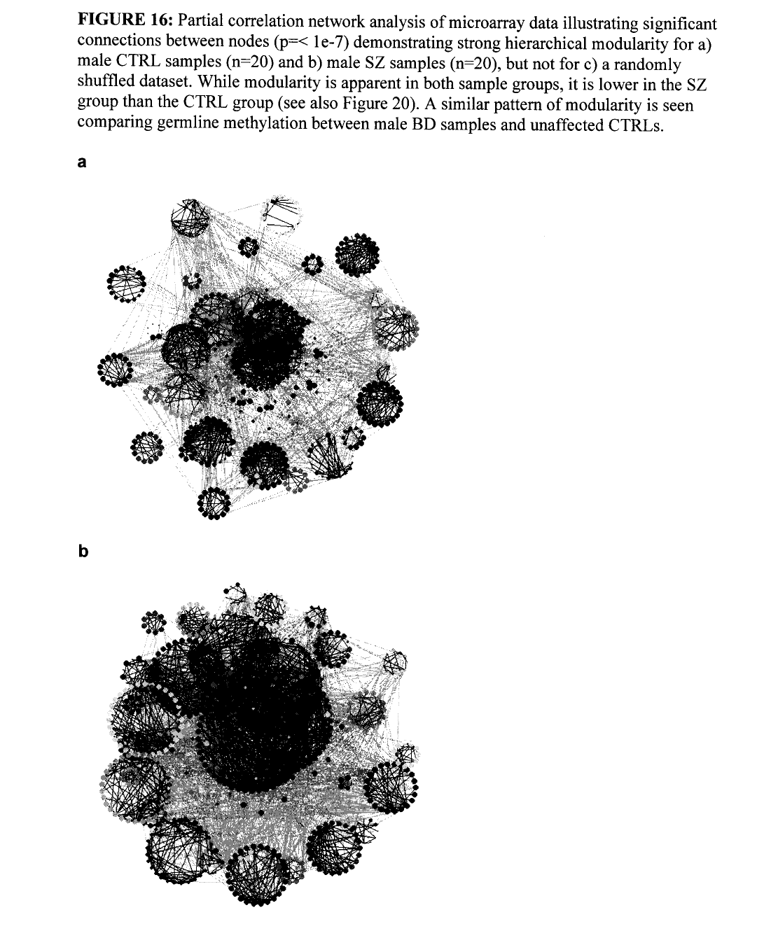

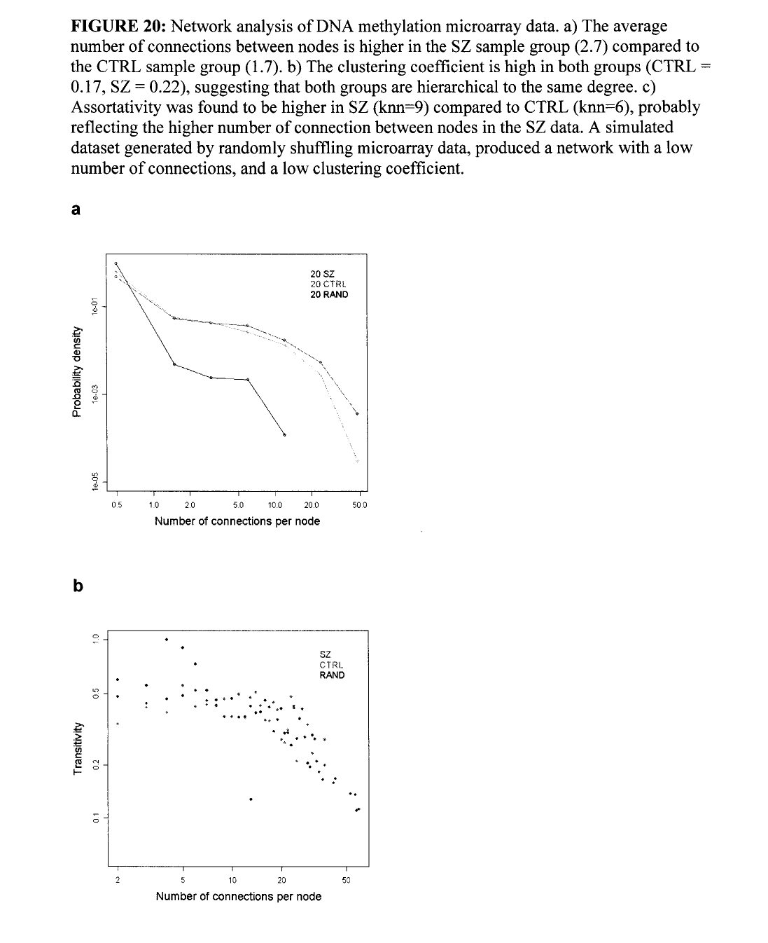

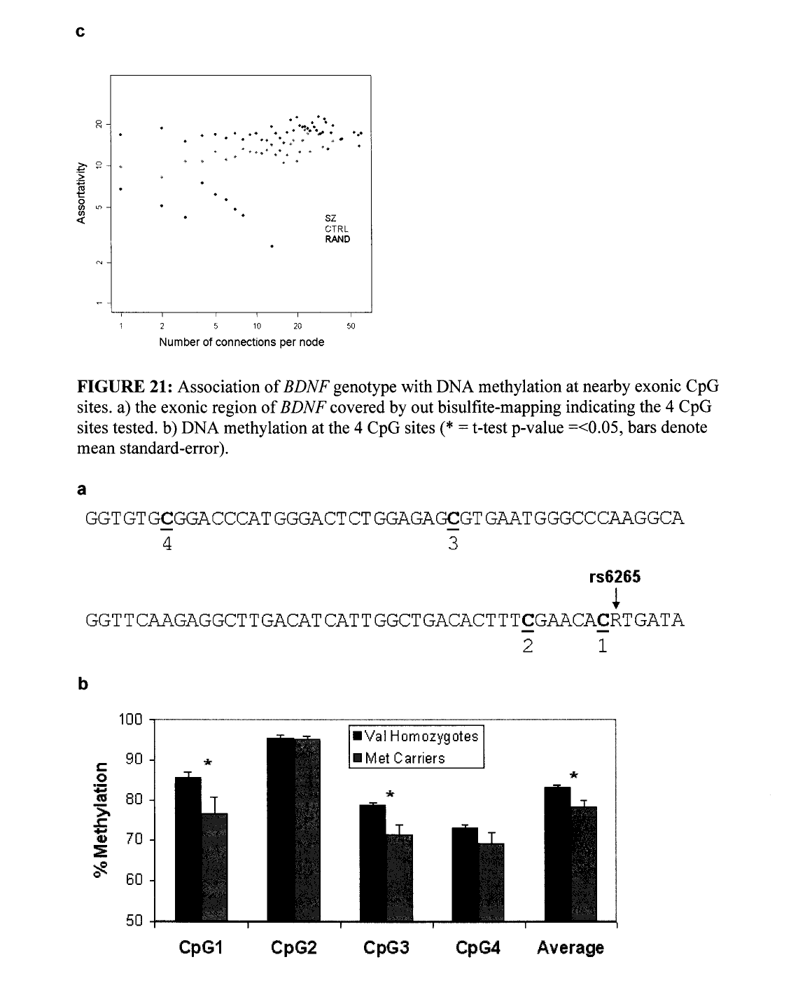

Modularity in DNA methylation microarray data: In the brain, the average number of connections between nodes (representing correlated methylation observed between different genomic loci) is higher in the SZ group compared to the CTRL group (2.7 vs 1.7) (FIG. 20). The large clustering coefficient in both sample groups (CTRL=0.17, SZ=0.22) suggests that both are modular. The lack of clustering in a series of simulated ‘random’ datasets, suggests that this modularity is likely to be a real biological phenomenon (FIG. 16 and FIG. 20). Assortativity is higher in SZ (knn=9) compared to CTRL (knn=6), reflecting the higher number of connections between nodes in SZ. Whilst the number of modules (CTRL=42, SZ=43) and median size of modules (CTRL=10, SZ=11) is approximately the same in both groups, the degree of modularity is higher in CTRL (0.0066) than in SZ (0.0051). A similar pattern is seen in the germline, with higher connectivity (3.9 vs 1.5) and assortativity (15 vs 7) in affected individuals compared to controls, a high degree of clustering in both groups (CTRL=0.14, BD=0.17), but higher modularity in unaffected individuals (0.0067 vs 0.0029).

DNA methylation analysis of psychosis candidate genes in brain DNA: We found little evidence of any psychosis-associated DNA methylation differences in any of the ten regions/genes tested (Table 12), including the promoter regions of COMT and RELN found to be differentially methylated in previous studies. Non-synonymous SNPs in COMT (rs4680—val108/158met) and BDNF (rs6265—val66met) both create/abolish exonic CpG sites. In COMT, surrounding CpG sites were highly methylated (>95%) in all samples tested, with no correlation between genotype and DNA methylation. In BDNF there is modest evidence for an association between genotype and DNA methylation. 74% of the samples tested were CC (val homozygotes), and 26% were CT or TT (met carriers). Val homozygotes had significantly higher DNA methylation across the exonic region profiled (average methylation=83% vs 78%, p=0.02).

Discussion

In this study we performed a microarray-based epigenomic scan using CpG-island microarrays and found psychosis-associated brain DNA methylation differences in numerous loci, including many genes that have been functionally-linked to disease etiology. Consistent with increasing evidence for altered glutamatergic and GABAergic neurotransmission in the pathogenesis of PSY20, we identified epigenetic changes in loci associated with both these neurotransmitter pathways.

Glutamate is the most abundant fast excitatory neurotransmitter in the mammalian nervous system, with a critical role in synaptic plasticity. Several lines of evidence link the glutamate system to psychosis, in particular the observation that glutamate receptor agonists can cause psychotic symptoms in unaffected individuals. Probes associated with two glutamate receptor genes—one near WDR18, located ˜10 kb upstream of the NMDA receptor subunit gene NR3B (also known as GR1N3B), and another in the promoter of the AMPA receptor subunit gene GRIA2—were found to be hypomethylated in SZ and PSY males. Dysregulation of both NMDA and AMPA glutamate receptors is important in the etiology of PSY21, and GRIA2 expression is altered in the brains of SZ patients22.

Various types of glutamate transporter are present in the plasma membranes of glial cells and neurons. Our data suggests that two vesicular glutamate transporters (VGLUTs), which pack glutamate into synaptic vesicles, are epigenetically altered in PSY. Given the link between DNA methylation and gene transcription, our data concur with data from gene expression studies and the observation that VGLUT1 and VGLUT2 are expressed in a complementary manner in cortical neurons23. VGLUT1, which were hypermethylated in SZ female samples, is down-regulated in the brains of SZ patients24. In addition, VGLUT2, which is up-regulated in SZ patients25, is hypomethylated in SZ females.

Several other glutamatergic genes showed evidence of epigenetically dysregulation in PSY. GLS2, which encodes a glutaminase enzyme that catalyses the hydrolysis of glutamine to glutamate, was hypermethylated in SZ male samples. Previous studies report that glutaminase expression is altered in the pathology of SZ26. The gene encoding Secretogranin II (SCG2), a secretory protein located in neuronal vesicles that is known to stimulate the release of glutamate, was hypomethylated in PSY females relative to unaffected controls. SCG2 expression is known to be modulated by both chronic PCP exposure, which mimics symptoms of PSY27 and lithium treatment28.

Unlike glutamate, which is a strong excitatory neurotransmitter, GABA acts as a potent inhibitory neurotransmitter. Hypofunctioning GABAergic interneurons appear to be important in the etiology of PSY29. Our data suggest that MARLIN-1, a RNA-binding protein widely expressed in the brain that regulates the production of functional GABA(B) receptors (30), is hypermethylated in SZ, BD, and PSY female samples. In addition, KCNJ6, a G protein-coupled inwardly rectifying potassium channel that has been linked to the regulation of GABA neurotransmission (31) was found to be hypermethylated in SZ and PSY males. Increasing evidence suggests that both the glutamate and GABA systems are synergistically involved PSY20, supporting our observation of increased HELT promoter methylation in SZ and BD female samples. HELT is known to determine GABAergic over glutamatergic neuronal fate in the developing mesencephalon (32).

We observed evidence for epigenetic dysregulation near several genes involved in neuronal development in the brain. WNT1, an integral part of the Wnt signaling pathway that is critical for neurodevelopment, which is differentially expressed in SZ brains (33), was significantly hypermethylated in PSY females relative to controls. The transcriptionally inducible nuclear receptor NR4A2, down-regulated in both SZ and BD (see FIG. 17B and (34)), was found to be hypermethylated in SZ females. FOSB, encoding a protein controlling cell proliferation in the brain known to be expressed following chronic antipsychotic treatment (35) was hypomethylated in PSY females relative to controls. Finally, the LIM homeobox transcription factors LMX1B and LHX5, linked to normal learning and motor functions (36), also showed significant methylation changes in female psychosis samples, with LMX1B demonstrating putative hypomethylation and LHX5 putative hypermethylation.

Several other genes with links to PSY were found to be epigenetically altered. Given that phospholipid metabolism is disturbed in SZ37, it is noteworthy that the phospholipase gene PLA2G4B was hypermethylated in SZ male, PSY male, and PSY female samples. RAIL hypermethylated in SZ female samples, is located in an unstable genomic region encoding a polymorphic polyglutamine tract associated with SZ and response to antipsychotic medication (38). AUTS2, hypermethylated in SZ male samples, spans a translocation breakpoint associated with mental retardation and autism (39). Finally, a probe located ˜90 kb upstream of one of our pre-nominated psychosis ‘candidate genes’, DTNBP1, was hypermethylated in affected females.

No correlation was found between any demographic variables or post-mortem brain parameters and DNA methylation. Given the dynamic nature of the epigenome, however, and evidence linking drug exposure to DNA methylation, we also examined the epigenetic effect of antipsychotic treatment. Methylation of a CpG island located upstream of MEK1 is strongly correlated with antipsychotic intake, particularly in SZ males. The link between MEK1 and antipsychotic exposure is striking given the involvement of mitogen-activated protein kinase (MAPK) signaling pathways in mediating intraneuronal signaling, and the observation that clozapine selectively activates this pathway via an interaction with MEK (140).

Gene ontology (GO) analysis allows the investigation of functionally-linked biological pathways in microarray datasets. Several interesting GO categories were highlighted by our analysis, including several involved in various epigenetic processes, transcription, and development. In addition we find an association with genes involved in “brain development” in both female BD and SZ samples and “response to stress” in male BD samples, consistent with the popular diathesis-stress hypothesis of psychosis susceptibility. In addition, given the postulated link between mitochondrial dysfunction, oxidative stress and psychosis19, it is interesting that a number of mitochondrial GO categories show significantly different distributions in affected individuals. Our methylome results are in close agreement with a parallel microarray-based transcriptomics, proteomics and metabolomics study, also performed on brain tissue obtained from the Stanley Foundation, in which genes/proteins associated with mitochondrial function and oxidative stress responses were the most altered group (19).

Traditional etiological studies of complex disease, both genetic and epigenetic, have tended to investigate discrete regions of DNA in isolation. It is plausible, however, that the epigenome, like many other biological systems, comprises a complex network of interacting processes and that DNA methylation in different genomic regions is inter-dependent. Understanding the system-level features of biological organization across the epigenome is an important aspect of elucidating the epigenetic changes associated with disease. In order to investigate if DNA methylation is coordinated across different loci, we utilized a novel network-based approach to test the modularity of our methylome data. In this way, a network comprises of distinct clusters of elements, termed ‘modules’, which are highly connected within themselves, but have fewer connections with the rest of the network (41). The study of interaction networks has proven fruitful in many areas of biological research, highlighting distinct modularity in metabolic networks (42), cellular networks (43), and protein interaction networks (44). Whilst such an approach has not been previously applied to the epigenome, recent evidence suggests the involvement of coordinated epigenetic silencing across large genomic regions in cancer (45).

The goal of our network analysis was twofold: first, to see whether there is modularity in the methylome; second, if such epigenetic modularity exists, to see whether there are any differences between affected and unaffected groups. For both brain and germline DNA, we found evidence for significant epigenetic modularity in both groups analyzed. No modules were observed in a series of simulated ‘random’ datasets, suggesting that the modular structure of the methylome is a real biological phenomenon and that the epigenome can be split into distinct groups of correlated loci, potentially corresponding to distinct functional pathways and/or physical regions. Whilst DNA methylation in both affected and unaffected groups is clearly modular, the number of interconnections between specific genomic regions is higher in the affected group compared to the CTRL group, resulting in more between-module interference, in both brain and germline DNA. Given that modules within such biological networks are likely to have specific functional tasks, separate to those of other modules (41), the lower degree of DNA methylation modularity observed in the PSY samples points to some degree of systemic epigenetic dysfunction associated with major psychosis.

The second approach utilized in this study focused on DNA methylation in the vicinity of genes with a priori evidence for an etiological role in PSY. These regions were profiled directly using bisulfite-modification and Pyrosequencing, with assays designed to span CpG-rich promoter regions, along with some exonic and intronic regions for several genes. Little evidence was found to suggest that DNA methylation in these genes is associated with either SZ or BD. Our analyses included the promoter regions of both COMT and RELN that have been previously shown to be epigenetically altered in psychosis in previous studies (8,9,46). Unlike these studies that report COMT hypomethylation and RELN hypermethylation in SZ samples, we found no evidence for DNA methylation changes in these genes associated with either SZ or BD. Our data are in agreement with a previous study on COMT reporting no association between promoter methylation and PSY11, and a recent study reporting very low levels of methylation across the RELN region, and no association with PSY10. It should be noted that some of the methods used in previous studies of these genes, for example methylation-specific PCR, can lead to biased assessment of methylated cytosines, and are not able to assess epigenetic changes in a truly quantitative manner as is possible with the Pyrosequencing methodology utilized in this study.

The observation of an association between genotype at a non-synonymous SNP (rs6265) in BDNF, and DNA methylation at surrounding CpG sites adds to the increasing evidence that DNA sequences can influence epigenetic profiles (e.g.14,47). Whilst DNA alleles and haplotypes can be subject to differential epigenetic modification, it appears that epigenetic status cannot be unequivocally deduced from DNA sequence data alone. The notion that epigenetic changes may be associated with DNA sequence variation is relevant to the inconsistent genetic association studies in complex diseases, and suggests that a comprehensive epigenetic analysis of candidate SNPs and haplotypes is warranted.

Our tandem use of two complementary approaches allowed us to test both a priori hypotheses and identify novel regions of the genome that may be epigenetically dysfunctional in PSY. The unbiased microarray approach was far more productive in identifying differentially methylated loci than the candidate gene approach; this has implications for the design of future epigenetic studies of complex disease. Of note is the observation that a high proportion of the microarray-nominated loci can be considered good functional/positional candidates. Given the relatively large number of differences observed between affected and unaffected individuals in our microarray screen, and the laborious nature of current bisulfite-based mapping approaches, it was unfeasible to further investigate each nominated gene at the level of specific CpG nucleotides in the course of this study. Our analyses were stringently controlled for multiple-testing using the FDR statistic, but as with all microarray-based experiments, it is possible that some of the genes uncovered are false-positives, and, more in-depth screening of specific gene regions will be needed to verify the specific DNA methylation changes involved.

To conclude, consistent with the epigenetic theory of PSY, a number of loci were found to be epigenetically altered in the brain of SZ and BD patients relative to unaffected controls.

### REFERENCES

1 Lander E S, Linton L M, Birren B, Nusbaum C, Zody M C, Baldwin J, Devon K et al. (2001) Initial sequencing and analysis of the human genome. Nature 409, 860-921.
2 Venter J C, Adams M D, Myers E W, Li P W, Mural R J, Sutton G G, Smith H O et al. (2001) The sequence of the human genome. Science 291, 1304-51.
3 Altshuler D, Brooks L D, Chakravarti A, Collins F S, Daly M J and Donnelly P. (2005) A haplotype map of the human genome. Nature 437, 1299-320.
4 CSAC CSaAC. (2005) Initial sequence of the chimpanzee genome and comparison with the human genome. Nature 437, 69-87.
5 Henikoff S and Matzke M A. (1997) Exploring and explaining epigenetic effects. Trends Genet 13, 293-5.
6 Li E, Bestor T H and Jaenisch R. (1992) Targeted mutation of the DNA methyltransferase gene results in embryonic lethality. Cell 69, 915-26.
7 Jaenisch R and Bird A. (2003) Epigenetic regulation of gene expression: how the genome integrates intrinsic and environmental signals. Nat Genet 33 Suppl, 245-54.
8 Rakyan V K, Hildmann T, Novik K L, Lewin J, Tost J, Cox A V, Andrews T D, Howe K L, Otto T, Olek A, Fischer J, Gut I G, Berlin K and Beck S. (2004) DNA methylation profiling of the human major histocompatibility complex: a pilot study for the human epigenome project. PLoS Biol 2, e405
9 Li E. (2002) Chromatin modification and epigenetic reprogramming in mammalian development. Nat Rev Genet 3, 662-73.
10 Reik W and Walter J. (2001) Genomic imprinting: parental influence on the genome. Nat Rev Genet 2, 21-32.
11 Suter C M, Martin D I and Ward R L. (2004) Germline epimutation of MLH1 in individuals with multiple cancers. Nat Genet 36, 497-501.
12 Martin D I, Ward R and Suter C M. (2005) Germline epimutation: a basis for epigenetic disease in humans. Ann NY Acad Sci 1054, 68-77.
13 Reik W, Dean W and Walter J. (2001) Epigenetic reprogramming in mammalian development. Science 293, 1089-93.
14 Morgan H D, Sutherland H G, Martin D I and Whitelaw E. (1999) Epigenetic inheritance at the agouti locus in the mouse. Nat Genet 23, 314-8.
15 Allegrucci C, Thurston A, Lucas E and Young L. (2005) Epigenetics and the germline. Reproduction 129, 137-49.
16 Kimmins S and Sassone-Corsi P. (2005) Chromatin remodelling and epigenetic features of germ cells. Nature 434, 583-9.
17 Zalensky A O, Siino J S, Gineitis A A, Zalenskaya I A, Tomilin N V, Yau P and Bradbury E M. (2002). Human testis/sperm-specific histone H2B (hTSH2B). Molecular cloning and characterization. J Biol Chem 277, 43474-80.
18 Churikov D, Zalenskaya I A and Zalensky A O. (2004) Male germline-specific histones in mouse and man. Cytogenet Genome Res 105, 203-14.
19 Churikov D, Siino J, Svetlova M, Zhang K, Gineitis A, Morton Bradbury E and Zalensky A. (2004) Novel human testis-specific histone H2B encoded by the interrupted gene on the X chromosome. Genomics 84, 745-56.
20 Rakyan V and Whitelaw E. (2003) Transgenerational epigenetic inheritance. Curr Biol 13, R6.
21 Chong S and Whitelaw E. (2004) Epigenetic germline inheritance. Curr Opin Genet Dev 14, 692-6.
22 Hajkova P, el-Maarri O, Engemann S, Oswald J, Olek A and Walter J. (2002) DNA-methylation analysis by the bisulfite-assisted genomic sequencing method. Methods Mol Biol 200, 143-54.
23 Yatabe Y, Tavare S and Shibata D. (2001) Investigating stem cells in human colon by using methylation patterns. Proc Natl Acad Sci USA 98, 10839-44.
24 Heisler L E, Torti D, Boutros P C, Watson J, Chan C, Winegarden N, Takahashi M, Yau P, Huang T H, Farnham P J, Jurisica I, Woodgett J R, Bremner R, Penn L Z and Der S D. (2005) CpG Island microarray probe sequences derived from a physical library are representative of CpG Islands annotated on the human genome. Nucleic Acids Res 33, 2952-61.
25 Schumacher A, Kapranov, P., Kaminsky, Z. A., Flanagan, F., Assadzadeh, A., Yau, P., Virtanen, C., Winegarden, N., Cheng, J., Gingeras, T., Petronis, A. (Submitted) Microarray-based DNA Methylation Profiling: Technology and Applications. Epigenetic variation in germ cells.
26 Kaminsky ZA, Assadzadeh A, Flanagan J and Petronis A. (2005) Single nucleotide extension technology for quantitative site-specific evaluation of metC/C in GC-rich regions. Nucleic Acids Res 33, e95.
27 Gardiner-Garden M and Frommer M. (1987) CpG islands in vertebrate genomes. J Mol Biol 196, 261-82.
28 Sebat J, Lakshmi B, Troge J, Alexander J, Young J, Lundin P, Maner S, Massa H, Walker M, Chi M, Navin N, Lucito R, Healy J, Hicks J, Ye K, Reiner A, Gilliam T C, Trask B, Patterson N, Zetterberg A and Wigler M. (2004) Large-scale copy number polymorphism in the human genome. Science 305, 525-8.
29 Tuzun E, Sharp A J, Bailey J A, Kaul R, Morrison V A, Pertz L M, Haugen E, Hayden H, Albertson D, Pinkel D, Olson M V and Eichler E E. (2005) Fine-scale structural variation of the human genome. Nat Genet 37, 727-32
30 Iafrate A J, Feuk L, Rivera M N, Listewnik M L, Donahoe P K, Qi Y, Scherer S W and Lee C. (2004) Detection of large-scale variation in the human genome. Nat Genet 36, 949-51.
31 Craig J M and Bickmore W A. (1993) Chromosome bands—flavours to savour. Bioessays 15, 349-54.
32 Furey T S and Haussler D. (2003) Integration of the cytogenetic map with the draft human genome sequence. Hum Mol Genet 12, 1037-44.
33 Yoder J A, Walsh C P and Bestor T H. (1997) Cytosine methylation and the ecology of intragenomic parasites. Trends Genet 13, 335-40.
34 Bird A P. (1986) CpG-rich islands and the function of DNA methylation. Nature 321, 209-13.
35 Waterston R H, Lindblad-Toh K, Birney E, Rogers J, Abril J F, Agarwal P, Agarwala R et al. (2002) Initial sequencing and comparative analysis of the mouse genome. Nature 420, 520-62.
36 Jabs E W, Goble C A and Cutting G R. (1989) Macromolecular organization of human centromeric regions reveals high-frequency, polymorphic macro DNA repeats. Proc Natl Acad Sci USA 86, 202-6.
37 Miniou P, Jeanpierre M, Bourc'his D, Coutinho Barbosa A C, Blanquet V and Viegas-Pequignot E. (1997) alpha-satellite DNA methylation in normal individuals and in ICF patients: heterogeneous methylation of constitutive heterochromatin in adult and fetal tissues. Hum Genet 99, 738-45.
38 Gisselsson D, Shao C, Tuck-Muller C M, Sogorovic S, Palsson E, Smeets D and Ehrlich M. (2005) Interphase chromosomal abnormalities and mitotic missegregation of hypomethylated sequences in ICF syndrome cells. Chromosoma 114, 118-26.
39 Lippman Z and Martienssen R. (2004) The role of RNA interference in heterochromatic silencing. Nature 431, 364-70.
40 Sandovici I, Kassovska-Bratinova S, Loredo-Osti J C, Leppert M, Suarez A, Stewart R, Bautista F D, Schiraldi M and Sapienza C. (2005) Interindividual variability and parent of origin DNA methylation differences at specific human Alu elements. Hum Mol Genet 14, 2135-43.
41 Wexler N S, Lorimer J, Porter J, Gomez F, Moskowitz C, Shackell E, Marder K et al. (2004). Venezuelan kindreds reveal that genetic and environmental factors modulate Huntington's disease age of onset. Proc Natl Acad Sci USA 101, 3498-503.
42 Lichtenstein P, Holm N V, Verkasalo P K, Iliadou A, Kaprio J, Koskenvuo M, Pukkala E, Skytthe A and Hemminki K. (2000) Environmental and heritable factors in the causation of cancer—analyses of cohorts of twins from Sweden, Denmark, and Finland. N Engl J Med 343, 78-85.
43 Taubes G. (1995) Epidemiology faces its limits. Science 269, 164-9.
44 Wong A H, Gottesman, II and Petronis A. (2005) Phenotypic differences in genetically identical organisms: the epigenetic perspective. Hum Mol Genet 14 Spec No 1, R11-8.
45 Hibi K, Kodera Y, Ito K, Akiyama S and Nakao A. (2004) Methylation pattern of CDH13 gene in digestive tract cancers. Br J Cancer 91, 1139-42.
46 Ogama Y, Ouchida M, Yoshino T, Ito S, Takimoto H, Shiote Y, Ishimaru F, Harada M, Tanimoto M and Shimizu K. (2004) Prevalent hyper-methylation of the CDH13 gene promoter in malignant B cell lymphomas. Int J Oncol 25, 685-91.
47 Sakai M, Hibi K, Koshikawa K, Inoue S, Takeda S, Kaneko T and Nakao A. (2004) Frequent promoter methylation and gene silencing of CDH13 in pancreatic cancer. Cancer Sci 95, 588-91.
48 Hibi K, Nakayama H, Kodera Y, Ito K, Akiyama S and Nakao A. (2004) CDH13 promoter region is specifically methylated in poorly differentiated colorectal cancer. Br J Cancer 90, 1030-3.
49 Widschwendter A, Ivarsson L, Blassnig A, Muller H M, Fiegl H, Wiedemair A, Muller-Holzner E, Goebel G, Marth C and Widschwendter M. (2004) CDH1 and CDH13 methylation in serum is an independent prognostic marker in cervical cancer patients. Int J Cancer 109, 163-6.
50 Roman-Gomez J, Castillejo J A, Jimenez A, Cervantes F, Boque C, Hermosin L, Leon A, Granena A, Colomer D, Heiniger A and Torres A. (2003) Cadherin-13, a mediator of calcium-dependent cell-cell adhesion, is silenced by methylation in chronic myeloid leukemia and correlates with pretreatment risk profile and cytogenetic response to interferon alfa. J Clin Oncol 21, 1472-9.
51 Murrell A, Heeson S, Cooper W N, Douglas E, Apostolidou S, Moore G E, Maher E R and Reik W. (2004) An association between variants in the IGF2 gene and Beckwith-Wiedemann syndrome: interaction between genotype and epigenotype. Hum Mol Genet 13, 247-55.
52 Malaspina D, Harlap S, Fennig S, Heiman D, Nahon D, Feldman D and Susser E S. (2001) Advancing paternal age and the risk of schizophrenia. Arch Gen Psychiatry 58, 361-7.
53 Byrne M, Agerbo E, Ewald H, Eaton W W and Mortensen P B. (2003) Parental age and risk of schizophrenia: a case-control study. Arch Gen Psychiatry 60, 673-8.
54 Montgomery N D, Yee D, Chen A, Kalantry S, Chamberlain S J, Otte A P and Magnuson T. (2005) The murine polycomb group protein Eed is required for global histone H3 lysine-27 methylation. Curr Biol 15, 942-7.
55 Smith A, Bourdeau I, Wang J and Bondy C A. (2005) Expression of Catenin family members CTNNA1, CTNNA2, CTNNB1 and JUP in the primate prefrontal cortex and hippocampus. Brain Res Mol Brain Res 135, 225-31.
56 Oswald J, Engemann S, Lane N, Mayer W, Olek A, Fundele R, Dean W, Reik W and Walter J. (2000) Active demethylation of the paternal genome in the mouse zygote. Curr Biol 10, 475-8.
57 Mayer W, Niveleau A, Walter J, Fundele R and Haaf T. (2000) Demethylation of the zygotic paternal genome. Nature 403, 501-2.
58 Walsh C P, Chaillet J R and Bestor T H. (1998) Transcription of IAP endogenous retroviruses is constrained by cytosine methylation. Nat Genet 20, 116-7.
59 Hajkova P, Erhardt S, Lane N, Haaf T, El-Maarri O, Reik W, Walter J and Surani M A. (2002) Epigenetic reprogramming in mouse primordial germ cells. Mech Dev 117, 15-23.
60 Czermin B and Imhof A. (2003) The sounds of silence—histone deacetylation meets histone methylation. Genetica 117, 159-64.
61 Klar A J. (1998) Propagating epigenetic states through meiosis: where Mendel's gene is more than a DNA moiety. Trends Genet 14, 299-301.
62 Mittelsten Scheid O, Afsar K and Paszkowski J. (2003) Formation of stable epialleles and their paramutation-like interaction in tetraploid Arabidopsis thaliana. Nat Genet 34, 450-4.
63 Cavalli G and Paro R. (1998) The Drosophila Fab-7 chromosomal element conveys epigenetic inheritance during mitosis and meiosis. Cell 93, 505-18.
64 Cavalli G and Paro R. (1999) Epigenetic inheritance of active chromatin after removal of the main transactivator. Science 286, 955-8.
65 Schumacher A, Kapranov P, Kaminsky Z, Flanagan J, Assadzadeh A, Yau P, Virtanen C, Winegarden N, Cheng J, Gingeras T, Petronis A. Microarray-based DNA methylation profiling: technology and applications. Nucleic Acids Res. 2006 Jan. 20;34(2):528-42.

### REFERENCES (INCLUDED IN EXAMPLE 3)

1. Craddock, N., O′Donovan, M. C. & Owen, M. J. The genetics of schizophrenia and bipolar disorder: dissecting psychosis. J Med Genet 42, 193-204 (2005).
2. Petronis, A. The origin of schizophrenia: genetic thesis, epigenetic antithesis, and resolving synthesis. Biol Psychiatry 55, 965-70 (2004).
3. Craddock, N. & Jones, I. Genetics of bipolar disorder. J Med Genet 36, 585-94 (1999).
4. McGuffin, P., Asherson, P., Owen, M. & Farmer, A. The strength of the genetic effect. Is there room for an environmental influence in the aetiology of schizophrenia? Br J Psychiatry 164, 593-9 (1994).
5. Henikoff, S. & Matzke, M. A. Exploring and explaining epigenetic effects. Trends Genet 13, 293-5 (1997).
6. Riggs, A. D., Xiong, Z., Wang, L. & LeBon, J. M. Methylation dynamics, epigenetic fidelity and X chromosome structure. Novartis Found Symp 214, 214-25; discussion 225-32 (1998).
7. Richards, E. J. Inherited epigenetic variation—revisiting soft inheritance. Nat Rev Genet 7, 395-401 (2006).
8. Abdolmaleky, H. M. et al. Hypomethylation of MB-COMT promoter is a major risk factor for schizophrenia and bipolar disorder. Hum Mol Genet 15, 3132-45 (2006).
9. Grayson, D. R. et al. Reelin promoter hypermethylation in schizophrenia. Proc Natl Acad Sci USA 102, 9341-6 (2005).
10. Tochigi, M. et al. Methylation status of the reelin promoter region in the brain of schizophrenic patients. Biological Psychiatry (In Press).
11. Dempster, E. L., Mill, J., Craig, I. W. & Collier, D. A. The quantification of COMT mRNA in post mortem cerebellum tissue: diagnosis, genotype, methylation and expression. BMC Med Genet 7, 10 (2006).
12. Johnston-Wilson, N. L. et al. Disease-specific alterations in frontal cortex brain proteins in schizophrenia, bipolar disorder, and major depressive disorder. The Stanley Neuropathology Consortium. Mol Psychiatry 5, 142-9 (2000).
13. Higgs, B. W., Elashoff, M., Richman, S. & Barci, B. An online database for brain disease research. BMC Genomics 7, 70 (2006).
14. Flanagan, J. M. et al. Intra- and interindividual epigenetic variation in human germ cells. Am J Hum Genet 79, 67-84 (2006).
15. Schumacher, A. et al. Microarray-based DNA methylation profiling: technology and applications. Nucleic Acids Res 34, 528-42 (2006).
16. Klempnauer, K. H. Methylation-sensitive DNA binding by v-myb and c-myb proteins. Oncogene 8, 111-5 (1993).
17. Maier, H., Colbert, J., Fitzsimmons, D., Clark, D. R. & Hagman, J. Activation of the early B-cell-specific mb-1 (Ig-alpha) gene by Pax-5 is dependent on an unmethylated Ets binding site. Mol Cell Biol 23, 1946-60 (2003).
18. Cowled, P., Kanter, I., Leonardos, L. & Jackson, P. Uroplakin Ib gene transcription in urothelial tumor cells is regulated by CpG methylation. Neoplasia 7, 1091-103 (2005).
19. Prabakaran, S. et al. Mitochondrial dysfunction in schizophrenia: evidence for compromised brain metabolism and oxidative stress. Mol Psychiatry 9, 684-97, 643 (2004).
20. Coyle, J. T. The GABA-glutamate connection in schizophrenia: which is the proximate cause? Biochem Pharmacol 68, 1507-14 (2004).
21. Lau, C. G. & Zukin, R. S. NMDA receptor trafficking in synaptic plasticity and neuropsychiatric disorders. Nat Rev Neurosci 8, 413-26 (2007).
22. Gupta, D. S. et al. Metabotropic glutamate receptor protein expression in the prefrontal cortex and striatum in schizophrenia. Synapse 57, 123-31 (2005).
23. Fremeau, R. T., Jr. et al. The expression of vesicular glutamate transporters defines two classes of excitatory synapse. Neuron 31, 247-60 (2001).
24. Eastwood, S. L. & Harrison, P. J. Decreased expression of vesicular glutamate transporter 1 and complexin II mRNAs in schizophrenia: further evidence for a synaptic pathology affecting glutamate neurons. Schizophr Res 73, 159-72 (2005).
25. Smith, R. E., Haroutunian, V., Davis, K. L. & Meador-Woodruff, J. H. Vesicular glutamate transporter transcript expression in the thalamus in schizophrenia. Neuroreport 12, 2885-7 (2001).
26. Bruneau, E. G., McCullumsmith, R. E., Haroutunian, V., Davis, K. L. & Meador-Woodruff, J. H. Increased expression of glutaminase and glutamine synthetase mRNA in the thalamus in schizophrenia. Schizophr Res 75, 27-34 (2005).
27. Marksteiner, J. et al. Differential regulation of chromogranin A, chromogranin B and secretogranin II in rat brain by phencyclidine treatment. Neuroscience 104, 325-33 (2001).
28. McQuillin, A., Rizig, M. & Gurling, H. M. A microarray gene expression study of the molecular pharmacology of lithium carbonate on mouse brain mRNA to understand the neurobiology of mood stabilization and treatment of bipolar affective disorder. Pharmacogenet Genomics 17, 605-17 (2007).
29. Benes, F. M. & Berretta, S. GABAergic interneurons: implications for understanding schizophrenia and bipolar disorder. Neuropsychopharmacology 25, 1-27 (2001).
30. Couve, A. et al. Marlin-1, a novel RNA-binding protein associates with GABA receptors. J Biol Chem 279, 13934-43 (2004).
31. Koyrakh, L. et al. Molecular and cellular diversity of neuronal G-protein-gated potassium channels. J Neurosci 25, 11468-78 (2005).
32. Nakatani, T., Minaki, Y., Kumai, M. & Ono, Y. Helt determines GABAergic over glutamatergic neuronal fate by repressing Ngn genes in the developing mesencephalon. Development (2007).
33. Miyaoka, T., Seno, H. & Ishino, H. Increased expression of Wnt-1 in schizophrenic brains. Schizophr Res 38, 1-6 (1999).
34. Xing, G., Zhang, L., Russell, S. & Post, R. Reduction of dopamine-related transcription factors Nurr1 and NGFI-B in the prefrontal cortex in schizophrenia and bipolar disorders. Schizophr Res 84, 36-56 (2006).
35. Kontkanen, O., Lakso, M., Wong, G. & Castren, E. Chronic antipsychotic drug treatment induces long-lasting expression of fos and jun family genes and activator protein 1 complex in the rat prefrontal cortex. Neuropsychopharmacology 27, 152-62 (2002).
36. Paylor, R., Zhao, Y., Libbey, M., Westphal, H. & Crawley, J. N. Learning impairments and motor dysfunctions in adult Lhx5-deficient mice displaying hippocampal disorganization. Physiol Behav 73, 781-92 (2001).
37. Horrobin, D. F., Glen, A. I. & Vaddadi, K. The membrane hypothesis of schizophrenia. Schizophr Res 13, 195-207 (1994).
38. Toulouse, A., Rochefort, D., Roussel, J., Joober, R. & Rouleau, G. A. Molecular cloning and characterization of human RAIL a gene associated with schizophrenia. Genomics 82, 162-71 (2003).
39. Kalscheuer, V. M. et al. Mutations in autism susceptibility candidate 2 (AUTS2) in patients with mental retardation. Hum Genet 121, 501-9 (2007).
40. Browning, J. L. et al. Clozapine and the mitogen-activated protein kinase signal transduction pathway: implications for antipsychotic actions. Biol Psychiatry 57, 617-23 (2005).
41. Newman, M. E. Modularity and community structure in networks. Proc Natl Acad Sci USA 103, 8577-82 (2006).
42. Ravasz, E., Somera, A. L., Mongru, D. A., Oltvai, Z. N. & Barabasi, A. L. Hierarchical organization of modularity in metabolic networks. Science 297, 1551-5 (2002).
43. Rives, A. W. & Galitski, T. Modular organization of cellular networks. Proc Natl Acad Sci USA 100, 1128-33 (2003).
44. Spirin, V. & Mirny, L. A. Protein complexes and functional modules in molecular networks. Proc Natl Acad Sci USA 100, 12123-8 (2003).
45. Frigola, J. et al. Epigenetic remodeling in colorectal cancer results in coordinate gene suppression across an entire chromosome band. Nat Genet 38, 540-9 (2006).
46. Abdolmaleky, H. M. et al. Hypermethylation of the reelin (RELN) promoter in the brain of schizophrenic patients: a preliminary report. Am J Med Genet B Neuropsychiatr Genet 134, 60-6 (2005).
47. Murrell, A. et al. An association between variants in the IGF2 gene and Beckwith-Wiedemann syndrome: interaction between genotype and epigenotype. Hum Mol Genet 13, 247-55 (2004).
48. Veldic, M., Guidotti, A., Maloku, E., Davis, J. M. & Costa, E. In psychosis, cortical interneurons overexpress DNA-methyltransferase 1. Proc Natl Acad Sci USA 102, 2152-7 (2005).
49. Suter, C. M., Martin, D. I. & Ward, R. L. Germline epimutation of MLH1 in individuals with multiple cancers. Nat Genet 36, 497-501 (2004).
50. Benjamini, Y. & Hochberg, Y. Controlling the false discovery rate: a practical and powerful approach to multiple testing. Journal of the Royal Statistical Society Series B 57, 289-300 (1995).
51. Schafer, J. & Strimmer, K. A shrinkage approach to large-scale covariance matrix estimation and implications for functional genomics. Stat Appl Genet Mol Biol 4, Article 32 (2005).

All citations are hereby incorporated by reference.

The present invention has been described with regard to one or more embodiments. However, it will be apparent to persons skilled in the art that a number of variations and modifications can be made without departing from the scope of the invention as defined in the claims.

TABLE 1
Statistical Analysis of microarray data from the sperm DNA-HHA,
sperm DNA-HpaII and the brain DNA-HpaII data sets.

| | | |
|---|---|---|
| Sperm DNA HHA data set | Sperm DNA HpaII data set | Brain DNA HpaII data set |
| (N = 25) | (N = 21) | (N = 22) |

Descriptive Statistics

| | | | |
|---|---|---|---|
| Mean | 6.72 (2.48) | 9.23 (4.92) | 10.89 (5.40) |
| CV (SD) | | | |
| Count of | 4969 | 4947 | 4952 |
| loci | | | |
| 10^{th}%, | <4.33, >9.53 | <5.16, >13.71 | <6.44, >16.33 |
| 90^{th}% | | | |

SNP Analysis

| | # with HHA | # with no - | # with HpaII | # with no | |
|---|---|---|---|---|---|
| | SNPs | HHA SNPs | SNPs | HpaII SNPs I | |
| 90^{th} | 78 | 72 | 32 | 118 | Not Done |
| Percentile | | | | | |
| 10^{th} | 74 | 76 | 22 | 128 | |
| Percentile | | | | | |

| | | |
|---|---|---|
| χ² | 0.12 | 1.829 |
| p-value | 0.729 | 0.176 |

CpG Island Analysis

| | CGI | Not CGI | CGI | Not CGI | CGI | Not CGI |
|---|---|---|---|---|---|---|
| Mean | 6.69 (2.57) | 6.79 (2.32) | 9.6 (5.28) | 8.97 (4.39) | 11.06 (4.94) | 11.05 (5.60) |
| CV (SD) | | | | | | |
| Count | 2523 | 2446 | 2512 | 2435 | 2478 | 2401 |

| | | | |
|---|---|---|---|
| t-test p- | 0.14815 | 4.92E−06 | 0.93995 |
| value | | | |

CG1 χ² test

| | # CGI | # Not CGI | # CGI | # Not CGI | # CGI | # Not CGI |
|---|---|---|---|---|---|---|
| 90^{th} | 235 | 217 | 296 | 198 | 256 | 238 |
| Percentile | | | | | | |
| 10^{th} | 226 | 209 | 218 | 277 | 255 | 238 |
| Percentile | | | | | | |

| | | | |
|---|---|---|---|
| χ² | 0.003 | 24.34 | 0.001 |
| p-value | 0.955 | 5.81E−07 | 0.974 |

Promoter χ² test

| | | # Promoter | # Not | |
|---|---|---|---|---|
| | | CGI | Promoter CGI | |
| 90^{th} | Not Applicable | 245 | 52 | Not Applicable |
| Percentile | | | | |
| 10^{th} | | 152 | 67 | |
| Percentile | | | | |

| | |
|---|---|
| χ² | 11.44 |
| p-value | 4.87E−04 |

The mean of the coefficient of variance (CV) in ratio (Cy5/Cy3) across the individuals (N) for each data set was calculated (+/− standard deviation, SD)
The Count represents the number of unique loci remaining after data trimming (see Methods).
Loci with CV above the 90th percentile are the top 10% methylation variable regions and loci below the 10th percentile are the least variable loci.
The single nucleotide polymorphism (SNP) analysis was performed to test for the effects of SNPs from our DNA methylation analysis.
300 loci were randomly selected from the 10th and 90th percentile loci and the clone sequence plus 1 kb flanking regions were screened for the presence of SNPs and in particular SNPs that create or disrupt HpaII, Hin6I, and AciI restriction sites.
The CpG island (CGI) analyses were performed by separating loci into either CpG islands (CGI) or non-CpG islands (not CGI).
A Students t-test was performed to analyze the difference in mean CV.
The number of loci within each group, within the 90th and 10th percentile loci, were counted and c2 analysis was performed. The CGI loci were further subdivided into loci within promoter regions of genes or not within promoters and the numbers of loci with the 90th and 10th percentiles were counted and c2 analysis was performed.

TABLE 2A
Age related variance/Correlation in sperm-Hpall data set

| | | | Distance | | |
|---|---|---|---|---|---|
| | | | from | | |
| | | | Gene | | |
| UHNID | Age corr. | Genome Location of Clone | (bp) | GeneID | Nearest Gene |
| UHNhscpg0007432 | −0.918421653 | chr14: 73296338-73296842 | 0 | 91748 | C14orf43 |
| UHNhscpg0004878 | −0.644590225 | chr18: 58533334-58533572 | 1366 | 23239 | PHLPP |
| UHNhscpg0001279 | −0.635499301 | chr2: 15682160-15683012 | 0 | 1653 | DDX1 |
| UHNhscpg0010337 | −0.633961562 | chr4: 24692990-24693129 | 0 | 55203 | LGI2 |
| UHNhscpg0000523 | −0.628700319 | chr14: 89918538-89919063 | 14066 | 801 | CALM1 |
| UHNhscpg0009687 | −0.61929126 | chr11: 71430395-71430772 | 0 | 4926 | NUMA1 |
| UHNhscpg0002060 | −0.607457801 | chr7: 117447932-117448126 | 10775 | 56311 | ANKRD7 |
| UHNhscpg0006282 | −0.603136381 | chr14: 50203675-50203889 | 0 | 60485 | SAV1 |
| UHNhscpg0008336 | −0.60134971 | chr3: 4211475-4211590 | 0 | | AY358092 |
| UHNhscpg0002269 | −0.594487623 | chr16: 18719890-18721070 | 0 | 23204 | ARL6IP |
| UHNhscpg0006883 | −0.590701745 | chr7: 20603836-20604518 | 4091 | 221833 | SP8 |
| UHNhscpg0003324 | −0.57876246 | chr8: 80412773-80412879 | 272989 | 11075 | STMN2 |
| UHNhscpg0007072 | −0.578612647 | chr13: 20930559-20930802 | 0 | 253832 | FLJ25952 |
| UHNhscpg0002392 | −0.577309667 | chr12: 55758348-55759109 | 0 | 23306 | AB006624 |
| UHNhscpg0010814 | −0.574206485 | chr9: 111503780-111504547 | 0 | 548645 | GNG10 |
| UHNhscpg0002833 | −0.568147866 | chr11: 62202212-62203556 | 0 | | LOC51035 |
| UHNhscpg0007366 | −0.562953631 | chr13: 20770028-20770950 | 73766 | | FLJ25952 |
| UHNhscpg0010640 | −0.55975393 | chr16: 81828071-81828454 | 0 | 1012 | CDH13 |
| UHNhscpg0003417 | −0.55808056 | chr8: 80412825-80412879 | 272989 | 11075 | STMN2 |
| UHNhscpg0007351 | −0.557144063 | chr11: 133162749-133163293 | 52433 | 219938 | SPATA19 |
| UHNhscpg0011679 | −0.553844494 | chr19: 5999835-6000028 | 0 | 5990 | RFX2 |
| UHNhscpg0011303 | −0.551957348 | chr1: 114639352-114639536 | 12748 | 51592 | TRIM33 |
| UHNhscpg0000547 | −0.551565288 | chr9: 86126053-86126501 | 0 | 81689 | HBLD2 |
| UHNhscpg0010993 | −0.550180272 | chr1: 46478949-46480024 | 0 | 10489 | AF370430 |
| UHNhscpg0000380 | −0.545084964 | chrX: 134996172-134996628 | 0 | 2273 | FHL1 |
| UHNhscpg0003556 | −0.544679103 | chrX: 62745590-62745765 | 0 | 23229 | ARHGEF9 |
| UHNhscpg0002314 | −0.539967585 | chr3: 157491673-157492091 | 0 | 7881 | KCNAB1 |
| UHNhscpg0003596 | −0.531788057 | chr4: 172320378-172320459 | 934465 | 51166 | AADAT |
| UHNhscpg0001705 | −0.526787621 | chr15: 99009623-99010314 | 2859 | 140460 | ASB7 |
| UHNhscpg0009822 | −0.526562678 | chr1: 21855364-21855679 | 370 | | AK026930 |
| UHNhscpg0000063 | −0.52421876 | chr3: 72584222-72584350 | 5758 | 23429 | RYBP |
| UHNhscpg0001205 | −0.521667613 | chr17: 38793230-38793666 | 8954 | | AK128207 |
| UHNhscpg0009465 | −0.518509703 | chr5: 92982058-92982196 | 0 | 83989 | DKFZP564D172 |
| UHNhscpg0009804 | −0.517599705 | chr6: 95040381-95040442 | 854395 | 2045 | EPHA7 |
| UHNhscpg0010111 | −0.516980672 | chr3: 45163070-45163300 | 152 | 64866 | CDCP1 |
| UHNhscpg0008321 | −0.51607039 | chr4: 32393339-32393439 | 1572342 | 5099 | PCDH7 |
| UHNhscpg0006596 | −0.515801694 | chr12: 6667579-6668374 | 0 | 171017 | ZNF384 |
| UHNhscpg0007045 | −0.514160426 | chr6: 39190376-39191144 | 0 | 55776 | C6orf64 |
| UHNhscpg0003064 | −0.513774745 | chr15: 50863986-50864266 | 0 | 3175 | ONECUT1 |
| UHNhscpg0008779 | −0.512617631 | chr12: 64004487-64004759 | 0 | 253827 | LOC253827 |
| UHNhscpg0002807 | −0.510358672 | chr9: 91266376-91266791 | 859 | 4783 | NFIL3 |
| UHNhscpg0007252 | −0.510001953 | chr13: 110249836-110250880 | 68680 | 283487 | LOC283487 |
| UHNhscpg0008979 | −0.509333714 | chr3: 97915795-97915891 | 100433 | | AY358738 |
| UHNhscpg0002130 | −0.508146229 | chr20: 61676052-61676691 | 6181 | 85441 | PRIC285 |
| UHNhscpg0000750 | −0.506824133 | chr11: 111449826-111451103 | 0 | 55216 | FLJ10726 |
| UHNhscpg0004733 | −0.506433015 | chr11: 77963050-77963233 | 0 | 79731 | FLJ23441 |
| UHNhscpg0007997 | −0.504269038 | chr11: 19691578-19692280 | 0 | 89797 | AJ488207 |
| UHNhscpg0011253 | −0.503229452 | chr5: 5123770-5123955 | 111307 | 170690 | ADAMTS16 |
| UHNhscpg0001019 | −0.502788473 | chr17: 38793230-38793666 | 8954 | | AK128207 |
| UHNhscpg0000830 | −0.502263623 | chr20: 11257478-11257784 | 588782 | 22903 | BTBD3 |
| UHNhscpg0008888 | −0.501752931 | chr14: 46575274-46575377 | 0 | 161357 | MAMDC1 |
| UHNhscpg0005027 | −0.501245356 | chr10: 127671190-127671371 | 0 | 92565 | AY251163 |
| UHNhscpg0008252 | 0.50313828 | chrX: 24795508-24795821 | 1997 | 170302 | ARX |
| UHNhscpg0005849 | 0.505284858 | chr9: 69169771-69170443 | 3351 | 9413 | C9orf61 |
| UHNhscpg0002396 | 0.505405308 | chr9: 15499859-15499960 | 0 | 11168 | PSIP1 |
| UHNhscpg0000331 | 0.505543224 | chr1: 114065861-114066820 | 0 | 54665 | FLJ11220 |
| UHNhscpg0004028 | 0.505850764 | chr6: 109920727-109920873 | 0 | | FLJ25791 |
| UHNhscpg0003238 | 0.508910182 | chr5: 72642966-72643484 | 134358 | 2297 | FOXD1 |
| UHNhscpg0005599 | 0.512086932 | chr11: 57161641-57162347 | 6789 | 219539 | YPEL4 |
| UHNhscpg0004696 | 0.515663002 | chr2: 26012699-26013466 | 0 | 55252 | ASXL2 |
| UHNhscpg0008470 | 0.516009324 | chr1: 225950806-225950903 | 0 | 55746 | NUP133 |
| UHNhscpg0008340 | 0.51741056 | chr4: 84313172-84313686 | 0 | 51138 | COPS4 |
| UHNhscpg0003990 | 0.517872516 | chr12: 43376666-43376983 | 0 | 4753 | NELL2 |
| UHNhscpg0008557 | 0.518846707 | chr10: 239933-240068 | 0 | 10771 | ZMYND11 |
| UHNhscpg0000482 | 0.520984085 | chr11: 85649369-85649478 | 0 | 8726 | EED |
| UHNhscpg0005884 | 0.520992457 | chr3: 128381578-128381626 | 13045 | 285311 | AK097460 |
| UHNhscpg0008792 | 0.521754696 | chr4: 145190267-145191203 | 5950 | 2996 | GYPE |
| UHNhscpg0009520 | 0.525317277 | chr1: 91678271-91678520 | 0 | 8317 | CDC7 |
| UHNhscpg0004355 | 0.526505907 | chr6: 27464330-27464706 | 0 | 441136 | AK092633 |
| UHNhscpg0001611 | 0.526967248 | chr6: 30289787-30290357 | 683 | 7726 | TRIM26 |
| UHNhscpg0004783 | 0.529777441 | chr22: 15638898-15638988 | 0 | 150165 | MGC57211 |
| UHNhscpg0000025 | 0.532588588 | chr10: 75370203-75371061 | 22943 | 5328 | PLAU |
| UHNhscpg0005928 | 0.535021909 | chr1: 212554059-212554157 | 0 | 7399 | USH2A |
| UHNhscpg0001828 | 0.537335195 | chr17: 17507093-17507808 | 17703 | 10743 | RAI1 |
| UHNhscpg0005479 | 0.543975527 | chr1: 215895774-215895935 | 122221 | 127018 | LYPLAL1 |
| UHNhscpg0002565 | 0.545509304 | chr6: 116998748-116999090 | 1641 | 51389 | RWDD1 |
| UHNhscpg0008258 | 0.545922515 | chr1: 114408830-114409330 | 316 | 148281 | SYT6 |
| UHNhscpg0010487 | 0.547254207 | chr12: 92273678-92274142 | 234 | 11163 | NUDT4 |
| UHNhscpg0002717 | 0.55323384 | chr12: 48302621-48302970 | 649 | | AK123353 |
| UHNhscpg0002673 | 0.553941933 | chr15: 81825124-81825456 | 80654 | 646 | BNC1 |
| UHNhscpg0010939 | 0.554017064 | chr10: 8108807-8109019 | 23399 | | FLJ45983 |
| UHNhscpg0001474 | 0.557227088 | chr8: 53488737-53489332 | 3881 | 9705 | ST18 |
| UHNhscpg0008206 | 0.559375672 | chr14: 89155480-89155723 | 41909 | 29018 | AF118074 |
| UHNhscpg0004409 | 0.560612707 | chr12: 63850471-63850714 | 0 | 23592 | MAN1 |
| UHNhscpg0005280 | 0.566882321 | chr8: 114808371-114808497 | 289953 | 114788 | CSMD3 |
| UHNhscpg0002623 | 0.566952142 | chr12: 14237814-14238207 | 171686 | 55729 | BC063855 |
| UHNhscpg0003738 | 0.567376726 | chr2: 88155376-88155675 | 10310 | 51315 | LOC51315 |
| UHNhscpg0008444 | 0.570746223 | chr10: 122728932-122729159 | 69907 | 55717 | WDR11 |
| UHNhscpg0007259 | 0.575414924 | chr1: 208139197-208139388 | 390 | 7779 | SLC30A1 |
| UHNhscpg0002607 | 0.582792699 | chr2: 26481086-26481443 | 0 | 165082 | GPR113 |
| UHNhscpg0008656 | 0.58347414 | chr1: 115293188-115293360 | 4205 | 7252 | TSHB |
| UHNhscpg0002406 | 0.584415421 | chr18: 48120746-48121468 | 0 | 1630 | DCC |
| UHNhscpg0009002 | 0.5849705 | chr16: 78361983-78362481 | 169871 | 4094 | MAF |
| UHNhscpg0007649 | 0.586330872 | chr14: 80296231-80296366 | 0 | 145508 | C14orf145 |
| UHNhscpg0004597 | 0.587453741 | chr22: 15638898-15638988 | 0 | | MGC57211 |
| UHNhscpg0003289 | 0.613126037 | chr14: 20640986-20641802 | 4239 | 554207 | BC031469 |
| UHNhscpg0002376 | 0.616193621 | chr18: 33116047-33117280 | 0 | 56853 | BRUNOL4 |
| UHNhscpg0002145 | 0.617261027 | chr1: 116672902-116673528 | 13466 | 476 | ATP1A1 |
| UHNhscpg0000928 | 0.628741946 | chr18: 32114987-32115360 | 12305 | 55034 | MOCOS |
| UHNhscpg0008601 | 0.636604373 | chr19: 40897102-40898011 | 0 | 27033 | TZFP |
| UHNhscpg0002312 | 0.640025401 | chr12: 52959901-52960341 | 457 | 3178 | HNRPA1 |
| UHNhscpg0004507 | 0.640711129 | chr2: 80187757-80187860 | 0 | 1496 | CTNNA2 |
| UHNhscpg0002864 | 0.644197159 | chr20: 20293918-20294056 | 2708 | 3642 | INSM1 |
| UHNhscpg0008280 | 0.693689486 | chr3: 111231569-111231663 | 692505 | 55211 | DPPA4 |
| UHNhscpg0003180 | 0.698353684 | chr2: 45078887-45079081 | 1606 | 6496 | SIX3 |

Italicized Genes Involved in Cancer or other disease related
Underlined Genes Brain/neuronal related genes
Dashed Underlined Genes Spermatogenesis, embryogenesis, development related
Age correlation Negative score = inc meth wrt age Positive score = dec meth wrt age

TABLE 2B
Age Related Correlation in Sperm-HHA data set

| | | | Nearest | | |
|---|---|---|---|---|---|
| | | | Gene | | |
| | | | Distance | Nearest | Nearest |
| UHNID | Age corr. | Genome Location | (bp) | GeneID | Gene |
| UHNhscpg0006311 | −0.571461 | chr16: 73575728-73575882 | 0 | 79726 | BC004519 |
| UHNhscpg0000757 | −0.5414097 | chr16: 3084575-3084850 | 1713 | 84891 | ZNF206 |
| UHNhscpg0002369 | −0.5080423 | chr12: 102467385-102467934 | 15583 | 55576 | STAB2 |
| UHNhscpg0001087 | −0.5049054 | chr2: 222992625-222992945 | 0 | 0 | FLJ32447 |
| UHNhscpg0000495 | −0.4991295 | chr2: 38215365-38216278 | 448 | 1545 | CYP1B1 |
| UHNhscpg0000562 | −0.4981613 | chr10: 21822844-21823534 | 18875 | 387640 | FLJ45187 |
| UHNhscpg0009206 | −0.4953882 | chr2: 120996167-120996370 | 56240 | 84931 | FLJ14816 |
| UHNhscpg0005717 | −0.4945551 | chr21: 46567171-46567396 | 1086 | 5116 | PCNT2 |
| UHNhscpg0007805 | −0.49356 | chr2: 223623949-223624362 | 0 | 2181 | ACSL3 |
| UHNhscpg0006512 | −0.4900278 | chr12: 21985242-21985363 | 0 | 10060 | BC033804 |
| UHNhscpg0001346 | −0.4890173 | chr12: 94687435-94687958 | 0 | 0 | METAP2 |
| UHNhscpg0000367 | −0.4675992 | chr9: 123770922-123771907 | 0 | 57706 | AK024782 |
| UHNhscpg0009946 | −0.4518449 | chr17: 70661243-70661746 | 0 | 51155 | HN1 |
| UHNhscpg0007913 | −0.449313 | chr3: 44011141-44011398 | 246983 | 375337 | AK093476 |
| UHNhscpg0002168 | −0.437548 | chr7: 13802034-13802474 | 0 | 2115 | ETV1 |
| UHNhscpg0000973 | −0.4372439 | chr18: 54013705-54014057 | 0 | 23327 | NEDD4L |
| UHNhscpg0001825 | −0.432339 | chr12: 94687426-94687979 | 0 | 0 | METAP2 |
| UHNhscpg0008872 | −0.428427 | chr3: 25681850-25682051 | 1058 | 7155 | TOP2B |
| UHNhscpg0004998 | −0.425298 | chr18: 24676574-24676664 | 665482 | 1000 | CDH2 |
| UHNhscpg0003543 | −0.4174782 | chr13: 93951550-93951661 | 21626 | 1638 | DCT |
| UHNhscpg0000752 | −0.4161375 | chr19: 52308085-52308632 | 0 | 23211 | C19orf7 |
| UHNhscpg0000402 | −0.4145143 | chr10: 70330981-70331591 | 0 | 0 | AK056044 |
| UHNhscpg0000851 | −0.4097783 | chr15: 43466690-43467020 | 8677 | 2628 | GATM |
| UHNhscpg0008103 | −0.4090966 | chr5: 78724780-78724896 | 0 | 9456 | HOMER1 |
| UHNhscpg0000874 | −0.4090138 | chr13: 45524409-45524808 | 0 | 23091 | BC019000 |
| UHNhscpg0003075 | −0.4080136 | chr12: 22379747-22379948 | 832 | 0 | SIAT8A |
| UHNhscpg0007389 | −0.405673 | chr1: 192571739-192571804 | 354765 | 343450 | SLICK |
| UHNhscpg0001303 | −0.405598 | chr12: 50749103-50749772 | 254 | 60673 | FLJ11773 |
| UHNhscpg0008902 | −0.4048977 | chr14: 46575274-46575748 | 0 | 161357 | MAMDC1 |
| UHNhscpg0001410 | −0.4042417 | chr15: 43466690-43467020 | 8677 | 2628 | GATM |
| UHNhscpg0001498 | −0.4035585 | chr19: 14052743-14053182 | 5870 | 113230 | BC011002 |
| UHNhscpg0001795 | −0.4022476 | chr22: 45478515-45478970 | 97 | 25771 | C22orf4 |
| UHNhscpg0000092 | −0.4017695 | chr1: 243700095-243700378 | 45036 | 317705 | VN1R5 |
| UHNhscpg0000407 | −0.4016029 | chr3: 159310225-159310601 | 0 | 51319 | MGC12197 |
| UHNhscpg0001152 | −0.401599 | chr4: 85773951-85774343 | 0 | 4825 | NKX6-1 |
| UHNhscpg0011244 | 0.400513 | chr1: 233050707-233050741 | 0 | 55127 | AK098212 |
| UHNhscpg0008036 | 0.4027472 | chr22: 22430339-22430668 | 0 | 150248 | FLJ36561 |
| UHNhscpg0009859 | 0.4084672 | chr20: 38753133-38753612 | 1843 | 9935 | MAFB |
| UHNhscpg0011733 | 0.4095515 | chr5: 78381324-78381406 | 0 | 29958 | DMGDH |
| UHNhscpg0010258 | 0.4143755 | chr1: 10393997-10394274 | 0 | 5226 | PGD |
| UHNhscpg0005083 | 0.4183838 | chr6: 122167296-122167347 | 354726 | 2697 | GJA1 |
| UHNhscpg0008843 | 0.422049 | chr3: 54282033-54282522 | 0 | 55799 | AF516696 |
| UHNhscpg0003506 | 0.4240404 | chr2: 36496443-36496693 | 0 | 0 | CRIM1 |
| UHNhscpg0005633 | 0.4240989 | chr18: 30341908-30341962 | 85357 | 1837 | DTNA |
| UHNhscpg0005683 | 0.4247309 | chr4: 84391329-84391815 | 0 | 51316 | PLAC8 |
| UHNhscpg0010991 | 0.4284163 | chr12: 78167517-78167778 | 0 | 6857 | SYT1 |
| UHNhscpg0010414 | 0.428641 | chr14: 69723455-69723658 | 0 | 6547 | SLC8A3 |
| UHNhscpg0011471 | 0.4289618 | chr19: 63431698-63431877 | 765 | 27300 | ZNF544 |
| UHNhscpg0011163 | 0.4336394 | chr19: 58188067-58188175 | 0 | 90338 | ZNF160 |
| UHNhscpg0003691 | 0.4439328 | chr8: 116422851-116423033 | 66866 | 7227 | TRPS1 |
| UHNhscpg0011833 | 0.4440889 | chr13: 99427912-99428530 | 3789 | 0 | ZIC2 |
| UHNhscpg0005658 | 0.4461968 | chr7: 4636276-4636727 | 0 | 55698 | FLJ10324 |
| UHNhscpg0003824 | 0.4462915 | chr2: 107673648-107673883 | 238538 | 285190 | BX537861 |
| UHNhscpg0006149 | 0.4473537 | chr1: 193309526-193310390 | 370 | 343450 | SLICK |
| UHNhscpg0005850 | 0.4475083 | chr11: 959952-960191 | 0 | 161 | AP2A2 |
| UHNhscpg0009680 | 0.4498291 | chr14: 38935426-38935732 | 978 | 254170 | FBXO33 |
| UHNhscpg0009704 | 0.4509138 | chr19: 45765931-45766515 | 0 | 57731 | SPTBN4 |
| UHNhscpg0007755 | 0.4520231 | chr15: 76517964-76518338 | 0 | 0 | IREB2 |
| UHNhscpg0008364 | 0.4540589 | chr3: 172679809-172679986 | 18989 | 23043 | AB011123 |
| UHNhscpg0008495 | 0.4544119 | chr21: 36354670-36354909 | 10 | 54093 | C21orf18 |
| UHNhscpg0006360 | 0.4566517 | chr10: 102972869-102973961 | 2762 | 10660 | LBX1 |
| UHNhscpg0005626 | 0.4603208 | chr1: 43493737-43494422 | 0 | 991 | CDC20 |
| UHNhscpg0011755 | 0.4645775 | chr11: 18684679-18684913 | 0 | 0 | FLJ37794 |
| UHNhscpg0009584 | 0.4827054 | chr14: 38935426-38935732 | 978 | 254170 | FBXO33 |
| UHNhscpg0010541 | 0.4897825 | chr20: 1875274-1875325 | 6734 | 140885 | PTPNS1 |
| UHNhscpg0007861 | 0.4988212 | chr19: 8563202-8563420 | 0 | 81794 | ADAMTS10 |
| UHNhscpg0010637 | 0.5227931 | chr20: 1875274-1875325 | 6734 | 140885 | PTPNS1 |
| UHNhscpg0005162 | 0.5267622 | chr4: 123228934-123229348 | 16718 | 0 | TRPC3 |
| UHNhscpg0011884 | 0.5376706 | chr8: 39748508-39748595 | 0 | 2515 | ADAM2 |
| UHNhscpg0005387 | 0.5521166 | chr19: 19634927-19635609 | 0 | 57130 | ATP13A |

Italicized Genes Involved in Cancer or other disease related
Underlined Genes Brain/neuronal related genes
Dashed Underlined Genes Spermatogenesis, embryogenesis, development related
Age correlation Negative score = inc meth wrt age Positive score = dec meth wrt age

TABLE 3
List of clones selected for bisulfite modification MS-SNuPE analysis

| | | | | # Total | # | # variable |
|---|---|---|---|---|---|---|
| | Within | | Chr | Enzyme | CpGs | Methylated |
| UHNID | Gene | Description | Location | CpGs | tested | CpGs |
| UHNhscpg0004931 | OLR1 | oxidised low density | 12p13.2 | 12 | 11 | 9 |
| | | lipoprotein receptor 1 | | | | |
| UHNhscpg0004063 | CDH13 | cadherin 13 preproprotein | 16q23.3 | 8 | 8 | 8 |
| UHNhscpg0002847 | SCAM1 | sorbin and SH3 domain | 8p21.3 | 13 | 6 | 5 |
| | | containing 3 | | | | |
| UHNhscpg0003990 | NELL2 | NEL-like 2 (chicken) | 12q12 | 2 | 2 | 2 |
| UHNhscpg0003907 | NEIL2 | (Nei like 2) (E. coli). | 8p23.1 | 7 | 6 | 4 |
| UHNhscpg0001947 | MKL2 | Megakaryoblastic | 16p13.12 | 10 | 5 | 2 |
| | | leukemia 2. | | | | |
| UHNhscpg0000823 | 2-PDE | 2′-phosphodiesterase | 3p14.3 | 20 | 6 | 1 |
| UHNhscpg0004641 | RHOQ | Ras-related GTP-binding | 2p21 | 12 | 6 | 1 |
| | | protein TC10 | | | | |
| UHNhscpg0002006 | DIRAS3 | ras homolog gene family, | 1p31.2 | 20 | 5 | 0 |
| | | member I | | | | |
| UHNhscpg0005090 | AHR | RWD domain containing 3 | 1p21.3 | 3 | 3 | 2 |
| UHNhscpg0009548 | DSCAM | Down syndrome cell | 21q22.2 | 4 | 3 | 3 |
| | | adhesion molecule | | | | |
| UHNhscpg0004745 | FBN1 | fibrillin 1 (Marfan | 15q21.1 | 2 | 2 | 2 |
| | | syndrome) | | | | |

TABLE 4
33 Significant Loci as described in Example 2.

| | | | | Within | Upstream | Downstream |
|---|---|---|---|---|---|---|
| FDR p-value | ID | UHNID | ChromoSome | Gene | Gene | Gene |
| 0.039503028 | 3_L_4 | UHNhscpg0001078 | 1 | | HLX1 | AK128488 |
| 0.039503028 | 1_M_12 | UHNhscpg0000174 | 16 | FLJ20898 | AL080069 | CRAMP1L |
| 0.088455631 | 4_L_4 | UHNhscpg0001452 | | | | |
| 0.088455631 | 22_L_1 | UHNhscpg0008040 | 1 | | C1orf27 | AF076463 |
| 0.093059438 | 3_F_21 | UHNhscpg0000961 | 5 | CXXC5 | UBE2D2 | CXXC5 |
| 0.093059438 | 23_D_23 | UHNhscpg0008387 | 5 | | TRIM7 | TRIM41 |
| 0.093059438 | 13_H_4 | UHNhscpg0004779 | 15 | HAPLN3 | AGC1 | MFGE8 |
| 0.132947027 | 26_A_3 | UHNhscpg0009325 | 4 | NKX6-1 | MGC11324 | CDS1 |
| 0.132947027 | 2_L_11 | UHNhscpg0000625 | 10 | | AK093639 | SFMBT2 |
| 0.154341647 | 24_D_21 | UHNhscpg0008770 | 6 | HIST1H3G | HIST1H2BH | HIST1H2BI |
| 0.167756484 | 9_M_5 | UHNhscpg0003036 | 2 | LOC84661 | CGI-27 | SPG4 |
| 0.191790532 | 8_C_1 | UHNhscpg0002608 | 9 | ATP6V1G1 | DFNB31 | C9orf91 |
| 0.191790532 | 7_P_18 | UHNhscpg0002592 | 2 | ERBB4 | CPS1 | ZNFN1A2 |
| 0.191790532 | 29_M_8 | UHNhscpg0010647 | 3 | ALCAM | LOC131368 | ALCAM |
| 0.191790532 | 15_P_20 | UHNhscpg0005572 | 16 | | KIAA1007 | FLJ10815 |
| 0.206542373 | 2_I_20 | UHNhscpg0000522 | 12 | IPO8 | ARG99 | C1QDC1 |
| 0.206542373 | 14_M_15 | UHNhscpg0004916 | 12 | SART3 | HYPE | ISCU |
| 0.207137404 | 1_K_12 | UHNhscpg0000162 | 17 | | HES7 | PER1 |
| 0.222755617 | 7_P_17 | UHNhscpg0002496 | 2 | ERBB4 | CPS1 | ZNFN1A2 |
| 0.225913734 | 8_E_5 | UHNhscpg0002622 | | | | |
| 0.225913734 | 6_O_19 | UHNhscpg0001942 | 19 | POLD1 | NR1H2 | SPIB |
| 0.225913734 | 6_H_1 | UHNhscpg0002067 | 11 | FN5 | FLJ25393 | AB051518 |
| 0.225913734 | 6_E_18 | UHNhscpg0001974 | 19 | RPL28 | MDAC1 | UBE2S |
| 0.225913734 | 5_F_12 | UHNhscpg0001784 | 1 | | HLX1 | AK128488 |
| 0.225913734 | 20_H_18 | UHNhscpg0007392 | | | | |
| 0.225913734 | 19_A_9 | UHNhscpg0006714 | 13 | COL4A2 | COL4A1 | AK130129 |
| 0.230896667 | 12_J_22 | UHNhscpg0004425 | 17 | | BCAS3 | TBX2 |
| 0.235298071 | 24_L_22 | UHNhscpg0008914 | 14 | MAMDC1 | RPL10L | RPS29 |
| 0.249926623 | 18_E_18 | UHNhscpg0006459 | 7 | MEST | TSGA14 | COPG2 |
| 0.253542193 | 18_E_9 | UHNhscpg0006360 | 10 | | TLX1 | LBX1 |
| 0.264154456 | 7_O_23 | UHNhscpg0002309 | 20 | C20orf185 | BPIL3 | C20orf186 |
| 0.293661105 | 24_I_15 | UHNhscpg0008611 | 2 | AK123152 | REPRIMO | AK090913 |
| 0.293661105 | 1_C_16 | UHNhscpg0000116 | 21 | ERG | ERG | AY204748 |

TABLE 5
Additional Loci information for bipolar subjects

| Raw p- | | | | | | Downstream |
|---|---|---|---|---|---|---|
| value | id | UHNID | Chr | Within Gene | Upstream Gene | Gene |
| 0.000032 | 3_L_4 | UHNhscpg0001078 | 1 | | HLX1 | AK128488 |
| 0.000072 | 20_A_17 | UHNhscpg0007087 | 19 | SLC1A5 | BC014403 | AP2S1 |
| 0.000096 | 2_L_11 | UHNhscpg0000625 | 10 | | AK093639 | SFMBT2 |
| 0.000221 | 7_P_17 | UHNhscpg0002496 | 2 | ERBB4 | CPS1 | ZNFN1A2 |
| 0.000225 | 26_A_3 | UHNhscpg0009325 | 4 | NKX6-1 | MGC11324 | CDS1 |
| 0.000225 | 15_P_20 | UHNhscpg0005572 | 16 | | KIAA1007 | FLJ10815 |
| 0.000226 | 5_F_12 | UHNhscpg0001784 | 1 | | HLX1 | AK128488 |
| 0.000325 | 12_J_22 | UHNhscpg0004425 | 17 | | BCAS3 | TBX2 |
| 0.000372 | 8_H_6 | UHNhscpg0002907 | 2 | FLJ38377 | GPR148 | ARHGEF4 |
| 0.00043 | 6_M_20 | UHNhscpg0002021 | 1 | AY499148 | LOC127540 | MGC15882 |
| 0.000443 | 7_P_18 | UHNhscpg0002592 | 2 | ERBB4 | CPS1 | ZNFN1A2 |
| 0.000499 | 4_L_4 | UHNhscpg0001452 | | | | |
| 0.000612 | 3_F_21 | UHNhscpg0000961 | 5 | CXXC5 | UBE2D2 | CXXC5 |
| 0.000674 | 31_P_21 | UHNhscpg0011530 | 16 | BC064026 | TNFRSF12A | AK128093 |
| 0.000694 | 32_K_4 | UHNhscpg0011785 | 16 | | FOXL1 | FBXO31 |
| 0.00072 | 8_O_17 | UHNhscpg0002684 | 2 | ERBB4 | CPS1 | ZNFN1A2 |
| 0.000732 | 7_P_11 | UHNhscpg0002493 | 5 | PCDHGA1 | PCDHGA5 | PCDHGA6 |
| 0.000791 | 10_A_2 | UHNhscpg0003428 | 15 | TORC3 | IQGAP1 | AK130544 |
| 0.000812 | 6_F_11 | UHNhscpg0002061 | 12 | IPO8 | ARG99 | C1QDC1 |
| 0.000868 | 6_I_20 | UHNhscpg0001998 | 21 | WRB | HMGN1 | MGC33295 |
| 0.000943 | 5_D_21 | UHNhscpg0001690 | 15 | | FAH | BC051335 |
| 0.00095 | 4_H_20 | UHNhscpg0001436 | 10 | | AK093639 | SFMBT2 |
| 0.001017 | 20_B_2 | UHNhscpg0007350 | 4 | | LPHN3 | EPHA5 |
| 0.00111 | 10_E_19 | UHNhscpg0003366 | 13 | | ESD | HTR2A |
| 0.001155 | 8_E_5 | UHNhscpg0002622 | | | | |
| 0.001173 | 24_I_15 | UHNhscpg0008611 | 2 | AK123152 | REPRIMO | AK090913 |
| 0.001269 | 20_H_18 | UHNhscpg0007392 | | | | |
| 0.001293 | 19_A_9 | UHNhscpg0006714 | 13 | COL4A2 | COL4A1 | AK130129 |
| 0.001295 | 26_F_24 | UHNhscpg0009647 | 5 | | AK026075 | DHX29 |
| 0.001303 | 1_K_12 | UHNhscpg0000162 | 17 | | HES7 | PER1 |
| 0.00137 | 14_M_15 | UHNhscpg0004916 | 12 | SART3 | HYPE | ISCU |
| 0.001383 | 4_P_10 | UHNhscpg0001478 | 1 | | APG4C | FOXD3 |
| 0.001388 | 16_F_8 | UHNhscpg0005885 | 1 | EPS15 | C1orf34 | OSBPL9 |
| 0.001399 | 1_A_21 | UHNhscpg0000011 | 16 | | CNTNAP4 | HSRG1 |
| 0.001563 | 24_P_8 | UHNhscpg0008931 | 5 | CDH12 | PMCHL1 | PRDM9 |
| 0.001614 | 28_C_1 | UHNhscpg0010104 | 17 | FLJ20920 | CHAD | FLJ11164 |
| 0.001635 | 7_O_12 | UHNhscpg0002399 | 5 | PCDHGA1 | PCDHGA5 | PCDHGA6 |
| 0.001886 | 1_M_12 | UHNhscpg0000174 | 16 | FLJ20898 | AL080069 | CRAMP1L |
| 0.002079 | 2_D_21 | UHNhscpg0000583 | 18 | MADH4 | ELAC1 | RKHD2 |
| 0.002088 | 22_N_21 | UHNhscpg0008062 | 10 | | LYZL2 | LOC220929 |
| 0.002212 | 3_G_23 | UHNhscpg0000789 | 17 | FLJ45455 | AY550194 | LOC284033 |
| 0.002215 | 28_I_23 | UHNhscpg0010151 | 1 | DEPDC1 | DEPDC1 | KIAA1365 |
| 0.002334 | 10_G_17 | UHNhscpg0003376 | 10 | | C10orf39 | DPYSL4 |
| 0.002375 | 9_A_13 | UHNhscpg0002968 | 13 | BC020814 | SLC25A15 | ELF1 |
| 0.002455 | 19_D_21 | UHNhscpg0006916 | 4 | | PCDH7 | CENTD1 |
| 0.002518 | 25_G_17 | UHNhscpg0008984 | 8 | PLAG1 | MOS | AK098285 |
| 0.002689 | 22_C_2 | UHNhscpg0007896 | 21 | | DSCR3 | DYRK1A |
| 0.00284 | 27_E_7 | UHNhscpg0009735 | 6 | MGC33835 | CLPS | SRPK1 |
| 0.00305 | 18_E_18 | UHNhscpg0006459 | 7 | MEST | TSGA14 | COPG2 |
| 0.003088 | 22_D_19 | UHNhscpg0008001 | 3 | | BC067737 | SLC9A9 |
| 0.003128 | 6_D_15 | UHNhscpg0002053 | 20 | C20orf85 | AK128005 | C20orf86 |
| 0.003195 | 7_J_11 | UHNhscpg0002458 | 12 | OAS3 | OAS1 | OAS2 |
| 0.003263 | 3_E_23 | UHNhscpg0000778 | 2 | | BRE | FOSL2 |
| 0.00343 | 29_M_8 | UHNhscpg0010647 | 3 | ALCAM | LOC131368 | ALCAM |
| 0.00349 | 12_I_15 | UHNhscpg0004138 | 6 | HIST1H3A | HIST1H1A | HIST1H4D |
| 0.003595 | 9_L_14 | UHNhscpg0003305 | 7 | EN2 | INSIG1 | AK124544 |
| 0.003595 | 13_H_5 | UHNhscpg0004689 | | | | |
| 0.00362 | 14_N_10 | UHNhscpg0005183 | 9 | | AL834325 | MAPKAP1 |
| 0.003749 | 30_M_23 | UHNhscpg0010943 | 14 | | STXBP6 | NOVA1 |
| 0.003796 | 8_H_5 | UHNhscpg0002818 | 5 | AK057601 | LOC375449 | AB002301 |
| 0.003843 | 18_F_24 | UHNhscpg0006654 | 4 | GRID2 | AY499148 | ATOH1 |
| 0.003881 | 2_C_15 | UHNhscpg0000397 | 5 | FBXW1B | FGF18 | STK10 |
| 0.004028 | 24_N_21 | UHNhscpg0008830 | 9 | C9orf40 | AK126029 | C9orf41 |
| 0.004105 | 24_N_10 | UHNhscpg0008920 | 5 | CDH12 | PMCHL1 | PRDM9 |
| 0.004177 | 8_E_24 | UHNhscpg0002723 | 9 | | SLC28A3 | AF174394 |
| 0.004214 | 6_H_9 | UHNhscpg0002071 | 22 | | CERK | C22orf4 |
| 0.004225 | 15_N_3 | UHNhscpg0005463 | 11 | | DLG2 | HT007 |
| 0.004307 | 7_P_20 | UHNhscpg0002593 | 3 | MLF1 | MGC12197 | MLF1 |
| 0.004359 | 26_J_7 | UHNhscpg0009567 | X | DMD | DMD | S71486 |
| 0.004511 | 31_F_17 | UHNhscpg0011468 | 1 | | CAPZA1 | MOV10 |
| 0.004567 | 25_D_1 | UHNhscpg0009144 | 3 | | AK092352 | CIDE-3 |
| 0.004795 | 11_O_21 | UHNhscpg0003793 | 1 | | FLJ10948 | BC067108 |
| 0.004893 | 1_O_1 | UHNhscpg0000085 | 17 | AY302137 | AK024458 | M-RIP |
| 0.004904 | 25_C_1 | UHNhscpg0008952 | 6 | | RING1 | VPS52 |
| 0.004988 | 28_M_12 | UHNhscpg0010265 | 12 | BC059370 | AF289571 | RBMS2 |
| 0.005021 | 6_N_3 | UHNhscpg0002104 | 2 | | SPR | EMX1 |
| 0.005122 | 29_J_3 | UHNhscpg0010717 | X | | PRDX4 | ACATE2 |
| 0.005179 | 6_O_4 | UHNhscpg0002024 | 3 | | ZIC1 | U87591 |
| 0.0052 | 19_D_2 | UHNhscpg0006998 | 4 | | NDST3 | PRSS12 |
| 0.00521 | 28_G_17 | UHNhscpg0010136 | 2 | | FIGN | GRB14 |
| 0.005227 | 20_F_5 | UHNhscpg0007286 | 4 | | NPY1R | NPY5R |
| 0.005339 | 26_M_17 | UHNhscpg0009404 | 9 | | C9orf62 | KIAA0649 |
| 0.005553 | 17_H_2 | UHNhscpg0006273 | 15 | CA12 | CA12 | USP3 |
| 0.005682 | 7_D_1 | UHNhscpg0002418 | 9 | ATP6V1G1 | DFNB31 | C9orf91 |
| 0.005993 | 32_O_7 | UHNhscpg0011715 | | | | |
| 0.006207 | 27_L_20 | UHNhscpg0010065 | 3 | | MGC26717 | EPHA3 |
| 0.006341 | 6_A_11 | UHNhscpg0001854 | 9 | PTPN3 | PTPN3 | PTPN3 |
| 0.006354 | 25_N_1 | UHNhscpg0009204 | 3 | KIAA0089 | OSBPL10 | CKLFSF8 |
| 0.006431 | 6_K_4 | UHNhscpg0002001 | 1 | LAMC2 | LAMC1 | NMNAT2 |
| 0.006539 | 22_C_19 | UHNhscpg0007809 | 3 | | PTPRG | HT021 |
| 0.006701 | 26_H_5 | UHNhscpg0009554 | 20 | | AB033098 | AF451990 |
| 0.006787 | 4_L_17 | UHNhscpg0001365 | | | | |
| 0.006862 | 19_C_1 | UHNhscpg0006721 | | | | |
| 0.006867 | 7_O_18 | UHNhscpg0002402 | 2 | ERBB4 | CPS1 | ZNFN1A2 |
| 0.006964 | 7_P_21 | UHNhscpg0002498 | 7 | CPVL | CPVL | CHN2 |
| 0.007128 | 8_O_19 | UHNhscpg0002685 | 3 | DBR1 | DBR1 | HSPC056 |
| 0.007153 | 4_K_3 | UHNhscpg0001173 | | | | |
| 0.007157 | 13_P_21 | UHNhscpg0004742 | | | | |
| 0.007385 | 3_H_22 | UHNhscpg0001063 | 15 | | BC066364 | PTPN9 |
| 0.007402 | 13_B_7 | UHNhscpg0004654 | 10 | | BCO27847 | DRD1IP |
| 0.007404 | 17_G_8 | UHNhscpg0006088 | 19 | ZNF582 | ZNF542 | ZNF583 |
| 0.007531 | 3_K_16 | UHNhscpg0000899 | 5 | AY358358 | BC062459 | OR2V2 |
| 0.007711 | 7_F_5 | UHNhscpg0002432 | 4 | DKFZP564O0823 | BTC | AK027257 |
| 0.007798 | 5_G_10 | UHNhscpg0001616 | 6 | | AK021951 | STXBP5 |
| 0.008117 | 5_F_17 | UHNhscpg0001700 | 3 | MRPL47 | GNB4 | MRPL47 |
| 0.008146 | 24_N_16 | UHNhscpg0008923 | 6 | MRPS18A | C6orf206 | VEGF |
| 0.008217 | 15_O_22 | UHNhscpg0005389 | 3 | AF351617 | AY070437 | SCHIP1 |
| 0.0083 | 13_M_5 | UHNhscpg0004535 | 12 | | CAPZA3 | PEPP2 |
| 0.0084 | 12_D_13 | UHNhscpg0004289 | 10 | | HIF1AN | PAX2 |
| 0.008507 | 28_B_18 | UHNhscpg0010388 | 12 | | U47671 | TBX3 |
| 0.008596 | 18_N_14 | UHNhscpg0006693 | 11 | | SCGB1A1 | MGC5395 |
| 0.008615 | 14_H_17 | UHNhscpg0005068 | 1 | TMEM9 | CACNA1S | DKFZp434B1231 |
| 0.008801 | 10_J_7 | UHNhscpg0003564 | 3 | | CNTN6 | CNTN4 |
| 0.00891 | 23_F_4 | UHNhscpg0008485 | 7 | AP4M1 | MCM7 | AP4M1 |
| 0.009035 | 12_D_10 | UHNhscpg0004383 | | | | |
| 0.009073 | 13_K_10 | UHNhscpg0004620 | 6 | NCOA7 | HEY2 | NCOA7 |
| 0.009175 | 17_M_19 | UHNhscpg0006035 | 6 | PAK1IP1 | C6orf52 | TMEM14C |
| 0.009293 | 31_F_18 | UHNhscpg0011564 | 1 | | CAPZA1 | MOV10 |
| 0.009331 | 14_D_1 | UHNhscpg0005042 | 11 | | MYEOV | CCND1 |
| 0.009334 | 11_P_18 | UHNhscpg0004079 | 12 | U80760 | ING4 | ZNF384 |
| 0.009352 | 17_B_10 | UHNhscpg0006241 | 1 | | JARID1B | RABIF |
| 0.009396 | 7_B_4 | UHNhscpg0002501 | 2 | | TANK | PSMD14 |
| 0.009423 | 18_B_22 | UHNhscpg0006629 | 12 | | AK126039 | SFRS8 |
| 0.009476 | 6_N_4 | UHNhscpg0002193 | 2 | | SPR | EMX1 |
| 0.009595 | 4_O_2 | UHNhscpg0001287 | 7 | | PODXL | PLXNA4 |
| 0.009606 | 2_L_12 | UHNhscpg0000715 | 6 | TAF11 | C6orf107 | ANKS1 |
| 0.009608 | 31_B_22 | UHNhscpg0011542 | 17 | CPD | UNQ9372 | GOSR1 |
| 0.009641 | 9_H_4 | UHNhscpg0003279 | 19 | | AXL | HNRPUL1 |
| 0.009678 | 16_A_23 | UHNhscpg0005586 | X | | IRS4 | GUCY2F |
| 0.009857 | 14_I_4 | UHNhscpg0004977 | | | | |
| 0.009862 | 24_J_18 | UHNhscpg0008900 | 14 | MAMDC1 | RPL10L | RPS29 |
| 0.009954 | 20_M_23 | UHNhscpg0007158 | 9 | GLDC | UHRF2 | JMJD2C |
| 0.010057 | 31_B_1 | UHNhscpg0011436 | 2 | | GBX2 | ASB18 |
| 0.010146 | 24_P_9 | UHNhscpg0008836 | 15 | SPG6 | SPG6 | FLJ35785 |
| 0.010315 | 28_D_18 | UHNhscpg0010400 | 12 | FLJ22789 | AK001156 | DKFZp762A217 |
| 0.010339 | 5_H_12 | UHNhscpg0001796 | 3 | ZNF35 | ZNF197 | ZNF35 |
| 0.01044 | 11_N_7 | UHNhscpg0003966 | | | | |
| 0.010497 | 1_F_20 | UHNhscpg0000319 | | | | |
| 0.01063 | 10_H_20 | UHNhscpg0003649 | 10 | | HSPA14 | SUV39H2 |
| 0.010639 | 1_J_23 | UHNhscpg0000250 | 6 | | PREP | PRDM1 |
| 0.010731 | 26_M_14 | UHNhscpg0009498 | 10 | | IDE | KIF11 |
| 0.010751 | 19_E_9 | UHNhscpg0006737 | 12 | | U47671 | TBX3 |
| 0.010834 | 21_K_8 | UHNhscpg0007580 | 1 | FUSIP1 | PNRC2 | FLJ35961 |
| 0.010854 | 30_B_18 | UHNhscpg0011156 | | | | |
| 0.010927 | 12_I_24 | UHNhscpg0004234 | 7 | U88666 | AK126439 | MLL5 |
| 0.011022 | 8_F_20 | UHNhscpg0002903 | 18 | RBBP8 | CTAGE-1 | RBBP8 |
| 0.011035 | 28_L_2 | UHNhscpg0010440 | 11 | | AK074166 | OSBP |
| 0.01107 | 21_B_5 | UHNhscpg0007613 | 15 | IREB2 | CRABP1 | IREB2 |
| 0.011115 | 6_H_5 | UHNhscpg0002069 | 20 | | TFAP2C | BMP7 |
| 0.011212 | 6_E_24 | UHNhscpg0001977 | 12 | | GCN1L1 | AF274958 |
| 0.011221 | 13_E_5 | UHNhscpg0004488 | Y | | NLGN4Y | XKRY |
| 0.011271 | 23_D_23 | UHNhscpg0008387 | 5 | | TRIM7 | TRIM41 |
| 0.011279 | 11_A_5 | UHNhscpg0003701 | | | | |
| 0.011492 | 12_P_23 | UHNhscpg0004366 | | | | |
| 0.011718 | 25_E_7 | UHNhscpg0008967 | 14 | | NKX2-8 | AY064415 |
| 0.011929 | 19_I_5 | UHNhscpg0006759 | | | | |
| 0.011992 | 27_A_2 | UHNhscpg0009804 | 6 | | EPHA7 | MANEA |
| 0.012157 | 26_O_22 | UHNhscpg0009514 | 1 | | PF6 | BX537778 |
| 0.012563 | 29_P_11 | UHNhscpg0010757 | 1 | IER5 | MR1 | AF387616 |
| 0.012753 | 20_E_1 | UHNhscpg0007102 | | | | |
| 0.012776 | 15_J_20 | UHNhscpg0005537 | 7 | | CENTG3 | ASB10 |
| 0.012844 | 4_P_22 | UHNhscpg0001484 | 4 | HIP2 | HIP2 | KIAA0648 |
| 0.01287 | 17_C_5 | UHNhscpg0005968 | 22 | FBXO7 | BPIL2 | SYN3 |
| 0.013007 | 12_C_14 | UHNhscpg0004193 | 11 | | RAB6A | MRPL48 |
| 0.013008 | 10_B_19 | UHNhscpg0003522 | 20 | | ANKRD5 | SNAP25 |
| 0.013028 | 20_E_21 | UHNhscpg0007111 | 1 | AY499148 | CSMD2 | MGC15882 |
| 0.013032 | 30_K_7 | UHNhscpg0010923 | | | | |
| 0.013033 | 5_H_18 | UHNhscpg0001799 | 20 | PXMP4 | E2F1 | ZNF341 |
| 0.013085 | 21_K_4 | UHNhscpg0007578 | 16 | | CNTNAP4 | HSRG1 |
| 0.013102 | 25_P_4 | UHNhscpg0009313 | 1 | | SSB1 | MGC4399 |
| 0.013264 | 25_P_5 | UHNhscpg0009218 | 18 | | BRUNOL4 | PIK3C3 |
| 0.013382 | 27_P_24 | UHNhscpg0010091 | 6 | AF116727 | SERPINB9 | DKFZp686I15217 |
| 0.013441 | 15_A_21 | UHNhscpg0005211 | | | | |
| 0.013475 | 4_J_10 | UHNhscpg0001443 | 2 | MTX2 | HOXD1 | BC013438 |
| 0.013508 | 8_G_23 | UHNhscpg0002640 | 2 | LOC284948 | CAPG | LOC284948 |
| 0.013544 | 22_P_18 | UHNhscpg0008168 | 4 | | ZCCHC4 | ANAPC4 |
| 0.013544 | 10_A_21 | UHNhscpg0003344 | 2 | ATF2 | ATF2 | ATP5G3 |
| 0.01369 | 5_O_22 | UHNhscpg0001667 | | | | |
| 0.013696 | 3_O_5 | UHNhscpg0000823 | 3 | DKFZp667B1218 | FLJ44290 | ARF4 |
| 0.013701 | 4_O_17 | UHNhscpg0001203 | 20 | SPAG4 | AK026266 | CPNE1 |
| 0.013977 | 14_H_6 | UHNhscpg0005150 | 6 | | TRERF1 | BC064512 |
| 0.014023 | 29_A_12 | UHNhscpg0010577 | | | | |
| 0.014126 | 25_C_21 | UHNhscpg0008962 | 9 | | DMRT3 | DMRT2 |
| 0.01414 | 25_N_11 | UHNhscpg0009209 | 10 | | ZNF11B | BMS1L |
| 0.014271 | 29_K_22 | UHNhscpg0010642 | 19 | BC067129 | AF161369 | AKAP8 |
| 0.01428 | 30_P_22 | UHNhscpg0011242 | 12 | | ERBB3 | PA2G4 |
| 0.014354 | 22_K_20 | UHNhscpg0007953 | 18 | | GALNT1 | C18orf21 |
| 0.014364 | 20_I_7 | UHNhscpg0007127 | 1 | CAP350 | LAP1B | AF387614 |
| 0.014477 | 22_P_4 | UHNhscpg0008161 | 12 | | AY070435 | BC032019 |
| 0.01459 | 7_H_4 | UHNhscpg0002537 | 5 | NR2F1 | ARRDC3 | AK126015 |
| 0.014676 | 20_I_17 | UHNhscpg0007132 | 13 | BC067898 | FLJ25952 | FLJ34588 |
| 0.014719 | 17_B_13 | UHNhscpg0006149 | 1 | | SLICK | HF1 |
| 0.014788 | 7_B_3 | UHNhscpg0002407 | 1 | | HLX1 | AK128488 |
| 0.014815 | 28_F_17 | UHNhscpg0010316 | 21 | DSCAM | PCP4 | BACE2 |
| 0.014851 | 9_K_2 | UHNhscpg0003113 | 22 | TAFA5 | AK128136 | AK124622 |
| 0.01488 | 11_A_20 | UHNhscpg0003804 | 3 | | TOP2B | NGLY1 |
| 0.014937 | 28_I_12 | UHNhscpg0010241 | X | | IRS4 | GUCY2F |
| 0.015026 | 30_H_6 | UHNhscpg0011186 | 10 | | TAF3 | FLJ45983 |
| 0.015142 | 3_H_16 | UHNhscpg0001060 | 5 | BC041694 | MYO10 | FLJ34047 |
| 0.015153 | 5_F_4 | UHNhscpg0001780 | 15 | | RaLP | KIAA0256 |
| 0.015268 | 14_C_16 | UHNhscpg0004949 | 6 | | STX11 | UTRN |
| 0.01535 | 10_N_6 | UHNhscpg0003677 | 12 | | EPS8 | STRAP |
| 0.015669 | 27_G_2 | UHNhscpg0009840 | 7 | | INHBA | GLI3 |
| 0.015702 | 11_M_11 | UHNhscpg0003776 | X | | TM4SF2 | MIG12 |
| 0.015705 | 27_P_9 | UHNhscpg0009988 | 1 | | KCNK2 | KCTD3 |
| 0.015708 | 18_L_5 | UHNhscpg0006585 | 7 | | AASS | CADPS2 |
| 0.015725 | 1_P_2 | UHNhscpg0000366 | 18 | BC031560 | MPPE1 | CIDEA |
| 0.015744 | 23_D_13 | UHNhscpg0008382 | 8 | | LACTB2 | EYA1 |
| 0.01587 | 24_C_12 | UHNhscpg0008669 | | | | |
| 0.016051 | 20_M_8 | UHNhscpg0007243 | | | | |
| 0.016097 | 28_L_17 | UHNhscpg0010352 | 14 | | MGEA6 | FBXO33 |
| 0.016293 | 4_B_1 | UHNhscpg0001299 | 12 | LOC144233 | AB046822 | FAIM2 |
| 0.016297 | 5_B_21 | UHNhscpg0001679 | 9 | TLE1 | TLE4 | AY129016 |
| 0.016335 | 15_O_1 | UHNhscpg0005283 | 2 | | LRP2 | BBS5 |
| 0.016338 | 29_N_1 | UHNhscpg0010740 | 4 | | SPOCK3 | ANXA10 |
| 0.016419 | 6_E_18 | UHNhscpg0001974 | 19 | RPL28 | MDAC1 | UBE2S |
| 0.016426 | 13_M_17 | UHNhscpg0004544 | 13 | AK092024 | DIAPH3 | TDRD3 |
| 0.016448 | 24_K_7 | UHNhscpg0008619 | 2 | AK095362 | DKFZP434B1727 | R3HDM |
| 0.016496 | 29_B_7 | UHNhscpg0010671 | 11 | | MMP26 | OR52A1 |
| 0.016547 | 18_O_8 | UHNhscpg0006514 | 3 | EXOSC7 | ZDHHC3 | AK090511 |
| 0.016627 | 6_C_16 | UHNhscpg0001962 | 20 | C20orf85 | AK128005 | C20orf86 |
| 0.016633 | 32_I_7 | UHNhscpg0011679 | 19 | RFX2 | RANBP3 | BGR |
| 0.016703 | 13_A_2 | UHNhscpg0004556 | 2 | IL1A | IL1A | IL1B |
| 0.0168 | 26_P_22 | UHNhscpg0009706 | 12 | | GRIP1 | TIP120A |
| 0.01681 | 27_H_1 | UHNhscpg0009936 | 7 | | INHBA | GLI3 |
| 0.016847 | 22_F_6 | UHNhscpg0008102 | 9 | | WDR34 | SET |
| 0.016867 | 18_I_24 | UHNhscpg0006486 | 1 | AF529206 | AIM2 | IGSF4B |
| 0.016927 | 28_L_24 | UHNhscpg0010451 | X | | ESX1L | IL1RAPL2 |
| 0.016977 | 27_B_6 | UHNhscpg0009998 | 11 | RAB38 | FLJ22104 | CTSC |
| 0.016979 | 4_P_17 | UHNhscpg0001388 | 5 | TRIM52 | AK026811 | AK128780 |
| 0.017076 | 5_O_2 | UHNhscpg0001658 | 3 | GORASP1 | KIAA1449 | STI2 |
| 0.017165 | 20_P_21 | UHNhscpg0007348 | 3 | GC20 | MYRIP | ENTPD3 |
| 0.017299 | 15_C_12 | UHNhscpg0005312 | 5 | BX640900 | LOC285671 | FLJ36754 |
| 0.017514 | 22_D_1 | UHNhscpg0007992 | 1 | | LOC128153 | CGI-115 |
| 0.01753 | 9_K_1 | UHNhscpg0003022 | 3 | BC015560 | AL050097 | AK094447 |
| 0.017641 | 14_J_7 | UHNhscpg0005072 | | | | |
| 0.017675 | 24_D_21 | UHNhscpg0008770 | 6 | HIST1H3G | HIST1H2BH | HIST1H2BI |
| 0.017719 | 12_F_7 | UHNhscpg0004298 | 11 | | AF338191 | SLC6A5 |
| 0.017853 | 13_K_18 | UHNhscpg0004624 | | | | |
| 0.017858 | 16_A_8 | UHNhscpg0005672 | 11 | | CSTF3 | HIPK3 |
| 0.018077 | 20_B_20 | UHNhscpg0007359 | | | | |
| 0.018279 | 23_I_23 | UHNhscpg0008231 | 12 | TAFA2 | SLC16A7 | USP15 |
| 0.018357 | 6_O_19 | UHNhscpg0001942 | 19 | POLD1 | NR1H2 | SPIB |
| 0.018414 | 13_L_14 | UHNhscpg0004808 | 11 | | CRSP6 | BX641008 |
| 0.018541 | 28_M_11 | UHNhscpg0010169 | | | | |
| 0.018649 | 20_J_4 | UHNhscpg0007396 | | | | |
| 0.018792 | 29_H_21 | UHNhscpg0010714 | 1 | | AK096396 | WARS2 |
| 0.018795 | 21_P_3 | UHNhscpg0007688 | 2 | | ADAM23 | MDH1B |
| 0.018826 | 4_P_11 | UHNhscpg0001385 | 9 | | DMRT3 | DMRT2 |
| 0.019173 | 11_D_19 | UHNhscpg0003912 | 12 | POP5 | RNF10 | CABP1 |
| 0.019201 | 12_D_20 | UHNhscpg0004388 | 15 | | FLJ35695 | FLJ39531 |
| 0.019232 | 22_M_9 | UHNhscpg0007864 | 6 | WTAP | SOD2 | WTAP |
| 0.019304 | 24_A_1 | UHNhscpg0008556 | 1 | LAP1B | LAP1B | LAP1B |
| 0.019337 | 21_C_5 | UHNhscpg0007453 | 16 | | MT1F | MT1K |
| 0.019525 | 12_C_13 | UHNhscpg0004101 | 11 | | RAB6A | MRPL48 |
| 0.019842 | 26_E_2 | UHNhscpg0009444 | 13 | ESD | CHDC1 | HTR2A |
| 0.019873 | 23_D_17 | UHNhscpg0008384 | | | | |
| 0.019897 | 7_D_2 | UHNhscpg0002512 | 9 | ATP6V1G1 | DFNB31 | C9orf91 |
| 0.019971 | 19_N_9 | UHNhscpg0006967 | | | | |
| 0.020068 | 7_A_4 | UHNhscpg0002311 | 1 | | HLX1 | AK128488 |
| 0.020079 | 9_H_8 | UHNhscpg0003281 | 13 | | GPR80 | MBNL2 |
| 0.020102 | 15_F_14 | UHNhscpg0005512 | | | | |
| 0.020283 | 22_L_17 | UHNhscpg0008048 | 5 | | ZFP62 | BTNL8 |
| 0.02031 | 7_B_16 | UHNhscpg0002507 | 6 | | SF3B5 | STX11 |
| 0.020348 | 12_G_12 | UHNhscpg0004216 | 11 | | WNT11 | PRKRIR |
| 0.020395 | 27_B_19 | UHNhscpg0009909 | 1 | | DISC1 | SIPA1L2 |
| 0.020503 | 3_L_16 | UHNhscpg0001083 | 4 | | AF130075 | HAND2 |
| 0.020801 | 20_G_14 | UHNhscpg0007211 | | | | |
| 0.020834 | 5_H_23 | UHNhscpg0001715 | 1 | | CSRP1 | BC030568 |
| 0.021026 | 1_I_7 | UHNhscpg0000052 | 4 | ENPP6 | AB037813 | MGC24125 |
| 0.021068 | 5_E_4 | UHNhscpg0001601 | 15 | | RaLP | KIAA0256 |
| 0.021296 | 17_M_11 | UHNhscpg0006031 | 8 | | AK128212 | GPR20 |
| 0.021298 | 10_L_22 | UHNhscpg0003673 | 5 | | BC011998 | AB032953 |
| 0.021431 | 4_P_1 | UHNhscpg0001380 | 7 | | PODXL | PLXNA4 |
| 0.021503 | 16_N_22 | UHNhscpg0005940 | 2 | | INSIG2 | EN1 |
| 0.021559 | 28_E_9 | UHNhscpg0010120 | | | | |
| 0.021596 | 14_J_24 | UHNhscpg0005168 | 10 | C10orf11 | AK024492 | KCNMA1 |
| 0.021632 | 10_H_3 | UHNhscpg0003550 | 2 | INSIG2 | FLJ10996 | INSIG2 |
| 0.021652 | 4_B_22 | UHNhscpg0001402 | 1 | NMNAT2 | NMNAT2 | AL050020 |
| 0.021676 | 32_K_23 | UHNhscpg0011699 | 15 | AKAP13 | FLJ40113 | AKAP13 |
| 0.022052 | 9_C_15 | UHNhscpg0002981 | 12 | | SIAT8A | KIAA0528 |
| 0.022105 | 16_P_6 | UHNhscpg0005944 | 16 | | MGC3121 | FBS1 |
| 0.022336 | 2_L_23 | UHNhscpg0000629 | 22 | CERK | AB018310 | CERK |
| 0.022434 | 10_E_18 | UHNhscpg0003458 | 4 | | BC039540 | PGM2 |
| 0.022752 | 32_C_11 | UHNhscpg0011645 | 3 | BC047734 | FHIT | ID2B |
| 0.022946 | 24_B_22 | UHNhscpg0008854 | 11 | | BC047021 | MGC13125 |
| 0.022959 | 6_H_1 | UHNhscpg0002067 | 11 | FN5 | FLJ25393 | AB051518 |
| 0.022974 | 20_C_9 | UHNhscpg0007095 | 6 | | C6orf167 | POU3F2 |
| 0.023079 | 14_P_4 | UHNhscpg0005191 | 16 | | A2BP1 | MGC2654 |
| 0.023131 | 6_J_12 | UHNhscpg0002175 | 3 | MGC2776 | PPARG | MGC2776 |
| 0.023172 | 18_E_9 | UHNhscpg0006360 | 10 | | TLX1 | LBX1 |
| 0.02332 | 18_H_5 | UHNhscpg0006561 | | | | |
| 0.023347 | 12_H_11 | UHNhscpg0004312 | 12 | | SMARCC2 | RNF41 |
| 0.023356 | 27_J_22 | UHNhscpg0010054 | 13 | | SOX21 | ABCC4 |
| 0.023359 | 7_A_18 | UHNhscpg0002318 | 19 | | U87593 | BC068609 |
| 0.023427 | 11_H_11 | UHNhscpg0003932 | 6 | FLJ25791 | ZBTB24 | AK124171 |
| 0.023459 | 14_G_4 | UHNhscpg0004966 | 2 | | AK126832 | SIX3 |
| 0.023593 | 28_I_7 | UHNhscpg0010143 | 21 | | SUMO3 | ITGB2 |
| 0.02366 | 30_M_16 | UHNhscpg0011035 | 10 | | TAF3 | FLJ45983 |
| 0.023705 | 22_C_22 | UHNhscpg0007906 | 1 | | FAM31B | LHX9 |
| 0.023783 | 29_M_7 | UHNhscpg0010551 | | | | |
| 0.024215 | 32_I_8 | UHNhscpg0011775 | | | | |
| 0.024252 | 23_L_7 | UHNhscpg0008427 | X | | FLJ34960 | SMPX |
| 0.024306 | 25_C_22 | UHNhscpg0009058 | 7 | FLJ32110 | MGC26647 | AK098759 |
| 0.024334 | 11_J_7 | UHNhscpg0003942 | 6 | PACRG | PARK2 | QKI |
| 0.02456 | 25_L_1 | UHNhscpg0009192 | 15 | | AK094481 | AK128197 |
| 0.024566 | 28_H_11 | UHNhscpg0010325 | 6 | MANEA | EPHA7 | MANEA |
| 0.024648 | 29_E_16 | UHNhscpg0010603 | 11 | ACAT1 | CUL5 | NPAT |
| 0.024874 | 6_G_11 | UHNhscpg0001890 | 12 | BT007195 | ESPL1 | C12orf10 |
| 0.025156 | 21_A_9 | UHNhscpg0007443 | 3 | LARS2 | RIS1 | LIMD1 |
| 0.025174 | 5_J_4 | UHNhscpg0001804 | 6 | PLA2G7 | TDRD6 | MEP1A |
| 0.025361 | 14_G_13 | UHNhscpg0004880 | 17 | | FLJ21865 | AK123188 |
| 0.025488 | 25_P_6 | UHNhscpg0009314 | 18 | | BRUNOL4 | PIK3C3 |
| 0.025928 | 23_O_20 | UHNhscpg0008361 | 6 | KNSL8 | MRPL2 | PTK7 |
| 0.025937 | 4_O_16 | UHNhscpg0001294 | 1 | | HSPA6 | FCGR3B |
| 0.026045 | 19_E_14 | UHNhscpg0006832 | 15 | BC038449 | TYRO3 | AK022696 |
| 0.026088 | 10_E_17 | UHNhscpg0003365 | 4 | | BC039540 | PGM2 |
| 0.026157 | 7_I_11 | UHNhscpg0002267 | 7 | | MTERF | AKAP9 |
| 0.026186 | 1_C_16 | UHNhscpg0000116 | 21 | ERG | ERG | AY204748 |
| 0.026233 | 31_M_13 | UHNhscpg0011322 | | | | |
| 0.026387 | 13_K_6 | UHNhscpg0004618 | 10 | LDB1 | LDB1 | PPRC1 |
| 0.026418 | 14_G_9 | UHNhscpg0004878 | 18 | | ZCCHC2 | PLEKHE1 |
| 0.026477 | 20_I_4 | UHNhscpg0007218 | 6 | C6orf122 | C6orf70 | DLL1 |
| 0.026784 | 27_C_24 | UHNhscpg0009827 | | | | |
| 0.026839 | 20_P_17 | UHNhscpg0007346 | 11 | RPS25 | DLNB14 | SLC37A4 |
| 0.026842 | 15_N_21 | UHNhscpg0005470 | 20 | | VIAAT | ACTR5 |
| 0.026855 | 28_L_20 | UHNhscpg0010449 | 9 | | NFIB | ZDHHC21 |
| 0.027163 | 18_B_13 | UHNhscpg0006529 | 4 | | HAND2 | AK021601 |
| 0.027198 | 9_M_1 | UHNhscpg0003034 | 2 | | BC057764 | AK096920 |
| 0.0272 | 26_P_9 | UHNhscpg0009604 | 2 | AK123485 | AK127400 | BC069026 |
| 0.027259 | 3_N_1 | UHNhscpg0000998 | 4 | CPZ | GPR78 | DUB3 |
| 0.027261 | 7_B_13 | UHNhscpg0002412 | 10 | C10orf69 | C10orf69 | CHUK |
| 0.027423 | 10_E_2 | UHNhscpg0003451 | 16 | FLJ21816 | FLJ21816 | MGC3248 |
| 0.027501 | 28_M_19 | UHNhscpg0010173 | | | | |
| 0.027605 | 30_N_8 | UHNhscpg0011223 | 2 | | KIAA1715 | HOXD13 |
| 0.027749 | 8_N_3 | UHNhscpg0002849 | 11 | | BXMAS2-10 | AF087878 |
| 0.027854 | 27_C_19 | UHNhscpg0009729 | 7 | | PPP1R3A | FOXP2 |
| 0.027926 | 27_D_13 | UHNhscpg0009918 | 1 | | AK026930 | AK127275 |
| 0.027977 | 31_I_24 | UHNhscpg0011399 | 5 | LNPEP | AF119888 | AF178574 |
| 0.028159 | 5_M_4 | UHNhscpg0001647 | 8 | | FLJ46365 | FLJ11767 |
| 0.028172 | 30_L_24 | UHNhscpg0011219 | 13 | | LGR8 | LOC196549 |
| 0.028185 | 3_L_15 | UHNhscpg0000993 | 5 | AY358358 | BC062459 | OR2V2 |
| 0.028281 | 7_K_18 | UHNhscpg0002378 | 8 | | FLJ20421 | AL080200 |
| 0.028303 | 16_C_9 | UHNhscpg0005591 | 1 | | PBX1 | BC066353 |
| 0.028307 | 6_G_10 | UHNhscpg0001982 | 22 | | CERK | C22orf4 |
| 0.028315 | 11_M_12 | UHNhscpg0003872 | X | | TM4SF2 | MIG12 |
| 0.028371 | 15_C_8 | UHNhscpg0005310 | X | PHEX | SMS | FLJ25735 |
| 0.028388 | 8_C_1 | UHNhscpg0002608 | 9 | ATP6V1G1 | DFNB31 | C9orf91 |
| 0.028401 | 14_L_10 | UHNhscpg0005172 | 5 | | CEI | IRX1 |
| 0.028432 | 3_O_9 | UHNhscpg0000825 | 11 | AK074061 | RBM21 | MTA2 |
| 0.028454 | 9_E_4 | UHNhscpg0003080 | 11 | TBRG1 | PANX3 | TBRG1 |
| 0.028517 | 26_I_12 | UHNhscpg0009473 | | | | |
| 0.028544 | 27_L_14 | UHNhscpg0010062 | | | | |
| 0.028582 | 2_C_2 | UHNhscpg0000480 | 7 | MTPN | AF130088 | CHRM2 |
| 0.028611 | 1_P_19 | UHNhscpg0000284 | 10 | NKX2-3 | GOT1 | SLC25A28 |
| 0.028633 | 25_M_17 | UHNhscpg0009020 | | | | |
| 0.028911 | 28_P_22 | UHNhscpg0010474 | 12 | | GRIP1 | TIP120A |
| 0.028989 | 7_F_3 | UHNhscpg0002431 | 8 | M13930 | AF268618 | X66258 |
| 0.029009 | 28_D_5 | UHNhscpg0010298 | 1 | KIAA0446 | SEMA4A | BGLAP |
| 0.02902 | 24_K_2 | UHNhscpg0008712 | 7 | CHCHD3 | FLJ40288 | AF119872 |
| 0.029246 | 2_H_7 | UHNhscpg0000600 | | | | |
| 0.029255 | 13_I_6 | UHNhscpg0004606 | 19 | LYL1 | NFIX | FLJ20244 |
| 0.02955 | 19_E_12 | UHNhscpg0006831 | 2 | LRP2 | DHRS9 | BBS5 |
| 0.02955 | 17_E_20 | UHNhscpg0006082 | 9 | | GAS1 | BC027471 |
| 0.029614 | 6_H_18 | UHNhscpg0002167 | 5 | | FLJ13231 | NUP155 |
| 0.029618 | 31_K_15 | UHNhscpg0011311 | 4 | | LOC132321 | PCDH10 |
| 0.029637 | 5_P_23 | UHNhscpg0001757 | | | | |
| 0.029779 | 7_A_8 | UHNhscpg0002313 | 6 | AF116727 | SERPINB9 | DKFZp686I15217 |
| 0.029802 | 3_J_18 | UHNhscpg0001073 | 18 | BC062338 | AK131011 | SLC14A2 |
| 0.029848 | 22_F_10 | UHNhscpg0008104 | 10 | | DNAJC1 | COMMD3 |
| 0.029954 | 22_H_10 | UHNhscpg0008116 | 2 | EMX1 | SPR | SFXN5 |
| 0.029971 | 23_L_18 | UHNhscpg0008528 | 12 | | C1QDC1 | AK126754 |
| 0.030057 | 23_P_15 | UHNhscpg0008455 | 1 | PRRX1 | AK130711 | FLJ23550 |
| 0.030175 | 10_M_7 | UHNhscpg0003407 | 15 | | BC059401 | KIAA1199 |
| 0.030216 | 2_K_3 | UHNhscpg0000438 | 11 | RNF141 | AMPD3 | BC028123 |
| 0.030264 | 29_P_17 | UHNhscpg0010760 | 15 | | VPS18 | DLL4 |
| 0.030309 | 19_I_22 | UHNhscpg0006859 | 19 | PVRL2 | LU | TOMM40 |
| 0.030449 | 5_D_5 | UHNhscpg0001683 | 1 | | CSRP1 | BC030568 |
| 0.030484 | 1_A_22 | UHNhscpg0000107 | 2 | | GAF1 | SMYD5 |
| 0.03058 | 22_P_24 | UHNhscpg0008171 | | | | |
| 0.030883 | 18_B_12 | UHNhscpg0006624 | 8 | MOS | RPS20 | PLAG1 |
| 0.031054 | 16_H_14 | UHNhscpg0005900 | 2 | | BC014369 | IRS1 |
| 0.031081 | 24_P_3 | UHNhscpg0008833 | 13 | NBEA | RFC3 | MAB21L1 |
| 0.031117 | 15_I_4 | UHNhscpg0005344 | | | | |
| 0.031237 | 25_C_17 | UHNhscpg0008960 | 7 | GSTK1 | TAS2R40 | FLJ90586 |
| 0.031248 | 16_I_14 | UHNhscpg0005723 | 14 | | C14orf101 | OTX2 |
| 0.031285 | 29_A_11 | UHNhscpg0010481 | 1 | PAX7 | FLJ38753 | TAS1R2 |
| 0.031299 | 15_K_6 | UHNhscpg0005357 | 11 | MGC12965 | KCNE3 | POLD3 |
| 0.031308 | 24_P_4 | UHNhscpg0008929 | 13 | NBEA | RFC3 | MAB21L1 |
| 0.031309 | 13_C_18 | UHNhscpg0004576 | 14 | | GARNL1 | BRMS1L |
| 0.031401 | 1_H_13 | UHNhscpg0000233 | 11 | ETS1 | S67063 | FLI1 |
| 0.031405 | 18_N_13 | UHNhscpg0006601 | | | | |
| 0.031477 | 14_I_8 | UHNhscpg0004979 | 19 | | U87593 | BC068609 |
| 0.031501 | 19_K_17 | UHNhscpg0006777 | | | | |
| 0.031587 | 7_J_24 | UHNhscpg0002559 | | | | |
| 0.031631 | 4_I_11 | UHNhscpg0001165 | 15 | | LOC90525 | SLC28A2 |
| 0.031839 | 25_C_2 | UHNhscpg0009048 | 19 | THAP8 | CLIPR-59 | AK090617 |
| 0.031997 | 13_D_13 | UHNhscpg0004669 | 19 | ZNF160 | FLJ12985 | FLJ45949 |
| 0.03201 | 10_B_3 | UHNhscpg0003514 | 6 | | BC067217 | IL20RA |
| 0.032026 | 17_F_15 | UHNhscpg0006173 | 8 | | STMN4 | TRIM35 |
| 0.03207 | 31_O_22 | UHNhscpg0011434 | 16 | BC064026 | TNFRSF12A | AK128093 |
| 0.032254 | 5_G_13 | UHNhscpg0001527 | 20 | | PLCB1 | PLCB4 |
| 0.032264 | 4_I_19 | UHNhscpg0001169 | | | | |
| 0.032272 | 10_L_21 | UHNhscpg0003582 | 13 | ELF1 | ELF1 | WBP4 |
| 0.032309 | 19_B_20 | UHNhscpg0006995 | 19 | | ZNF579 | FLJ14768 |
| 0.032361 | 7_C_1 | UHNhscpg0002226 | | | | |
| 0.03237 | 10_A_13 | UHNhscpg0003340 | 3 | | POU1F1 | HTR1F |
| 0.032392 | 20_G_17 | UHNhscpg0007121 | 1 | CAP350 | LAP1B | AF387614 |
| 0.032409 | 16_M_13 | UHNhscpg0005651 | 16 | | AL137562 | FOXF1 |
| 0.032542 | 22_E_23 | UHNhscpg0007823 | | | | |
| 0.032584 | 6_J_4 | UHNhscpg0002171 | 19 | HSPC023 | AY358176 | ZSWIM4 |
| 0.032793 | 21_O_14 | UHNhscpg0007606 | 19 | LOC91661 | AB075859 | LOC91661 |
| 0.032961 | 2_C_6 | UHNhscpg0000482 | 11 | EED | PICALM | HSPC138 |
| 0.033158 | 26_D_5 | UHNhscpg0009530 | 1 | PMF1 | KIAA0446 | BGLAP |
| 0.033218 | 16_O_16 | UHNhscpg0005758 | | | | |
| 0.033224 | 12_J_21 | UHNhscpg0004329 | 17 | | BCAS3 | TBX2 |
| 0.033558 | 22_N_24 | UHNhscpg0008159 | 11 | | RAB6A | MRPL48 |
| 0.0336 | 30_L_18 | UHNhscpg0011216 | 7 | | PPP1R3A | FOXP2 |
| 0.033607 | 19_K_20 | UHNhscpg0006870 | 1 | | PTPN14 | CENPF |
| 0.03361 | 19_A_2 | UHNhscpg0006804 | 20 | | SEC23B | HARS2 |
| 0.03403 | 20_C_17 | UHNhscpg0007098 | 16 | FLJ31606 | BC045670 | CDK10 |
| 0.034079 | 24_P_14 | UHNhscpg0008934 | | | | |
| 0.034211 | 19_D_15 | UHNhscpg0006913 | 19 | AF322648 | AK128554 | ZNF576 |
| 0.034323 | 28_B_19 | UHNhscpg0010293 | 5 | TAF7 | SLC25A2 | PCDHGA1 |
| 0.03452 | 23_E_16 | UHNhscpg0008299 | 8 | MGC1136 | MGC1136 | AY358147 |
| 0.034637 | 21_H_14 | UHNhscpg0007737 | 11 | BC006136 | DIBD1 | CRYAB |
| 0.034676 | 15_E_13 | UHNhscpg0005231 | 8 | BC054009 | LOC83690 | AF315715 |
| 0.03482 | 15_N_5 | UHNhscpg0005464 | 18 | HFL-EDDG1 | AFG3L2 | HFL-EDDG1 |
| 0.03498 | 23_N_1 | UHNhscpg0008436 | 19 | BCL3 | AK128234 | CBLC |
| 0.035099 | 20_L_17 | UHNhscpg0007327 | 4 | | PCDH10 | PCDH18 |
| 0.035379 | 9_C_3 | UHNhscpg0002975 | 12 | | CNTN1 | PDZRN4 |
| 0.035408 | 3_J_7 | UHNhscpg0000977 | | | | |
| 0.03546 | 11_N_1 | UHNhscpg0003963 | 16 | | SIAH1 | FLJ31821 |
| 0.035617 | 27_B_20 | UHNhscpg0010005 | 2 | | PTHR2 | MAP2 |
| 0.035801 | 9_G_8 | UHNhscpg0003093 | | | | |
| 0.035881 | 12_I_7 | UHNhscpg0004134 | 6 | PACRG | PARK2 | QKI |
| 0.03593 | 26_G_10 | UHNhscpg0009460 | 6 | | AJ504716 | HLA-E |
| 0.036179 | 13_A_7 | UHNhscpg0004465 | 7 | FLJ32110 | MGC26647 | AK098759 |
| 0.036195 | 4_G_22 | UHNhscpg0001252 | 1 | DUSP10 | DUSP10 | AK126238 |
| 0.036239 | 3_H_12 | UHNhscpg0001058 | 19 | | MGC62100 | ZNF345 |
| 0.036309 | 14_L_2 | UHNhscpg0005169 | | | | |
| 0.036444 | 1_B_4 | UHNhscpg0000288 | 13 | | BC062461 | ADPRHL1 |
| 0.036513 | 2_O_8 | UHNhscpg0000552 | 9 | TTF1 | KIAA0625 | FLJ46082 |
| 0.036533 | 20_B_18 | UHNhscpg0007358 | 10 | PAX2 | AY153483 | C10orf6 |
| 0.036542 | 20_A_19 | UHNhscpg0007088 | 5 | | SLIT3 | BC032027 |
| 0.036828 | 3_B_4 | UHNhscpg0001022 | 7 | | ABCB5 | SP8 |
| 0.03683 | 16_E_12 | UHNhscpg0005698 | 2 | AK095266 | MAP1D | DLX1 |
| 0.036917 | 16_F_4 | UHNhscpg0005883 | 1 | PGD | KIF1B | CORT |
| 0.037012 | 29_P_21 | UHNhscpg0010762 | 20 | | SIRPB2 | PTPNS1 |
| 0.037071 | 1_J_19 | UHNhscpg0000248 | 14 | C14orf29 | AF223938 | C14orf29 |
| 0.037126 | 9_A_17 | UHNhscpg0002970 | 6 | BC064365 | SESN1 | C6orf182 |
| 0.037361 | 28_C_16 | UHNhscpg0010207 | 3 | | BBX | CD47 |
| 0.037375 | 23_H_11 | UHNhscpg0008405 | | | | |
| 0.037458 | 31_A_2 | UHNhscpg0011340 | 2 | | GBX2 | ASB18 |
| 0.03753 | 3_L_14 | UHNhscpg0001082 | 16 | MGC2654 | A2BP1 | AY203956 |
| 0.037601 | 7_N_16 | UHNhscpg0002579 | 9 | | KIAA1539 | UNC13B |
| 0.037621 | 13_I_17 | UHNhscpg0004521 | 18 | | EPB41L3 | L3MBTL4 |
| 0.037643 | 6_J_24 | UHNhscpg0002181 | 8 | D63477 | ADCY8 | KCNQ3 |
| 0.037702 | 20_H_8 | UHNhscpg0007387 | 11 | | AK027747 | CRY2 |
| 0.037756 | 30_L_21 | UHNhscpg0011122 | 3 | | NME6 | LOC285231 |
| 0.037869 | 30_M_22 | UHNhscpg0011038 | 2 | | KIAA1715 | HOXD13 |
| 0.037981 | 16_L_9 | UHNhscpg0005826 | 17 | MGC10561 | ENPP7 | CBX8 |
| 0.038235 | 14_P_9 | UHNhscpg0005106 | 10 | ADAM12 | FANK1 | AF435960 |
| 0.038274 | 28_C_17 | UHNhscpg0010112 | 13 | TUBGCP3 | TUBGCP3 | FLJ26443 |
| 0.03851 | 12_F_20 | UHNhscpg0004400 | | | | |
| 0.038557 | 16_A_2 | UHNhscpg0005669 | 6 | RPL10A | FANCE | TEAD3 |
| 0.038697 | 20_F_7 | UHNhscpg0007287 | 12 | FLJ22662 | FLJ22662 | GUCY2C |
| 0.038746 | 6_P_12 | UHNhscpg0002208 | 2 | | FLJ36175 | BAZ2B |
| 0.038865 | 17_A_22 | UHNhscpg0006060 | 1 | | ZP4 | CHRM3 |
| 0.038987 | 15_L_13 | UHNhscpg0005456 | | | | |
| 0.039097 | 8_A_22 | UHNhscpg0002698 | 13 | IPF1 | GSH1 | CDX2 |
| 0.0393 | 28_J_3 | UHNhscpg0010333 | 20 | | AB033098 | AF451990 |
| 0.03941 | 18_D_24 | UHNhscpg0006642 | 10 | IDE | AF336887 | KIF11 |
| 0.039458 | 1_G_20 | UHNhscpg0000142 | | | | |
| 0.039464 | 4_F_22 | UHNhscpg0001426 | 8 | AK130123 | AK129682 | PPP2R2A |
| 0.039612 | 10_P_5 | UHNhscpg0003597 | 18 | | RNMT | MC5R |
| 0.039616 | 24_M_10 | UHNhscpg0008728 | 6 | | PECI | CDYL |
| 0.039667 | 18_M_24 | UHNhscpg0006510 | 10 | | LGI1 | TMEM20 |
| 0.039698 | 5_O_11 | UHNhscpg0001569 | 6 | TNFAIP3 | OLIG3 | TNFAIP3 |
| 0.039833 | 26_P_10 | UHNhscpg0009700 | 17 | | AK055561 | HT008 |
| 0.040086 | 9_P_19 | UHNhscpg0003240 | | | | |
| 0.04009 | 20_G_22 | UHNhscpg0007215 | 3 | LOC51244 | GRIP2 | DKFZP434N1817 |
| 0.040283 | 12_D_24 | UHNhscpg0004390 | 18 | | RAX | AK123184 |
| 0.040409 | 12_P_12 | UHNhscpg0004456 | 3 | TIPARP | SSR3 | TIPARP |
| 0.040551 | 17_K_24 | UHNhscpg0006118 | 7 | SHH | MGC20460 | AK124321 |
| 0.040657 | 24_L_7 | UHNhscpg0008811 | 1 | LOC57821 | BLZF1 | SLC19A2 |
| 0.040712 | 9_F_19 | UHNhscpg0003180 | 2 | | AK126832 | SIX3 |
| 0.040786 | 20_M_7 | UHNhscpg0007150 | 6 | ECHDC1 | RNF146 | KIAA0408 |
| 0.040861 | 25_H_18 | UHNhscpg0009272 | 14 | | RIPK3 | NFATC4 |
| 0.040916 | 23_K_17 | UHNhscpg0008240 | X | | IRS4 | GUCY2F |
| 0.041029 | 20_C_5 | UHNhscpg0007093 | 1 | FLJ20519 | BNIPL | SPEC1 |
| 0.041204 | 23_F_18 | UHNhscpg0008492 | 9 | | AF091236 | ACO1 |
| 0.041207 | 27_A_13 | UHNhscpg0009714 | 1 | | D50923 | GALNT2 |
| 0.041353 | 15_H_9 | UHNhscpg0005431 | 9 | | AL834325 | MAPKAP1 |
| 0.041379 | 17_H_8 | UHNhscpg0006276 | 14 | | C14orf102 | CALM1 |
| 0.04138 | 1_C_8 | UHNhscpg0000112 | 11 | | SESN3 | MGC33371 |
| 0.0415 | 4_F_13 | UHNhscpg0001329 | 3 | PLXND1 | PLXND1 | AK123339 |
| 0.04154 | 10_O_9 | UHNhscpg0003420 | 9 | C9orf40 | AK126029 | C9orf41 |
| 0.04154 | 15_O_10 | UHNhscpg0005383 | 2 | PTPN4 | MGC10993 | EPB41L5 |
| 0.041597 | 9_J_2 | UHNhscpg0003289 | 14 | | ZNF219 | BC031469 |
| 0.041602 | 28_N_4 | UHNhscpg0010453 | 10 | | NEBL | FLJ45187 |
| 0.04164 | 4_E_16 | UHNhscpg0001237 | 15 | | RPLP1 | AK127674 |
| 0.041726 | 23_K_8 | UHNhscpg0008331 | X | | FLJ34960 | SMPX |
| 0.041849 | 2_H_8 | UHNhscpg0000691 | 15 | AP3S2 | ANPEP | AK055252 |
| 0.041961 | 21_H_11 | UHNhscpg0007649 | 14 | AK125925 | DIO2 | BC036939 |
| 0.04204 | 16_P_23 | UHNhscpg0005857 | | | | |
| 0.042054 | 14_K_16 | UHNhscpg0004995 | 1 | | FAAH | DMBX1 |
| 0.042075 | 20_L_15 | UHNhscpg0007326 | 17 | MSF | AK123491 | MSF |
| 0.042076 | 13_A_17 | UHNhscpg0004473 | 6 | DJ467N11.1 | HELIC1 | GRIK2 |
| 0.042094 | 4_J_20 | UHNhscpg0001448 | 3 | BC033771 | MGC34132 | UBE1C |
| 0.042254 | 14_K_17 | UHNhscpg0004905 | | | | |
| 0.042693 | 4_O_15 | UHNhscpg0001202 | 1 | | HSPA6 | FCGR3B |
| 0.043038 | 5_A_22 | UHNhscpg0001586 | 9 | TLE1 | TLE4 | AY129016 |
| 0.043119 | 6_L_8 | UHNhscpg0002183 | | | | |
| 0.043172 | 16_D_23 | UHNhscpg0005786 | 2 | | AB051465 | FLJ20701 |
| 0.043279 | 25_J_17 | UHNhscpg0009188 | 3 | | AK127782 | KIF9 |
| 0.043366 | 32_C_12 | UHNhscpg0011741 | 3 | BC047734 | FHIT | ID2B |
| 0.043577 | 9_F_12 | UHNhscpg0003271 | 10 | | CRTAC1 | C10orf28 |
| 0.043623 | 6_M_17 | UHNhscpg0001932 | 16 | | BX537874 | RBL2 |
| 0.043638 | 24_P_6 | UHNhscpg0008930 | 5 | CDH12 | PMCHL1 | PRDM9 |
| 0.043787 | 3_A_1 | UHNhscpg0000745 | 18 | | MOCOS | AK128053 |
| 0.043902 | 29_A_6 | UHNhscpg0010574 | 4 | | PCDH10 | PCDH18 |
| 0.044031 | 19_L_14 | UHNhscpg0007051 | 9 | MPDZ | AK098775 | NFIB |
| 0.044216 | 14_K_7 | UHNhscpg0004900 | 2 | MRPL35 | IMMT | C2orf23 |
| 0.044493 | 29_J_10 | UHNhscpg0010816 | | | | |
| 0.044564 | 31_P_4 | UHNhscpg0011617 | 13 | AK056283 | BC030118 | WDFY2 |
| 0.044576 | 14_M_21 | UHNhscpg0004919 | 21 | C21orf29 | KRTAP18-12 | UBE2G2 |
| 0.044657 | 27_D_23 | UHNhscpg0009923 | 3 | | RBMS3 | TGFBR2 |
| 0.044718 | 10_B_22 | UHNhscpg0003616 | 7 | | MGC33530 | SEC61G |
| 0.044835 | 29_A_19 | UHNhscpg0010485 | 14 | | NOVA1 | FOXG1B |
| 0.04487 | 19_C_5 | UHNhscpg0006723 | 2 | VRK2 | AB067499 | FANCL |
| 0.044901 | 14_I_14 | UHNhscpg0004982 | 11 | | CALCB | SOX6 |
| 0.044963 | 17_G_24 | UHNhscpg0006096 | 12 | FLJ40089 | SNRPF | MGC35366 |
| 0.045052 | 4_I_8 | UHNhscpg0001257 | 12 | | AB006624 | NAB2 |
| 0.045076 | 12_P_2 | UHNhscpg0004450 | 4 | | AF151033 | FBXW7 |
| 0.045136 | 5_E_5 | UHNhscpg0001511 | 2 | | TANK | PSMD14 |
| 0.045144 | 24_J_11 | UHNhscpg0008801 | | | | |
| 0.045282 | 7_P_23 | UHNhscpg0002499 | 15 | LACTB | TPM1 | RPS27L |
| 0.045296 | 24_L_5 | UHNhscpg0008810 | 13 | ALG5 | SMAD9 | EXOSC8 |
| 0.045422 | 5_I_1 | UHNhscpg0001533 | 18 | CCDC5 | ATP5A1 | C18orf25 |
| 0.045521 | 6_O_11 | UHNhscpg0001937 | 13 | | AF144054 | LIG4 |
| 0.045598 | 12_G_9 | UHNhscpg0004123 | 22 | BC052239 | MGC11256 | AK123987 |
| 0.045829 | 20_J_7 | UHNhscpg0007310 | 2 | | G6PC2 | ABCB11 |
| 0.04586 | 27_K_19 | UHNhscpg0009777 | 2 | | COL5A2 | FLJ12519 |
| 0.045959 | 8_O_5 | UHNhscpg0002678 | 9 | BC064135 | PSIP1 | C9orf93 |
| 0.046033 | 13_B_12 | UHNhscpg0004749 | 5 | AMACR | MATP | C1QTNF3 |
| 0.046101 | 8_G_6 | UHNhscpg0002726 | 5 | AK057601 | LOC375449 | AB002301 |
| 0.046186 | 16_I_18 | UHNhscpg0005725 | 4 | | LOC285513 | SNCA |
| 0.046489 | 32_A_21 | UHNhscpg0011638 | | | | |
| 0.046497 | 13_L_20 | UHNhscpg0004811 | 1 | KCNH1 | MART2 | RCOR3 |
| 0.046568 | 5_A_21 | UHNhscpg0001496 | 9 | TLE1 | TLE4 | AY129016 |
| 0.046688 | 3_P_3 | UHNhscpg0001011 | 16 | SHCBP1 | SHCBP1 | VPS35 |
| 0.046702 | 13_E_7 | UHNhscpg0004489 | 14 | BMP4 | DDHD1 | BC064965 |
| 0.046837 | 25_B_13 | UHNhscpg0009138 | 1 | | TGS | ADPRT |
| 0.046861 | 10_B_16 | UHNhscpg0003613 | | | | |
| 0.046894 | 16_J_8 | UHNhscpg0005909 | 17 | | MMD | FLJ10970 |
| 0.04699 | 16_P_15 | UHNhscpg0005853 | 10 | TCF7L2 | AK027209 | HABP2 |
| 0.047053 | 21_H_24 | UHNhscpg0007742 | 4 | | CSN3 | NYD-SP26 |
| 0.047359 | 11_L_3 | UHNhscpg0003952 | 7 | | AY129022 | AK125308 |
| 0.047399 | 17_C_3 | UHNhscpg0005967 | 1 | NGFB | TSPAN-2 | VANGL1 |
| 0.047449 | 12_B_12 | UHNhscpg0004372 | 2 | LOC129401 | DUSP19 | LOC91752 |
| 0.04764 | 24_J_15 | UHNhscpg0008803 | 2 | AK123152 | REPRIMO | AK090913 |
| 0.047705 | 17_A_5 | UHNhscpg0005956 | 7 | DLD | SLC26A3 | LAMB1 |
| 0.047745 | 5_E_3 | UHNhscpg0001510 | | | | |
| 0.047776 | 16_J_5 | UHNhscpg0005812 | 17 | | KCNJ2 | BC034818 |
| 0.047839 | 29_E_21 | UHNhscpg0010510 | 2 | | MGC43122 | GPR66 |
| 0.048083 | 15_H_18 | UHNhscpg0005525 | 1 | ALDH4A1 | TAS1R2 | AK024480 |
| 0.048166 | 25_J_16 | UHNhscpg0009283 | 6 | AKAP12 | FTHFSDC1 | ZBTB2 |
| 0.048271 | 19_B_5 | UHNhscpg0006897 | 11 | | RRM1 | SSA1 |
| 0.048622 | 22_D_13 | UHNhscpg0007998 | 22 | MAPK1 | YPEL1 | PPM1F |
| 0.04889 | 30_A_20 | UHNhscpg0010965 | | | | |
| 0.048951 | 23_E_14 | UHNhscpg0008298 | | | | |
| 0.048971 | 14_O_16 | UHNhscpg0005019 | 6 | | IFNGR1 | OLIG3 |
| 0.048988 | 30_F_18 | UHNhscpg0011180 | 6 | TTLL2 | UNC93A | TCP10 |
| 0.049143 | 6_A_19 | UHNhscpg0001858 | 12 | METAP2 | NTN4 | AF336880 |
| 0.049163 | 1_M_13 | UHNhscpg0000079 | 7 | | IGFBP3 | AF119858 |
| 0.049255 | 17_F_7 | UHNhscpg0006169 | 8 | | DUSP4 | BC003524 |
| 0.049442 | 13_J_20 | UHNhscpg0004799 | 2 | FANCL | VRK2 | BCL11A |
| 0.049728 | 15_F_3 | UHNhscpg0005416 | 12 | FLJ38663 | MPHOSPH9 | FLJ38663 |
| 0.049773 | 7_F_16 | UHNhscpg0002531 | | | | |
| 0.049854 | 18_N_1 | UHNhscpg0006595 | 20 | | AY203949 | AK127957 |
| 0.049961 | 15_B_2 | UHNhscpg0005484 | 19 | RFXANK | MEF2B | TRA16 |

TABLE 6
FDR-significant DNA methylation changes observed for genes that are
functionally- or positionally-linked to major psychosis.
See Table 9 for more information on these loci, and
Table 10 and FIG. 17 for a full list of FDR-significant loci.

| Gene name and symbol | Methylation change(s) observed |
|---|---|
| Autism Susceptibility Candidate 2 (AUTS2 or | SZ male ↑ |
| KIAA0442) | |
| Dysbindin (DTNBP1) | BD female ↑, PSY female ↑ |
| Fbj Murine Osteosarcoma Viral Oncogene Homolog B | PSY female ↓ |
| (FOSB) | |
| Glutamate Receptor, Ionotropic, AmpA 2 (GRIA2) | SZ male ↓, PSY male ↓ |
| Glutaminase 2 (GLS2) | SZ male ↑ |
| Homo sapiens Hey-like transcriptional repressor | SZ female ↑, BD female ↑ |
| (HELT) | |
| HLA Complex Group 9 (HCG9) | SZ female ↑, PSY female ↑ |
| Islet 2 Transcription Factor, Lim/Homeodomain (ISL2) | PSY female ↑ |
| Lim Homeobox Protein 5 (LHX5) | SZ female ↑, BD female ↑, PSY female ↑ |
| Lim Homeobox Transcription Factor 1, Beta (LMX1B) | SZ female ↓, PSY female ↓ |
| Multiple Alpha Helices and RNA-Linker protein-1 | SZ female ↑, BD female ↑, PSY female ↑ |
| (MARLIN-1) | |
| Neuregulin 2 (NRG2) | BD female ↓, PSY female ↓ |
| Nuclear Receptor Subfamily 4, Group A, Member 2 | SZ female ↑ |
| (NR4A2 or NURR1) | |
| Phospholipase A2, Group 4B (PLA2G4B) | SZ male ↑, PSY male ↑, PSY female ↑ |
| Potassium Channel, Inwardly Rectifying, Subfamily J, | SZ male ↑, PSY male ↑ |
| Member 6 (KCNJ6 or GIRK2) | |
| Retinoic Acid Inducible-1 (RAI1) | SZ female ↑ |
| Ribosomal Protein L39 (RPL39) | BD female ↑, PSY female ↑ |
| Secretogranin II (SCG2) | PSY female ↓ |
| Solute Carrier Family 17, Member 6 (SLC17A6 or | SZ female ↓ |
| VGLUT2) | |
| Solute Carrier Family 17, Member 7 (SLC17A7 or | SZ female ↑, PSY female ↑ |
| VGLUT1) | |
| Vacuolar Protein Sorting 33B (VPS33B) | PSY male ↑ |
| WD Repeat Domain 18 (WDR18) | SZ male ↓, PSY male ↓ |
| Wingless-Type Mmtv Integration Site Family, Member | PSY female ↑ |
| 1 (WNT1) | |

TABLE 7
Description of the samples utilized in this study.
A

| | CTRL | SZ | BD |
|---|---|---|---|
| Number | 35 | 35 | 35 |
| Mean age and range | 44.1 (31-59) | 42.6 (19-59) | 45.3 (19-64) |
| Race | 35 white | 35 white | 33 white |
| | | | 1 Black |
| | | | 1 Native American |
| Sex | 26 M, 9 F | 26 M, 9 F | 17 M, 18 F |
| Diagnosis | no axis I | 27 undifferentiated | 26 BP I |
| | | 7 paranoid | 4 BP II |
| | | 1 disorganized | 4 BP NOS |
| | | | 1 BP-SA |
| Psychotic Features | 0 | 35+ | 20+, 11−, |
| | | | 4 unclear |
| Cause of death | 32 cardiac | 14 cardiac | 12 cardiac |
| | 3 other medical | 13 other medical | 4 other medical |
| | | 1 accident | 4 accidents |
| | | 7 suicide | 15 suicide |

B

| | | Male | | | Female | |
|---|---|---|---|---|---|---|
| | CTRL | SZ | BD | CTRL | SZ | BD |
| | (N = 22) | (N = 26) | (N = 15) | (N = 6) | (N = 9) | (N = 17) |
| Postmortem | 26.9 (11.7) | 31.4 (17.0) | 37.8 (20.6) | 35.50 (9.6) | 30.88 (9.1) | 39.59 (17.7) |
| Interval | | | | | | |
| Refrigerator | 2.9 (1.6) | 5.67 (4.3)* | 7.80 (7.4)* | 2.83 (0.4) | 6.25 (4.1) | 13.18 (12.7)* |
| Interval | | | | | | |
| Brain pH | 6.65 (0.26) | 6.44 (0.25)* | 6.46 (0.26)* | 6.45 (0.30) | 6.61 (0.17) | 6.44 (0.29) |
| Brain Weight | 1486.4 (137.9) | 1444.6 (104.8) | 1467.6 (112.9) | 1314.2 (106.3) | 1418.1 (128.8) | 1316.2 (111.4) |
| Lifetime | 0.73 (0.94) | 2.36 (2.00)** | 2.86 (1.70)** | 1.00 (1.55) | 1.38 (2.00) | 2.29 (1.86)* |
| Alcohol Use | | | | | | |
| Lifetime | 0.27 (0.70) | 2.08 (2.00)** | 2.93 (2.05)** | 0.17 (0.41) | 0.29 (0.76) | 1.76 (1.8)* |
| Drug Use | | | | | | |

C

| | CTRL | BD |
|---|---|---|
| Number | 19 | 20 |
| Age | 41.3 (26-60) | 44.2 (22-60) |
| Sex | All male | All male |

A) Summary of demographic data for the brain samples obtained from the Stanley Foundation. Additional information can be obtained from http://www.stanleyresearch.org/programs/brain_collection.asp.
B) Comparison of brain samples utilized for microarray-based epigenomic profiling. N refers to the number in each group passing microarray quality control. Given are the mean for each group with SD. *= t-test p-value <0.05; **= t-test p-value <0.001. Affected individuals were found to have significantly lower brain pH (male SZ, male BD), and higher lifetime alcohol and drug use (male SZ, male BD, female BD) compared to unaffected controls of the same sex. These data mirror those reported elsewhere^{1,2}.
C) Summary of available demographic data for the germline samples used in this study.

TABLE 8
Primer sequences used for testing of
selected loci by pyrosequencing of
bisulfite modified DNA samples

| Primer Name | Oligo Sequence |
|---|---|
| ARVCF-INT3-F | BIOTIN-AAGAGGAGGGTTAAATTGTTA |
| ARVCF-INT3-PYRO | TCAAACTAAAACCAAAAC |
| ARVCF-INT3-R | ATTAACTTAAAAAAACCCTAACC |
| BDNF-EXON2-F | BIOTIN-TGTTTTTATGAAAGAAGTAAATATT |
| BDNF-EXON2-PYRO | AATCCTCATCCAACAA |
| BDNF-EXON2-R | TCCTTATTATTTTCTTCATTAAAC |
| BDNF-INT1A-F | GATGTTTTATTGAGTTTAGGTT |
| BDNF-INT1A-PYRO | TTGGGAGTAGAAGGTTT |
| BDNF-INT1A-R | BIOTIN-AACTAATTAATAACTCTATCCAA |
| BDNF-INT1B-F | BIOTIN-GGGTTAGATATTATTTAGTTT |
| BDNF-INT1B-PYRO | AAAAATAAAAACAAACCC |
| BDNF-INT1B-R | ACTAAAACTAAAACTAAAACAC |
| BDNF-PRM-F | TAGGGTTTTTTGGGAGAGTT |
| BDNF-PRM-PYRO | TTATTTTAGTTTTGGTTTT |
| BDNF-PRM-R | BIOTIN-ATTACCCACAAAAACCTATATAAA |
| COMT-EXON4-F | BIOTIN-GTGTTTGGGGATTTAAGTTT |
| COMT-EXON4-PYRO | TCAAACATACACACCTTA |
| COMT-EXON4-R | ACCCTTTTTCCAAATCTAAC |
| COMT-PRM-F | TTTGAGTAAGATTAGATTAAGAGGT |
| COMT-PRM-PYRO | GGGATATTTTGGTTAT |
| COMT-PRM-R | BIOTIN-ACAACCCTAACTACCCCAAAAAC |
| DRD4-F | GGTAGAGTTTGAGTTTAGGTT |
| DRD4-PYRO | TAGATATTAGGTGGAT |
| DRD4-R | BIOTIN-ACCAAACCAAACCCTAAAAC |
| DTNBP1-ARRAY-F | BIOTIN-TTGGGAAGTGTGGTTTGTAGGAA |
| DTNBP1-ARRAY-F | BIOTIN-TTGGGAAGTGTGGTTTGTAGGAA |
| DTNBP1-ARRAY-PYRO | CACCTTTAAACCTCCTATT |
| DTNBP1-ARRAY-PYRO | CACCTTTAAACCTCCTATT |
| DTNBP1-ARRAY-R | ACCTCCAAATATAACCACCATCTC |
| DTNBP1-ARRAY-R | ACCTCCAAATATAACCACCATCTC |
| DTNBP1-INT1-F | TTTTTTTTGTTTAGGAGTTTTTT |
| DTNBP1-INT1-PYRO | GGTAAAGGTAGAGAAAGGA |
| DTNBP1-INT1-R | BIOTIN-CTAAAACTAAACCAACCACCCTC |
| DTNBP1-PRM-F | BIOTIN-GAAGGGTTTTTAGTATTGT |
| DTNBP1-PRM-PYRO | AAAAAAACTAAAATTAC |
| DTNBP1-PRM-R | AAAAACTACTAACCCTCTC |
| GAD1-INT1-F | BIOTIN-GTTAGGTATTTGTAGAGGAGTT |
| GAD1-INT1-PYRO | CTAATTCCCTCTC |
| GAD1-INT1-R | TCACCTCCAACTACTTCCTC |
| GAD1-INT3-F | BIOTIN-TAGTTGAGTGATTTTGGTTGAAT |
| GAD1-INT3-PYRO | TACAAAAAACACCCAAA |
| GAD1-INT3-R | CTCTACTCTAACTACAAACTA |
| GAD1-PRM-F | BIOTIN-GAAGGTATGAAGAGGTAAGT |
| GAD1-PRM-PYRO | AAATTCCCACCAAAAA |
| GAD1-PRM-R | AAAATTCTCCCTTTACAATATTTAA |
| GRIA2-F | AAGATAGTAGGGTTTGGTGAGAGG |
| GRIA2-PYRO | ATAATTAGTAATTAGGTTTTTATAT |
| GRIA2-R | BIOTIN-TCTCTTCTCCCTCTCTCCTCTCT |
| GRIA2-SS1 | ATACAACAAAACTAATCTCC |
| GRIA2-SS2 | GAGTTGTGTTTTTTTAG |
| GRIN2B-F | GGTTTGTGTTGAATGGGTTT |
| GRIN2B-PYRO1 | TGAATGGGTTTTGAT |
| GRIN2B-PYRO2 | GGGTTTTATTTGTAA |
| GRIN2B-R | BIOTIN-TCATCCCTTCACCTAACAAAAA |
| HCG9-F | BIOTIN-GGATTTTAGGGAGAGGATAGGG |
| HCG9-PYRO | CTAAACTATTCCTATAAATAACATT |
| HCG9-R | CCCCACCCCCTACACTTT |
| HELT-PRM-F | BIOTIN-AGTGTGTATGGAATGAAATGTGGT |
| HELT-PRM-PYRO | CCCACTCCCATTTTTA |
| HELT-PRM-R | CCCTCCCAAATTACTCTACCA |
| KCNJ6-F | TTTTAGTTTTAGAAATAAAATAGAAA |
| KCNJ6-R | ATAATCTCTTACTCAACAAAAACTC |
| KCNJ6-SS | GGAGAGTTGAATTTAGAGAGT |
| LHX5-F | TTATAAATTTAGGAGGTGTAGGGATTT |
| LHX5-R | CCCAAAACTCAACAAAAAAAATAAAT |
| LHX5-SS1 | TGGGGTTTTGAAGGATTGA |
| LHX5-SS2 | ATTTTGTATTAGGTATT |
| MARLIN1-F | TAAGGTTTTAGTGTGGGGTGGTTT |
| MARLIN1-R | AAACAAATATAATCCCCACCTTCA |
| MARLIN1-SS | AGTTATTTTGTGAATGT |
| MTHFR-PRMA-F | GTTAAGTATTGGGATATTAAGTT |
| MTHFR-PRMA-PYRO | GATTTTTAGAAAGGTTT |
| MTHFR-PRMA-R | BIOTIN-ATAACTCAATAACCTAATAACTAA |
| MTHFR-PRMB-F | TAGTTATTGGGAGTTATATTAATT |
| MTHFR-PRMB-PYRO | GGGAGGTTGTTTGT |
| MTHFR-PRMB-R | BIOTIN-CTCCAACAACCTAACACCTA |
| NR4A2-F | TGTGGGGAGGGTGTAATAAAAGTA |
| NR4A2-PYRO | GGGGAGGGTGTAATAAA |
| NR4A2-R | BIOTIN-CACTCCCATTCCCTTTCAAATA |
| NRG1-INTRON1-F | BIOTIN-GAGTGGGATTTGGGTTATAGGAGT |
| NRG1-INTRON1-PYRO | CTTACCCTATACCCCAAA |
| NRG1-INTRON1-R | ACACAAAACTAAATCAAAATAAACC |
| NRG1-PRM-F | GGAGATTTTGTTTGGGGTAT |
| NRG1-PRM-PYRO | GAGTAGTTTTTTTAGG |
| NRG1-PRM-R | BIOTIN-CAACCCCTTTTCCTCCC |
| RELN-PRMA-F | BIOTIN-ACTCCCAAAATTACTTTAAACC |
| RELN-PRMA-PYRO | TTTTAAGAAGGTGTGGAG |
| RELN-PRMA-R | GGGGTTTTAAGAAGGTGTG |
| RELN-PRMB-F | GTTTGAAGGGGAAGGTTAGTT |
| RELN-PRMB-PYRO | AGGGAAGGAGAGG |
| RELN-PRMB-R | BIOTIN-AAAATCCTCTACAAATAAAACTCTA |
| RELN-PRMC-F | GGTTGTTATGGTTTTTGTTTTTAAG |
| RELN-PRMC-PYRO | AAGGGATGAGAAAGGTG |
| RELN-PRMC-R | BIOTIN-AAATACTCATTTCCCTACATATTAC |
| RPL39-F | BIOTIN-GGTAGTGTGTTAGGGGTATTTTGT |
| RPL39-PYRO | CCTCTAAAAAAATAACACTTACTC |
| RPL39-R | ACTATCCCTTCCCACACCTC |
| SLC17A7-F | AGGAGGGTGATTTTTTTTTTATTA |
| SLC17A7-PYRO | GGGTGGGAGGAGTAGA |
| SLC17A7-R | BIOTIN-AAACCCAAAAACACAACCAATC |
| THEM59-F | BIOTIN-GGGTTATTAATTAATTATTTGTGG |
| THEM59-PYRO | AAATTTATCCTACACTACCCT |
| THEM59-R | ACTCCTATTTTCCTCCCTAATCC |
| U-CG1A | CGTGGAGACTGACTACCAGAT |
| U-CG1B | AGTTACATCTGGTAGTCAGTCTCCA |
| WDR18-F | TTGGGAGGATTATTTGAGTTTAGG |
| WDR18-PYRO | AAATGTTTAGGAGGAAAAG |
| WDR18-R | BIOTIN-ACTTCTTCCAAAACCCAAAA |

TABLE 9
FDR significant microarray differences for spots located near to genes
that can be putatively linked to the etiology of PSY.

| | | FDR-Significant | |
|---|---|---|---|
| | Probe | Methylation | |
| Gene name and symbol | Location | change* | Link to psychosis |
| Autism | 7q11.22 | SZ MALE↑ | Spans a translocation |
| Susceptibility | | | breakpoint associated with |
| Candidate 2 (AUTS2 | | | mental retardation and autism; |
| or | | | highly expressed in frontal |
| KIAA0442) | | | cortex of brain³ |
| Dysbindin | 6p22.3 | BD FEMALE↑ | A compelling candidate gene |
| (DTNBP1)\hat{ } | | PSY FEMALE↑ | for PSY (see also Table 1) |
| | | | nominated by both association |
| | | | and linkage studies^{4,5} |
| Fbj Murine | 19q13.32 | PSY FEMALE↓ | FOS proteins have been |
| Osteosarcoma Viral | | | implicated as regulators of cell |
| Oncogene Homolog | | | proliferation, differentiation, |
| B (FOSB) | | | and transformation. FOSB is |
| | | | expressed following chronic |
| | | | antipsychotic drug treatment⁶ |
| Glutamate Receptor, | 4q31.1 | SZ MALE↓ | One of four ionotropic |
| Ionotropic, AmpA 2 | | PSY MALE↓ | glutamate receptor subunits, |
| (GRIA2) | | | found to be differentially |
| | | | expressed in the brains of SZ |
| | | | patients^{7,8} |
| Glutaminase 2 | 12q13.2 | SZ MALE↑ | Catalyzes the hydrolysis of |
| (GLS2) | | | glutamine to glutamate; found |
| | | | to be elevated in brains of SZ |
| | | | patients⁹ |
| Homo sapiens Hey- | 4q35.1 | SZ FEMALE↑ | HELT determines GABAergic |
| like transcriptional | | BD FEMALE↑ | over glutamatergic neuronal |
| repressor (HELT) | | | fate in the developing |
| | | | mesencephalon¹⁰ |
| HLA Complex | 6p21.33 | SZ FEMALE↑ | Located within the MHC class |
| Group 9 (HCG9) | | PSY FEMALE↑ | I region on chromosome 6p |
| | | | implicated in a genome-scan |
| | | | meta-analysis of |
| | | | schizophrenia⁵. The function |
| | | | of the encoded protein has not |
| | | | been determined, but immune- |
| | | | system disruption reported in |
| | | | SZ. |
| Islet 2 Transcription | 15q24.3 | PSY FEMALE↑ | A transcriptional factor that |
| Factor, | | | defines subclasses of |
| Lim/Homeodomain | | | motoneurons in the nervous |
| (ISL2) | | | system. 15q24.3 falls within a |
| | | | region implicated in a genome- |
| | | | scan meta-analysis of |
| | | | schizophrenia⁵ |
| Lim Homeobox | 12q24.13 | SZ FEMALE↑ | A transcriptional regulator |
| Protein 5 (LHX5) | | BD FEMALE↑ | involved in the control of |
| | | PSY FEMALE↑ | differentiation and |
| | | | development of the forebrain |
| | | | and knockout mice show |
| | | | learning impairments and |
| | | | motor dysfunction¹¹ |
| Lim Homeobox | 9q33.3 | SZ FEMALE↓ | A transcription factor |
| Transcription Factor | | PSY FEMALE↓ | important for the development |
| 1, Beta (LMX1B) | | | of dopaminergic neurons in the |
| | | | brain. |
| Multiple Alpha | 4p16.1 | SZ FEMALE↑ | A RNA-binding protein widely |
| Helices and RNA- | | BD FEMALE↑ | expressed in the brain that |
| Linker protein-1 | | PSY FEMALE↑ | associates with GABA(B) |
| (MARLIN-1 or | | | receptors¹². The 4p16.1 region |
| JAKMIP1) | | | has been linked to both BD |
| | | | and SZ¹³ |
| Neuregulin 2 | 5q31.3 | BD FEMALE↓ | Neuregulins are a family of |
| (NRG2) | | PSY FEMALE↓ | growth and differentiation |
| | | | factors that interact with |
| | | | ERBB receptors to induce the |
| | | | growth and differentiation of |
| | | | epithelial, neuronal, and glial |
| | | | cells. The gene for another |
| | | | neuregulin, NRG1, has been |
| | | | widely implicated in SZ¹⁴. |
| | | | 5q31.1 falls within a region |
| | | | implicated in a genome-scan |
| | | | meta-analysis of SZ⁵ |
| Nuclear Receptor | 2q24.1 | SZ FEMALE↑ | Plays a critical role in the |
| Subfamily 4, Group | | | development of midbrain |
| A, Member 2 | | | dopaminergic neurons. |
| (NR4A2 or NURR1) | | | Reduced expression observed |
| | | | in BD and SZ brains¹⁵. |
| Phospholipase A2, | 15q15.1 | SZ MALE↑ | Phospholipid metabolism |
| Group 4B | | PSY MALE↑ | shown to be disturbed in SZ; |
| (PLA2G4B) | | PSY FEMALE↑ | the phospholipid structure of |
| | | | neuronal membranes is |
| | | | essential for normal |
| | | | functioning¹⁶. |
| Potassium Channel, | 21q22.13 | SZ MALE↑ | G protein-coupled inwardly |
| Inwardly Rectifying, | | PSY MALE↑ | rectifying potassium channels |
| Subfamily J, | | | (GIRKs) link numerous |
| Member 6 (KCNJ6 | | | neurotransmitter receptors to |
| or GIRK2) | | | the regulation of synaptic |
| | | | transmission in the brain |
| Retinoic Acid | 17p11.2 | SZ FEMALE↑ | Located in a very unstable |
| Inducible-1 (RAI1) | | | genomic region containing a |
| | | | polyglutamine tract associated |
| | | | with SZ¹⁷. Highly suggestive |
| | | | evidence of linkage to this |
| | | | region with SZ has been |
| | | | reported¹⁸ |
| Ribosomal Protein | Xq24 | BD FEMALE↑ | Located at Xq24 in a region on |
| L39 (RPL39) | | PSY FEMALE↑ | the X-chromosome found to be |
| | | | linked to BD in several |
| | | | studies^{19,20} |
| Secretogranin II | 2q36.1 | PSY FEMALE↓ | A secretory protein located in |
| (SCG2) | | | the vesicles of many endocrine |
| | | | cells and neurons that has been |
| | | | shown to stimulate |
| | | | neurotransmitter release²¹. |
| | | | Chronic PCP exposure, which |
| | | | produces signs of persistently |
| | | | altered frontal brain activity |
| | | | and related behaviors that are |
| | | | also seen in patients with SZ, |
| | | | modulates SCG2 expression²². |
| | | | Expression is also altered |
| | | | following lithium treatment, a |
| | | | common medication for BD²³ |
| Solute Carrier | 11p14.3 | SZ FEMALE↓ | Encodes a vesicular glutamate |
| Family 17, Member | | | transporter (VGLUT), co- |
| 6 (SLC17A6 or | | | expressed with VGLUT1 |
| VGLUT2) | | | |
| Solute Carrier | 19q13.33 | SZ FEMALE↑ | Encodes a vesicular glutamate |
| Family 17, Member | | PSY FEMALE↑ | transporter (VGLUT) found to |
| 7 (SLC17A7 or | | | be downregulated in SZ |
| VGLUT1) | | | brains²⁴. Region implicated in |
| | | | a genome-scan meta-analysis |
| | | | of BD²⁵ |
| Vacuolar Protein | 15q26.1 | PSY MALE↑ | VPS33B plays an important |
| Sorting 33B | | | role in vesilcular transport in |
| (VPS33B) | | | numerous tissues. 15q26.1 |
| | | | falls within a region implicated |
| | | | in a genome-scan meta- |
| | | | analysis of SZ⁵ |
| WD Repeat Domain | 19p13.3 | SZ MALE↓ | Probe located 10 kb upstream |
| 18 (WDR18) | | PSY MALE↓ | of NMDA receptor subunit |
| | | | gene (NR3B), postulated to be |
| | | | involved in schizophrenia^{26,27} |
| Wingless-Type | 12q13.12 | PSY FEMALE↑ | The Wnt pathway is critical for |
| Mmtv Integration | | | neurodevelopment and |
| Site Family, | | | regulates cell adhesion, |
| Member 1 (WNT1) | | | synaptic rearrangement, and |
| | | | plasticity; found to be over- |
| | | | expressed in the brains of SZ |
| | | | patients²⁸ |

*↓= relative hypomethylation in affected group (increased array intensity following enrichment), ↑= relative hypermethylation in affected group (decreased array intensity following enrichment).
\hat{ }The probe found to be differently enriched is located ~80 KB upstream of the transcription start-site and does not overlap with the region investigated in our candidate-gene approach

TABLE 10
Genes associated with all FDR-significant clones nominated
from microarray analysis (see also FIG. 17).

| | | | | |
|---|---|---|---|---|
| Rank | Associated Gene | Location | FDR-value | cFold-hange |

Schizophrenia Males

| | | | | |
|---|---|---|---|---|
| 1 | EXOSC7 | 3p21.31 | 0.0005 | 0.2468 |
| 2 | GRIA2 | 4q31.1 | 0.0006 | 0.2301 |
| 3 | ELMOD1 | 11q22.3 | 0.0006 | 0.1658 |
| 4 | KCNJ6 | 21q22.13 | 0.0007 | −0.2132 |
| 5 | WDR18 | 19p13.3 | 0.0011 | 0.1380 |
| 6 | PLA2G4B | 15q15.1 | 0.0014 | −0.2045 |
| 7 | PPP2CA | 5q31.1 | 0.0020 | 0.2089 |
| 8 | C13orf24 | 13q22.1 | 0.0032 | −0.1995 |
| 9 | FLJ90579 | 12q21.31 | 0.0057 | 0.2340 |
| 10 | LRRC61 | 7q36.1 | 0.0092 | 0.1496 |
| 11 | NXPH4 | 12q13.3 | 0.0129 | 0.1368 |
| 12 | MICAL-L2 | 7p22.3 | 0.0142 | −0.0956 |
| 13 | FLJ45721 | 4p15.2 | 0.0196 | −0.2059 |
| 14 | PRR5 | 22q13.31 | 0.0216 | 0.2842 |
| 15 | ADAMTS16 | 5p15.32 | 0.0232 | 0.1685 |
| 16 | AUTS2 | 7q11.22 | 0.0232 | −0.0948 |
| 17 | TPD52 | 8q21.13 | 0.0232 | −0.1617 |
| 18 | MYOZ1 | 10q22.2 | 0.0232 | 0.1990 |
| 19 | MRPS14 | 1q25.1 | 0.0250 | −0.1671 |
| 20 | RPP21 | 6p21.33 | 0.0250 | −0.1804 |
| 21 | HNRPR | 1p36.12 | 0.0250 | −0.1288 |
| 22 | THBS1 | 15q14 | 0.0261 | −0.1564 |
| 23 | FLJ23861 | 2q34 | 0.0261 | 0.1517 |
| 24 | C1orf110 | 1q23.3 | 0.0261 | −0.0929 |
| 25 | MLL5 | 7q22.2 | 0.0301 | −0.1629 |
| 26 | GLS2 | 12q13.2 | 0.0301 | −0.1618 |
| 27 | HOXD13 | 2q31.1 | 0.0328 | −0.1796 |
| 28 | BC032332 | 20q13.33 | 0.0339 | 0.1755 |
| 29 | SLC31A1 | 9q32 | 0.0341 | −0.1390 |
| 30 | C9orf40 | 9q21.13 | 0.0357 | −0.1421 |
| 31 | NAG | 2p24.3 | 0.0360 | −0.1696 |
| 32 | UNC5A | 5q35.2 | 0.0360 | −0.1356 |
| 33 | C6orf62 | 6p22.2 | 0.0361 | 0.1213 |
| 34 | CDC42BPA | 1q42.13 | 0.0369 | 0.1101 |
| 35 | CEBPZ | 2p22.2 | 0.0369 | 0.1755 |
| 36 | KEL | 7q34 | 0.0394 | −0.2105 |
| 37 | GLRX5 | 14q32.13 | 0.0395 | −0.1615 |
| 38 | AIG1 | 6q24.2 | 0.0416 | 0.1583 |
| 39 | SOX1 | 13q34 | 0.0416 | −0.1343 |
| 40 | MET | 7q31.2 | 0.0452 | 0.1906 |
| 41 | GLRX5 | 14q32.13 | 0.0460 | −0.1051 |
| 42 | PLAG1 | 8q12.1 | 0.0470 | −0.2080 |
| 43 | PANX1 | 11q21 | 0.0470 | 0.2048 |
| 44 | POLR3A | 10q22.3 | 0.0470 | −0.1598 |
| 45 | TRERF1 | 6p21.1 | 0.0471 | −0.1955 |
| 46 | PWP1 | 12q23.3 | 0.0484 | −0.1601 |
| 47 | COL9A1 | 6q13 | 0.0484 | 0.1563 |
| 48 | HLA-E | 6p21.33 | 0.0484 | −0.1608 |
| 49 | SMCHD1 | 18p11.32 | 0.0484 | 0.1918 |
| 50 | FBXO31 | 16q24.1 | 0.0484 | −0.2240 |
| 51 | LYST | 1q42.3 | 0.0484 | 0.1498 |

Schizophrenia Females

| | | | | |
|---|---|---|---|---|
| 1 | C6orf84 | 6q14.3 | 0.0209 | 0.4436 |
| 2 | HCG9 | 6p21.33 | 0.0209 | −0.5535 |
| 3 | SLC17A7 | 19q13.33 | 0.0209 | −0.5397 |
| 4 | NR4A2 | 2q24.1 | 0.0209 | −0.5515 |
| 5 | AB051500 | 18q12.1 | 0.0214 | −0.4895 |
| 6 | RPP21 | 6p21.33 | 0.0214 | −0.5238 |
| 7 | FN5 | 11q21 | 0.0214 | −0.5033 |
| 8 | NKX2-3 | 10q24.2 | 0.0222 | −0.5524 |
| 9 | ADAMTSL1 | 9p22.2 | 0.0275 | −0.4097 |
| 10 | MARLIN1 | 4p16.1 | 0.0321 | −0.5787 |
| 11 | TMEM59 | 1p32.3 | 0.0330 | 0.4103 |
| 12 | MTPN | 7q33 | 0.0330 | 0.5117 |
| 13 | LMX1B | 9q33.3 | 0.0330 | 0.5869 |
| 14 | SLC25A4 | 4q35.1 | 0.0352 | −0.4203 |
| 15 | LHX8 | 1P31.1 | 0.0352 | 0.3597 |
| 16 | LHX5 | 12q24.13 | 0.0352 | −0.3929 |
| 17 | CRTC2 | 1q21.3 | 0.0352 | −0.4635 |
| 18 | PGRMC1 | xq24 | 0.0352 | −0.3961 |
| 19 | BC037986 | 22q12.2 | 0.0352 | 0.4233 |
| 20 | Bmp7 | 20q13.31 | 0.0352 | −0.3563 |
| 21 | SIX2 | 2p21 | 0.0366 | −0.4339 |
| 22 | CCL1 | 17q12 | 0.0366 | −0.3109 |
| 23 | C16orf45 | 16p13.11 | 0.0366 | −0.3878 |
| 24 | LHX5 | 12q24.13 | 0.0403 | −0.4214 |
| 25 | PICALM | 11q14.2 | 0.0406 | −0.5156 |
| 26 | KIAA1787 | 17p13.1 | 0.0430 | 0.3267 |
| 27 | SLC17A6 | 11p14.3 | 0.0430 | 0.2542 |
| 28 | SF3B5 | 6q24.2 | 0.0435 | −0.5020 |
| 29 | SWAP70 | 11p15.4 | 0.0435 | 0.5038 |
| 30 | KEL | 7q34 | 0.0435 | −0.4062 |
| 31 | B3GALT3 | 3q26.1 | 0.0435 | 0.5117 |
| 32 | FLJ45455 | 17p13.1 | 0.0436 | 0.3658 |
| 33 | RAI1 | 17p11.2 | 0.0443 | −0.3083 |
| 34 | AK126832 | 2p21 | 0.0443 | 0.3683 |
| 35 | IGFL2 | 19q13.32 | 0.0443 | −0.3096 |
| 36 | CGI-115 | 1q41 | 0.0447 | −0.4902 |
| 37 | PRKCA | 17q24.2 | 0.0453 | 0.4270 |

Bipolar Males

| | | | | |
|---|---|---|---|---|
| 1 | THBS1 | 15q14 | 0.0403 | −0.2361 |
| 2 | MCM4 | 8q11.21 | 0.0403 | −0.2426 |

Bipolar Females

| | | | | |
|---|---|---|---|---|
| 1 | CGI-115 | 1q41 | 0.0200 | −0.4032 |
| 2 | RPL39 | xq24 | 0.0341 | −0.4401 |
| 3 | CGI-115 | 1q41 | 0.0341 | −0.2332 |
| 4 | AY831680 | 3q13.12 | 0.0341 | 0.4692 |
| 5 | NUDT9 | 4q22.1 | 0.0341 | −0.3460 |
| 6 | DTNBP1 | 6p22.3 | 0.0341 | −0.4966 |
| 7 | ADAMTSL1 | 9p22.2 | 0.0341 | −0.3663 |
| 8 | HELT | 4q35.1 | 0.0341 | −0.3538 |
| 9 | MARLIN1 | 4p16.1 | 0.0341 | −0.4673 |
| 10 | NKX2-3 | 10q24.2 | 0.0341 | −0.4604 |
| 11 | EFHD1 | 2q37.1 | 0.0341 | −0.3370 |
| 12 | DLL1 | 6q27 | 0.0341 | −0.2755 |
| 13 | ZCWPW2 | 3p24.1 | 0.0341 | −0.3065 |
| 14 | SORCS3 | 10q25.1 | 0.0354 | −0.3901 |
| 15 | VAX1 | 10q25.3 | 0.0424 | 0.2414 |
| 16 | AK129895 | 10p11.23 | 0.0424 | 0.4238 |
| 17 | DIPA | 11q13.1 | 0.0454 | −0.3539 |
| 18 | KIAA0859 | 1q24.3 | 0.0454 | −0.2527 |
| 19 | PPP2CA | 5q31.1 | 0.0454 | 0.2903 |
| 20 | EPHA5 | 4q13.1 | 0.0454 | 0.5550 |
| 21 | RPP21 | 6p21.33 | 0.0454 | −0.3775 |
| 22 | FNBP1L | 1p22.1 | 0.0454 | 0.3731 |
| 23 | C21orf29 | 21q22.3 | 0.0454 | −0.3262 |
| 24 | TMEM59 | 1p32.3 | 0.0454 | 0.3886 |
| 25 | SIL1 | 5q31.2 | 0.0454 | −0.4085 |
| 26 | RPAP1 | 15q15.1 | 0.0454 | −0.2490 |
| 27 | C16orf45 | 16p13.11 | 0.0454 | −0.3231 |
| 28 | NRG2 | 5q31.3 | 0.0454 | 0.2295 |
| 29 | KEL | 7q34 | 0.0454 | −0.3094 |
| 30 | LOC285513 | 4q22.1 | 0.0454 | 0.3411 |
| 31 | FLJ23861 | 2q34 | 0.0454 | 0.2402 |
| 32 | FLJ43505 | 1q41 | 0.0454 | −0.2876 |
| 33 | FOXP1 | 3p14.1 | 0.0454 | 0.3213 |
| 34 | PRKCA | 17q24.2 | 0.0454 | 0.3429 |
| 35 | LHX5 | 12q24.13 | 0.0460 | −0.3573 |

Psychosis Males

| | | | | |
|---|---|---|---|---|
| 1 | KCNJ6 | 21q22.13 | 0.0008 | −0.2027 |
| 2 | ELMOD1 | 11q22.3 | 0.0022 | 0.1388 |
| 3 | EXOSC7 | 3p21.31 | 0.0022 | 0.2067 |
| 4 | GRIA2 | 4q32.1 | 0.0034 | 0.1948 |
| 5 | C13orf24 | 13q22.1 | 0.0035 | −0.1788 |
| 6 | THBS1 | 15q14 | 0.0047 | −0.1782 |
| 7 | WDR18 | 19p13.3 | 0.0047 | 0.1117 |
| 8 | PPP2CA | 5q31.1 | 0.0054 | 0.1728 |
| 9 | STIM2 | 4p15.2 | 0.0087 | −0.2180 |
| 10 | C9orf40 | 9q21.13 | 0.0145 | −0.1537 |
| 11 | ADAMTS16 | 5p15.32 | 0.0173 | 0.1605 |
| 12 | LYST | 1q42.3 | 0.0173 | 0.1500 |
| 13 | MRPS14 | 1q25.1 | 0.0173 | −0.1516 |
| 14 | FBXO31 | 16q24.1 | 0.0173 | −0.2262 |
| 15 | CDC42BPA | 1q42.13 | 0.0183 | 0.1149 |
| 16 | C6orf62 | 6p22.2 | 0.0183 | 0.1100 |
| 17 | ZFAND2A | 7p22.3 | 0.0206 | −0.0848 |
| 18 | BC032332 | 20q13.33 | 0.0206 | 0.1601 |
| 19 | PRR5 | 22q13.31 | 0.0221 | 0.2722 |
| 20 | FLJ90579 | 12q21.31 | 0.0223 | 0.1968 |
| 21 | FLJ23861 | 2q34 | 0.0223 | 0.1361 |
| 22 | PLA2G4B | 15q15.1 | 0.0226 | −0.1705 |
| 23 | MYOZ1 | 10q22.2 | 0.0233 | 0.1751 |
| 24 | IKIP | 12q23.1 | 0.0237 | −0.1802 |
| 25 | TPD52 | 8q21.13 | 0.0240 | −0.1442 |
| 26 | GGN | 19q13.2 | 0.0269 | 0.2440 |
| 27 | LRRC61 | 7q36.1 | 0.0270 | 0.1186 |
| 28 | CEBPZ | 2p22.2 | 0.0292 | 0.1741 |
| 29 | MLL5 | 7q22.2 | 0.0309 | −0.1581 |
| 30 | HSD17B7 | 1q23.3 | 0.0350 | −0.0794 |
| 31 | VPS33B | 15q26.1 | 0.0350 | −0.1462 |
| 32 | FLJ41423 | 11p11.2 | 0.0382 | −0.1648 |
| 33 | ZNF195 | 11p15.4 | 0.0382 | 0.1879 |
| 34 | NAG | 2p24.3 | 0.0478 | −0.1622 |
| 35 | C16orf45 | 16p13.11 | 0.0482 | −0.1702 |
| 36 | POLR3A | 10q22.3 | 0.0486 | −0.1455 |
| 37 | CRSP6 | 11q21 | 0.0496 | 0.1825 |

Psychosis Females

| | | | | |
|---|---|---|---|---|
| 1 | CGI-115 | 1q41 | 0.0079 | −0.4380 |
| 2 | DHRS8 | 4q22.1 | 0.0079 | −0.3533 |
| 3 | ADAMTSL1 | 9p22.2 | 0.0079 | −0.3802 |
| 4 | DTNBP1 | 6p22.3 | 0.0079 | −0.4971 |
| 5 | CGI-115 | 1q24.2 | 0.0107 | −0.2389 |
| 6 | MARLIN1 | 4p16.1 | 0.0107 | −0.5081 |
| 7 | NKX2-3 | 10q41 | 0.0107 | −0.4801 |
| 8 | DLL1 | 6q27 | 0.0107 | −0.2728 |
| 9 | RPP21 | 6p21.33 | 0.0107 | −0.4191 |
| 10 | HELT | 4q35.1 | 0.0107 | −0.3663 |
| 11 | TMEM59 | 1p32.3 | 0.0115 | 0.3861 |
| 12 | PRKCA | 17q24.2 | 0.0131 | 0.3767 |
| 13 | RPAP1 | 15q15.1 | 0.0140 | −0.2389 |
| 14 | ISL2 | 15q24.3 | 0.0140 | −0.2466 |
| 15 | SORCS3 | 10q25.1 | 0.0140 | −0.3916 |
| 16 | RPL39 | xq24 | 0.0149 | −0.4359 |
| 17 | AK129895 | 10q37.1 | 0.0175 | 0.4103 |
| 18 | EFHD1 | 2p11.23 | 0.0175 | −0.3065 |
| 19 | LHX5 | 12q24.13 | 0.0175 | −0.3701 |
| 20 | SLC17A7 | 19q13.33 | 0.0175 | −0.4788 |
| 21 | FOXP1 | 3p14.1 | 0.0176 | 0.3151 |
| 22 | C16orf45 | 16p13.11 | 0.0186 | −0.3356 |
| 23 | EPHA5 | 4q13.1 | 0.0186 | 0.5457 |
| 24 | VAX1 | 10q25.3 | 0.0186 | 0.2354 |
| 25 | SMUG1 | 12q13.13 | 0.0190 | −0.4064 |
| 26 | ZNF582 | 19q13.43 | 0.0195 | −0.3132 |
| 27 | MTPN | 7q33 | 0.0195 | 0.5284 |
| 28 | KEL | 7q34 | 0.0195 | −0.3375 |
| 29 | FLJ43505 | 1q41 | 0.0195 | −0.2912 |
| 30 | RPP21 | 6p21.33 | 0.0195 | −0.3981 |
| 31 | GLRX5 | 14q32.13 | 0.0233 | −0.3571 |
| 32 | AK127494 | 19p13.2 | 0.0241 | −0.3386 |
| 33 | FNBP1L | 1p22.1 | 0.0241 | 0.3647 |
| 34 | FLJ45455 | 17p13.1 | 0.0241 | 0.3359 |
| 35 | CBX8 | 17q25.3 | 0.0241 | −0.4036 |
| 36 | ATF7IP | 12p13.1 | 0.0241 | −0.2177 |
| 37 | PSIP1 | 9p22.3 | 0.0241 | −0.2317 |
| 38 | SIL1 | 5q31.2 | 0.0249 | −0.3742 |
| 39 | SWAP70 | 11p15.4 | 0.0249 | 0.4102 |
| 40 | ATOH8 | 2p11.2 | 0.0249 | −0.3876 |
| 41 | UXS1 | 2q12.2 | 0.0249 | −0.4706 |
| 42 | PPP2CA | 5q31.1 | 0.0266 | 0.2886 |
| 43 | HCG9 | 6p21.33 | 0.0290 | −0.4265 |
| 44 | C9orf40 | 9q21.13 | 0.0327 | −0.3146 |
| 45 | COQ5 | 12q24.31 | 0.0327 | −0.2538 |
| 46 | FN5 | 11q21 | 0.0339 | −0.3808 |
| 47 | KIAA0859 | 1q24.3 | 0.0343 | −0.2402 |
| 48 | TCF7L2 | 10q25.2 | 0.0375 | 0.2205 |
| 49 | GATAD2A | 19p13.11 | 0.0378 | −0.6567 |
| 50 | FLJ20643 | 19q13.33 | 0.0382 | −0.2953 |
| 51 | CLK2 | 1q22 | 0.0382 | 0.5518 |
| 52 | LMX1B | 9q33.3 | 0.0415 | 0.5844 |
| 53 | PLA2G4B | 15q15.1 | 0.0415 | −0.3618 |
| 54 | PSMB7 | 9q33.3 | 0.0415 | 0.5638 |
| 55 | NRG2 | 5q31.3 | 0.0415 | 0.2370 |
| 56 | LHX8 | 1p31.1 | 0.0418 | 0.3255 |
| 57 | B3GALT3 | 3q26.1 | 0.0426 | 0.4555 |
| 58 | WNT1 | 12q13.12 | 0.0426 | −0.2894 |
| 59 | EBPL | 13q14.3 | 0.0426 | 0.2948 |
| 60 | ZCWPW2 | 3p24.1 | 0.0426 | −0.2747 |
| 61 | HLX1 | 1q41 | 0.0426 | −0.2878 |
| 62 | FOSB | 19q13.32 | 0.0426 | 0.3011 |
| 63 | CYB5R4 | 6q14.2 | 0.0428 | 0.3911 |
| 64 | BC037986 | 22q12.2 | 0.0428 | 0.3570 |
| 65 | C14orf138 | 14q22.1 | 0.0428 | −0.1832 |
| 66 | TYMS | 18p11.32 | 0.0428 | −0.1812 |
| 67 | AY831680 | 3q13.12 | 0.0428 | 0.4401 |
| 68 | CNTN5 | 11q22.1 | 0.0438 | 0.3437 |
| 69 | TRPS1 | 8q23.3 | 0.0438 | −0.2733 |
| 70 | PGRMC1 | xq24 | 0.0438 | −0.3393 |
| 71 | OCIAD1 | 4p12 | 0.0465 | −0.3200 |
| 72 | RAB38 | 11q14.2 | 0.0475 | 0.2384 |
| 73 | SCG2 | 2q36.1 | 0.0497 | 0.3157 |
| 74 | KIAA1787 | 17p13.1 | 0.0497 | 0.3435 |

TABLE 11
Gene ontology analysis of microarray data.

| | | | | |
|---|---|---|---|---|
| | | Mean | | |
| GO Category | N | Diff | P-value | Description |

Male Bipolar Disorder

| | | | | |
|---|---|---|---|---|
| GO: 0008168 | 4 | 0.098 | 0.00006* | methyltransferase activity |
| GO: 0005737 | 50 | −0.080 | 0.00007* | cytoplasm |
| GO: 0005515 | 139 | −0.047 | 0.00031* | protein binding |
| GO: 0005634 | 158 | −0.037 | 0.00074* | nucleus\hat{ } |
| GO: 0006950 | 5 | −0.171 | 0.00168* | response to stress |
| GO: 0016481 | 5 | −0.156 | 0.00309 | negative regulation of transcription |
| GO: 0008134 | 5 | −0.153 | 0.00401 | transcription factor binding |
| GO: 0007275 | 37 | −0.073 | 0.00418 | multicellular organismal development |
| GO: 0008104 | 3 | 0.136 | 0.00480 | protein localization\hat{ } |
| GO: 0005216 | 3 | 0.092 | 0.00564 | ion channel activity |
| GO: 0005488 | 10 | −0.116 | 0.00766 | binding\hat{ } |
| GO: 0005643 | 6 | −0.159 | 0.00845 | nuclear pore |

Female Bipolar Disorder

| | | | | |
|---|---|---|---|---|
| GO: 0042773 | 9 | 0.940 | 0.00000* | ATP synthesis coupled electron transport\hat{ } |
| GO: 0006879 | 11 | 0.817 | 0.00000* | iron ion homeostasis\hat{ } |
| GO: 0030595 | 11 | 0.817 | 0.00000* | leukocyte chemotaxis\hat{ } |
| GO: 0048019 | 11 | 0.817 | 0.00000* | receptor antagonist activity\hat{ } |
| GO: 0048471 | 11 | 0.817 | 0.00000* | perinuclear region of cytoplasm\hat{ } |
| GO: 0005747 | 13 | 0.730 | 0.00002* | mitochondrial respiratory chain complex |
| | | | | I\hat{ } |
| GO: 0006120 | 13 | 0.730 | 0.00002* | mitochondrial electron transport, NADH |
| | | | | to ubiquinone\hat{ } |
| GO: 0008137 | 13 | 0.730 | 0.00002* | NADH dehydrogenase (ubiquinone) |
| | | | | activity\hat{ } |
| GO: 0016491 | 23 | 0.487 | 0.00003* | oxidoreductase activity\hat{ } |
| GO: 0006916 | 17 | 0.576 | 0.00006* | anti-apoptosis\hat{ } |
| GO: 0016021 | 72 | 0.194 | 0.00013* | integral to membrane\hat{ } |
| GO: 0007420 | 6 | 0.297 | 0.00017* | brain development\hat{ } |
| GO: 0005488 | 5 | −0.142 | 0.00039* | binding\hat{ } |
| GO: 0005576 | 20 | 0.459 | 0.00051* | extracellular region\hat{ } |
| GO: 0003674 | 15 | −0.201 | 0.00055* | molecular_function\hat{ } |
| GO: 0005739 | 33 | 0.302 | 0.00069* | mitochondrion\hat{ } |
| GO: 0004759 | 4 | −0.191 | 0.00107* | serine esterase activity\hat{ } |
| GO: 0005496 | 3 | −0.306 | 0.00112* | steroid binding\hat{ } |
| GO: 0008289 | 3 | −0.306 | 0.00112* | lipid binding\hat{ } |
| GO: 0005634 | 153 | 0.069 | 0.00133* | nucleus\hat{ } |
| GO: 0008150 | 13 | −0.200 | 0.00163* | biological_process\hat{ } |
| GO: 0016020 | 108 | 0.113 | 0.00199* | membrane\hat{ } |
| GO: 0006935 | 4 | 0.493 | 0.00278* | chemotaxis |
| GO: 0005886 | 16 | 0.166 | 0.00360* | plasma membrane |
| GO: 0009887 | 9 | 0.264 | 0.00446* | organ morphogenesis |
| GO: 0007389 | 3 | −0.173 | 0.00749 | pattern specification process |
| GO: 0016023 | 3 | −0.199 | 0.00768 | cytoplasmic membrane-bound vesicle |
| GO: 0016788 | 3 | −0.199 | 0.00768 | hydrolase activity, acting on ester bonds |
| GO: 0018738 | 3 | −0.199 | 0.00768 | S-formylglutathione hydrolase activity |
| GO: 0006350 | 44 | 0.103 | 0.00923 | transcription |
| GO: 0006355 | 72 | 0.084 | 0.00966 | regulation of transcription, DNA- |
| | | | | dependent |

Male Schizophrenia

| | | | | |
|---|---|---|---|---|
| GO: 0007165 | 33 | 0.059 | 0.00865 | signal transduction |

Female Schizophrenia

| | | | | |
|---|---|---|---|---|
| GO: 0005747 | 20 | 0.712 | 0.00000* | mitochondrial respiratory chain complex |
| | | | | I\hat{ } |
| GO: 0006120 | 20 | 0.712 | 0.00000* | mitochondrial electron transport, NADH |
| | | | | to ubiquinone\hat{ } |
| GO: 0008137 | 20 | 0.712 | 0.00000* | NADH dehydrogenase (ubiquinone) |
| | | | | activity\hat{ } |
| GO: 0006879 | 18 | 0.737 | 0.00000* | iron ion homeostasis\hat{ } |
| GO: 0030595 | 18 | 0.737 | 0.00000* | leukocyte chemotaxis\hat{ } |
| GO: 0048019 | 18 | 0.737 | 0.00000* | receptor antagonist activity\hat{ } |
| GO: 0048471 | 18 | 0.737 | 0.00000* | perinuclear region of cytoplasm\hat{ } |
| GO: 0042773 | 13 | 0.754 | 0.00000* | ATP synthesis coupled electron transport\hat{ } |
| GO: 0006916 | 25 | 0.577 | 0.00000* | anti-apoptosis\hat{ } |
| GO: 0016491 | 30 | 0.490 | 0.00000* | oxidoreductase activity\hat{ } |
| GO: 0005576 | 29 | 0.490 | 0.00000* | extracellular region\hat{ } |
| GO: 0005739 | 40 | 0.351 | 0.00001* | mitochondrion\hat{ } |
| GO: 0016021 | 89 | 0.179 | 0.00002* | integral to membrane\hat{ } |
| GO: 0016020 | 130 | 0.137 | 0.00003* | membrane\hat{ } |
| GO: 0005215 | 7 | 0.266 | 0.00012* | transporter activity |
| GO: 0045944 | 7 | −0.273 | 0.00013* | positive regulation of transcription from |
| | | | | RNA polymerase II promoter |
| GO: 0003674 | 15 | −0.241 | 0.00066* | molecular_function\hat{ } |
| GO: 0007420 | 5 | 0.262 | 0.00110* | brain development\hat{ } |
| GO: 0031966 | 5 | 0.690 | 0.00137* | mitochondrial membrane |
| GO: 0007067 | 4 | −0.247 | 0.00151* | mitosis |
| GO: 0051301 | 4 | −0.247 | 0.00151* | cell division |
| GO: 0008104 | 3 | −0.178 | 0.00220* | protein localization\hat{ } |
| GO: 0005496 | 3 | −0.371 | 0.00241* | steroid binding\hat{ } |
| GO: 0008289 | 3 | −0.371 | 0.00241* | lipid binding\hat{ } |
| GO: 0016251 | 5 | 0.165 | 0.00244* | general RNA polymerase II transcription |
| | | | | factor activity |
| GO: 0008150 | 13 | −0.233 | 0.00270* | biological_process\hat{ } |
| GO: 0016757 | 4 | 0.292 | 0.00288* | transferase activity, transferring glycosyl |
| | | | | groups |
| GO: 0004759 | 3 | −0.172 | 0.00524* | serine esterase activity\hat{ } |

| | Link to | | | | | | |
|---|---|---|---|---|---|---|---|
| Gene | major | | Amplicon | Assay | Mean amplicon methylation (SD) | | |
| (Loc) | psychosis | Amplicon | Location* | CGs | SZ | BD | CTRL |
| ARVCF | Located next | Intron 3 | 22: 18354314-18354584 | 10 | 4.53 | 5.02 (0.95) | 4.69 (0.53) |
| 22q11 | to COMT in | | | | (0.78) | | |
| | a region | | | | | | |
| | deleted in | | | | | | |
| | velocardiofacial | | | | | | |
| | syndrome | | | | | | |
| BDNF | A | Exon 2\hat{ } | 11: 27636431-27636634 | 4 | 82.48 | 81.14 (5.24) | 82.11 (2.72) |
| 11p13 | prosurvival | | | | (2.03) | | |
| | factor | Intron 1(a) | 11: 27697459-27697652 | 10 | 4.19 | 2.93 (1.57) | 4.43 (3.35) |
| | necessary | | | | (0.72) | | |
| | for survival | Intron 1(b) | 11: 27678599-27678889 | 12 | 6.40 | 5.97 (0.48) | 6.40 (0.96) |
| | of striatal | | | | (1.39) | | |
| | neurons in | Promoter | 11: 27700523-27700854 | 15 | 2.64 | 2.95 (0.58) | 2.52 (0.36) |
| | the brain | | | | (0.72) | | |
| COMT | Catalyzes | Exon 4 | 22: 18331021-18331322 | 9 | 74.4 | 74.05 (3.21) | 75.17 (2.56) |
| 22q11 | the transfer | | | | (1.65) | | |
| | of a methyl | Promoter | 22: 18309077-18309496 | 12 | 2.35 | 2.79 (2.71) | 3.12 (3.44) |
| | group to | | | | (1.53) | | |
| | catecholamine | | | | | | |
| | neurotransmitters | | | | | | |
| | including | | | | | | |
| | dopamine, | | | | | | |
| | epinephrine, | | | | | | |
| | and | | | | | | |
| | norepinephrine. | | | | | | |
| DRD4 | Shows high | Promoter\hat{ }\hat{ } | 11: 626685-626839 | 12 | 12.23 | 12.46 (2.96) | 13.67 (2.51) |
| 11p15 | affinity for | | | | (4.61) | | |
| | the | | | | | | |
| | antipsychotic | | | | | | |
| | clozapine. | | | | | | |
| DTNBP1 | A | Intron 1^{#} | 6: 15770400-15770612 | 12 | 5.02 | 3.87 (1.76) | 4.50 (2.15) |
| 6p22 | ubiquitously | | | | (3.46) | | |
| | expressed | Promoter^{#} | 6: 15771064-15771358 | 15 | 5.92 | 5.24 (1.84) | 5.11 (1.80) |
| | protein that | | | | (1.98) | | |
| | binds to | | | | | | |
| | alpha- and | | | | | | |
| | beta- | | | | | | |
| | dystrobrevins, | | | | | | |
| | and has | | | | | | |
| | been widely | | | | | | |
| | associated | | | | | | |
| | with | | | | | | |
| | schizophrenia. | | | | | | |
| GAD1 | Catalyzes | Intron 1 | 2: 171381495-171381705 | 10 | 3.13 | 3.49 (1.24) | 3.57 (0.78) |
| 2q31 | the | | | | (0.54) | | |
| | conversion | Intron 3 | 2: 171387778-171388116 | 13 | 11.05 | 11.21 (1.69) | 11.56 (3.18) |
| | of glutamic | | | | (1.82) | | |
| | acid to | Promoter | 2: 171380534-171380762 | 12 | 6.00 | 7.22 (1.98) | 5.97 (0.88) |
| | gamma- | | | | (0.47) | | |
| | aminobutyric | | | | | | |
| | acid | | | | | | |
| | (GABA), the | | | | | | |
| | major | | | | | | |
| | inhibitory | | | | | | |
| | neurotransmitter | | | | | | |
| | in the | | | | | | |
| | vertebral | | | | | | |
| | central | | | | | | |
| | nervous | | | | | | |
| | system. | | | | | | |
| GRIN2B | A critical | Promoter | 12: 14025024-14025307 | 10 | 5.60 | 7.19 (2.00) | 5.97 (1.49) |
| 12p12 | structural | | | | (1.89) | | |
| | and | | | | | | |
| | functional | | | | | | |
| | subunit of | | | | | | |
| | the NMDA | | | | | | |
| | glutamate | | | | | | |
| | receptor that | | | | | | |
| | has been | | | | | | |
| | widely | | | | | | |
| | implicated in | | | | | | |
| | psychosis | | | | | | |
| MTHFR | Polymorphisms | Promoter | 1: 11788017-11788228 | 12 | 1.19 | 1.12 (0.31) | 1.03 (0.21) |
| 1p36 | in the | (a) | | | (0.78) | | |
| | 5,10- | Promoter | 1: 11788356-11788642 | 18 | 1.27 | 1.29 (0.23) | 1.48 (0.18) |
| | @Methylene | (b) | | | (0.15) | | |
| | tetrahydrofolate | | | | | | |
| | Reductase | | | | | | |
| | gene have | | | | | | |
| | been | | | | | | |
| | associated | | | | | | |
| | with both | | | | | | |
| | bipolar | | | | | | |
| | disorder and | | | | | | |
| | schizophrenia. | | | | | | |
| NRG1 | One of four | Intron 1 | 8: 32525871-32526230 | 8 | 5.16 | 4.63 (0.70) | 3.99 (1.56) |
| 8p22-11 | neuregulin | | | | (1.26) | | |
| | growth | Promoter | 8: 32524919-32525183 | 14 | 2.54 | 3.98 (2.52) | 2.41 (0.47) |
| | factor genes | | | | (0.39) | | |
| | that signal | | | | | | |
| | through the | | | | | | |
| | erbB | | | | | | |
| | receptor | | | | | | |
| | kinase | | | | | | |
| | pathways, | | | | | | |
| | and is | | | | | | |
| | known to be | | | | | | |
| | involved in | | | | | | |
| | neuronal | | | | | | |
| | migration | | | | | | |
| | and cellular | | | | | | |
| | differentiation | | | | | | |
| | in the | | | | | | |
| | developing | | | | | | |
| | brain. | | | | | | |
| RELN | Thought to | Promoter | 7: 103417221-103417716 | 11 | 3.51 | 3.98 (0.67) | 3.15 (0.34) |
| 7q22 | control cell- | (a) | | | (0.98) | | |
| | cell | Promoter | 7: 103417669-103417968 | 11 | 1.89 | 2.74 (1.28) | 2.31 (0.56) |
| | interactions | (b) | | | (0.47) | | |
| | critical for | Promoter | 7: 103416649-103416879 | 12 | 3.64 | 4.60 (1.70) | 3.69 (1.85) |
| | cell | (c) | | | (1.59) | | |
| | positioning | | | | | | |
| | in the brain. | | | | | | |
| | Hypermethylation | | | | | | |
| | of the | | | | | | |
| | promoter | | | | | | |
| | region of | | | | | | |
| | RELN has | | | | | | |
| | been found | | | | | | |
| | in some | | | | | | |
| | schizophrenic | | | | | | |
| | brain | | | | | | |
| | samples | | | | | | |

Shown are all GO categories with a p-value <0.01 (*= categories with FDR value <0.05; \hat{ }= categories present in more than one diagnostic group).
A positive mean difference suggests hypomethylation in the affected group relative to unaffected controls.

TABLE 12
Average % CpG methylation assessed using Pyrosequencing across genes.

| | |
|---|---|
| * | May 2006 UCSC Genome Assembly |
| \hat{ } | DNA methylation found to correlate with genotype |
| \hat{ }\hat{ } | 2 individual CpG locations within DRD4_Prm amplicons were |
| | significantly associated with diagnosis but did not survive correction |
| | for multiple testing |
| # | A region further upstream of DTNBP1 was found to be significantly |
| | hypermethylated in affected females relative to controls by |
| | microarray analysis |

What is claimed is:

1. A method of identifying one or more epigenetic markers associated with a psychosis-associated disease, the method comprising,
a) obtaining a first group of samples comprising genomic DNA from a plurality of subjects exhibiting a psychosis-associated disease and a second group of samples comprising genomic DNA from a plurality of control subjects;
b) performing DNA methylation analysis to determine methylation differences in one or more DNA regions between the first group and second group of samples, wherein a methylation difference in a DNA region is indicative of an epigenetic marker associated with the psychosis-associated disease.

2. The method of claim 1, wherein the psychosis-associated disease is bipolar disorder or schizophrenia.

3. The method of claim 2 wherein the disease is bipolar disorder.

4. The method of claim 2, wherein the disease is schizophrenia.

5. The method of claim 1, wherein the DNA methylation analysis is DNA microarray analysis.

6. The method of claim 1, wherein the samples are blood, brain, sperm or any other tissue or a sample that provides genomic DNA.

7. The method of claim 5, wherein DNA microarray analysis comprises hybridization of differentially epigenetically modified DNA from each subject of said first and second groups to a genomic microarray.

8. The method of claim 1, wherein the differences comprise hypermethylation differences, hypomethylation differences or both.

9. The method of claim 1, wherein said step of performing identifies a set of epigenetic markers, the set providing an increased correlation of association with bipolar disorder or schizophrenia as compared to a single epigenetic marker.

10. The method of claim 1, wherein said method further comprises identifying one or more genes associated with the epigenetic markers.

11. A method of determining the risk of a subject having or developing a psychosis-associated disease comprising,
a) obtaining a genomic DNA sample from the subject,
b) determining the methylation status of one or more epigenetic markers in the genomic DNA sample from the subject, and;
c) comparing the methylation status of said one or more epigenetic markers to the methylation status of a control group of epigenetic markers associated with a psychosis-associated disease, wherein similar or identical methylation status profiles are indicative of an increased risk of having or developing the psychosis-associated disease.

12. The method of claim 11 wherein the psychosis-associated disease is bipolar disorder or schizophrenia.

13. One or more epigenetic markers associated with a psychosis-associated disease, the markers identified by the method of claim 1.

14. The markers as defined in claim 13, wherein the disease is bipolar disorder or schizophrenia.

15. A nucleotide sequence array comprising one or more epigenetic markers associated with a psychosis-associated disease or disorder.

16. The array of claim 15, wherein the disease or disorder is bipolar disorder or schizophrenia.

17. One or more epigenetic markers associated with bipolar disorder or schizophrenia, wherein each of said one or more markers comprises a methylated cytosine.

18. A set of epigenetic markers associated with bipolar disorder or schizophrenia , the markers comprising a plurality of nucleotide sequences that are differentially epigenetically modified and that are positively associated with bipolar disorder or schizophrenia.
